SEQUENCE LISTING (1 sequence)

SEQ ID NO: 1 - protein, 1210 aa, Artificial Sequence
  homo sapiens - phosphorylase kinase catalytic subunit alpha, skeletal (PHKA1) - NP_001116142.1
MRSRSNSGVRLDGYARLVQQTILCHQNPVTGLLPASYDQKDAWVRDNVYSILAVWGLGLAYRKNADRDEDKAKAYELEQSVVKLMRGLLHCMIRQVDKVESFKYSQSTKDSLHAKYNTKTCATVVGDDQWGHLQLDATSVYLLFLAQMTASGLHIIHSLDEVNFIQNLVFYIEAAYKTADFGIWERGDKTNQGISELNASSVGMAKAALEALDELDLFGVKGGPQSVIHVLADEVQHCQSILNSLLPRASTSKEVDASLLSVVSFPAFAVEDSQLVELTKQEIITKLQGRYGCCRFLRDGYKTPKEDPNRLYYEPAELKLFENIECEWPLFWTYFILDGVFSGNAEQVQEYKEALEAVLIKGKNGVPLLPELYSVPPDRVDEEYQNPHTVDRVPMGKLPHMWGQSLYILGSLMAEGFLAPGEIDPLNRRFSTVPKPDVVVQVSILAETEEIKTILKDKGIYVETIAEVYPIRVQPARILSHIYSSLGCNNRMKLSGRPYRHMGVLGTSKLYDIRKTIFTFTPQFIDQQQFYLALDNKMIVEMLRTDLSYLCSRWRMTGQPTITFPISHSMLDEDGTSLNSSILAALRKMQDGYFGGARVQTGKLSEFLTTSCCTHLSFMDPGPEGKLYSEDYDDNYDYLESGNWMNDYDSTSHARCGDEVARYLDHLLAHTAPHPKLAPTSQKGGLDRFQAAVQTTCDLMSLVTKAKELHVQNVHMYLPTKLFQASRPSFNLLDSPHPRQENQVPSVRVEIHLPRDQSGEVDFKALVLQLKETSSLQEQADILYMLYTMKGPDWNTELYNERSATVRELLTELYGKVGEIRHWGLIRYISGILRKKVEALDEACTDLLSHQKHLTVGLPPEPREKTISAPLPYEALTQLIDEASEGDMSISILTQEIMVYLAMYMRTQPGLFAEMFRLRIGLIIQVMATELAHSLRCSAEEATEGLMNLSPSAMKNLLHHILSGKEFGVERSVRPTDSNVSPAISIHEIGAVGATKTERTGIMQLKSEIKQSPGTSMTPSSGSFPSAYDQQSSKDSRQGQWQRRRRLDGALNRVPVGFYQKVWKVLQKCHGLSVEGFVLPSSTTREMTPGEIKFSVHVESVLNRVPQPEYRQLLVEAILVLTMLADIEIHSIGSIIAVEKIVHIANDLFLQEQKTLGADDTMLAKDPASGICTLLYDSAPSGRFGTMTYLSKAAATYVQEFLPHSICAMQ

(19) United States
(12) Patent Application Publication
(10) Pub. No.: US 2025/0319204 A1
(43) Pub. Date: Oct. 16, 2025

(54) COMPOSITIONS FOR AND METHODS OF TREATING AND/OR PREVENTING GLYCOGEN STORAGE DISEASE TYPE VI AND TYPE IX

(71) Applicant: Duke University, Durham, NC (US)

(72) Inventors: Priya S. Kishnani, Durham, NC (US); Baodong Sun, Durham, NC (US); Aravind Asokan, Durham, NC (US); Rebecca Gibson, Durham, NC (US); Jeong-A Lim, Durham, NC (US)

(73) Assignee: Duke University, Durham, NC (US)

(21) Appl. No.: 18/269,686

(22) PCT Filed: Dec. 30, 2021
(86) PCT No.: PCT/US21/65640

§ 371 Date: Jun. 26, 2023

Related U.S. Application Data
(60) Provisional application No. 63/132,395, filed on Dec. 30, 2020.
(60) Provisional application No. 63/243,134, filed on Sep. 11, 2021.

(51) Int. Cl. A61K 48/00; A61K 31/085; A61K 31/436; A61K 31/519; A61K 31/69; A61K 38/45; A61K 39/395; A61P 1/16

(52) U.S. Cl. CPC A61K 48/0058; A61K 31/085; A61K 31/436; A61K 31/519; A61K 31/69; A61K 38/45; A61K 39/39516; A61K 39/3955; A61K 48/0033; A61K 48/0075; A61P 1/16

(57) ABSTRACT

Glycogen storage disease (GSD) types VI and IX are caused by phosphorylase system deficiencies and these GSDs are often clinically indistinguishable from one another. Disclosed herein are compositions for and methods of treating and/or preventing GSD VI and GSD IX disease progression with gene therapy alone or in combination with other therapies.

### I. CROSS-REFERENCE TO RELATED APPLICATIONS

This application claim priority to U.S. Provisional Application No. 63/132,395 filed 30 Dec. 2020 and U.S. Provisional Application No. 63/243,134 filed 11 Sep. 2021, both of which are incorporated herein in their entirety.

### II. REFERENCE TO THE SEQUENCE LISTING

The Sequence Listing submitted 30 Dec. 2021 as a text file named “20_2006_WO_Sequence_Listing”, created on 30 Dec. 2021 and having a size of 431 kilobytes is hereby incorporated by reference pursuant to 37 C.F.R. § 1.52(e)(5).

### III. BACKGROUND

Glycogen, a highly branched polymer of glucose molecules, is the body's main storage form of glucose. During the fasted state, glycogen is broken down into its glucose monomers to maintain glucose levels in the blood. Inherited abnormalities in the genes encoding enzymes that enable glycogen synthesis and breakdown are collectively referred to as Glycogen Storage Diseases (GSDs). GSDs are a group of genetic disorders associated with abnormal accumulation of glycogen. The group of disorders are generally numbered 0-15 in association with the respective enzymes for glycogen synthesis or breakdown and are identified by affected tissue type (generally muscle and/or liver). (Adeva-Andany M M, et al. (2016) BBA Clin. 5:85-100; Kanungo S, et al. (2018) Ann. Transl. Med. 6:1-18). Examples of GSDs include, but are not limited to, GSD Type I-VII, IX, XI, XII, XIII, and XV.

Glycogen Storage Disease VI (GSD VI) is the result of a deficiency of liver glycogen phosphorylase, which is encoded by the PYGL gene. Glycogen Storage Disease IX, liver form (GSD IX), results from deficiency of liver phosphorylase kinase (PhK). GSD VI and GSD IX are often clinically indistinguishable, with combined diagnosis and management guidelines for both indications. The estimated prevalence of GSD IX is 1 in 100,000 individuals and the estimated prevalence of GSD VI is 1 in 65,000 to 1 in 85,000 individuals (Wilson L H, et al. (2019) Hepatol. Commun. 3:1544-1555).

Both liver GSD VI and GSD IX result in impaired glycogenolysis. Patients experience hepatomegaly due to increased glycogen storage, hypoglycemia, ketosis, growth retardation, and elevated liver enzyme levels (ALT and AST) in the blood. Moreover, as the disease advances, liver fibrosis and then liver cirrhosis plague the patient.

Currently, there are no disease-modifying therapies for GSDs like GSD VI and GSD IX. Consequently, there remains an urgent need for a minimally invasive, definitive therapy to address the underlying cause of as well as the sequelae of symptoms associated with GSDs including GSD VI and GSD IX. The present disclosure provides compositions for and methods of treating and preventing GSD VI and/or GSD IX disease progression, which can be used alone or in combination with other treatments.

### IV. BRIEF DESCRIPTION OF THE FIGURES

FIG. 1 provides an illustrative example of the metabolic pathways of glycogen metabolic and glycogenolysis including the sites of enzymatic defects that result in clinical GSDs.

FIG. 2A-FIG. 2D show the verification of the generation of the Phkg2^{-/-} mouse model with FIG. 2A providing an illustration of the Phkg2^{tm1.1} knockout allele, FIG. 2B showing the genotyping by PCR, FIG. 2C showing the expression of liver PhK subunits, and FIG. 2D showing the PhK enzyme activity.

FIG. 3A-FIG. 3B show the morphological assessment of Phkg2^{-/-} mice with FIG. 3A showing the body weight growth curve for KO and WT mice and FIG. 3B showing percent liver weight normalized to body weight as a quantitative marker of hepatomegaly in KO and WT mice.

FIG. 3C-FIG. 3D show the evaluation of glycogen levels in Phkg2^{-/-} mice with FIG. 3C showing the liver glycogen content and FIG. 3D showing PAS staining to identify the presence of glycogen. FIG. 3E shows the GSD hepatocyte architectural changes and early perisinusoidal fibrosis in Phkg2^{-/-} KO mice (bottom panels) and WT mice (top panels) with H&E staining (left panels) to identify hepatocyte architecture and Masson's Trichrome staining (right panels) to identify tissue fibrosis.

FIG. 4A-FIG. 4F show the blood and urine analyses for Phkg2^{-/-} KO mice and WT mice with FIG. 4A showing the blood glucose levels, FIG. 4B showing the blood ketone levels, FIG. 4C showing the urine Hex₄ levels, FIG. 4D showing the alanine aminotransferase (ALT) levels, FIG. 4E showing the aspartate aminotransferase (AST) levels, and FIG. 4F showing the alkaline phosphatase (ALP) levels.

FIG. 5A-FIG. 5F shows the liver histology slides from humanized mice injected with AAV8 and AAVhum.8 capsids carrying GFP transgenes with FIG. 5A showing albumin staining for human hepatocytes in red, FIG. 5B showing GFP expression in green (indicating AAV8 transduction), FIG. 5C showing the merged image demonstrating that the AAV8 capsid transduced better in mouse hepatocytes, FIG. 5D showing albumin staining for human hepatocytes in red, FIG. 5E showing GFP expression in green (indicating AAVhum.8 transduction), and FIG. 5F showing the merged image demonstrating that the AAVhum.8 capsid more equally transduced human and mouse hepatocytes. FIG. 5G shows that the AAVhum.8 capsid transduces as well in mice and better in human hepatocytes than does AAV8.

FIG. 6A shows PHKG2 expression in HEK293 cells following transfection with pAV-CB-hPHKG2 or pAV-CB-mPhkg2 while FIG. 6B shows PHKG expression in HEK293 cells following transfection with pAV-CB-hPHKG2 or pAV-CB-hPHKG2^{CpGfree}.

FIG. 7A-FIG. 7G show the data generated by an in vivo experiment using AA9-LSP-mPhkg2 in 3-month-old mice. FIG. 7A shows the restoration of PhK activity following AAV9 treatment while FIG. 7B shows the reduction of liver glycogen content in treated mice. The PAS staining in FIG. 7C demonstrates that treated mice had a reduction of liver glycogen. FIG. 7D shows that treatment reduced the percent liver weight of GSD IX γ2 mice while FIG. 7E provides a representative image of the liver from an untreated animal and a treated animal. AAV treatment also improved the level of serum ALT (FIG. 7F) and serum AST (FIG. 7G).

FIG. 8 shows that treatment restored hepatocyte architecture in the AAV treated mice as revealed by H&E staining.

FIG. 9A-FIG. 9D show that AAV treatment restored the glycogenolysis metabolic pathway. FIG. 9A shows that treated mice had a relative intensity of the PhK γ2 subunit that approached the wild-type level. The treated mice and wild-type mice had similar relative intensities for the PhK α2 subunit (FIG. 9B) and the PhK β subunit (FIG. 9C). FIG. 9D shows that treated mice and wild-type mice have similar levels of P-PYGL/PYGL.

FIG. 10A-FIG. 10C show that AAV treatment restored the glycogenesis metabolic pathway. Treated mice and wild-type mice had similar levels of PGS/GS (FIG. 10A), PGSK3A/GSK3A (FIG. 10B), and PGSK3B/GSK3B (FIG. 10C), all of which were significantly reduced when compared to the untreated mice.

FIG. 11A-FIG. 11C show the data generated by an in vivo experiment using AA9-LSP-mPhkg2 in 3-month-old mice. FIG. 11A shows that the percent liver weight (LW/BW*100) in treated mice was the same or nearly the same as the wild-type mice, both of which were significantly reduced compared to the untreated mice. FIG. 11B shows that treatment decreased the liver glycogen content so that the level approached the wild-type level. FIG. 11C shows that AAV treatment reduced the level of serum ALT such that it resembled the wild-type level.

FIG. 12A-FIG. 12C show the data generated by an in vivo experiment using AA9-LSP-mPhkg2 in 6-month-old mice. FIG. 12A shows that the percent liver weight (LW/BW*100) in treated mice was the same or nearly the same level as that of the wild-type mice, both of which were significantly reduced compared to the untreated mice. FIG. 12B shows that treatment decreased the liver glycogen content so that the level approached the wild-type level. FIG. 12C shows that AAV treatment reduced the level of serum ALT such that it resembled the wild-type level.

FIG. 13A-FIG. 13E show the data generated by an in vivo experiment using AA9-LSP-hPHKG2 in 3-month-old mice. FIG. 13A shows that treated mice (treated with AAV-LSP-mPhkg2 or AAV-LSP-hPHKG2) had reduced liver glycogen content such that the level of glycogen content was similar to that of wild-type mice and significantly less than that of the untreated mice. FIG. 13B shows that treatment with AAV-LSP-hPHKG2 reduced urine Hex4 levels when compared to untreated mice (KO). FIG. 13C shows that the treated GSD IX γ2 mice (treated with AAV-LSP-mPhkg2 or AAV-LSP-hPHKG2) had significantly percent liver weight when compared to the untreated mice. The percent liver weight of GSD IX γ2 mice treated with either mPhkg2 or hPHKG2 was the same or nearly the same as that of the wild-type mice. FIG. 13D shows a representative liver from the untreated group (left), a representative liver from the AAV9-LSP-mPhkg2 treated group (middle), and a representative liver from the AAV9-LSP-hPHKG2 treated group (right). FIG. 13E shows that treated GSD IX γ2 mice treated with either AAV-LSP-mPhkg2 or AAV-LSP-hPHKG2 had a reduced level of serum AST.

FIG. 14A-FIG. 14B show that the 2-week treatment with AAV-LSP-hPHKG2 in 3-month-old mice restored glycogenolysis metabolic pathway as measured by the relative intensity of the PhK γ2 subunit (FIG. 14A), the PhK α2 subunit (FIG. 14B), and the PhK β subunit (FIG. 14C).

FIG. 15A-FIG. 15B show that the AAV9-LSP-hPHKG2 treatment in 3-month-old mice restored PhK enzyme activity to wild-type levels and reduced serum ALT. FIG. 15A shows that the treated GSD IX γ2 mice had PhK activity similar to that of wild-type mice while FIG. 15B shows that the AAV9-LSP-hPHKG2 treatment reduced the level of serum ALT.

FIG. 16 shows a series of Western blots comparing the hepatic protein levels for enzymes involved in the glycogen metabolic pathway (FIG. 1), indicating that AAV9 treated GSD IX γ2 mice had levels similar to the levels wild-type mice.

FIG. 17A-FIG. 17D shows the results of an in vivo experiment using AAV9-LSP-hPHKG2^{CpG-Free} in 3-month old GSD IX γ2 mice in a 2-week treatment protocol. FIG. 17A shows that the GSD IX γ2 mice treated with AAV9-LSP-hPHKG2^{CpG-Free} had significantly reduced percent liver weight when compared to untreated mice while FIG. 17B shows a representative liver from the AAV9-LSP-hPHKG2^{CpG-Free} group. FIG. 17C shows that GSD IX γ2 mice treated with AAV-LSP-hPHKG2^{CpG-Free} had a level of liver glycogen content similar to that of wild-type mice while FIG. 17D shows that treated GSD IX γ2 mice had a serum ALT level similar to that of wild-type mice, both of which were significantly less than that of untreated mice.

FIG. 18 shows the generation of Pygl-deficient mice by providing a schematic representation of the Pygl-knockout and WT alleles. Insertion of the FRT/loxP cassette in the intron between exon 2-3 disrupts Pygl mRNA expression. Arrows indicate WT or lacZ primers used for genotyping.

FIG. 19A-FIG. 19B show Western blot results demonstrating the overexpression of human glycogen phosphorylase L (hPYGL) and human phosphorylase kinase regulatory subunit alpha 2 (hPHKA2) in HEK293T cells. These Western blots show that the untreated animals had no appreciable amount of PHKA2 (FIG. 19A) or PYGL (FIG. 19B) while the transfected cells had demonstrable expression of PHKA2 (FIG. 19A) and PYGL (FIG. 19B).

FIG. 20A-FIG. 20F show the various constructs employed by the experiments described herein with FIG. 20A showing pAV-CB-mPhkg2, FIG. 20B showing pAV-CB-hPHKG2, FIG. 20C showing pAV-CB-hPHKG2^{CPGfree}, FIG. 20D showing pAV-LSP-hPHKG2^{CPGfree}, FIG. 20E showing pAV-CB-hPYGL, and FIG. 20F shows pAV-CB-hPHKA2.

### V. BRIEF SUMMARY

Disclosed herein is an isolated nucleic acid molecule comprising a nucleic acid sequence encoding a polypeptide capable of restoring one or more aspects of the glycogen metabolic pathway. Disclosed herein is an isolated nucleic acid molecule comprising a nucleic acid sequence encoding a polypeptide capable of restoring one or more aspects of the glycogenolysis metabolic pathway. Disclosed herein is an isolated nucleic acid molecule comprising a nucleic acid sequence encoding a polypeptide capable of restoring PhK subunit activity and/or functionality.

Disclosed herein is a vector comprising an isolated nucleic acid molecule comprising a nucleic acid sequence encoding a polypeptide capable of restoring one or more aspects of the glycogen metabolic pathway. Disclosed herein is a vector comprising an isolated nucleic acid molecule comprising a nucleic acid sequence encoding a polypeptide capable of restoring one or more aspects of the glycogenolysis metabolic pathway. Disclosed herein is a vector comprising an isolated nucleic acid molecule comprising a nucleic acid sequence encoding a polypeptide capable of restoring PhK subunit activity and/or functionality.

Disclosed herein is a pharmaceutical formulation comprising a disclosed vector and/or or a disclosed isolated nucleic acid molecule.

Disclosed herein is a method of treating and/or preventing disease progression comprising administering to a subject having GSD IX and/or GSD VI a therapeutically effective amount of a disclosed isolated nucleic acid molecule, a disclosed vector, a disclosed pharmaceutical formulation, or a combination thereof.

Disclosed herein is a method of treating and/or preventing disease progression comprising administering to a subject having GSD IX and/or GSD VI a therapeutically effective amount of a disclosed isolated nucleic acid molecule, a disclosed vector, a disclosed pharmaceutical formulation, or a combination thereof, wherein glycogen accumulation is prevented and/or accumulated glycogen is degraded in the subject.

Disclosed herein is a method of treating and/or preventing disease progression comprising administering to a subject having GSD IX and/or GSD VI a therapeutically effective amount of a disclosed isolated nucleic acid molecule, a disclosed vector, a disclosed pharmaceutical formulation, or a combination thereof, wherein one or more aspects of the glycogen metabolic pathway are restored.

Disclosed herein is a method of treating and/or preventing disease progression comprising administering to a subject having GSD IX and/or GSD VI a therapeutically effective amount of a disclosed isolated nucleic acid molecule, a disclosed vector, a disclosed pharmaceutical formulation, or a combination thereof, wherein one or more aspects of the glycogenolysis metabolic pathway are restored.

Disclosed herein is a method of treating and/or preventing disease progression comprising administering to a subject having GSD IX and/or GSD VI a therapeutically effective amount of a disclosed isolated nucleic acid molecule, a disclosed vector, a disclosed pharmaceutical formulation, or a combination thereof, wherein PhK subunit activity and/or functionality are restored.

Disclosed herein is a method of treating and/or preventing disease progression comprising administering to a subject having GSD IX and/or GSD VI a therapeutically effective amount of a disclosed isolated nucleic acid molecule, a disclosed vector, a disclosed pharmaceutical formulation, or a combination thereof, and reducing the expression level and/or activity level of glycogen synthase.

Disclosed herein is a method of treating and/or reducing liver disease comprising administering to a subject having GSD IX and/or GSD VI a therapeutically effective amount of a disclosed isolated nucleic acid molecule, a disclosed vector, a disclosed pharmaceutical formulation, or a combination thereof, and reducing the expression level and/or activity level of glycogen synthase, wherein one or more aspects of the glycogen metabolic pathway are restored.

Disclosed herein is a method of treating and/or reducing liver disease comprising administering to a subject having GSD IX and/or GSD VI a therapeutically effective amount of a disclosed isolated nucleic acid molecule, a disclosed vector, a disclosed pharmaceutical formulation, or a combination thereof, and reducing the expression level and/or activity level of glycogen synthase, wherein one or more aspects of the glycogenolysis metabolic pathway are restored.

Disclosed herein is a method of treating and/or preventing disease progression comprising administering to a subject having GSD IX and/or GSD VI a therapeutically effective amount of a disclosed isolated nucleic acid molecule, a disclosed vector, a disclosed pharmaceutical formulation, or a combination thereof, and reducing the expression level and/or activity level of glycogen synthase, wherein PhK subunit activity and/or functionality are restored.

Disclosed herein is a method of restoring the balance of glycogen metabolism comprising administering to a subject having GSD IX and/or GSD VI a therapeutically effective amount of a disclosed isolated nucleic acid molecule, a disclosed vector, a disclosed pharmaceutical formulation, or a combination thereof, wherein glycogen metabolism comprises glycogen synthesis and breakdown.

### VI. DETAILED DESCRIPTION

The present disclosure describes formulations, compounded compositions, kits, capsules, containers, and/or methods thereof. It is to be understood that the inventive aspects of which are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

### A. Definitions

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

This disclosure describes inventive concepts with reference to specific examples. However, the intent is to cover all modifications, equivalents, and alternatives of the inventive concepts that are consistent with this disclosure.

As used in the specification and the appended claims, the singular forms “a”, “an”, and “the” include plural referents unless the context clearly dictates otherwise.

The phrase “consisting essentially of” limits the scope of a claim to the recited components in a composition or the recited steps in a method as well as those that do not materially affect the basic and novel characteristic or characteristics of the claimed composition or claimed method. The phrase “consisting of” excludes any component, step, or element that is not recited in the claim. The phrase “comprising” is synonymous with “including”, “containing”, or “characterized by”, and is inclusive or open-ended. “Comprising” does not exclude additional, unrecited components or steps.

As used herein, when referring to any numerical value, the term “about” means a value falling within a range that is ±10% of the stated value.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight component X and 5 parts by weight component Y, X and Y are present at a weight ratio of 2:5, and are present in such ratio regardless of whether additional components are contained in the compound.

As used herein, the terms “optional” or “optionally” means that the subsequently described event or circumstance can or cannot occur, and that the description includes instances where said event or circumstance occurs and instances where it does not. In an aspect, a disclosed method can optionally comprise one or more additional steps, such as, for example, repeating an administering step or altering an administering step.

As used herein, the term “subject” refers to the target of administration, e.g., a human being. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig, or rodent. The term does not denote a particular age or sex, and thus, adult and child subjects, as well as fetuses, whether male or female, are intended to be covered. In an aspect, a subject can be a human patient. In an aspect, a subject can have a glycogen storage disease, be suspected of having a glycogen storage disease, or be at risk of developing a glycogen storage disease. In an aspect, a glycogen storage disease can be GSD IX and/or GSD VI.

As used herein, the term “diagnosed” means having been subjected to an examination by a person of skill, for example, a physician, and found to have a condition that can be diagnosed or treated by one or more of the disclosed isolated nucleic acid molecules, disclosed vectors, disclosed pharmaceutical formulations, or a combination thereof, or by one or more of the disclosed methods. For example, “diagnosed with a glycogen storage disease” means having been subjected to an examination by a person of skill, for example, a physician, and found to have a condition that can be treated by one or more of the disclosed isolated nucleic acid molecules, disclosed vectors, disclosed pharmaceutical formulations, or a combination thereof, or by one or more of the disclosed methods. For example, “suspected of having a glycogen storage disease” can mean having been subjected to an examination by a person of skill, for example, a physician, and found to have a condition that can likely be treated by one or more of by one or more of the disclosed isolated nucleic acid molecules, disclosed vectors, disclosed pharmaceutical formulations, or a combination thereof, or by one or more of the disclosed methods. In an aspect, an examination can be physical, can involve various tests (e.g., blood tests, genotyping, biopsies, etc.) and assays (e.g., enzymatic assay), or a combination thereof.

A “patient” refers to a subject afflicted with a glycogen storage disease. In an aspect, a patient can refer to a subject that has been diagnosed with or is suspected of having a glycogen storage disease. In an aspect, a patient can refer to a subject that has been diagnosed with or is suspected of having a glycogen storage disease (GSD) and is seeking treatment or receiving treatment for a GSD (such as GSD IX and/or GSD VI).

As used herein, the phrase “identified to be in need of treatment for a disorder,” or the like, refers to selection of a subject based upon need for treatment of the disorder. For example, a subject can be identified as having a need for treatment of a disorder (e.g., such as GSD VI or GSD IX) based upon an earlier diagnosis by a person of skill and thereafter subjected to treatment for the disorder (e.g., such as GSD IX and/or GSD VI). In an aspect, the identification can be performed by a person different from the person making the diagnosis. In an aspect, the administration can be performed by one who performed the diagnosis.

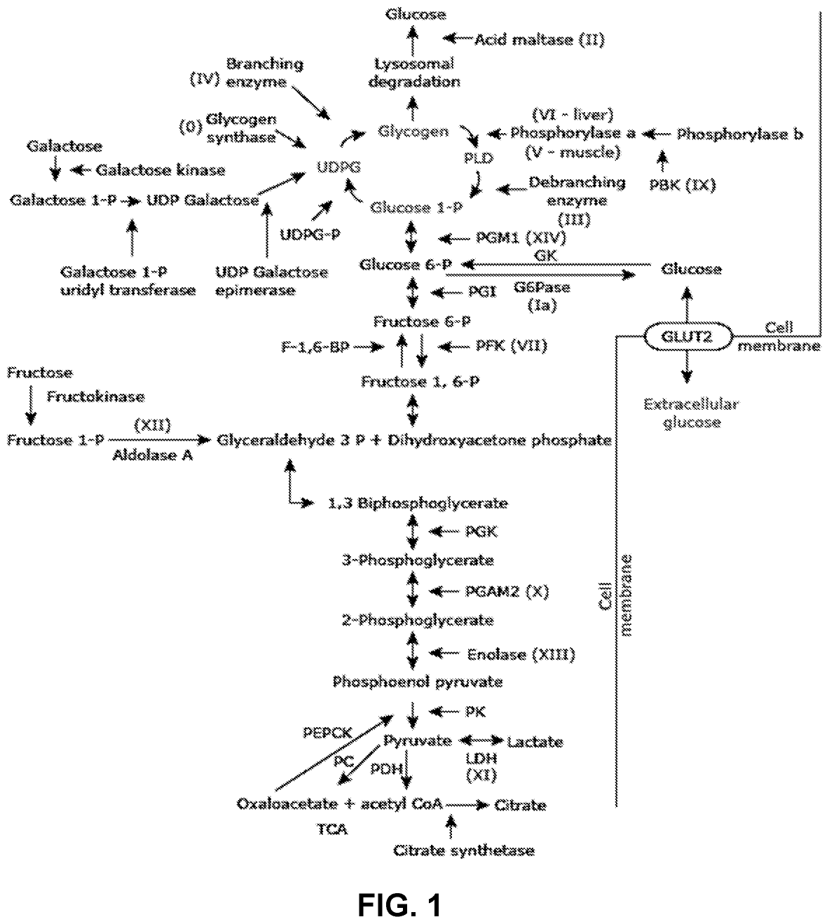

As used herein, “glycogenosis” (plural is glycogenoses) refers to a metabolic disorder caused by a defective glycogen metabolism resulting in the extra glycogen storage in cells. For example, FIG. 1 provides an illustrative example of the metabolic pathways of glycogen metabolism, including glycogen synthesis and breakdown, and including the sites of enzymatic defects that result in clinical glycogenoses.

As used herein, “inhibit,” “inhibiting”, and “inhibition” mean to diminish or decrease an activity, level, response, condition, severity, disease, or other biological parameter. This can include, but is not limited to, the complete ablation of the activity, level, response, condition, severity, disease, or other biological parameter. This can also include, for example, a 10% inhibition or reduction in the activity, level, response, condition, severity, disease, or other biological parameter as compared to the native or control level (e.g., a subject not having a GSD such as GSD IX and/or GSD VI). Thus, in an aspect, the inhibition or reduction can be a 10%, 20%, 30%, 40%, 50%, 60%, 70%, 80%, 90%, 100%, or any amount of reduction in between as compared to native or control levels. In an aspect, the inhibition or reduction can be 10-20%, 20-30%, 30-40%, 40-50%, 50-60%, 60-70%, 70-80%, 80-90%, or 90-100% as compared to native or control levels. In an aspect, the inhibition or reduction can be 0-25%, 25-50%, 50-75%, or 75-100% as compared to native or control levels.

The words “treat” or “treating” or “treatment” include palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In an aspect, the terms cover any treatment of a subject, including a mammal (e.g., a human), and includes: (i) preventing the undesired physiological change, disease, pathological condition, or disorder from occurring in a subject that can be predisposed to the disease but has not yet been diagnosed as having it; (ii) inhibiting the physiological change, disease, pathological condition, or disorder, i.e., arresting its development; or (iii) relieving the physiological change, disease, pathological condition, or disorder, i.e., causing regression of the disease. For example, in an aspect, treating a GSD (such as GSD IX and/or GSD VI) can reduce the severity of an established GSD in a subject by 1%-100% as compared to a control (such as, for example, an individual not having a glycogen storage disease). In an aspect, treating can refer to a 1%, 2%, 3%, 4%, 5%, 6%, 7%, 8%, 9%, 10%, 20%, 30%, 40%, 50%, 60%, 70%, 80%, 90%, or 100% reduction in the severity of a GSD (such as GSD IX and/or GSD VI). For example, treating a GSD can reduce one or more symptoms of a GSD in a subject by 1%-100% as compared to a control (such as, for example, an individual not having a glycogen storage disease). In an aspect, treating can refer to 1%, 2%, 3%, 4%, 5%, 6%, 7%, 8%, 9%, 10%, 20%, 30%, 40%, 50%, 60%, 70%, 80%, 90%, 100% reduction of one or more symptoms of an established GSD (such as GSD IX and/or GSD VI). It is understood that treatment does not necessarily refer to a cure or complete ablation or eradication of a GSD (such as GSD VI and/or GSD IX). However, in an aspect, treatment can refer to a cure or complete ablation or eradication of a GSD.

As used herein, the term “prevent” or “preventing” or “prevention” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed. In an aspect, preventing a GSD is intended. The words “prevent” and “preventing” and “prevention” also refer to prophylactic or preventative measures for protecting or precluding a subject (e.g., an individual) not having a given GSD or GSD-related complication from progressing to that complication (such as, for example, GSD IX and/or GSD VI).

As used herein, the terms “administering” and “administration” refer to any method of providing one or more of the disclosed isolated nucleic acid molecules, disclosed vectors, disclosed pharmaceutical formulations, or a combination thereof to a subject. Such methods are well-known to those skilled in the art and include, but are not limited to, the following: oral administration, transdermal administration, administration by inhalation, nasal administration, topical administration, in utero administration, intrahepatic administration, intravaginal administration, intracerebroventricular (ICV) administration, ophthalmic administration, intraaural administration, otic administration, intracerebral administration, rectal administration, sublingual administration, buccal administration, and parenteral administration, including injectable such as intravenous administration, intra-CSF administration, intra-cistem magna (ICM) administration, intra-arterial administration, intrathecal (ITH) administration, intramuscular administration, and subcutaneous administration. Administration can also include hepatic intra-arterial administration or administration through the hepatic portal vein (HPV). Administration of a disclosed isolated nucleic acid molecule, a disclosed vector, a disclosed pharmaceutical composition, a disclosed therapeutic agent, a disclosed immune modulator, a disclosed proteasome inhibitor, a disclosed small molecule, a disclosed endonuclease, a disclosed oligonucleotide, and/or a disclosed RNA therapeutic can comprise administration directly into the CNS or the PNS. Administration can be continuous or intermittent. Administration can comprise a combination of one or more route. In an aspect, a disclosed nucleic acid, a disclosed vector, a disclosed pharmaceutical formulation, or any combination thereof can be concurrently and/or serially administered to a subject via multiple routes of administration. For example, in an aspect, administering a disclosed nucleic acid, a disclosed vector, a disclosed pharmaceutical formulation, or any combination thereof can comprise intravenous administration and intra-cistern magna (ICM) administration. In an aspect, administering a disclosed nucleic acid, a disclosed vector, a disclosed pharmaceutical formulation, or any combination thereof can comprise IV administration and intrathecal (ITH) administration. Various combinations of administration are known to the skilled person.

In an aspect, a therapeutically effective amount of disclosed vector can be delivered intravenously and can comprise a range of about 1×10¹⁰ vg/kg to about 2×10¹⁴ vg/kg.

In an aspect, the skilled person can determine an efficacious dose, an efficacious schedule, and an efficacious route of administration for one or more of the disclosed isolated nucleic acid molecules, disclosed vectors, disclosed pharmaceutical formulations, or a combination thereof so as to treat or prevent an GSD (such as GSD IX and/or GSD VI). In an aspect, the skilled person can also alter, change, or modify an aspect of an administering step to improve efficacy of one or more of the disclosed isolated nucleic acid molecules, disclosed vectors, disclosed pharmaceutical formulations, or a combination thereof.

As used herein, “modifying the method” can comprise modifying or changing one or more features or aspects of one or more steps of a disclosed method. For example, in an aspect, a method can be altered by changing the amount of one or more of the disclosed isolated nucleic acid molecules, disclosed vectors, disclosed pharmaceutical formulations, or a combination thereof administered to a subject, or by changing the frequency of administration of one or more of the disclosed isolated nucleic acid molecules, disclosed vectors, disclosed pharmaceutical formulations, or a combination thereof to a subject, or by changing the duration of time one or more of the disclosed isolated nucleic acid molecules, disclosed vectors, disclosed pharmaceutical formulations, or a combination are administered to a subject.

As used herein, “concurrently” means (1) simultaneously in time, or (2) at different times during the course of a common treatment schedule.

The term “contacting” as used herein refers to bringing one or more of the disclosed isolated nucleic acid molecules, disclosed vectors, disclosed pharmaceutical formulations, or a combination thereof together with a target area or intended target area in such a manner that the one or more of the disclosed isolated nucleic acid molecules, disclosed vectors, disclosed pharmaceutical formulations, or a combination thereof exert an effect on the intended target or targeted area either directly or indirectly. A target area or intended target area can be one or more of a subject's organs (e.g., lungs, heart, liver, muscle, kidney, brain, etc.). In an aspect, a target area or intended target area can be any cell or any organ infected by a GSD (such as GSD IX and/or GSD VI). In an aspect, a target area or intended target area can be the liver.

As used herein, “determining” can refer to measuring or ascertaining the presence and severity of a glycogen storage disease, such as, for example, GSD IX and/or GSD VI. Methods and techniques used to determine the presence and/or severity of a GSD are typically known to the medical arts. For example, the art is familiar with the ways to identify and/or diagnose the presence, severity, or both of a GSD (such as, for example, GSD IX and/or GSD VI).

As used herein, “effective amount” and “amount effective” can refer to an amount that is sufficient to achieve the desired result such as, for example, the treatment and/or prevention of a glycogen storage disease (such as GSD IX and/or GSD VI) or a suspected a glycogen storage disease. As used herein, the terms “effective amount” and “amount effective” can refer to an amount that is sufficient to achieve the desired an effect on an undesired condition (e.g., a GSD). For example, a “therapeutically effective amount” refers to an amount that is sufficient to achieve the desired therapeutic result or to have an effect on undesired symptoms but is generally insufficient to cause adverse side effects. In an aspect, “therapeutically effective amount” means an amount of a disclosed isolated nucleic acid molecule, a disclosed vector, or a disclosed pharmaceutical formulation; that (i) treats the particular disease, condition, or (such as GSD IX and/or GSD VI), (ii) attenuates, ameliorates, or eliminates one or more symptoms of the particular disease, condition, or disorder e.g., a glycogen storage disease), or (iii) delays the onset of one or more symptoms of the particular disease, condition, or disorder described herein (e.g., a GSD). The specific therapeutically effective dose level for any particular patient will depend upon a variety of factors including the disorder being treated and the severity of the disorder; the disclosed isolated nucleic acid molecules, disclosed vectors, disclosed pharmaceutical formulations employed; the disclosed methods employed; the age, body weight, general health, sex and diet of the patient; the time of administration; the route of administration; the rate of excretion of the disclosed isolated nucleic acid molecules, disclosed vectors, or disclosed pharmaceutical formulations employed; the duration of the treatment; drugs used in combination or coincidental with the disclosed isolated nucleic acid molecules, disclosed vectors, or disclosed pharmaceutical formulations employed, and other like factors well-known in the medical arts. For example, it is well within the skill of the art to start doses of the disclosed isolated nucleic acid molecules, disclosed vectors, or disclosed pharmaceutical formulations at levels lower than those required to achieve the desired therapeutic effect and to gradually increase the dosage until the desired effect is achieved. If desired, then the effective daily dose can be divided into multiple doses for purposes of administration. Consequently, a single dose of the disclosed isolated nucleic acid molecules, disclosed vectors, or disclosed pharmaceutical formulations can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition, such as, for example, a glycogen storage disease.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents, or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. In an aspect, a pharmaceutical carrier employed can be a solid, liquid, or gas. In an aspect, examples of solid carriers can include lactose, terra alba, sucrose, talc, gelatin, agar, pectin, acacia, magnesium stearate, and stearic acid. In an aspect, examples ofliquid carriers can include sugar syrup, peanut oil, olive oil, and water. In an aspect, examples of gaseous carriers can include carbon dioxide and nitrogen. In preparing a disclosed composition for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “excipient” refers to an inert substance which is commonly used as a diluent, vehicle, preservative, binder, or stabilizing agent, and includes, but is not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). See, also, for reference, Remington's Pharmaceutical Sciences, (1990) Mack Publishing Co., Easton, Pa., which is hereby incorporated by reference in its entirety.

As used herein, “RNA therapeutics” can refer to the use of oligonucleotides to target RNA. RNA therapeutics can offer the promise of uniquely targeting the precise nucleic acids involved in a particular disease with greater specificity, improved potency, and decreased toxicity. This could be particularly powerful for genetic diseases where it is most advantageous to aim for the RNA as opposed to the protein. In an aspect, a therapeutic RNA can comprise one or more expression sequences. As known to the art, expression sequences can comprise an RNAi, shRNA, mRNA, non-coding RNA (ncRNA), an antisense such as an antisense RNA, miRNA, morpholino oligonucleotide, peptide-nucleic acid (PNA) or ssDNA (with natural, and modified nucleotides, including but not limited to, LNA, BNA, 2′-O-Me-RNA, 2′-MEO-RNA, 2′-F-RNA), or analog or conjugate thereof. In an aspect, a disclosed therapeutic RNA can comprise one or more long non-coding RNA (lncRNA), such as, for example, a long intergenic non-coding RNA (lincRNA), pre-transcript, pre-miRNA, pre-mRNA, competing endogenous RNA (ceRNA), small nuclear RNA (snRNA), small nucleolar RNA (snoRNA), pseudo-gene, rRNA, or tRNA. In an aspect, ncRNA can be piwi-interacting RNA (piRNA), primary miRNA (pri-miRNA), or premature miRNA (pre-miRNA). In an aspect, a disclosed therapeutic RNA or a RNA therapeutic can comprise antisense oligonucleotides (ASOs) that inhibit mRNA translation, oligonucleotides that function via RNA interference (RNAi) pathway, RNA molecules that behave like enzymes (ribozymes), RNA oligonucleotides that bind to proteins and other cellular molecules, and ASOs that bind to mRNA and form a structure that is recognized by RNase H resulting in cleavage of the mRNA target. In an aspect, RNA therapeutics can comprise RNAi and ASOs that inhibit mRNA translation of liver or muscle glycogen synthase (e.g., GYS1 and/or GYS2). Generally speaking, as known to the art, RNAi operates sequence specifically and post-transcriptionally by activating ribonucleases which, along with other enzymes and complexes, coordinately degrade the RNA after the original RNA target has been cut into smaller pieces while antisense oligonucleotides bind to their target nucleic acid via Watson-Crick base pairing, and inhibit or alter gene expression via steric hindrance, splicing alterations, initiation of target degradation, or other events.

As used herein, “small molecule” can refer to any organic or inorganic material that is not a polymer. Small molecules exclude large macromolecules, such as large proteins (e.g., proteins with molecular weights over 2,000, 3,000, 4,000, 5,000, 6,000, 7,000, 8,000, 9,000, or 10,000), large nucleic acids (e.g., nucleic acids with molecular weights of over 2,000, 3,000, 4,000, 5,000, 6,000, 7,000, 8,000, 9,000, or 10,000), or large polysaccharides (e.g., polysaccharides with a molecular weight of over 2,000, 3,000, 4,000, 5,000, 6,000, 7,000, 8,000, 9,000, or 10,000). In an aspect, a “small molecule”, for example, can be a drug that can enter cells easily because it has a low molecular weight.

As used herein, “CpG-free” can mean completely free of CpGs or partially free of CpGs. In an aspect, “CpG-free” can mean “CpG-depleted”. In an aspect, “CpG-depleted” can mean completely depleted of CpGs or partially depleted of CpGs. In an aspect, “CpG-free” can mean “CpG-optimized” for a desired and/or ideal expression level. CpG depletion and/or optimization is known to the skilled person in the art.

As used herein, “guaiacol” refers to a small molecule having a MW of 124.14. Guaiacol is a monomethoxybenzene comprising phenol with a methoxy substituent at the ortho position (C₇H₈O₂). In an aspect, guaiacol can increase inactivating GYS1 phosphorylation and/or can increase phosphorylation of the master activator of catabolism, AMP-dependent protein kinase. In an aspect, guaiacol can be a competitive inhibitor of purified GYS1 and GYS2 and a mixed inhibitor of the enzymes in cell lysates. In an aspect, guaiacol can reduce the expression level and/or activity level of glycogen synthase (such as GYS1 and/or GYS2).

As known to the art, miRNAs are small non-coding RNAs that are about 17 to about 25 nucleotide bases (nt) in length in their biologically active form. In an aspect, a disclosed miRNA can regulate gene expression post transcriptionally by decreasing target mRNA translation. In an aspect, a disclosed miRNA can function as a negative regulator. In an aspect, a disclosed miRNA is about 17 to about 25, about 17 to about 24, about 17 to about 23, about 17 to about 22, about 17 to about 21, about 17 to about 20, about 17 to about 19, about 18 to about 25, about 18 to about 24, about 18 to about 23, about 18 to about 22, about 18 to about 21, about 18 to about 20, about 19 to about 25, about 19 to about 24, about 19 to about 23, about 19 to about 22, about 19 to about 21, about 20 to about 25, about 20 to about 24, about 20 to about 23, about 20 to about 22, about 21 to about 25, about 21 to about 24, about 21 to about 23, about 22 to about 25, about 22 to about 24, or about 22 nucleotides in length. Generally, there are three forms of miRNAs: primary miRNAs (pri-miRNAs), premature miRNAs (pre-miRNAs), and mature miRNAs, all of which are within the scope of the present disclosure.

As used herein, “operably linked” means that expression of a gene or a transgene is under the control of a promoter with which it is spatially connected. A promoter can be positioned 5′ (upstream) or 3′ (downstream) of a gene under its control. The distance between the promoter and a gene can be approximately the same as the distance between that promoter and the gene it controls in the gene from which the promoter is derived. As is known in the art, variation in this distance can be accommodated without loss of promoter function.

As used herein, an “enhancer” such as a transcription or transcriptional enhancer refers to regulatory DNA segment that is typically found in multicellular eukaryotes. An enhancer can strongly stimulate (“enhance”) the transcription of a linked transcription unit, i.e., it acts in cis. An enhancer can activate transcription over very long distances of many thousand base pairs, and from a position upstream or downstream of the site of transcription initiation. An enhancers can have a modular structure by being composed of multiple binding sites for transcriptional activator proteins. Many enhancers control gene expression in a cell type-specific fashion. Several remote enhancers can control the expression of a singular gene while a singular enhance can stimulate the transcription of one or more genes.

As used herein, “expression cassette” or “transgene cassette” can refer to a distinct component of vector DNA comprising a transgene and one or more regulatory sequences to be expressed by a transfected cell. Generally, an expression cassette or transgene cassette can comprise a promoter sequence, an open reading frame (i.e., the transgene), and a 3′ untranslated region (e.g., in eukaryotes a polyadenylation site).

As used herein, “promoter” or “promoters” are known to the art. Depending on the level and tissue-specific expression desired, a variety of promoter elements can be used. A promoter can be tissue-specific or ubiquitous and can be constitutive or inducible, depending on the pattern of the gene expression desired. A promoter can be native (endogenous) or foreign (exogenous) and can be a natural or a synthetic sequence. By foreign or exogenous, it is intended that the transcriptional initiation region is not found in the wild-type host into which the transcriptional initiation region is introduced.

“Tissue-specific promoters” are known to the art and include, but are not limited to, neuron-specific promoters, muscle-specific promoters, liver-specific promoters, skeletal muscle-specific promoters, and heart-specific promoters.

“Liver-specific promoters” are known to the art and include, but are not limited to, the thyroxin binding globulin (TBG) promoter, the α1-microglobulin/bikunin enhancer/thyroid hormone-binding globulin promoter, the human albumin (hALB) promoter, the thyroid hormone-binding globulin promoter, the α-1-anti-trypsin promoter, the bovine albumin (bAlb) promoter, the murine albumin (mAlb) promoter, the human α1-antitrypsin (hAAT) promoter, the ApoEhAAT promoter comprising the ApoE enhancer and the hAAT promoter, the transthyretin (TTR) promoter, the liver fatty acid binding protein promoter, the hepatitis B virus (HBV) promoter, the DC172 promoter comprising the hAAT promoter and the α1-microglobulin enhancer, the DC190 promoter comprising the human albumin promoter and the prothrombin enhancer, or any other natural or synthetic liver-specific promoter.

In an aspect, a disclosed liver-specific promoter can comprise any liver-specific promoter known to the art. In an aspect, a liver specific promoter can comprise about 845-bp and comprise the thyroid hormone-binding globulin promoter sequences (2382 to 13), two copies of α1-microglobulinybikunin enhancer sequences (22,804 through 22,704), and a 71-bp leader sequence (Ill C R, et al. (1997) Blood Coagul Fibrinolysis. 8 Suppl 2:S23-S30). In an aspect, a disclosed liver-specific promoter can comprise the sequence set forth in SEQ ID NO:56. In an aspect, a disclosed liver-specific promoter can comprise a sequence having at least 40%, 50%, 60%, 70%, 80%, 85%, 86%, 87%, 88%, 89%, 90%, 91%, 92%, 93%, 94%, 95%, 96%, 97%, 98%, or 99% identity to the sequence set forth in SEQ ID NO:56. In an aspect, a disclosed liver-specific promoter can comprise a sequence having at least 40%-60%, at least 60%-80%, at least 80%-90%, or at least 90%-100% identity to the sequence set forth in SEQ ID NO:56.

In an aspect, a disclosed ubiquitous promoter can be a CMV enhancer/chicken β-actin promoter (CB promoter).

As used herein, an “inducible promoter” refers to a promoter that can be regulated by positive or negative control. Factors that can regulate an inducible promoter include, but are not limited to, chemical agents (e.g., the metallothionein promoter or a hormone inducible promoter), temperature, and light.

In an aspect, a disclosed promoter can be a promoter/enhancer. As used herein, the term promoter/enhancer can refer to a segment of DNA that contains nucleotide sequences capable of providing both promoter and enhancer functions.

As discussed above, a disclosed promoter can be an endogenous promoter. Endogenous refers to a disclosed promoter or disclosed promoter/enhancer that is naturally linked with its gene. In an aspect, a disclosed endogenous promoter can generally be obtained from a non-coding region upstream of a transcription initiation site of a gene (such as, for example, a disclosed phosphorylase kinase, phosphorylase, or some other enzyme involved in the glycogen metabolic pathway). In an aspect, a disclosed endogenous promoter can be used for constitutive and efficient expression of a disclosed transgene (e.g., a nucleic acid sequence encoding a polypeptide capable of preventing glycogen accumulation and/or degrading accumulated glycogen).

As discussed above, a disclosed promoter can be an exogenous promoter. Exogenous (or heterologous) refers to a disclosed promoter or a disclosed promoter/enhancer that can be placed in juxtaposition to a gene by means of molecular biology techniques such that the transcription of that gene can be directed by the linked promoter or linked promoter/enhancer. In an aspect, a disclosed endogenous promoter can be an endogenous promoter/enhancer.

As used herein, the term “serotype” is a distinction used to refer to an AAV having a capsid that is serologically distinct from other AAV serotypes. Serologic distinctiveness can be determined on the basis of the lack of cross-reactivity between antibodies to one AAV as compared to another AAV. Such cross-reactivity differences are usually due to differences in capsid protein sequences/antigenic determinants (e.g., due to VP1, VP2, and/or VP3 sequence differences of AAV serotypes).

As used herein, “tropism” refers to the specificity of an AAV capsid protein present in an AAV viral particle, for infecting a particular type of cell or tissue. The tropism of an AAV capsid for a particular type of cell or tissue may be determined by measuring the ability of AAV vector particles comprising the hybrid AAV capsid protein to infect or to transduce a particular type of cell or tissue, using standard assays that are well-known in the art such as those disclosed in the examples of the present application. As used herein, the term “liver tropism” or “hepatic tropism” refers to the tropism for liver or hepatic tissue and cells, including hepatocytes.

“Sequence identity” and “sequence similarity” can be determined by alignment of two peptide or two nucleotide sequences using global or local alignment algorithms. Sequences may then be referred to as “substantially identical” or “essentially similar” when they are optimally aligned. For example, sequence similarity or identity can be determined by searching against databases such as FASTA, BLAST, etc., but hits should be retrieved and aligned pairwise to compare sequence identity. Two proteins or two protein domains, or two nucleic acid sequences can have “substantial sequence identity” if the percentage sequence identity is at least 70%, 75%, 80%, 85%, 90%, 95%, 98%, 99% or more, preferably 90%, 95%, 98%, 99% or more. Such sequences are also referred to as “variants” herein, e.g., other variants of glycogen branching enzymes and amylases. It should be understood that sequence with substantial sequence identity do not necessarily have the same length and may differ in length. For example, sequences that have the same nucleotide sequence but of which one has additional nucleotides on the 3′- and/or 5′-side are 100% identical.

As used herein, “codon optimization” can refer to a process of modifying a nucleic acid sequence for enhanced expression in the host cells of interest by replacing one or more codons or more of the native sequence with codons that are more frequently or most frequently used in the genes of that host cell while maintaining the native amino acid sequence. Various species exhibit particular bias for certain codons of a particular amino acid. As contemplated herein, genes can be tailored for optimal gene expression in a given organism based on codon optimization. Codon usage tables are readily available, for example, at the “Codon Usage Database.” Many methods and software tools for codon optimization have been reported previously. (See, for example, genomes.urv.es/OPTIMIZER/).

As used herein, “GYS1” refers to glycogen synthase (muscle), which is an enzyme that transfers the glycosyl residue from UDP-Glc to the non-reducing end of alpha-1,4-glucan while “GYS2” refers to glycogen synthase (liver), which is an enzyme that transfers the glycosyl residue from UDP-Glc to the non-reducing end of alpha-1,4-glucan.

As used herein, “substrate reduction therapy” or “SRT” refers to methods of reducing the level of the substrate to a point where residual degradative activity of one or more enzymes is sufficient to prevent substrate accumulation. Generally, SRT aims to use small molecule inhibitors of biosynthesis to reduce the concentration of accumulating substrate to a level where the residual degradative enzymes can maintain homeostasis. For example, in an aspect, SRT refers to a method of inhibiting glycogen synthase (i.e., GYS1 and/or GYS2) in a cell or a subject to reduce glycogen synthesis and/or glycogen accumulation in cells and tissues (e.g., skeletal muscle, lung tissue, liver tissue, brain tissue, or any other tissue having glycogen accumulation) when PHKA1, PHKA2, PHKB, CALM1, CALM2, CALM3, PHKG2, and/or PYGL activity and/or expression levels are reduced. In an aspect, SRT can be used to reduce activity and/or expression of GYS1 and/or GYS2 in view of the reduced activity and/or expression level of PHKA1, PHKA2, PHKB, CALM1, CALM2, CALM3, PHKG2, and/or PYGL, and/or one or more other enzymes in the metabolic pathways of glycogen synthesis and breakdown. In an aspect, SRT can comprise siRNA-based therapies, shRNA-based therapies, antisense therapies, gene-editing therapies, and therapies using one or more small molecules or peptide drugs. In an aspect, SRT can comprise administration of one or more small molecules that can traverse the blood-brain barrier in quantities that are therapeutic for a subject having neuropathic glycogen storage disease. In an aspect, SRT can comprise administration of one or more small molecules that do not traverse the blood-brain barrier in quantities but are nonetheless therapeutic for a subject having neuropathic glycogen storage disease. In an aspect, a disclosed small molecule that inhibits glycogen synthase (GYS1) in SRT can be orally delivered.

As used herein, “CRISPR or clustered regularly interspaced short palindromic repeat” is an ideal tool for correction of genetic abnormalities as the system can be designed to target genomic DNA directly. A CRISPR system involves two main components—a Cas9 enzyme and a guide (gRNA). The gRNA contains a targeting sequence for DNA binding and a scaffold sequence for Cas9 binding. Cas9 nuclease is often used to “knockout” target genes hence it can be applied for deletion or suppression of oncogenes that are essential for cancer initiation or progression. Similar to ASOs and siRNAs, CRISPR offers a great flexibility in targeting any gene of interest hence, potential CRISPR based therapies can be designed based on the genetic mutation in individual patients. An advantage of CRISPR is its ability to completely ablate the expression of disease genes which can only be suppressed partially by RNA interference methods with ASOs or siRNAs. Furthermore, multiple gRNAs can be employed to suppress or activate multiple genes simultaneously, hence increasing the treatment efficacy and reducing resistance potentially caused by new mutations in the target genes.

As used herein, “CRISPR-based endonucleases” include RNA-guided endonucleases that comprise at least one nuclease domain and at least one domain that interacts with a guide RNA. As known to the art, a guide RNA directs the CRISPR-based endonucleases to a targeted site in a nucleic acid at which site the CRISPR-based endonucleases cleaves at least one strand of the targeted nucleic acid sequence. As the guide RNA provides the specificity for the targeted cleavage, the CRISPR-based endonuclease is universal and can be used with different guide RNAs to cleave different target nucleic acid sequences. CRISPR-based endonucleases are RNA-guided endonucleases derived from CRISPR/Cas systems. Bacteria and archaea have evolved an RNA-based adaptive immune system that uses CRISPR (clustered regularly interspersed short palindromic repeat) and Cas (CRISPR-associated) proteins to detect and destroy invading viruses or plasmids. CRISPR/Cas endonucleases can be programmed to introduce targeted site-specific double-strand breaks by providing target-specific synthetic guide RNAs (Jinek et al. (2012) Science. 337:816-821).

In an aspect, a disclosed CRISPR-based endonuclease can be derived from a CRISPR/Cas type I, type II, or type III system. Non-limiting examples of suitable CRISPR/Cas proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966.

In an aspect, a disclosed CRISPR-based endonuclease can be derived from a type II CRISPR/Cas system. For example, in an aspect, a CRISPR-based endonuclease can be derived from a Cas9 protein. The Cas9 protein can be from Streptococcus pyogenes, Streptococcus thermophilus, Streptococcus sp, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina. In an aspect, the CRISPR-based nuclease can be derived from a Cas9 protein from Staphylococcus Aureus (SEQ ID NO:32) or Streptococcus pyogenes (SEQ ID NO:33).

In general, CRISPR/Cas proteins can comprise at least one RNA recognition and/or RNA binding domain. RNA recognition and/or RNA binding domains can interact with the guide RNA such that the CRISPR/Cas protein is directed to a specific genomic or genomic sequence. CRISPR/Cas proteins can also comprise nuclease domains (i.e., DNase or RNase domains), DNA binding domains, helicase domains, protein-protein interaction domains, dimerization domains, as well as other domains.

The CRISPR-based endonuclease can be a wild type CRISPR/Cas protein (such as for example, SEQ ID NO:32 and SEQ ID NO:33), a modified CRISPR/Cas protein, or a fragment of a wild type or modified CRISPR/Cas protein. The CRISPR/Cas protein can be modified to increase nucleic acid binding affinity and/or specificity, alter an enzymatic activity, and/or change another property of the protein. For example, in an aspect, nuclease (i.e., DNase, RNase) domains of the CRISPR/Cas protein can be modified, deleted, or inactivated. A CRISPR/Cas protein can be truncated to remove domains that are not essential for the function of the protein. A CRISPR/Cas protein also can be truncated or modified to optimize the activity of the protein or an effector domain fused with a CRISPR/Cas protein.

In an aspect, a disclosed CRISPR-based endonuclease can be derived from a wild type Cas9 protein (such as, for example, SEQ ID NO:32 or SEQ ID NO:33) or fragment thereof. In an aspect, a disclosed CRISPR-based endonuclease can be derived from a modified Cas9 protein. For example, the amino acid sequence of a disclosed Cas9 protein can be modified to alter one or more properties (e.g., nuclease activity, affinity, stability, etc.) of the protein. Alternatively, domains of the Cas9 protein not involved in RNA-guided cleavage can be eliminated from the protein such that the modified Cas9 protein is smaller than the wild type Cas9 protein.

As used herein, “immune tolerance,” “immunological tolerance,” and “immunotolerance” refers to a state of unresponsiveness or blunted response of the immune system to substances (e.g., a disclosed isolated nucleic acid molecule, a disclosed vector, a disclosed transgene product, a disclosed pharmaceutical formulation, a disclosed therapeutic agent, etc.) that have the capacity to elicit an immune response in a subject. Immune tolerance is induced by prior exposure to a specific antigen. Immune tolerance can be determined in a subject by measuring antibodies against a particular antigen or by liver-restricted transgene expression with an AAV vector. Low or absent antibody titers over time is an indicator of immune tolerance. For example, in some embodiments, immune tolerance can be established by having IgG antibody titers of less than or equal to about 12,000, 11,500, 11,000, 10,500, 10,000, 9,500, 9,000, 8,500, 8,000, 7,500, 7,000, 6,500, or 6,000 within following gene therapy (such as the administration of the transgene encoding, for example, PHKA1, PHKA2, PHKB, CALM1, CALM2, CALM3, PHKG2, PYGL, and/or GAA) or a CpG-depleted and codon optimized ORF for PHKA1, PHKA2, PHKB, CALM1, CALM2, CALM3, PHKG2, PYGL and/or GAA.

As known to the art, antibodies (Abs) can mitigate AAV infection through multiple mechanisms by binding to AAV capsids and blocking critical steps in transduction such as cell surface attachment and uptake, endosomal escape, productive trafficking to the nucleus, or uncoating as well as promoting AAV opsonization by phagocytic cells, thereby mediating their rapid clearance from the circulation. For example, in humans, serological studies reveal a high prevalence of NAbs in the worldwide population, with about 67% of people having antibodies against AAV1, 72% against AAV2, and approximately 40% against AAV serotypes 5 through 9. Vector immunogenicity represents a major challenge in re-administration of AAV vectors.

In an aspect, also disclosed herein are partial self-complementary parvovirus (e.g., a disclosed AAV) genomes, plasmid vectors encoding the parvovirus genomes, and parvovirus (e.g., a disclosed AAV) particles including such genomes. In an aspect, provided herein is a plasmid vector comprising a nucleotide sequence encoding a disclosed parvovirus genome such as for example, a disclosed AAV. In an aspect, provided herein is a partial self-complementary parvovirus genome including a payload construct, parvovirus ITRs flanking the payload construct, and a self-complementary region flanking one of the ITRs. A self-complementary region can comprise a nucleotide sequence that is complementary to the payload construct. A disclosed self-complementary region can have a length that is less the entire length of the payload construct.

In an aspect, a disclosed self-complementary region of a disclosed parvovirus genome can comprise a minimum length, while still having a length that is less the entire length of the payload construct. In an aspect, a disclosed self-complementary region can comprise at least 50 bases in length, at least 100 bases in length, at least 200 in length, at least 300 bases in length, at least 400 bases in length, at least 500 bases in length, at least 600 bases in length, at least 700 bases in length, at least 800 bases in length, at least 900 bases in length, or at least 1,000 bases in length.

In an aspect, a “self-complementary parvovirus genome” can be a single stranded polynucleotide having, in the 5′ to 3′ direction, a first parvovirus ITR sequence, a heterologous sequence (e.g., payload construct comprising, for example, PHKA1, PHKA2, PHKB, PHKG2, PYGL and/or GAA), a second parvovirus ITR sequence, a second heterologous sequence, wherein the second heterologous sequence is complementary to the first heterologous sequence, and a third parvovirus ITR sequence. In contrast to a self-complementary genome, a “partial self-complementary genome” does not include three parvovirus ITRs and the second heterologous sequence that is complementary to the first heterologous sequence has a length that is less than the entire length of the first heterologous sequence (e.g., payload construct). Accordingly, a partial self-complementary genome is a single stranded polynucleotide having, in the 5′ to 3′ direction or the 3′ to 5′ direction, a first parvovirus ITR sequence, a heterologous sequence (e.g., payload construct), a second parvovirus ITR sequence, and a self-complementary region that is complementary to a portion of the heterologous sequence and has a length that is less than the entire length the heterologous sequence.

As used herein, “immune-modulating” refers to the ability of a disclosed isolated nucleic acid molecules, a disclosed vector, a disclosed pharmaceutical formulation, or a disclosed agent to alter (modulate) one or more aspects of the immune system. The immune system functions to protect the organism from infection and from foreign antigens by cellular and humoral mechanisms involving lymphocytes, macrophages, and other antigen-presenting cells that regulate each other by means of multiple cell-cell interactions and by elaborating soluble factors, including lymphokines and antibodies, that have autocrine, paracrine, and endocrine effects on immune cells.

As used herein, “immune modulator” refers to an agent that is capable of adjusting a given immune response to a desired level (e.g. as in immunopotentiation, immunosuppression, or induction of immunologic tolerance). Examples of immune modulators include but are not limited to, a disclosed immune modulator can comprise aspirin, azathioprine, belimumab, betamethasone dipropionate, betamethasone valerate, bortezomib, bredinin, cyazathioprine, cyclophosphamide, cyclosporine, deoxyspergualin, didemnin B, fluocinolone acetonide, folinic acid, ibuprofen, IL6 inhibitors (such as sarilumab) indomethacin, inebilizumab, intravenous gamma globulin (IVIG), methotrexate, methylprednisolone, mycophenolate mofetil, naproxen, prednisolone, prednisone, prednisolone indomethacin, rapamycin, rituximab, sirolimus, sulindac, synthetic vaccine particles containing rapamycin (SVP-Rapamycin or ImmTOR), thalidomide, tocilizumab, tolmetin, triamcinolone acetonide, anti-CD3 antibodies, anti-CD4 antibodies, anti-CD19 antibodies, anti-CD20 antibodies, anti-CD22 antibodies, anti-CD40 antibodies, anti-FcRN antibodies, anti-IL6 antibodies, anti-IGF1R antibodies, an IL2 mutein, a BTK inhibitor, or a combination thereof. In an aspect, a disclosed immune modulator can comprise one or more Treg (regulatory T cells) infusions (e.g., antigen specific Treg cells to AAV). In an aspect, a disclosed immune modulator can be bortezomib or SVP-Rapamycin. In an aspect, a disclosed immune modulator can be Tacrolimus. In an aspect, an immune modulator can be administered by any suitable route of administration including, but not limited to, in utero, intra-CSF, intrathecally, intravenously, subcutaneously, transdermally, intradermally, intramuscularly, orally, transcutaneously, intraperitoneally (IP), or intravaginally. In an aspect, a disclosed immune modulator can be administered using a combination of routes. Administration can also include hepatic intra-arterial administration or administration through the hepatic portal vein (HPV). Administration of an immune modulator can be continuous or intermittent, and administration can comprise a combination of one or more routes.

As used herein, the term “immunotolerant” refers to unresponsiveness to an antigen (e.g., a vector, a therapeutic protein, a transgene product, etc.). An immunotolerant promoter can reduce, ameliorate, or prevent transgene-induced immune responses that can be associated with gene therapy. Assays known in the art to measure immune responses, such as immunohistochemical detection of cytotoxic T cell responses, can be used to determine whether one or more promoters can confer immunotolerant properties.

As used herein, the term “package insert” is used to refer to instructions customarily included in commercial packages of therapeutic products, that contain information about the indications, usage, dosage, administration, contraindications and/or warnings concerning the use of such therapeutic products.

As used herein, the term “in combination” in the context of the administration of other therapies (e.g., other agents) includes the use of more than one therapy (e.g., drug therapy). Administration “in combination with” one or more further therapeutic agents includes simultaneous (e.g., concurrent) and consecutive administration in any order. The use of the term “in combination” does not restrict the order in which therapies are administered to a subject. By way of non-limiting example, a first therapy (e.g., a disclosed isolated nucleic acid molecule, a disclosed vector, a disclosed pharmaceutical formulation, or a combination thereof) may be administered prior to (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 8 weeks, 8 weeks, 9 weeks, 10 weeks, 11 weeks, or 12 weeks), concurrently, or after (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 7 weeks, 8 weeks, 9 weeks, 10 weeks, 11 weeks, or 12 weeks or longer) the administration of a second therapy (e.g., agent) to a subject having or diagnosed with a GSD (such as GSD IX al, GSD IX α2, GSD IX β, GSD IX δ, GSD IX γ2, and/or GSD VI).

Disclosed are the components to be used to prepare the disclosed isolated nucleic acid molecules, disclosed vectors, or disclosed pharmaceutical formulations as well as the disclosed isolated nucleic acid molecules, disclosed vectors, or disclosed pharmaceutical formulations used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

### B. Glycogen Storage Diseases

As used herein, “glycogen” refers to a branched polysaccharide with a molecular weight of 9-10 million Daltons. The average glycogen molecule contains about 55,000 glucosyl residues linked by α-1,4 (92%) and α-1,6 (8%) glycosidic bonds. Glycogen synthesis is catalyzed by the actions of 3 enzymes: (a) glycogenin (GYG), the initiating enzyme that starts a primer of glucose chain attached to itself, (b) glycogen synthase (GYS), which strings glucose to extend linear chains; and (c) glycogen-branching enzyme (GBE), which attaches a short new branch to a linear chain.

Glycogen storage disease (GSD) types VI and IX are caused by phosphorylase system deficiencies. The estimated prevalence of GSD IX is 1 in 100,000 individuals and the estimated prevalence of GSD VI is 1 in 65,000 to 1 in 85,000 individuals. The liver form of GSD IX is often clinically indistinguishable from GSD VI.

Most of the GSD VI and GSD IX patients present with hepatomegaly and short stature within the first 2 years of life. The phenotype ranges from mild (hepatomegaly and elevated liver enzymes) to severe (hypoglycemia, short stature, mild gross motor delays, progressive liver disease and liver cirrhosis). Biochemically, patients have elevated liver enzymes and an increased risk of fasting hypoglycemia. Clinical and biochemical features tend to improve with age. Non-specific clinical and biochemical features at initial presentation may delay referral to a metabolic genetics clinic. Confirmation of the diagnosis is based on the molecular genetic studies for specific genes, which are available on a clinical basis in many North American and European clinical molecular diagnostic laboratories. Enzyme activity measurement in red blood cells (RBCs) is available for GSD IXa1, GSD IXb, and GSD IXc, but normal RBC PhK does not rule out the diagnosis of GSD IX. If mutation analysis is not able to identify underlying genetic defect, invasive liver biopsy is an important diagnostic method for the confirmation of the diagnosis by enzyme activity measurement in liver biopsy specimens. The principal differential diagnosis for GSD VI and the liver GSD IXs includes other forms of GSD associated with hepatomegaly and hypoglycemia, especially GSD I and III (Table 1).

TABLE 1
Differential Diagnosis of GSD VI and GSD IX

| | Similarity with GSD VI | |
|---|---|---|
| Disorder | and GSD IX | Distinguishing Features |
| GSD type 0 (glycogen | Fasting hypoglycemia and | Absence of hepatomegaly; |
| synthase deficiency) | ketosis | postprandial hyperglycemia and |
| | | hyperlactatemia |
| GSD I (glucose-6- | Hepatomegaly, fasting | Severe fasting lactic acidosis, |
| phosphatase) | hypoglycemia, ↑ AST, ALT in | hyperuricemia, neutropenia (type |
| | early stage when initially | 1b) |
| | diagnosed, typically normalizes | |
| | with treatment, hyperlipidemia | |
| GSD III (glycogen | Hepatomegaly, fasting | Hypoglycemia usually less |
| debrancher enzyme | hypoglycemia, ↑↑ AST, ALT, | severe, presence of ketosis and |
| deficiency) | hyperlipidemia | absence of hyperlactatemia and |
| | | hyperuricemia; ↑ AST, ALT can |
| | | be higher (may be >500 U/L); |
| | | muscle involvement with ↑ CK |
| | | concentrations in GSD IIIa |
| GSD IV (branching | Hepatomegaly, ↑ AST, ALT, | Lack of hypoglycemia until end- |
| enzyme deficiency) | prolonged PT, low albumin | stage liver disease |
| | (latter two in advanced stage of | |
| | disease) | |
| GSD XI (Glut-2 | Hepatomegaly, fasting | Postprandial hyperglycemia; |
| deficiency) | hypoglycemia and ketosis, ↑ | gastrointestinal symptoms |
| | AST, ALT, Fanconi-like renal | (chronic diarrhea from |
| | tubular dysfunction (glucosuria, | carbohydrate malabsorption); |
| | proteinuria, phosphaturia, | hypophosphatemic rickets |
| | generalized aminoaciduria) | |
| Disorders of | Hepatomegaly, fasting | Hypoglycemia after more |
| gluconeogenesis (e.g., | hypoglycemia and | prolonged (e.g., overnight) |
| fructose-1,6- | hyperlacticacidemia, ↑ uric acid, | fasting or during intercurrent |
| bisphosphatase | AST, ALT | illness with reduced carbohydrate |
| deficiency) | | intake |
| Primary liver disease | Hepatomegaly, ↑ AST, ALT | Lack of fasting hypoglycemia |
| (e.g., α1-antitrypsin, | | and hyperlacticacidemia |
| hepatitis) | | |
| Mitochondrial | Hepatomegaly, transaminitis | Less significant glycogen |
| disorder | | accumulation, more severe lactic |
| | | acidosis, often multisystem |
| | | manifestations |
| Glycerol kinase | Hypoglycemia | Ketoacidosis and extremely |
| deficiency | | elevated glycerol |
| PRKAG2 deficiency | Nonlysosomal glycogen | Ventricular pre-excitation and |
| | accumulation primarily in | mild to severe cardiac |
| | skeletal and cardiac muscle; | hypertrophy, no hypoglycemia |
| | decrease in activity of | |
| | phosphorylase kinase | |
| Other storage | Hepatomegaly, growth failure, | Lack of fasting hypoglycemia, |
| (metabolic) diseases | hyperlipidemia | significant splenomegaly; storage |
| (Niemann-Pick B, | | cells characteristic of the disease, |
| Gaucher) | | other features like bone and |
| | | pulmonary involvement |

Table 2 compares the liver patient populations for GSD VI, GSD IX α2, and GSD IX γ2 and identifies a relevant animal model for each GSD.

TABLE 2
Differential Diagnosis of GSD VI and GSD IX

| Indication | GSD IX γ2 | GSD IX α2 | GSD VI |
|---|---|---|---|
| Gene | PHKG2 | PHKA2 | PYGL |
| Enzyme | Phosphorylase kinase | Phosphorylase kinase | Liver glycogen |
| | (PhK) | (PhK) | phosphorylase |
| Disease | Significant liver | Progressive, | Progressive, |
| Burden | involvement, fibrosis, | smoldering liver | smoldering liver |
| | and cirrhosis | fibrosis | fibrosis |
| | | Subset develop | Subset develop |
| | | progressive fibrosis | progressive fibrosis |
| | | and cirrhosis | and cirrhosis |
| Est. Patients in | ~2,500 | ~7,500 | ~13,500 |
| US and Europe | | | |
| Relevant | Phkg2^{-/-} KO mouse | Phka2^{-/-} KO mouse | Pygl^{-/-} KO mouse |
| Animal Model | model¹ | model² | model³ |

¹Gibson et al., 2021 (Mol. Genet. Metab.)
²Model Under Development
³Wilson et al., 2019 (Hepatol. Commun.)

Overlapping clinical features across liver GSDs such as GSD VI and GSD IX can allow for a “plug-and-play” approach for developing treatment strategies.

### 1. GSD VI

GSD VI is an autosomal recessive genetic disease that affects approximately 1 in 65,000 to 85,000 live births. At least three human glycogen phosphorylases exist, each of which is preferentially expressed in a different tissue; muscle, liver, and brain isoforms have been identified. GSD VI is the result of a deficiency of liver glycogen phosphorylase, which is encoded by the PYGL (OMIM *613741) gene located on chromosome 14q21-q22.3. The PYGL gene containing 20 exons is the only gene responsible for encoding hepatic glycogen phosphorylase. PYGL is the only gene known to be associated with GSD VI.

An integral enzyme in glucose metabolism, PYGL catalyzes the rate-limiting step of glycogenolysis, converting glycogen into glucose 1-phosphate (GIP). Breakdown of glycogen in the liver requires the stepwise activation of several cytosolic liver enzymes. Phosphorylase kinase, which phosphorylates liver PYGL, triggers a conformation switch from phosphorylase b (inactive form) to phosphorylase a (active form), which catalyzes the breakdown of glycogen into chains of GIP monomers. Through the addition of the glycogen debranching enzyme, which assists to further cleave α-1,6 glycosidic linkages, free GIP monomers are converted to glucose 6-phosphate, which can be released from the liver or shunted into alternative pathways. Additionally, glycogen is processed through the autophagy-lysosomal pathway, mediated by the enzyme acid α-glucosidase, to produce low quantities of free glucose.

GSD VI has variable severity and can present in infancy/early childhood with hepatomegaly, distended abdomen, and growth retardation. Rarely, hypoglycemia may manifest after prolonged fasting or during an illness. Ketotic hypoglycemia after an overnight fast may be seen in this disorder. Developmental delay, particularly for the motor milestones may occur in untreated children. Intellectual development is normal in most children. Severe and recurrent hypoglycemia, severe hepatomegaly, and postprandial lactic acidosis have been described in some cases. Although previously believed to be a benign condition, recent reports suggest that this is not the case. Liver fibrosis and hepatocellular carcinoma have been reported in patients with GSD VI. Presence of these complications suggests that long-term monitoring of hepatic status is necessary in GSD VI patients.

### 2. GSD IX

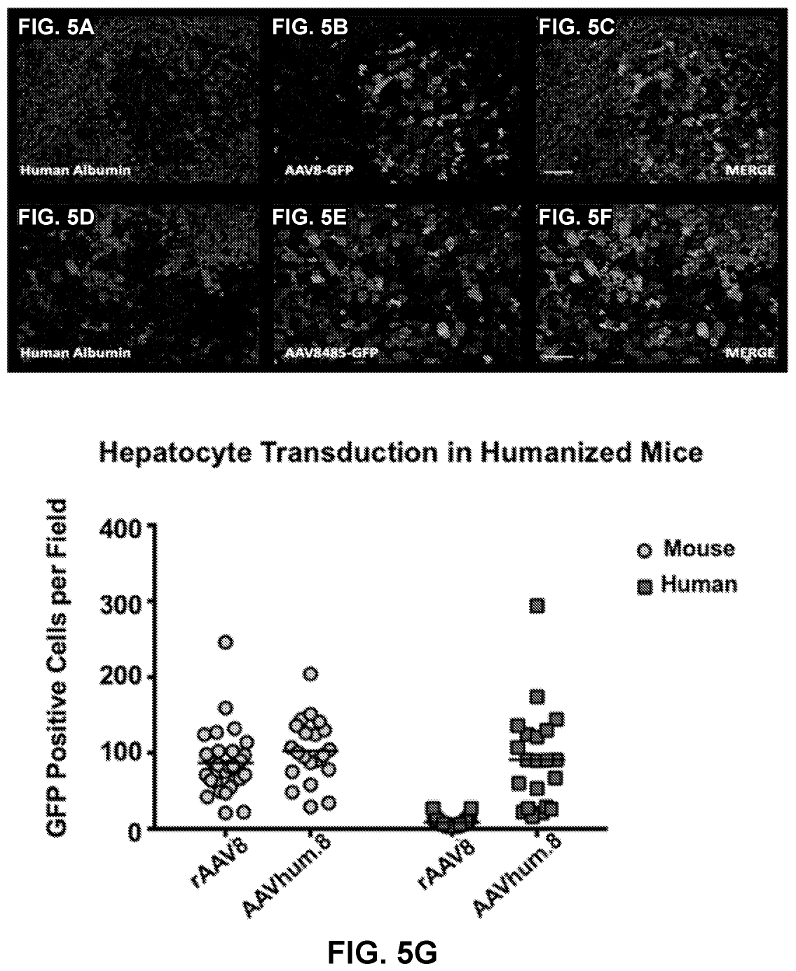

GSD IX results from deficiency of phosphorylase kinase (PhK). Specifically, PhK activates phosphorylase that catalyzes the sequential cleavage of the terminal unites from the glycogen chains, liberating glucose-1-phosphate, which is then converted to glucose-6-phosphate (FIG. 5). PhK is a protein kinase that phosphorylates the inactive form of glycogen phosphorylase, phosphorylase b, to produce the active form, phosphorylase a. The deficiency of liver PhK prevents adequate breakdown of glycogen into glucose, leading to hypoglycemia, ketosis, increased glycogen in the liver, hepatomegaly, growth delay, and elevated liver enzymes. (Willems P J, et al. (1990) Eur. J. Pediatr. 149:268-271; Li L, et al. (2018) J. Pediatr. Endocrinol. Metab. 31 (2018) 331-338; Kishnani P S, et al. (2019) Genet. Med. 21:772-789; Roscher, et al. (2014) Mol. Genet. Metab. 113:171-176; Herbert M, et al. (2011) University of Washington; Wolfsdorf J I, et al. (1999) Endocrinol. Metab. Clin. North Am. 28:801-823).

Research into liver GSD IX is severely limited, which is due, in part, to the complexity of the PhK enzyme. PhK is a heterotetramer composed of four copies each of α, β, γ, and δ subunits. The γ subunit contains the catalytic site. Its activity is regulated by the phosphorylation state of the regulatory α and β subunits, and by the δ subunit (calmodulin) via calcium levels. The α-subunit is encoded by the PHKA1 (OMIM *311870) gene in muscle and by the PHKA2 (OMIM *300798) gene in liver. There are also muscle and liver isoforms of the γ-subunit, each also encoded by different genes: PHKG1 (OMIM *172470) in muscle and PHKG2 (OMIM *172471) in liver. There is only one gene encoding the 0-subunit, PHKB (OMIM *172490), but it is differentially spliced in different tissues including muscle, liver, and brain. The genes PHKA1 and PHKG1 encode the muscle specific isoform of the al subunit and γ1 subunit, respectively. The genes PHKA2 and PHKG2 encode the liver isoform α2 and γ2 subunits, respectively, while the gene PHKB encodes the β subunit in both the liver and muscle isoforms. Moreover, the 6-subunit of PhK, calmodulin, is encoded by three different genes—CALM1 (OMIM *114180), CALM2 (OMIM *114182), and CALM3 (OMIM *114183)—which are ubiquitously expressed and involved in other cellular processes as well. (Brushia R J, et al. (1999) Front. Biosci. 4:618-641; Venien-Bryan C, et al. (2009) Structure. 17:117-127; Skamnaki V T, et al. (1999) Biochemistry. 38:14718-14730; Lowe E D, et al. (1997) EMBO J. 16:6646-6658; Owen D J, et al. (1995) Structure. 3:467-482). Pathogenic variants in the PHKA2, PHKB, and PHKG2 genes have been identified in patients with liver GSD IX. (Table 3).

TABLE 3
Phosphorylase Kinase (PhK) Subunit
Genes Known to Cause PhK Deficiency

| | PhK | | | Primarily Affected |
|---|---|---|---|---|
| Gene | Subunit | Location | Inheritance | Tissue/Organ |
| PHKA1 | α | Xq13.1 | X-linked | Muscle |
| PHKA2 | α | Xp22.13 | X-linked | Liver |
| PHKB | β | 16q12.1 | Autosomal recessive | Liver/muscle |
| PHKG2 | γ | 16p11.2 | Autosomal recessive | Liver |

Mutations in the α2 subunit are most common, X-linked recessive, and responsible for roughly 75% of liver PhK deficiency. Mutations in the γ2 subunit are second most common, autosomal recessive, and responsible for almost 25% of liver PhK deficiency. Finally, mutations in the β subunit are also autosomal recessive and are far less common. Accordingly, liver GSD IX can be divided into three subtypes based on the gene in which pathogenic variants occur (PHKA2, PHKB, and PHKG2). Until the recent availability of gene panels and exome sequencing, the diagnosis of liver GSD IX did not allow for differentiation of these subtypes. But, it is now known that pathogenic variants in three of the eight genes that encode PhK subunits are associated with liver PhK deficiency—PHKA2 GSD IX (GSD IX α2), PHKB GSD IX (GSD IX β), and PHKG2 GSD IX (GSD IX γ²), respectively.

a. Mutations in the α2 Subunit

As stated above, the most common subtype of liver PhK deficiency, accounting for about 75% of GSD IX cases, is caused by pathogenic variants in the X-linked PHKA2 gene (and is also known as X-linked glycogenosis (XLG)). While XLG was historically described as a mild or even benign condition, a wide range of clinical severity resulting from pathogenic variants in PHKA2 has emerged over recent years, even among individuals with the same pathogenic variant. As this is an X-linked condition, symptoms of liver PhK deficiency are more often seen in males. However, some female carriers also exhibit symptoms ranging from mild hepatomegaly to more severe manifestations based on X inactivation. Affected male children typically present in the first year or two of life with hepatomegaly, of varying degrees, and growth delay/deceleration. Further investigation often reveals mild to markedly elevated serum transaminases and hyperlipidemia. Ketotic hypoglycemia, if present, varies from occasional (only occurring after long fasts or during times of reduced intake when ill) to recurrent in some cases. Some patients have mild hypotonia in early childhood. Developmental delay has been reported. The clinical symptoms and laboratory abnormalities tend to improve with age. Puberty may be delayed, but normal height and complete sexual development can be eventually achieved. Most adults with X-linked liver PhK deficiency are reportedly asymptomatic. Some patients have a relatively mild course with reports of asymptomatic hepatomegaly. However, at the other end of the spectrum, there are patients with severe recurrent hypoglycemia, requiring nighttime tube feeding, and patients with liver cirrhosis due to PHKA2 and PHKG2-related GSD IX. Table 4 shows a comparison of the clinical presentation of patients with GSD IX α2 and GSD IX γ2.

TABLE 4
Comparison of Clinical Presentation of GSD
IX α2 and GSD IX γ2 Patients

| | GSD IX α2 | | GSD IX γ2 | |
|---|---|---|---|---|
| Age at Diagnosis (y) | 4 | | 1.81 | |
| Patients with hepatomegaly | 164/176 | (93.2%) | 30/30 | (100%) |
| Patients with hypoglycemia | 53/121 | (44%) | 18/19 | (94.7%) |
| Patients with ↑ AST/ALT | 125/138 | (90.6%) | 22/22 | (100%) |
| Age at Biopsy (y) | 3 | | 3 | |
| # of Patients with Biopsy | 46 | | 24 | |
| Mean Score using IASL (0-4) | 1.13 | | 2.7 | |
| Patients with Liver | 22/46 | (47.8%) | 23/24 | (95.8%) |
| Fibrosis/Cirrhosis | | | | |

b. Mutations in the β Subunit

Pathogenic variants in the PHKB gene cause an autosomal recessive form of PhK deficiency. The clinical symptoms of fewer than 20 patients have been reported all of whom have liver involvement ranging from less severe to severe. Patients typically come to medical attention due to hepatomegaly. Hypoglycemia can be mild. Liver fibrosis was reported in one patient and an adenoma-like mass was described in another. Interventricular septal hypertrophy was found in one patient. The PHKB gene is widely expressed and differentially spliced in different tissues; exon 26 is muscle specific, and exon 27 is present in non-muscle PhK B transcripts, including liver. Therefore, most pathogenic variants in PHKB are expected to cause PhK deficiency in liver and muscle. Despite this, muscle symptoms are either mild or absent, and patients with this subtype cannot be distinguished from those with PHKA2 or PHKG2 pathogenic variants on clinical basis alone.

c. Mutations in the γ2 Subunit

Pathogenic variants in the PHKG2 gene cause an autosomal recessive form of PhK deficiency. Pathogenic variants in the PHKG2 gene are associated with more severe clinical and biochemical abnormalities including increased risk for liver fibrosis and cirrhosis. About 25 cases have been described in the literature. Where information is available, most the cases reported show evidence of fibrosis on liver biopsy, and about 50% have evidence of cirrhosis. Liver cirrhosis can develop as early as the first few years of life. Occasional findings include bile duct proliferation, cholestasis, cirrhosis related esophageal varices, and splenomegaly. Several patients with PHKG2 pathogenic variants have been reported with liver adenomas, one with renal tubulopathy related to the development of rickets, and one with significant hypocalcemia. Muscle symptoms, including mild to moderate hypotonia, weakness, and amyotrophy, as well as delayed gross motor milestones have been reported in some patients. There is growing evidence from the literature that GSD IX γ2 is associated with a severe pathological phenotype. (Fernandes S A, et al. (2020) Mol. Genet. Metab. 131(3):299-305). Secondary to liver glycogen accumulation, GSD IX γ2 patients present with liver-specific symptoms including hypoglycemia, growth delay, hepatomegaly, and elevated liver enzymes. The majority of individuals with GSD IX γ2 progress to develop severe liver disease. (Kishnani P S, et al. (2019a); Kishnani P S, et al. (2019) Hum. Mol. Genet. 28(R1):R31-R41; Roscher, et al. (2014) Mol. Genet. Metab. 113:171-176; Herbert M, et al. (2011) University of Washington, Seattle; Femandes S A, et al. (2020); Beauchamp N J, et al. (2007) Mol. Genet. Metab. 92:88-99; Bali D S, et al. (2014) Mol. Genet. Metab. 111:309-313; Burwinkel et al. (1998) Hum. Mol. Genet. 7:149-154; Burwinkel, et al. (2000) J. Med. Genet. 37:376-7; Sovik O, et al. (1982) Eur. J. Pediatr. 139:210; Albash F, et al. (2014) Eur. J. Pediatr. 173:647-653; Van Beurden EACM, et al. (1997) Biochem. Biophys. Res. Commun. 236:544-548.) Of published case reports for patients with GSD IX γ2, 95.8% of patients that received a liver biopsy showed liver fibrosis and/or cirrhosis. (Femandes S A, et al. (2020)). Ultimately, individuals with GSD IX γ2 are at high risk for developing severe progressive liver disease including progressive liver fibrosis, elevated liver enzymes, and decline in liver function, potentially necessitating liver transplant. (Kishnani P S, et al. (2019a); Herbert M, et al. (2011); Fernandes S A, et al. (2020); Roscher et al. (2014) Mol. Genet. Metab. 113:171-176; Beauchamp N J, et al. (2007) Mol. Genet. Metab. 92:88-99; Burwinkel et al. (1998); Sovik O, et al. (1982) Eur. J. Pediatr. 139:210; Albash F, et al. (2014) Eur. J. Pediatr. 173:647-653; Van Beurden EACM, et al. (1997) Biochem. Biophys. Res. Commun. 236:544-548).

The standard of care for individuals with GSD VI and/or GSD IX is dietary modification via consumption of high protein meals with supplements of uncooked cornstarch. High protein intake provides repletion of protein precursors necessary for maintaining gluconeogenesis. (Kishnani P S, et al. (2019b); Herbert M, et al. (2011) University of Washington, Seattle; Wolfsdorf et al., 1999) and reducing carbohydrate intake. Cornstarch, a source of the plant-based glycogen analogue amylopectin, is broken down in the gastrointestinal tract and provides a slow-release form of glucose in-between meals. (Kishnani et al., 2019; Herbert et al., 2011; Wolfsdorf J I, et al. (1999) Metab. Clin. North Am. 28:801-823). Dietary modification provides symptomatic improvement of hypoglycemic episodes (hypoglycemia can be with and without ketosis), but does not address the continued buildup of glycogen in the liver, the underlying pathophysiology of the disease. Despite the life-threatening severity of GSD VI and GSD IX, there are currently no non-surgical, minimally invasive, long-term, therapeutic options for patients.

### C. Compositions for Treating and/or Preventing GSD IX and/or GSD VI Disease Progression

### 1. Nucleic Acid Molecules

Disclosed herein is an isolated nucleic acid molecule comprising a nucleic acid sequence encoding a polypeptide capable of preventing glycogen accumulation and/or degrading accumulated glycogen. Disclosed herein is an isolated nucleic acid molecule comprising a nucleic acid sequence encoding a polypeptide capable of reducing the expression level and/or activity level of glycogen synthase. Disclosed herein is an isolated nucleic acid molecule comprising a nucleic acid sequence encoding a polypeptide capable of preventing glycogen accumulation and/or degrading accumulated glycogen, wherein the nucleic acid sequence is CpG depleted and codon-optimized for expression in a human cell.

Disclosed herein is an isolated nucleic acid molecule comprising a nucleic acid sequence encoding a polypeptide capable of reducing the expression level and/or activity level of glycogen synthase wherein the nucleic acid sequence is CpG depleted and codon-optimized for expression in a human cell. Disclosed herein is an isolated nucleic acid molecule comprising a nucleic acid sequence encoding a polypeptide capable of preventing glycogen accumulation and/or degrading accumulated glycogen, and a nucleic acid sequence encoding a polypeptide capable of reducing the expression level and/or activity level of glycogen synthase.

Disclosed herein is an isolated nucleic acid molecule comprising a nucleic acid sequence encoding a polypeptide capable of restoring one or more aspects of the glycogen metabolic pathway. For example, in an aspect, a disclosed isolated nucleic acid can restore the balance of glycogen synthesis and degradation. Disclosed herein is an isolated nucleic acid molecule comprising a nucleic acid sequence encoding a polypeptide capable restoring one or more aspects of the glycogen metabolic pathway, wherein the nucleic acid sequence is CpG-depleted and codon-optimized for expression in a human cell. Disclosed herein is an isolated nucleic acid molecule comprising a nucleic acid sequence encoding a polypeptide capable of restoring one or more aspects of the glycogen metabolic pathway, and a nucleic acid sequence encoding a polypeptide capable of reducing the expression level and/or activity level of glycogen synthase.

Disclosed herein is an isolated nucleic acid molecule comprising a nucleic acid sequence encoding a polypeptide capable of restoring one or more aspects of the glycogenolysis metabolic pathway. Disclosed herein is an isolated nucleic acid molecule comprising a nucleic acid sequence encoding a polypeptide capable restoring one or more aspects of the glycogenolysis metabolic pathway, wherein the nucleic acid sequence is CpG-depleted and codon-optimized for expression in a human cell. Disclosed herein is an isolated nucleic acid molecule comprising a nucleic acid sequence encoding a polypeptide capable of restoring one or more aspects of the glycogenolysis metabolic pathway, and a nucleic acid sequence encoding a polypeptide capable of reducing the expression level and/or activity level of glycogen synthase.

Disclosed herein is an isolated nucleic acid molecule comprising a nucleic acid sequence encoding a polypeptide capable of restoring one or more aspects of the glycogenesis metabolic pathway. Disclosed herein is an isolated nucleic acid molecule comprising a nucleic acid sequence encoding a polypeptide capable restoring one or more aspects of the glycogenesis metabolic pathway, wherein the nucleic acid sequence is CpG-depleted and codon-optimized for expression in a human cell. Disclosed herein is an isolated nucleic acid molecule comprising a nucleic acid sequence encoding a polypeptide capable of restoring one or more aspects of the glycogenesis metabolic pathway, and a nucleic acid sequence encoding a polypeptide capable of reducing the expression level and/or activity level of glycogen synthase.

Disclosed herein is an isolated nucleic acid molecule comprising a nucleic acid sequence encoding a polypeptide capable of restoring PhK subunit activity and/or functionality. Disclosed herein is an isolated nucleic acid molecule comprising a nucleic acid sequence encoding a polypeptide capable restoring PhK subunit activity and/or functionality, wherein the nucleic acid sequence is CpG-depleted and codon-optimized for expression in a human cell. Disclosed herein is an isolated nucleic acid molecule comprising a nucleic acid sequence encoding a polypeptide capable of restoring PhK subunit activity and/or functionality, and a nucleic acid sequence encoding a polypeptide capable of reducing the expression level and/or activity level of glycogen synthase.

In an aspect, a disclosed nucleic acid molecule can restore the functionality and/or structural integrity of the PhK complex. For example, in an aspect, by restoring the functionality and/or structural integrity of a subunit (such as α2, δ, β, and γ2), then the functionality and/or structural integrity of the PhK complex (the heterotetramer) can be restored.

In an aspect, a disclosed nucleic acid sequence can comprise a coding sequence that is less than about 4.5 kilobases. In an aspect, a disclosed encoded polypeptide can degrade glycogen.

In an aspect, a disclosed encoded polypeptide can comprise a phosphorylase kinase. In an aspect, a disclosed encoded polypeptide can comprise a subunit of a phosphorylase kinase. In an aspect, an encoded polypeptide can be glycogen phosphorylase kinase regulatory subunit alpha 1 (PhK al). In an aspect, an encoded polypeptide can be glycogen phosphorylase kinase regulatory subunit alpha 2 (PhK α2). In an aspect, an encoded polypeptide can be glycogen phosphorylase kinase regulatory subunit beta (PhK β). In an aspect, a disclosed nucleic acid sequence can comprise the sequence for the glycogen phosphorylase kinase regulatory subunit delta (CALM1, CALM2, and/or CALM3). In an aspect, an encoded polypeptide can be glycogen phosphorylase kinase catalytic subunit gamma 2 (PhK γ2). In an aspect, an encoded polypeptide can be glycogen phosphorylase liver form (PYGL). In an aspect, a disclosed encoded polypeptide can be derived from human or non-human. In an aspect, a disclosed encoded phosphorylase kinase polypeptide or a disclosed encoded phosphorylase can be derived from human or non-human.

In an aspect, a disclosed encoded PhK al polypeptide can comprise the sequence set forth in SEQ ID NO:01, SEQ ID NO:02, SEQ ID NO:03, or a fragment thereof. In an aspect, a disclosed encoded PhK al can comprise a sequence having at least 40-59%, at least 60-79%, or at least 80-99% identity to the sequence set forth in SEQ ID NO:01, SEQ ID NO:02, SEQ ID NO:03, or a fragment thereof. In an aspect, a disclosed encoded PhK al can comprise the sequence set forth in Accession No. NP 001116142.1, NP 001165907.1, NP_002628.2, XP_006724724.1, or a fragment thereof. In an aspect, a disclosed encoded PhK al can comprise a sequence having at least 40-59%, at least 60-79%, or at least 80-99% identity to the sequence set forth in Accession No. NP_001116142.1, NP_001165907.1, NP_002628.2, XP_006724724.1, or a fragment thereof.

In an aspect, a disclosed encoded PhK α2 can comprise the sequence set forth in SEQ ID NO:04 or a fragment thereof. In an aspect, a disclosed encoded PhK α2 can comprise a sequence having at least 30%, at least 40%, at least 50%, at least 60%, at least 70%, at least 80%, or at least 90% identity to the sequence set forth in SEQ ID NO:04 or a fragment thereof. In an aspect, a disclosed encoded PhK α2 can comprise the sequence set forth in Accession No. NP_000283.1, XP 005274605.1, XP 005274607.1, XP 006724559.1, XP_006724561.1, XP_011543839.1, XP_011543840.1, XP_016885069.1, or a fragment thereof. In an aspect, a disclosed encoded PhK α2 can comprise a sequence having at least 30%, at least 40%, at least 50%, at least 60%, at least 70%, at least 80%, or at least 90% identity to the sequence set forth in Accession No. NP_000283.1, XP_005274605.1, XP_005274607.1, XP_006724559.1, XP_006724561.1, XP_011543839.1, XP_011543840.1, XP_016885069.1, or a fragment thereof.

In an aspect, a disclosed encoded PhK β can comprise the sequence set forth in SEQ ID NO:05, SEQ ID NO:06, SEQ ID NO:07, or a fragment thereof. In an aspect, a disclosed encoded PhK β can comprise a sequence having at least 30%, at least 40%, at least 50%, at least 60%, at least 70%, at least 80%, or at least 90% identity to the sequence set forth in SEQ ID NO:05, SEQ ID NO:06, SEQ ID NO:07, or a fragment thereof. In an aspect, a disclosed encoded PhK β can comprise the sequence set forth in Accession No. NP_000284.1, NP_001027005.1, NP_001350766.1, or a fragment thereof. In an aspect, a disclosed encoded PhK β can comprise a sequence having at least 30%, at least 40%, at least 50%, at least 60%, at least 70%, at least 80%, or at least 90% identity to the sequence set forth in Accession No. NP_000284.1, NP_001027005.1, NP_001350766.1, or a fragment thereof.

In an aspect, a disclosed encoded PhK δ can comprise the sequence set forth in SEQ ID NO:57, SEQ ID NO:58, SEQ ID NO:59, or a fragment thereof. In an aspect, a disclosed encoded PhK β can comprise a sequence having at least 30%, at least 40%, at least 50%, at least 60%, at least 70%, at least 80%, or at least 90% identity to the sequence set forth in SEQ ID NO:57, SEQ ID NO:58, SEQ ID NO:59, or a fragment thereof. In an aspect, a disclosed encoded PhK δ can comprise the sequence set forth in Accession No. NP_001350599, NP_008819, NP_001350598, or a fragment thereof. In an aspect, a disclosed encoded PhK β can comprise a sequence having at least 30%, at least 40%, at least 50%, at least 60%, at least 70%, at least 80%, or at least 90% identity to the sequence set forth in Accession No. NP_001350599, NP_008819, NP_001350598, or a fragment thereof.

In an aspect, a disclosed encoded PhK γ2 can comprise the sequence set forth in SEQ ID NO:08, SEQ ID NO:09, or a fragment thereof. In an aspect, a disclosed encoded PhK γ2 can comprise a sequence having at least 30%, at least 40%, at least 50%, at least 60%, at least 70%, at least 80%, or at least 90% identity to the sequence set forth in SEQ ID NO:08, SEQ ID NO:09, or a fragment thereof. In an aspect, a disclosed encoded PhK γ2 can comprise the sequence set forth in Accession No. NP_000285.1, NP_001165903.1, or a fragment thereof. In an aspect, a disclosed encoded PhK γ2 can comprise a sequence having at least 30%, at least 40%, at least 50%, at least 60%, at least 70%, at least 80%, or at least 90% identity to the sequence set forth in Accession No. NP_000285.1, NP_001165903.1, or a fragment thereof.

In an aspect, a disclosed encoded PYGL can comprise the sequence set forth in SEQ ID NO:10, [0162] In an aspect, a disclosed CALM1 can comprise the sequence set forth in SEQ ID NO:60 or a fragment thereof. In an aspect, a disclosed CALM2 can comprise the sequence set forth in SEQ ID NO:61 or a fragment thereof. In an aspect, a disclosed CALM3 can comprise the sequence set forth in SEQ ID NO:62 or a fragment thereof. In an aspect, a disclosed CALM1 can comprise a sequence having about 30%, about 40%, about 50%, about 60%, about 70%, about 80%, about 85%, about 90%, about 95%, or greater than 95% identity than the sequence set forth in SEQ ID NO:60, SEQ ID NO:61, or SEQ ID NO:62, or a fragment thereof. In an aspect, a disclosed encoded PYGL can comprise a sequence having at least 30%, at least 40%, at least 50%, at least 60%, at least 70%, at least 80%, or at least 90% identity to the sequence set forth in SEQ ID NO:10, SEQ ID NO:11, or a fragment thereof. In an aspect, a disclosed encoded PYGL can comprise the sequence set forth in Accession No. NP_001157412.1, NP_002854.3, or a fragment thereof. In an aspect, a disclosed encoded PYGL can comprise a sequence having at least 30%, at least 40%, at least 50%, at least 60%, at least 70%, at least 80%, or at least 90% identity to the sequence set forth in Accession No. NP_001157412.1, NP_002854.3, or a fragment thereof.

In an aspect, a disclosed encoded GYS1 can comprise the sequence set forth in SEQ ID NO:41, SEQ ID NO:42, or a fragment thereof. In an aspect, a disclosed encoded GYS1 can comprise a sequence having at least 30%, at least 40%, at least 50%, at least 60%, at least 70%, at least 80%, or at least 90% identity to the sequence set forth in SEQ ID NO:41, SEQ ID NO:42, or a fragment thereof. In an aspect, a disclosed encoded GYS1 can comprise the sequence set forth in Accession No. NP_001155059.1, NP_002094.2, or a fragment thereof. In an aspect, a disclosed encoded GYS1 can comprise a sequence having at least 30%, at least 40%, at least 50%, at least 60%, at least 70%, at least 80%, or at least 90% identity to the sequence set forth in Accession No. NP_001155059.1, NP_002094.2, or a fragment thereof.

In an aspect, a disclosed encoded GYS2 can comprise the sequence set forth in SEQ ID NO:43 or a fragment thereof. In an aspect, a disclosed encoded GYS2 can comprise a sequence having at least 30%, at least 40%, at least 50%, at least 60%, at least 70%, at least 80%, or at least 90% identity to the sequence set forth in SEQ ID NO:43 or a fragment thereof. In an aspect, a disclosed encoded GYS2 can comprise the sequence set forth in Accession No. NP_068776.2, XP_006719126.1, XP_016874734.1, XP_024304728.1, or a fragment thereof. In an aspect, a disclosed encoded GYS2 can comprise a sequence having at least 30%, at least 40%, at least 50%, at least 60%, at least 70%, at least 80%, or at least 90% identity to the sequence set forth in Accession No. NP_068776.2, XP_006719126.1, XP 016874734.1, XP_024304728.1, or a fragment thereof.

In an aspect, a disclosed nucleic acid sequence can comprise the sequence for a phosphorylase kinase. In an aspect, a disclosed nucleic acid sequence can comprise the sequence for a subunit of a phosphorylase kinase. In an aspect, a disclosed nucleic acid sequence can comprise the sequence for the glycogen phosphorylase kinase regulatory subunit alpha 1 (PHKA1). In an aspect, a disclosed nucleic acid sequence can comprise the sequence for the glycogen phosphorylase kinase regulatory subunit alpha 2 (PHKA2). In an aspect, a disclosed nucleic acid sequence can comprise the sequence for the glycogen phosphorylase kinase regulatory subunit beta (PHKB). In an aspect, a disclosed nucleic acid sequence can comprise the sequence for the glycogen phosphorylase kinase regulatory subunit delta (CALM1, CALM2, and/or CALM3). In an aspect, a disclosed nucleic acid sequence can comprise the sequence for the glycogen phosphorylase kinase catalytic subunit gamma 2 (PHKG2). In an aspect, a disclosed nucleic acid sequence can comprise the sequence for glycogen phosphorylase liver form (PYGL). In an aspect, a disclosed nucleic acid sequence can be derived from human or non-human. In an aspect, a disclosed nucleic acid sequence for phosphorylase kinase or a disclosed phosphorylase can be derived from human or non-human.

In an aspect, a disclosed nucleic acid sequence for PHKA1 can comprise the sequence set forth in SEQ ID NO:12, SEQ ID NO:13, SEQ ID NO:14, or a fragment thereof. In an aspect, a disclosed nucleic acid sequence for PHKA1 can comprise a sequence having at least 40-59%, at least 60-79%, or at least 80-99% identity to the sequence set forth in Accession No. SEQ ID NO:12, SEQ ID NO:13, SEQ ID NO:14, or a fragment thereof. In an aspect, a disclosed nucleic acid sequence for PHKA1 can comprise the sequence set forth in NM_001122670.2, NM_001172436.2, NM_002637.4, XM_006724661.2, or a fragment thereof. In an aspect, a disclosed nucleic acid sequence for PHKA1 can comprise a sequence having at least 40-59%, at least 60-79%, or at least 80-99% identity to the sequence set forth in NM_001122670.2, NM_001172436.2, NM_002637.4, XM_006724661.2, or a fragment thereof. In an aspect, a disclosed PHKA1 gene can comprise the sequence set forth in Accession No. NG_016599.2.

In an aspect, a disclosed nucleic acid sequence for PHKA2 can comprise the sequence set forth in SEQ ID NO:15, SEQ ID NO:16, or a fragment thereof. In an aspect, a disclosed nucleic acid sequence for PHKA2 can comprise a sequence having at least 30%, at least 40%, at least 50%, at least 60%, at least 70%, at least 80%, or at least 90% identity to the sequence set forth in SEQ ID NO:15, SEQ ID NO:16, or a fragment thereof. In an aspect, a disclosed nucleic acid sequence for PHKA2 can comprise the sequence set forth in Accession No. NM_000292.3, XM_011545537.3, XM 005274550.5, XM 006724496.4, XM 006724498.4, XM_017029580.2, XM_011545538.3, XM_005274548.5, or a fragment thereof. In an aspect, a disclosed nucleic acid sequence for PHKA2 can comprise a sequence having at least 30%, at least 40%, at least 50%, at least 60%, at least 70%, at least 80%, or at least 90% identity to the sequence set forth in Accession No. NM_000292.3, XM_011545537.3, XM_005274550.5, XM_006724496.4, XM_006724498.4, XM_017029580.2, XM_011545538.3, XM_005274548.5, or a fragment thereof. In an aspect, a disclosed PHKA2 gene can comprise the sequence set forth in Accession No. NG_016622.1.

In an aspect, a disclosed nucleic acid sequence for PHKB can comprise the sequence set forth in SEQ ID NO:17 or a fragment thereof. In an aspect, a disclosed nucleic acid sequence for PHKB can comprise a sequence having at least 30%, at least 40%, at least 50%, at least 60%, at least 70%, at least 80%, or at least 90% identity to the sequence set forth in SEQ ID NO:17 or a fragment thereof. In an aspect, a disclosed nucleic acid sequence for PHKB can comprise the sequence set forth in Accession No. NM_000293.3, NM_001031835.3, NM_001363837.1, or a fragment thereof. In an aspect, a disclosed nucleic acid sequence for PHKB can comprise a sequence having at least 30%, at least 40%, at least 50%, at least 60%, at least 70%, at least 80%, or at least 90% identity to the sequence set forth in Accession No. NM_000293.3, NM_001031835.3, NM_001363837.1, or a fragment thereof. In an aspect, a disclosed PHKB gene can comprise the sequence set forth in Accession No. NG_016598.1.

In an aspect, a disclosed nucleic acid sequence for CALM1 an comprise the sequence set forth in SEQ ID NO:60 or a fragment thereof. In an aspect, a disclosed nucleic acid sequence for CALM1 can comprise a sequence having at least 30%, at least 40%, at least 50%, at least 60%, at least 70%, at least 80%, or at least 90% identity to the sequence set forth in SEQ ID NO:60 or a fragment thereof. In an aspect, a disclosed nucleic acid sequence for CALM2 an comprise the sequence set forth in SEQ ID NO:61 or a fragment thereof. In an aspect, a disclosed nucleic acid sequence for CALM2 can comprise a sequence having at least 30%, at least 40%, at least 50%, at least 60%, at least 70%, at least 80%, or at least 90% identity to the sequence set forth in SEQ ID NO:61 or a fragment thereof. In an aspect, a disclosed nucleic acid sequence for CALM3 an comprise the sequence set forth in SEQ ID NO:62 or a fragment thereof. In an aspect, a disclosed nucleic acid sequence for CALM3 can comprise a sequence having at least 30%, at least 40%, at least 50%, at least 60%, at least 70%, at least 80%, or at least 90% identity to the sequence set forth in SEQ ID NO:62 or a fragment thereof.

In an aspect, a disclosed nucleic acid sequence for PHKG2 can comprise the sequence set forth in SEQ ID NO:18, SEQ ID NO:19, SEQ ID NO:20, or a fragment thereof. In an aspect, a disclosed nucleic acid sequence for PHKG2 can comprise a sequence having at least 30%, at least 40%, at least 50%, at least 60%, at least 70%, at least 80%, or at least 90% identity to the sequence set forth in SEQ ID NO:18, SEQ ID NO:19, SEQ ID NO:20, or a fragment thereof. In an aspect, a disclosed nucleic acid sequence for PHKG2 can comprise the sequence set forth in Accession No. NM_000294.3, NM_001172432.2, or a fragment thereof. In an aspect, a disclosed nucleic acid sequence for PHKG2 can comprise a sequence having at least 30%, at least 40%, at least 50%, at least 60%, at least 70%, at least 80%, or at least 90% identity to the sequence set forth in Accession No. NM_000294.3, NM_001172432.2, or a fragment thereof. In an aspect, a disclosed PHKG2 gene can comprise the sequence set forth in Accession No. NG_016616.2.

In an aspect, a disclosed nucleic acid sequence for PYGL can comprise the sequence set forth in SEQ ID NO:21, SEQ ID NO:22, or a fragment thereof. In an aspect, a disclosed nucleic acid sequence for PYGL can comprise a sequence having at least 30%, at least 40%, at least 50%, at least 60%, at least 70%, at least 80%, or at least 90% identity to the sequence set forth in SEQ ID NO:21, SEQ ID NO:22, or a fragment thereof. In an aspect, a disclosed nucleic acid sequence for PYGL can comprise the sequence set forth in Accession No. NM_001163940.2, NM_002863.5, or a fragment thereof. In an aspect, a disclosed nucleic acid sequence for PYGL can comprise a sequence having at least 30%, at least 40%, at least 50%, at least 60%, at least 70%, at least 80%, or at least 90% identity to the sequence set forth in Accession No. NM_001163940.2, NM_002863.5, or a fragment thereof. In an aspect, a disclosed PYGL gene can comprise the sequence set forth in Accession No. NG_012796.1.

In an aspect, a disclosed nucleic acid sequence for GYS1 can comprise the sequence set forth in Accession No. NM_001161587.2, NM_002103.5, or a fragment thereof. In an aspect, a disclosed nucleic acid sequence for GYS1 can comprise a sequence having at least 30%, at least 40%, at least 50%, at least 60%, at least 70%, at least 80%, or at least 90% identity to the sequence set forth in Accession No. NM_001161587.2, NM_002103.5, or a fragment thereof.

In an aspect, a disclosed nucleic acid sequence for GYS2 can comprise the sequence set forth in Accession No. NM 021957.4, XM_024448960.1, XM 006719063.3, XM_017019245.2, or a fragment thereof. In an aspect, a disclosed nucleic acid sequence for GYS2 can comprise a sequence having at least 30%, at least 40%, at least 50%, at least 60%, at least 70%, at least 80%, or at least 90% identity to the sequence set forth in Accession No. NM_021957.4, XM_024448960.1, XM_006719063.3, XM_017019245.2, or a fragment thereof.

a. Polypeptide Sequence
In an aspect, a disclosed PhK α1 subunit can comprise the following sequence or a
fragment thereof:
MRSRSNSGVRLDGYARLVQQTILCHQNPVTGLLPASYDQKDAWVRDNVYSILAVWGL
GLAYRKNADRDEDKAKAYELEQSVVKLMRGLLHCMIRQVDKVESFKYSQSTKDSLHA
KYNTKTCATVVGDDQWGHLQLDATSVYLLFLAQMTASGLHIIHSLDEVNFIQNLVFYIE
AAYKTADFGIWERGDKTNQGISELNASSVGMAKAALEALDELDLFGVKGGPQSVIHVL
ADEVQHCQSILNSLLPRASTSKEVDASLLSVVSFPAFAVEDSQLVELTKQEIITKLQGRY
GCCRFLRDGYKTPKEDPNRLYYEPAELKLFENIECEWPLFWTYFILDGVFSGNAEQVQE
YKEALEAVLIKGKNGVPLLPELYSVPPDRVDEEYQNPHTVDRVPMGKLPHMWGQSLYI
LGSLMAEGFLAPGEIDPLNRRFSTVPKPDVVVQVSILAETEEIKTILKDKGIYVETIAEVY
PIRVQPARILSHIYSSLGCNNRMKLSGRPYRHMGVLGTSKLYDIRKTIFTFTPQFIDQQQF
YLALDNKMIVEMLRTDLSYLCSRWRMTGQPTITFPISHSMLDEDGTSLNSSILAALRKM
QDGYFGGARVQTGKLSEFLTTSCCTHLSFMDPGPEGKLYSEDYDDNYDYLESGNWMN
DYDSTSHARCGDEVARYLDHLLAHTAPHPKLAPTSQKGGLDRFQAAVQTTCDLMSLV
TKAKELHVQNVHMYLPTKLFQASRPSFNLLDSPHPRQENQVPSVRVEIHLPRDQSGEVD
FKALVLQLKETSSLQEQADILYMLYTMKGPDWNTELYNERSATVRELLTELYGKVGEI
RHWGLIRYISGILRKKVEALDEACTDLLSHQKHLTVGLPPEPREKTISAPLPYEALTQLID
EASEGDMSISILTQEIMVYLAMYMRTQPGLFAEMFRLRIGLIIQVMATELAHSLRCSAEE
ATEGLMNLSPSAMKNLLHHILSGKEFGVERSVRPTDSNVSPAISIHEIGAVGATKTERTGI
MQLKSEIKQSPGTSMTPSSGSFPSAYDQQSSKDSRQGQWQRRRRLDGALNRVPVGFYQ
KVWKVLQKCHGLSVEGFVLPSSTTREMTPGEIKFSVHVESVLNRVPQPEYRQLLVEAIL
VLTMLADIEIHSIGSIIAVEKIVHIANDLFLQEQKTLGADDTMLAKDPASGICTLLYDSAP
SGRFGTMTYLSKAAATYVQEFLPHSICAMQ (SEQ ID NO: 01).
In aspect, a disclosed PhK α1 subunit can comprise the following sequence or a fragment
thereof:
MRSRSNSGVRLDGYARLVQQTILCHQNPVTGLLPASYDQKDAWVRDNVYSILAVWGL
GLAYRKNADRDEDKAKAYELEQSVVKLMRGLLHCMIRQVDKVESFKYSQSTKDSLHA
KYNTKTCATVVGDDQWGHLQLDATSVYLLFLAQMTASGLHIIHSLDEVNFIQNLVFYIE
AAYKTADFGIWERGDKTNQGISELNASSVGMAKAALEALDELDLFGVKGGPQSVIHVL
ADEVQHCQSILNSLLPRASTSKEVDASLLSVVSFPAFAVEDSQLVELTKQEIITKLQGRY
GCCRFLRDGYKTPKEDPNRLYYEPAELKLFENIECEWPLFWTYFILDGVFSGNAEQVQE
YKEALEAVLIKGKNGVPLLPELYSVPPDRVDEEYQNPHTVDRVPMGKLPHMWGQSLYI
LGSLMAEGFLAPGEIDPLNRRFSTVPKPDVVVQVSILAETEEIKTILKDKGIYVETIAEVY
PIRVQPARILSHIYSSLGCNNRMKLSGRPYRHMGVLGTSKLYDIRKTIFTFTPQFIDQQQF
YLALDNKMIVEMLRTDLSYLCSRWRMTGQPTITFPISHSMLDEDGTSLNSSILAALRKM
QDGYFGGARVQTGKLSEFLTTSCCTHLSFMDPGPEGKLYSEDYDDNYDYLESGNWMN
DYDSTSHARCGDEVARYLDHLLAHTAPHPKLAPTSQKGGLDRFQAAVQTTCDLMSLV
TKAKELHVQNVHMYLPTKLFQASRPSFNLLDSPHPRQENQVPSVRVEIHLPRDQSGEVD
FKALVLQLKETSSLQEQADILYMLYTMKGPDWNTELYNERSATVRELLTELYGKVGEI
RHWGLIRYISGILRKKVEALDEACTDLLSHQKHLTVGLPPEPREKTISAPLPYEALTQLID
EASEGDMSISILTQEIMVYLAMYMRTQPGLFAEMFRLRIGLIIQVMATELAHSLRCSAEE
ATEGLMNLSPSAMKNLLHHILSGKEFGVERSVRPTDSNVSPAISIHEIGAVGATKTERTGI
MQLKSEIKQVEFRRLSISAESQSPGTSMTPSSGSFPSAYDQQSSKDSRQGQWQRRRRLD
GALNRVPVGFYQKVWKVLQKCHGLSVEGFVLPSSTTREMTPGEIKFSVHVESVLNRVP
QPEYRQLLVEAILVLTMLADIEIHSIGSIIAVEKIVHIANDLFLQEQKTLGADDTMLAKDP
ASGICTLLYDSAPSGRFGTMTYLSKAAATYVQEFLPHSICAMQ (SEQ ID NO: 02).
In an aspect, a disclosed PhK α1 subunit can comprise the following sequence or a
fragment thereof:
MRSRSNSGVRLDGYARLVQQTILCHQNPVTGLLPASYDQKDAWVRDNVYSILAVWGL
GLAYRKNADRDEDKAKAYELEQSVVKLMRGLLHCMIRQVDKVESFKYSQSTKDSLHA
KYNTKTCATVVGDDQWGHLQLDATSVYLLFLAQMTASGLHIIHSLDEVNFIQNLVFYIE
AAYKTADFGIWERGDKTNQGISELNASSVGMAKAALEALDELDLFGVKGGPQSVIHVL
ADEVQHCQSILNSLLPRASTSKEVDASLLSVVSFPAFAVEDSQLVELTKQEIITKLQGRY
GCCRFLRDGYKTPKEDPNRLYYEPAELKLFENIECEWPLFWTYFILDGVFSGNAEQVQE
YKEALEAVLIKGKNGVPLLPELYSVPPDRVDEEYQNPHTVDRVPMGKLPHMWGQSLYI
LGSLMAEGFLAPGEIDPLNRRFSTVPKPDVVVQVSILAETEEIKTILKDKGIYVETIAEVY
PIRVQPARILSHIYSSLGCNNRMKLSGRPYRHMGVLGTSKLYDIRKTIFTFTPQFIDQQQF
YLALDNKMIVEMLRTDLSYLCSRWRMTGQPTITFPISHSMLDEDGTSLNSSILAALRKM
QDGYFGGARVQTGKLSEFLTTSCCTHLSFMDPGPEGKLYSEDYDDNYDYLESGNWMN
DYDSTSHDVHMYLPTKLFQASRPSFNLLDSPHPRQENQVPSVRVEIHLPRDQSGEVDFK
ALVLQLKETSSLQEQADILYMLYTMKGPDWNTELYNERSATVRELLTELYGKVGEIRH
WGLIRYISGILRKKVEALDEACTDLLSHQKHLTVGLPPEPREKTISAPLPYEALTQLIDEA
SEGDMSISILTQEIMVYLAMYMRTQPGLFAEMFRLRIGLIIQVMATELAHSLRCSAEEAT
EGLMNLSPSAMKNLLHHILSGKEFGVERSVRPTDSNVSPAISIHEIGAVGATKTERTGIM
QLKSEIKQSPGTSMTPSSGSFPSAYDQQSSKDSRQGQWQRRRRLDGALNRVPVGFYQK
VWKVLQKCHGLSVEGFVLPSSTTREMTPGEIKFSVHVESVLNRVPQPEYRQLLVEAILV
LTMLADIEIHSIGSIIAVEKIVHIANDLFLQEQKTLGADDTMLAKDPASGICTLLYDSAPS
GRFGTMTYLSKAAATYVQEFLPHSICAMQ (SEQ ID NO: 03).
In an aspect, a disclosed PhK α2 subunit can comprise the following sequence or a
fragment thereof:
MRSRSNSGVRLDGYARLVQQTILCYQNPVTGLLSASHEQKDAWVRDNIYSILAVWGLG
MAYRKNADRDEDKAKAYELEQNVVKLMRGLLQCMMRQVAKVEKFKHTQSTKDSLH
AKYNTATCGTVVGDDQWGHLQVDATSLFLLFLAQMTASGLRIIFTLDEVAFIQNLVFYI
EAAYKVADYGMWERGDKTNQGIPELNASSVGMAKAALEAIDELDLFGAHGGRKSVIH
VLPDEVEHCQSILFSMLPRASTSKEIDAGLLSIISFPAFAVEDVNLVNVTKNEIISKLQGRY
GCCRFLRDGYKTPREDPNRLHYDPAELKLFENIECEWPVFWTYFIIDGVFSGDAVQVQE
YREALEGILIRGKNGIRLVPELYAVPPNKVDEEYKNPHTVDRVPMGKVPHLWGQSLYIL
SSLLAEGFLAAGEIDPLNRRFSTSVKPDVVVQVTVLAENNHIKDLLRKHGVNVQSIADIH
PIQVQPGRILSHIY AKLGRNKNMNLSGRPYRHIGVLGTSKLYVIRNQIFTFTPQFTDQHHF
YLALDNEMIVEMLRIELAYLCTCWRMTGRPTLTFPISRTMLTNDGSDIHSAVLSTIRKLE
DGYFGGARVKLGNLSEFLTTSFYTYLTFLDPDCDEKLFDNASEGTFSPDSDSDLVGYLE
DTCNQESQDELDHYINHLLQSTSLRSYLPPLCKNTEDRHVFSAIHSTRDILSVMAKAKGL
EVPFVPMTLPTKVLSAHRKSLNLVDSPQPLLEKVPESDFQWPRDDHGDVDCEKLVEQL
KDCSNLQDQADILYILYVIKGPSWDTNLSGQHGVTVQNLLGELYGKAGLNQEWGLIRY
ISGLLRKKVEVLAEACTDLLSHQKQLTVGLPPEPREKIISAPLPPEELTKLIYEASGQDISI
AVLTQEIVVYLAMYVRAQPSLFVEMLRLRIGLIIQVMATELARSLNCSGEEASESLMNL
SPFDMKNLLHHILSGKEFGVERSVRPIHSSTSSPTISIHEVGHTGVTKTERSGINRLRSEM
KQMTRRFSADEQFFSVGQAASSSAHSSKSARSSTPSSPTGTSSSDSGGHHIGWGERQGQ
WLRRRRLDGAINRVPVGFYQRVWKILQKCHGLSIDGYVLPSSTTREMTPHEIKFAVHVE
SVLNRVPQPEYRQLLVEAIMVLTLLSDTEMTSIGGIIHVDQIVQMASQLFLQDQVSIGAM
DTLEKDQATGICHFFYDSAPSGAYGTMTYLTRAVASYLQELLPNSGCQMQ (SEQ ID
NO: 04).
In an aspect, a disclosed PhK β subunit can comprise the following sequence or a fragment
thereof:
MAGAAGLTAEVSWKVLERRARTKRSGSVYEPLKSINLPRPDNETLWDKLDHYYRIVKS
TLLLYQSPTTGLFPTKTCGGDQKAKIQDSLYCAAGAWALALAYRRIDDDKGRTHELEH
SAIKCMRGILYCYMRQADKVQQFKQDPRPTTCLHSVFNVHTGDELLSYEEYGHLQINA
VSLYLLYLVEMISSGLQIIYNTDEVSFIQNLVFCVERVYRVPDFGVWERGSKYNNGSTEL
HSSSVGLAKAALEAINGFNLFGNQGCSWSVIFVDLDAHNRNRQTLCSLLPRESRSHNTD
AALLPCISYPAFALDDEVLFSQTLDKVVRKLKGKYGFKRFLRDGYRTSLEDPNRCYYKP
AEIKLFDGIECEFPIFFLYMMIDGVFRGNPKQVQEYQDLLTPVLHHTTEGYPVVPKYYY
VPADFVEYEKNNPGSQKRFPSNCGRDGKLFLWGQALYIIAKLLADELISPKDIDPVQRY
VPLKDQRNVSMRFSNQGPLENDLVVHVALIAESQRLQVFLNTYGIQTQTPQQVEPIQIW
PQQELVKAYLQLGINEKLGLSGRPDRPIGCLGTSKIYRILGKTVVCYPIIFDLSDFYMSQD
VFLLIDDIKNALQFIKQYWKMHGRPLFLVLIREDNIRGSRFNPILDMLAALKKGIIGGVK
VHVDRLQTLISGAVVEQLDFLRISDTEELPEFKSFEELEPPKHSKVKRQSSTPSAPELGQQ
PDVNISEWKDKPTHEILQKLNDCSCLASQAILLGILLKREGPNFITKEGTVSDHIERVYRR
AGSQKLWLAVRYGAAFTQKFSSSIAPHITTFLVHGKQVTLGAFGHEEEVISNPLSPRVIQ
NIIYYKCNTHDEREAVIQQELVIHIGWIISNNPELFSGMLKIRIGWIIHAMEYELQIRGGDK
PALDLYQLSPSEVKQLLLDILQPQQNGRCWLNRRQIDGSLNRTPTGFYDRVWQILERTP
NGIIVAGKHLPQQPTLSDMTMYEMNFSLLVEDTLGNIDQPQYRQIVVELLMVVSIVLER
NPELEFQDKVDLDRLVKEAFNEFQKDQSRLKEIEKQDDMTSFYNTPPLGKRGTCSYLTK
AVMNLLLEGEVKPNNDDPCLIS (SEQ ID NO: 05).
In an aspect, a disclosed PhK β subunit can comprise the following sequence or a fragment
thereof:
MACSPDAVVSPSSAFLRSGSVYEPLKSINLPRPDNETLWDKLDHYYRIVKSTLLLYQSPT
TGLFPTKTCGGDQKAKIQDSLYCAAGAWALALAYRRIDDDKGRTHELEHSAIKCMRGI
LYCYMRQADKVQQFKQDPRPTTCLHSVFNVHTGDELLSYEEYGHLQINAVSLYLLYLV
EMISSGLQIIYNTDEVSFIQNLVFCVERVYRVPDFGVWERGSKYNNGSTELHSSSVGLAK
AALEAINGFNLFGNQGCSWSVIFVDLDAHNRNRQTLCSLLPRESRSHNTDAALLPCISYP
AFALDDEVLFSQTLDKVVRKLKGKYGFKRFLRDGYRTSLEDPNRCYYKPAEIKLFDGIE
CEFPIFFLYMMIDGVFRGNPKQVQEYQDLLTPVLHHTTEGYPVVPKYYYVPADFVEYE
KNNPGSQKRFPSNCGRDGKLFLWGQALYIIAKLLADELISPKDIDPVQRYVPLKDQRNV
SMRFSNQGPLENDLVVHVALIAESQRLQVFLNTYGIQTQTPQQVEPIQIWPQQELVKAY
LQLGINEKLGLSGRPDRPIGCLGTSKIYRILGKTVVCYPIIFDLSDFYMSQDVFLLIDDIKN
ALQFIKQYWKMHGRPLFLVLIREDNIRGSRFNPILDMLAALKKGIIGGVKVHVDRLQTLI
SGAVVEQLDFLRISDTEELPEFKSFEELEPPKHSKVKRQSSTPSAPELGQQPDVNISEWKD
KPTHEILQKLNDCSCLASQAILLGILLKREGPNFITKEGTVSDHIERVYRRAGSQKLWSV
VRRAASLLSKVVDSLAPSITNVLVQGKQVTLGAFGHEEEVISNPLSPRVIQNIIYYKCNT
HDEREAVIQQELVIHIGWIISNNPELFSGMLKIRIGWIIHAMEYELQIRGGDKPALDLYQL
SPSEVKQLLLDILQPQQNGRCWLNRRQIDGSLNRTPTGFYDRVWQILERTPNGIIVAGKH
LPQQPTLSDMTMYEMNFSLLVEDTLGNIDQPQYRQIVVELLMVVSIVLERNPELEFQDK
VDLDRLVKEAFNEFQKDQSRLKEIEKQDDMTSFYNTPPLGKRGTCSYLTKAVMNLLLE
GEVKPNNDDPCLIS (SEQ ID NO: 06).
In an aspect, a disclosed PhK β subunit can comprise the following sequence or a fragment
thereof:
MAGAAGLTAEVSWKVLERRARTKRSGSVYEPLKSINLPRPDNETLWDKLDHYYRIVKS
TLLLYQSPTTGLFPTKTCGGDQKAKIQDSLYCAAGAWALALAYRRIDDDKGRTHELEH
SAIKCMRGILYCYMRQADKVQQFKQDPRPTTCLHSVFNVHTGDELLSYEEYGHLQINA
VSLYLLYLVEMISSGLQIIYNTDEVSFIQNLVFCVERVYRVPDFGVWERGSKYNNGSTEL
HSSSVGLAKAALEAINGFNLFGNQGCSWSVIFVDLDAHNRNRQTLCSLLPRESRSHNTD
AALLPCISYPAFALDDEVLFSQTLDKVVRKLKGKYGFKRFLRDGYRTSLEDPNRCYYKP
AEIKLFDGIECEFPIFFLYMMIDGVFRGNPKQVQEYQDLLTPVLHHTTEGYPVVPKYYY
VPADFVEYEKNNPGSQKRFPSNCGRDGKLFLWGQALYIIAKLLADELISPKDIDPVQRY
VPLKDQRNVSMRFSNQGPLENDLVVHVALIAESQRLQVFLNTYGIQTQTPQQVEPIQIW
PQQELVKAYLQLGINEKLGLSGRPDRPIGCLGTSKIYRILGKTVVCYPIIFDLSDFYMSQD
VFLLIDDIKNALQFIKQYWKMHGRPLFLVLIREDNIRGSRFNPILDMLAALKKGIIGGVK
VHVDRLQTLISGAVVEQLDFLRISDTEELPEFKSFEELEPPKHSKVKRQSSTPSAPELGQQ
PDVNISEWKDKPTHEILQKLNDCSCLASQAILLGILLKREGPNFITKEGTVSDHIERVYRR
AGSQKLWSVVRRAASLLSKVVDSLAPSITNVLVQGKQVTLGAFGHEEEVISNPLSPRVI
QNIIYYKCNTHDEREAVIQQELVIHIGWIISNNPELFSGMLKIRIGWIIHAMEYELQIRGGD
KPALDLYQLSPSEVKQLLLDILQPQQNGRCWLNRRQIDGSLNRTPTGFYDRVWQILERT
PNGIIVAGKHLPQQPTLSDMTMYEMNFSLLVEDTLGNIDQPQYRQIVVELLMVVSIVLE
RNPELEFQDKVDLDRLVKEAFNEFQKDQSRLKEIEKQDDMTSFYNTPPLGKRGTCSYLT
KAVMNLLLEGEVKPNNDDPCLIS (SEQ ID NO: 07).
In an aspect, a disclosed PhK δ subunit can comprise the following sequence or a fragment
thereof.
MQADQLTEEQIAEFKEAFSLFDKDGDGTITTKELGTVMRSLGQNPTEAELQDMINEVDA
DGNGTIDFPEFLTMMARKMKDTDSEEEIREAFRVFDKDGNGYISAAELRHVMTNLGEK
LTDEEVDEMIREADIDGDGQVNYEEFVQMMTAK (SEQ ID NO: 57).
In an aspect, a disclosed PhK δ subunit can comprise the following sequence or a fragment
thereof.
MADQLTEEQIAEFKEAFSLFDKDGDGTITTKELGTVMRSLGQNPTEAELQDMINEVDAD
GNGTIDFPEFLTMMARKMKDTDSEEEIREAFRVFDKDGNGYISAAELRHVMTNLGEKL
TDEEVDEMIREADIDGDGQVNYEEFVQMMTAK (SEQ ID NO: 58).
In an aspect, a disclosed PhK δ subunit can comprise the following sequence or a fragment
thereof.
MRSLGQNPTEAELQDMINEVDADGNGTIDFPEFLTMMARKMKDTDSEEEIREAFRVFD
KDGNGYISAAELRHVMTNLGEKLTDEEVDEMIREADIDGDGQVNYEEFVQMMTAK
(SEQ ID NO: 59).
In an aspect, a disclosed PhK γ2 subunit can comprise the following sequence or a
fragment thereof:
MTLDVGPEDELPDWAAAKEFYQKYDPKDVIGRGVSSVVRRCVHRATGHEFAVKIMEV
TAERLSPEQLEEVREATRRETHILRQVAGHPHIITLIDSYESSSFMFLVFDLMRKGELFDY
LTEKVALSEKETRSIMRSLLEAVSFLHANNIVHRDLKPENILLDDNMQIRLSDFGFSCHL
EPGEKLRELCGTPGYLAPEILKCSMDETHPGYGKEVDLWACGVILFTLLAGSPPFWHRR
QILMLRMIMEGQYQFSSPEWDDRSSTVKDLISRLLQVDPEARLTAEQALQHPFFERCEG
SQPWNLTPRQRFRVAVWTVLAAGRVALSTHRVRPLTKNALLRDPYALRSVRHLIDNCA
FRLYGHWIRKQWIGKLMACV (SEQ ID NO: 08).
In an aspect, a disclosed PhK γ2 subunit can comprise the following sequence or a
fragment thereof:
MTLDVGPEDELPDWAAAKEFYQKYDPKDVIGRGVSSVVRRCVHRATGHEFAVKIMEV
TAERLSPEQLEEVREATRRETHILRQVAGHPHIITLIDSYESSSFMFLVFDLMRKGELFDY
LTEKVALSEKETRSIMRSLLEAVSFLHANNIVHRDLKPENILLDDNMQIRLSDFGFSCHL
EPGEKLRELCGTPGYLAPEILKCSMDETHPGYGKEVDLWACGVILFTLLAGSPPFWHRR
QILMLRMIMEGQYQFSSPEWDDRSSTVKDLISRLLQVDPEARLTAEQALQHPFFERCEG
SQPWNLTPRQRFRVAVWTVLAAGRVALSTHRVRPLTKNALLRDPYALRSVRHLIDNCA
FRLYGHWVKKGEQQNRAALFQHRPPGPFPIMGPEEEGDSAAITEDEAVLVLG (SEQ ID
NO: 09).
In an aspect, a disclosed PYGL can comprise the following sequence or a fragment thereof:
MAKPLTDQEKRRQISIRGIVGVENVAELKKSFNRHLHFTLVKDRNVATTRDYYFALAH
TVRDHLVGRWIRTQQHYYDKCPKRVYYLSLEFYMGRTLQNTMINLGLQNACDEAIYQ
LGLDIEELEEIEEDAGLGNGGLGRLAACFLDSMATLGLAAYGYGIRYEYGIFNQKIRDG
WQVEEADDWLRYGNPWEKSRPEFMLPVHFYGKVEHTNTGTKWIDTQVVLALPYDTP
VPGYMNNTVNTMRLWSARAPNDFNLRDFNVGDYIQAVLDRNLAENISRVLYPNDNFF
EGKELRLKQEYFVVAATLQDIIRRFKASKFGSTRGAGTVFDAFPDQVAIQLNDTHPALAI
PELMRIFVDIEKLPWSKAWELTQKTFAYTNHTVLPEALERWPVDLVEKLLPRHLEIIYEI
NQKHLDRIVALFPKDVDRLRRMSLIEEEGSKRINMAHLCIVGSHAVNGVAKIHSDIVKT
KVFKDFSELEPDKFQNKTNGITPRRWLLLCNPGLAELIAEKIGEDYVKDLSQLTKLHSFL
GDDVFLRELAKVKQENKLKFSQFLETEYKVKINPSSMFDVQVKRIHEYKRQLLNCLHVI
TMYNRIKKDPKKLFVPRTVIIGGKAAPGYHMAKMIIKLITSVADVVNNDPMVGSKLKVI
FLENYRVSLAEKVIPATDLSEQISTAGTEASGTGNMKFMLNGALTIGTMDGANVEMAE
EAGEENLFIFGMRIDDVAALDKKGYEAKEYYEALPELKLVIDQIDNGFFSPKQPDLFKDI
INMLFYHDRFKVFADYEAYVKCQDKVSQLYMNPKAWNTMVLKNIAASGKFSSDRTIK
EYAQNIWNVEPSDLKISLSNESNKVNGN (SEQ ID NO: 10).
In an aspect, a disclosed PYGL can comprise the following sequence or a fragment thereof:
MAKPLTDQEKRRQISIRGIVGVENVAELKKSFNRHLHFTLVKDRNVATTRDYYFALAH
TVRDHLVGRWIRTQQHYYDKCPKLGLDIEELEEIEEDAGLGNGGLGRLAACFLDSMAT
LGLAAYGYGIRYEYGIFNQKIRDGWQVEEADDWLRYGNPWEKSRPEFMLPVHFYGKV
EHTNTGTKWIDTQVVLALPYDTPVPGYMNNTVNTMRLWSARAPNDFNLRDFNVGDYI
QAVLDRNLAENISRVLYPNDNFFEGKELRLKQEYFVVAATLQDIIRRFKASKFGSTRGA
GTVFDAFPDQVAIQLNDTHPALAIPELMRIFVDIEKLPWSKAWELTQKTFAYTNHTVLP
EALERWPVDLVEKLLPRHLEIIYEINQKHLDRIVALFPKDVDRLRRMSLIEEEGSKRINM
AHLCIVGSHAVNGVAKIHSDIVKTKVFKDFSELEPDKFQNKTNGITPRRWLLLCNPGLA
ELIAEKIGEDYVKDLSQLTKLHSFLGDDVFLRELAKVKQENKLKFSQFLETEYKVKINPS
SMFDVQVKRIHEYKRQLLNCLHVITMYNRIKKDPKKLFVPRTVIIGGKAAPGYHMAKM
IIKLITSVADVVNNDPMVGSKLKVIFLENYRVSLAEKVIPATDLSEQISTAGTEASGTGN
MKFMLNGALTIGTMDGANVEMAEEAGEENLFIFGMRIDDVAALDKKGYEAKEYYEAL
PELKLVIDQIDNGFFSPKQPDLFKDIINMLFYHDRFKVFADYEAYVKCQDKVSQLYMNP
KAWNTMVLKNIAASGKFSSDRTIKEYAQNIWNVEPSDLKISLSNESNKVNGN (SEQ ID
NO: 11).
In an aspect, a disclosed Cas9 can comprise the following sequence or a fragment thereof:
MKRNYILGLDIGITSVGYGIIDYETRDVIDAGVRLFKEANVENNEGRRSKRGARRLKRR
RRHRIQRVKKLLFDYNLLTDHSELSGINPYEARVKGLSQKLSEEEFSAALLHLAKRRGV
HNVNEVEEDTGNELSTKEQISRNSKALEEKYVAELQLERLKKDGEVRGSINRFKTSDYV
KEAKQLLKVQKAYHQLDQSFIDTYIDLLETRRTYYEGPGEGSPFGWKDIKEWYEMLMG
HCTYFPEELRSVKYAYNADLYNALNDLNNLVITRDENEKLEYYEKFQIIENVFKQKKKP
TLKQIAKEILVNEEDIKGYRVTSTGKPEFTNLKVYHDIKDITARKEIIENAELLDQIAKILT
IYQSSEDIQEELTNLNSELTQEEIEQISNLKGYTGTHNLSLKAINLILDELWHTNDNQIAIF
NRLKLVPKKVDLSQQKEIPTTLVDDFILSPVVKRSFIQSIKVINAIIKKYGLPNDIIIELARE
KNSKDAQKMINEMQKRNRQTNERIEEIIRTTGKENAKYLIEKIKLHDMQEGKCLYSLEA
IPLEDLLNNPFNYEVDHIIPRSVSFDNSFNNKVLVKQEENSKKGNRTPFQYLSSSDSKISY
ETFKKHILNLAKGKGRISKTKKEYLLEERDINRFSVQKDFINRNLVDTRYATRGLMNLL
RSYFRVNNLDVKVKSINGGFTSFLRRKWKFKKERNKGYKHHAEDALIIANADFIFKEW
KKLDKAKKVMENQMFEEKQAESMPEIETEQEYKEIFITPHQIKHIKDFKDYKYSHRVDK
KPNRELINDTLYSTRKDDKGNTLIVNNLNGLYDKDNDKLKKLINKSPEKLLMYHHDPQ
TYQKLKLIMEQYGDEKNPLYKYYEETGNYLTKYSKKDNGPVIKKIKYYGNKLNAHLDI
TDDYPNSRNKVVKLSLKPYRFDVYLDNGVYKFVTVKNLDVIKKENYYEVNSKCYEEA
KKLKKISNQAEFIASFYNNDLIKINGELYRVIGVNNDLLNRIEVNMIDITYREYLENMND
KRPPRIIKTIASKTQSIKKYSTDILGNLYEVKSKKHPQIIKKG (SEQ ID NO: 32).
In an aspect, a disclosed Cas9 can comprise the following sequence or a fragment thereof:
MDKKYSIGLDIGTNSVGWAVITDDYKVPSKKFKVLGNTDRHSIKKNLIGALLFDSGETA
EATRLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFLIEGDLNPDN
SDVDKLFIQLVQTYNQLFEENPINASGVDAKAILSARLSKSRRLENLIAQLPGEKKNGLF
GNLIALSLGLTPNFKSNFDLAEDAKLQLSKDTYDDDLDNLLAQIGDQYADLFLAAKNLS
DAILLSDILRLNSEITKAPLSASMIKRYDEHHQDLTLLKALVRQQLPEKYKEIFFDQSKN
GYAGYIDGGASQEEFYKFIKPILEKMDGTEELLAKLNREDLLRKQRTFDNGSIPHQIHLG
ELHAILRRQEDFYPFLKDNREKIEKILTFRIPYYVGPLARGNSRFAWMTRKSEETITPWN
FEEVVDKGASAQSFIERMTNFDKNLPNEKVLPKHSLLYEYFTVYNELTKVKYVTEGMR
KPAFLSGEQKKAIVDLLFKTNRKVTVKQLKEDYFKKIECFDSVEISGVEDRFNASLGTY
HDLLKIIKDKDFLDNEENEDILEDIVLTLTLFEDREMIEERLKTYAHLFDDKVMKQLKRR
RYTGWGRLSRKLINGIRDKQSGKTILDFLKSDGFANRNFMQLIHDDSLTFKEDIQKAQV
SGQGDSLHEHIANLAGSPAIKKGILQTVKVVDELVKVMGRHKPENIVIEMARENQTTQK
GQKNSRERMKRIEEGIKELGSQILKEHPVENTQLQNEKLYLYYLQNGRDMYVDQELDI
NRLSDYDVDHIVPQSFIKDDSIDNKVLTRSDKNRGKSDNVPSEEVVKKMKNYWRQLLN
AKLITQRKFDNLTKAERGGLSELDKAGFIKRQLVETRQITKHVAQILDSRMNTKYDEND
KLIREVKVITLKSKLVSDFRKDFQFYKVREINNYHHAHDAYLNAVVGTALIKKYPKLES
EFVYGDYKVYDVRKMIAKSEQEIGKATAKYFFYSNIMNFFKTEITLANGEIRKRPLIETN
GETGEIVWDKGRDFATVRKVLSMPQVNIVKKTEVQTGGFSKESILPKRNSDKLIARKKD
WDPKKYGGFDSPTVAYSVLVVAKVEKGKSKKLKSVKELLGLTIMERSSFEKNPIDFLEA
KGYKEVKKDLIIKLPKYSLFELENGRKRMLASAGELQKGNELALPSKYVNFLYLASHYE
KLKGSPEDNEQKQLFVEQHKHYLDEIIEQISEFSKRVILADANLDKVLSAYNKHRDKPIR
EQAENIIHLFTLTNLGAPAAFKYFDTTIDRKRYTSTKEVLDATLIHQSITGLYETRIDLSQL
GGD (SEQ ID NO: 33).
In an aspect, a disclosed GAA can comprise the following sequence or a fragment thereof:
MGVRHPPCSHRLLAVCALVSLATAALLGHILLHDFLLVPRELSGSSPVLEETHPAHQQG
ASRPGPRDAQAHPGRPRAVPTQCDVPPNSRFDCAPDKAITQEQCEARGCCYIPAKQGLQ
GAQMGQPWCFFPPSYPSYKLENLSSSEMGYTATLTRTTPTFFPKDILTLRLDVMMETEN
RLHFTIKDPANRRYEVPLETPRVHSRAPSPLYSVEFSEEPFGVIVHRQLDGRVLLNTTVA
PLFFADQFLQLSTSLPSQYITGLAEHLSPLMLSTSWTRITLWNRDLAPTPGANLYGSHPF
YLALEDGGSAHGVFLLNSNAMDVVLQPSPALSWRSTGGILDVYIFLGPEPKSVVQQYL
DVVGYPFMPPYWGLGFHLCRWGYSSTAITRQVVENMTRAHFPLDVQWNDLDYMDSR
RDFTFNKDGFRDFPAMVQELHQGGRRYMMIVDPAISSSGPAGSYRPYDEGLRRGVFITN
ETGQPLIGKVWPGSTAFPDFTNPTALAWWEDMVAEFHDQVPFDGMWIDMNEPSNFIRG
SEDGCPNNELENPPYVPGVVGGTLQAATICASSHQFLSTHYNLHNLYGLTEAIASHRAL
VKARGTRPFVISRSTFAGHGRYAGHWTGDVWSSWEQLASSVPEILQFNLLGVPLVGAD
VCGFLGNTSEELCVRWTQLGAFYPFMRNHNSLLSLPQEPYSFSEPAQQAMRKALTLRY
ALLPHLYTLFHQAHVAGETVARPLFLEFPKDSSTWTVDHQLLWGEALLITPVLQAGKA
EVTGYFPLGTWYDLQTVPIEALGSLPPPPAAPREPAIHSEGQWVTLPAPLDTINVHLRAG
YIIPLQGPGLTTTESRQQPMALAVALTKGGEARGELFWDDGESLEVLERGAYTQVIFLA
RNNTIVNELVRVTSEGAGLQLQKVTVLGVATAPQQVLSNGVPVSNFTYSPDTKVLDICV
SLLMGEQFLVSWC (SEQ ID NO: 34).
In an aspect, a disclosed GAA can comprise the following sequence or a fragment thereof:
MGVRHPPCSHRLLAVCALVSLATAALLGHILLHDFLLVPRELSGSSPVLEETHP AHQQG
ASRPGPRDAQAHPGRPRAVPTQCDVPPNSRFDCAPDKAITQEQCEARGCCYIPAKQGLQ
GAQMGQPWCFFPPSYPSYKLENLSSSEMGYTATLTRTTPTFFPKDILTLRLDVMMETEN
RLHFTIKDPANRRYEVPLETPRVHSRAPSPLYSVEFSEEPFGVIVHRQLDGRVLLNTTVA
PLFFADQFLQLSTSLPSQYITGLAEHLSPLMLSTSWTRITLWNRDLAPTPGANLYGSHPF
YLALEDGGSAHGVFLLNSNAMDVVLQPSPALSWRSTGGILDVYIFLGPEPKSVVQQYL
DVVGYPFMPPYWGLGFHLCRWGYSSTAITRQVVENMTRAHFPLDVQWNDLDYMDSR
RDFTFNKDGFRDFPAMVQELHQGGRRYMMIVDPAISSSGPAGSYRLYDEGLRRGVFITN
ETGQPLIGKVWPGSTAFPDFTNPTALAWWEDMVAEFHDQVPFDGMWIDMNEPSNFIRG
SEDGCPNNELENPPYVPGVVGGTLQAATICASSHQFLSTHYNLHNLYGLTEAIASHRAL
VKARGTRPFVISRSTFAGHGRYAGHWTGDVWSSWEQLASSVPEILQFNLLGVPLVGAD
VCGFLGNTSEELCVRWTQLGAFYPFMRNHNSLLSLPQEPYSFSEPAQQAMRKALTLRY
ALLPHLYTLFHQAHVAGETVARPLFLEFPKDSSTWTVDHQLLWGEALLITPVLQAGKA
EVTGYFPLGTWYDLQTVPIEALGSLPPPPAAPREPAIHSEGQWVTLPAPLDTINVHLRAG
YIIPLQGPGLTTTESRQQPMALAVALTKGGEARGELFWDDGESLEVLERGAYTQVIFLA
RNNTIVNELVRVTSEGAGLQLQKVTVLGVATAPQQVLSNGVPVSNFTYSPDTKVLDICV
SLLMGEQFLVSWC (SEQ ID NO: 35).
In an aspect, a disclosed GAA can comprise the following sequence or a fragment thereof:
MGVRHPPCSHRLLAVCALVSLATAALLGHILLHDFLLVPRELSGSSPVLEETHP AHQQG
ASRPGPRDAQAHPGRPRAVPTQCDVPPNSRFDCAPDKAITQEQCEARGCCYIPAKQGLQ
GAQMGQPWCFFPPSYPSYKLENLSSSEMGYTATLTRTTPTFFPKDILTLRLDVMMETEN
RLHFTIKDPANRRYEVPLETPHVHSRAPSPLYSVEFSEEPFGVIVRRQLDGRVLLNTTVA
PLFFADQFLQLSTSLPSQYITGLAEHLSPLMLSTSWTRITLWNRDLAPTPGANLYGSHPF
YLALEDGGSAHGVFLLNSNAMDVVLQPSPALSWRSTGGILDVYIFLGPEPKSVVQQYL
DVVGYPFMPPYWGLGFHLCRWGYSSTAITRQVVENMTRAHFPLDVQWNDLDYMDSR
RDFTFNKDGFRDFPAMVQELHQGGRRYMMIVDPAISSSGPAGSYRPYDEGLRRGVFITN
ETGQPLIGKVWPGSTAFPDFTNPTALAWWEDMVAEFHDQVPFDGMWIDMNEPSNFIRG
SEDGCPNNELENPPYVPGVVGGTLQAATICASSHQFLSTHYNLHNLYGLTEAIASHRAL
VKARGTRPFVISRSTFAGHGRYAGHWTGDVWSSWEQLASSVPEILQFNLLGVPLVGAD
VCGFLGNTSEELCVRWTQLGAFYPFMRNHNSLLSLPQEPYSFSEPAQQAMRKALTLRY
ALLPHLYTLFHQAHVAGETVARPLFLEFPKDSSTWTVDHQLLWGEALLITPVLQAGKA
EVTGYFPLGTWYDLQTVPVEALGSLPPPPAAPREPAIHSEGQWVTLPAPLDTINVHLRA
GYIIPLQGPGLTTTESRQQPMALAVALTKGGEARGELFWDDGESLEVLERGAYTQVIFL
ARNNTIVNELVRVTSEGAGLQLQKVTVLGVATAPQQVLSNGVPVSNFTYSPDTKVLDIC
VSLLMGEQFLVSWC (SEQ ID NO: 36).
In an aspect, a disclosed GAA can comprise the following sequence or a fragment thereof:
MGVRHPPCSHRLLAVCALVSLATAALLGHILLHDFLLVPRELSGSSPVLEETHPAHQQG
ASRPGPRDAQAHPGRPRAVPTQCDVPPNSRFDCAPDKAITQEQCEARGCCYIPAKQGLQ
GAQMGQPWCFFPPSYPSYKLENLSSSEMGYTATLTRTTPTFFPKDILTLRLDVMMETEN
RLHFTIKDPANRRYEVPLETPHVHSRAPSPLYSVEFSEEPFGVIVRRQLDGRVLLNTTVA
PLFFADQFLQLSTSLPSQYITGLAEHLSPLMLSTSWTRITLWNRDLAPTPGANLYGSHPF
YLALEDGGSAHGVFLLNSNAMDVVLQPSPALSWRSTGGILDVYIFLGPEPKSVVQQYL
DVVGYPFMPPYWGLGFHLCRWGYSSTAITRQVVENMTRAHFPLDVQWNDLDYMDSR
RDFTFNKDGFRDFPAMVQELHQGGRRYMMIVDPAISSSGPAGSYRPYDEGLRRGVFITN
ETGQPLIGKVWPGSTAFPDFTNPTALAWWEDMVAEFHDQVPFDGMWIDMNEPSNFIRG
SEDGCPNNELENPPYVPGVVGGTLQAATICASSHQFLSTHYNLHNLYGLTEAIASHRAL
VKARGTRPFVISRSTFAGHGRYAGHWTGDVWSSWEQLASSVPEILQFNLLGVPLVGAD
VCGFLGNTSEELCVRWTQLGAFYPFMRNHNSLLSLPQEPYSFSEPAQQAMRKALTLRY
ALLPHLYTLFHQAHVAGETVARPLFLEFPKDSSTWTVDHQLLWGEALLITPVLQAGKA
EVTGYFPLGTWYDLQTVPIEALGSLPPPPAAPREPAIHSEGQWVTLPAPLDTINVHLRAG
YIIPLQGPGLTTTESRQQPMALAVALTKGGEARGELFWDDGESLEVLERGAYTQVIFLA
RNNTIVNELVRVTSEGAGLQLQKVTVLGVATAPQQVLSNGPVSNFTYSPDTKVLDICVS
LLMGEQFLVSWC (SEQ ID NO: 37).
In an aspect, a disclosed GAA can comprise the following sequence or a fragment thereof:
AHPGRPRAVPTQCDVPPNSRFDCAPDKAITQEQCEARGCCYIPAKQGLQGAQMGQPWC
FFPPSYPSYKLENLSSSEMGYTATLTRTTPTFFPKDILTLRLDVMMETENRLHFTIKDPAN
RRYEVPLETPHVHSRAPSPLYSVEFSEEPFGVIVRRQLDGRVLLNTTVAPLFFADQFLQL
STSLPSQYITGLAEHLSPLMLSTSWTRITLWNRDLAPTPGANLYGSHPFYLALEDGGSAH
GVFLLNSNAMDVVLQPSPALSWRSTGGILDVYIFLGPEPKSVVQQYLDVVGYPFMPPY
WGLGFHLCRWGYSSTAITRQVVENMTRAHFPLDVQWNDLDYMDSRRDFTFNKDGFR
DFPAMVQELHQGGRRYMMIVDPAISSSGPAGSYRPYDEGLRRGVFITNETGQPLIGKVW
PGSTAFPDFTNPTALAWWEDMVAEFHDQVPFDGMWIDMNEPSNFIRGSEDGCPNNELE
NPPYVPGVVGGTLQAATICASSHQFLSTHYNLHNLYGLTEAIASHRALVKARGTRPFVIS
RSTFAGHGRYAGHWTGDVWSSWEQLASSVPEILQFNLLGVPLVGADVCGFLGNTSEEL
CVRWTQLGAFYPFMRNHNSLLSLPQEPYSFSEPAQQAMRKALTLRYALLPHLYTLFHQ
AHVAGETVARPLFLEFPKDSSTWTVDHQLLWGEALLITPVLQAGKAEVTGYFPLGTWY
DLQTVPVEALGSLPPPPAAPREPAIHSEGQWVTLPAPLDTINVHLRAGYIIPLQGPGLTTT
ESRQQPMALAVALTKGGEARGELFWDDGESLEVLERGAYTQVIFLARNNTIVNELVRV
TSEGAGLQLQKVTVLGVATAPQQVLSNGVPVSNFTYSPDTKVLDICVSLLMGEQFLVS
WC (SEQ ID NO: 38).
In an aspect, a disclosed GYS1 can comprise the following sequence or a fragment thereof:
MPLNRTLSMSSLPGLEDWEDEFDLENAVLFEVAWEVANKVGGIYTVLQTKAKVTGDE
WGDNYFLVGPYTEQGVRTQVELLEAPTPALKRTLDSMNSKGCKFLAQSEEKPHVVAHF
HEWLAGVGLCLCRARRLPVATIFTTHATLLGRYLCAGAVDFYNNLENFNVDKEAGER
QIYHRYCMERAAAHCAHVFTTVSQITAIEAQHLLKRKPDIVTPNGLNVKKFSAMHEFQ
NLHAQSKARIQEFVRGHFYGHLDFNLDKTLYFFIAGRYEFSNKGADVFLEALARLNYLL
RVNGSEQTVVAFFIMPARTNNFNVETLKGQAVRKQLWDTANTVKEKFGRKLYESLLV
GSLPDMNKMLDKEDFTMMKRAIFATQRQSFPPVCTHNMLDDSSDPILTTIRRIGLENSSA
DRVKVIFHPEFLSSTSPLLPVDYEEFVRGCHLGVFPSYYEPWGYTPAECTVMGIPSISTNL
SGFGCFMEEHIADPSAYGIYILDRRFRSLDDSCSQLTSFLYSFCQQSRRQRIIQRNRTERLS
DLLDWKYLGRYYMSARHMALSKAFPEHFTYEPNEADAAQGYRYPRPASVPPSPSLSRH
SSPHQSEDEEDPRNGPLEEDGERYDEDEEAAKDRRNIRAPEWPRRASCTSSTSGSKRNS
VDTATSSSLSTPSEPLSPTSSLGEERN (SEQ ID NO: 41).
In an aspect, a disclosed GYS1 can comprise the following sequence or a fragment thereof:
MPLNRTLSMSSLPGLEDWEDEFDLENAVLFEVAWEVANKVGGIYTVLQTKAKVTGDE
WGDNYFLVGPYTEQGVRTQVELLEAPTPALKRTLDSMNSKGCKVYFGRWLIEGGPLV
VLLDVGASAWALERWKGELWDTCNIGVPWYDREANDAVLFGFLTTWFLGEFLAQSEE
KPHVVAHFHEWLAGVGLCLCRARRLPVATIFTTHATLLGRYLCAGAVDFYNNLENFNV
DKEAGERQIYHRYCMERAAAHCAHVFTTVSQITAIEAQHLLKRKPDIVTPNGLNVKKFS
AMHEFQNLHAQSKARIQEFVRGHFYGHLDFNLDKTLYFFIAGRYEFSNKGADVFLEAL
ARLNYLLRVNGSEQTVVAFFIMPARTNNFNVETLKGQAVRKQLWDTANTVKEKFGRK
LYESLLVGSLPDMNKMLDKEDFTMMKRAIFATQRQSFPPVCTHNMLDDSSDPILTTIRRI
GLFNSSADRVKVIFHPEFLSSTSPLLPVDYEEFVRGCHLGVFPSYYEPWGYTPAECTVM
GIPSISTNLSGFGCFMEEHIADPSAYGIYILDRRFRSLDDSCSQLTSFLYSFCQQSRRQRIIQ
RNRTERLSDLLDWKYLGRYYMSARHMALSKAFPEHFTYEPNEADAAQGYRYPRPASV
PPSPSLSRHSSPHQSEDEEDPRNGPLEEDGERYDEDEEAAKDRRNIRAPEWPRRASCTSS
TSGSKRNSVDTATSSSLSTPSEPLSPTSSLGEERN (SEQ ID NO: 42).
In an aspect, a disclosed GYS2 can comprise the following sequence or a fragment thereof:
MLRGRSLSVTSLGGLPQWEVEELPVEELLLFEVAWEVTNKVGGIYTVIQTKAKTTADE
WGENYFLIGPYFEHNMKTQVEQCEPVNDAVRRAVDAMNKHGCQVHFGRWLIEGSPY
VVLFDIGYSAWNLDRWKGDLWEACSVGIPYHDREANDMLIFGSLTAWFLKEVTDHAD
GKYVVAQFHEWQAGIGLILSRARKLPIATIFTTHATLLGRYLCAANIDFYNHLDKFNIDK
EAGERQIYHRYCMERASVHCAHVFTTVSEITAIEAEHMLKRKPDVVTPNGLNVKKFSA
VHEFQNLHAMYKARIQDFVRGHFYGHLDFDLEKTLFLFIAGRYEFSNKGADIFLESLSR
LNFLLRMHKSDITVMVFFIMPAKTNNFNVETLKGQAVRKQLWDVAHSVKEKFGKKLY
DALLRGEIPDLNDILDRDDLTIMKRAIFSTQRQSLPPVTTHNMIDDSTDPILSTIRRIGLFN
NRTDRVKVILHPEFLSSTSPLLPMDYEEFVRGCHLGVFPSYYEPWGYTPAECTVMGIPSV
TTNLSGFGCFMQEHVADPTAYGIYIVDRRFRSPDDSCNQLTKFLYGFCKQSRRQRIIQRN
RTERLSDLLDWRYLGRYYQHARHLTLSRAFPDKFHVELTSPPTTEGFKYPRPSSVPPSPS
GSQASSPQSSDVEDEVEDERYDEEEEAERDRLNIKSPFSLSHVPHGKKKLHGEYKN
(SEQ ID NO: 43).
b. Nucleotide Sequences
In an aspect, a disclosed PHKA1 can comprise the following sequence or a fragment
thereof:
GCCTCCCGCTCGCGTCTCCCGCAAGGTGGAGGCGGCGCAGCGGTCGTTGACCTGTT
GGCGGCGGCCGGGCGGGAGGCTGTGGGCCGCGGCGGTCTCCCGGGGTTGAGGGGA
GGATGCAGGAGTCACACGCGGAGGCCGGACTGGACTAAGGGGGCAGGAGCGGCGC
CTGGGGCGCTGCGGCGGGGCTCTAGACCGCGGGAAGCCAGTGCTAGCGCTGGCGCG
TGGTGGGCCCCTGAGGCCGCCGGAGTCCGGAGAGGTACGCGGAATCCGCGGCGCCA
GGCCTGAGCGGTGGGAGGGCTCTGCGGGGCCTGGTGTTCAGGCGTCCCACCACGAG
GGTGGAGCAGCGTTGGATACTTGTTCCTTAGGGACCGAAGGCTCCGGTGGCACCCG
GGCTATTTCTCAGAGGACAATTAGTAACGTGTCGCCATGAGGAGCCGGAGTAACTC
CGGGGTCCGGCTGGACGGCTACGCTCGACTGGTGCAACAGACCATCCTGTGCCATC
AGAATCCAGTGACTGGCTTGCTTCCAGCCAGCTATGATCAGAAAGATGCTTGGGTC
CGAGATAATGTGTACAGCATCTTGGCTGTGTGGGGTTTGGGCCTGGCCTATCGGAA
GAATGCAGACCGGGATGAGGATAAGGCAAAGGCCTATGAATTGGAGCAGAGTGTA
GTGAAGCTGATGAGAGGACTACTGCACTGCATGATCAGACAGGTGGATAAAGTAGA
ATCCTTCAAATATAGTCAGAGTACTAAGGATAGCCTCCATGCAAAGTACAACACCA
AAACCTGTGCCACTGTAGTGGGTGATGATCAATGGGGACACCTGCAGTTGGATGCT
ACCTCTGTGTACCTGCTCTTCTTAGCCCAAATGACTGCCTCAGGACTCCATATCATC
CACAGCCTAGATGAAGTCAATTTCATACAGAACCTTGTGTTTTACATTGAAGCTGCA
TATAAAACTGCTGACTTCGGGATATGGGAACGTGGAGACAAGACCAACCAAGGGAT
CTCAGAGTTGAATGCCAGTTCAGTTGGAATGGCAAAGGCAGCCCTGGAAGCATTAG
ATGAACTGGATCTGTTTGGTGTGAAAGGTGGGCCTCAATCAGTTATCCATGTCCTGG
CTGATGAAGTACAGCACTGCCAGTCTATCCTAAATTCACTACTGCCCCGTGCTTCAA
CATCAAAAGAGGTTGATGCTAGTCTACTCTCAGTGGTTTCCTTCCCTGCCTTTGCAG
TAGAGGATAGCCAGTTGGTGGAGCTCACAAAACAGGAAATCATCACCAAGCTTCAG
GGTCGTTATGGTTGCTGTCGCTTTCTACGAGATGGATATAAAACTCCTAAAGAGGAT
CCCAATCGTCTGTACTATGAACCAGCTGAGCTGAAGCTATTTGAAAACATTGAGTGT
GAATGGCCATTGTTCTGGACATACTTTATTCTTGATGGGGTCTTCAGTGGCAATGCA
GAACAGGTTCAAGAATATAAAGAGGCTCTTGAAGCAGTCCTCATCAAGGGCAAAAA
TGGAGTCCCACTTCTGCCAGAGCTGTACAGTGTTCCTCCTGACAGGGTCGATGAAGA
ATATCAGAATCCTCACACTGTGGACCGAGTCCCCATGGGGAAATTGCCTCACATGT
GGGGTCAGTCTCTATACATTTTAGGAAGCTTGATGGCAGAGGGATTTTTAGCCCCTG
GAGAAATTGATCCCCTGAATCGCAGGTTTTCTACTGTACCGAAGCCCGATGTTGTGG
TTCAAGTCTCCATTCTAGCTGAAACAGAAGAAATCAAGACCATTTTGAAGGACAAG
GGAATTTACGTGGAGACCATTGCTGAGGTATACCCCATCAGAGTACAACCAGCTCG
TATTCTCAGCCACATTTATTCCAGCCTAGGATGCAACAATAGAATGAAACTCAGTGG
ACGACCCTACAGACACATGGGAGTGCTTGGAACTTCAAAACTCTATGACATTCGGA
AAACTATCTTTACTTTCACTCCACAGTTTATAGACCAGCAACAGTTCTACCTGGCTC
TGGACAACAAGATGATAGTGGAAATGCTTAGAACAGACCTCTCCTACCTCTGTAGC
CGCTGGCGGATGACAGGCCAGCCCACCATCACCTTCCCCATCTCACACAGCATGCTT
GATGAAGATGGAACAAGCTTGAATTCAAGTATCCTGGCAGCACTCCGAAAAATGCA
AGATGGGTATTTTGGTGGGGCAAGGGTTCAAACAGGTAAATTGTCAGAGTTTTTGA
CAACATCTTGTTGCACACACTTGAGCTTCATGGACCCTGGACCTGAGGGTAAGCTGT
ACAGTGAAGATTATGATGACAACTATGATTACCTGGAATCTGGCAACTGGATGAAT
GATTATGATTCAACCAGTCATGCTCGCTGTGGTGATGAAGTTGCTCGTTATTTAGAT
CACCTTTTGGCGCACACTGCTCCCCATCCTAAACTAGCCCCTACCTCACAGAAGGGA
GGGCTAGATCGGTTCCAAGCTGCTGTGCAAACAACCTGCGACTTAATGTCCTTGGTG
ACCAAGGCCAAGGAACTGCATGTACAGAATGTTCACATGTATCTTCCTACGAAGTT
ATTTCAGGCTTCCCGGCCTTCATTCAACTTACTTGATTCACCTCATCCCCGACAGGA
GAACCAGGTTCCCTCTGTTCGTGTAGAAATACATCTTCCTAGAGACCAGTCTGGGGA
GGTGGACTTTAAAGCACTGGTTTTACAGTTGAAGGAGACCTCAAGCTTACAGGAAC
AAGCTGATATCCTCTATATGCTGTATACTATGAAAGGACCTGACTGGAACACTGAAT
TGTATAATGAACGGAGTGCTACAGTGAGAGAGCTTCTTACCGAGCTGTATGGCAAA
GTGGGAGAAATTCGTCACTGGGGCCTGATCCGATACATTTCTGGGATCTTAAGGAA
GAAAGTGGAAGCACTTGATGAGGCCTGCACAGACCTTCTCTCCCACCAGAAACATT
TGACAGTAGGACTTCCTCCAGAACCTCGAGAAAAGACTATCTCTGCACCTCTGCCCT
ATGAGGCGCTCACTCAGCTGATAGATGAAGCCAGTGAAGGGGATATGAGCATTTCA
ATCCTTACACAGGAAATAATGGTATATCTAGCCATGTATATGCGAACCCAGCCTGG
CCTCTTTGCTGAAATGTTTCGACTTCGAATTGGTCTGATCATACAAGTTATGGCAAC
AGAACTGGCCCACTCCCTTCGATGCTCAGCTGAGGAAGCCACAGAGGGCCTGATGA
ATCTCAGTCCTTCGGCCATGAAGAATCTCCTGCATCACATTCTCAGCGGCAAGGAGT
TTGGAGTGGAACGAAGCGTTCGTCCCACTGATTCAAATGTCAGTCCTGCTATTTCTA
TCCACGAGATTGGTGCTGTTGGAGCAACCAAAACAGAACGAACTGGGATCATGCAG
TTAAAAAGTGAGATAAAGCAGGTGGAATTTCGTAGACTGTCAATCTCAGCTGAGAG
TCAGTCACCTGGAACCTCTATGACTCCAAGTAGTGGGTCCTTTCCTAGTGCATATGA
TCAGCAGTCATCTAAAGATAGTCGTCAAGGTCAATGGCAACGCCGAAGAAGGCTGG
ATGGGGCACTGAATAGAGTTCCAGTTGGATTTTATCAGAAAGTATGGAAAGTTTTG
CAGAAGTGTCACGGACTTTCTGTTGAAGGGTTTGTCCTTCCTTCCTCTACCACTAGA
GAGATGACTCCAGGTGAGATTAAATTCTCTGTTCATGTGGAGTCTGTCCTGAATCGT
GTACCTCAGCCAGAGTACCGTCAGCTGCTGGTTGAAGCCATCCTTGTCCTCACCATG
CTGGCAGATATTGAAATTCATAGCATCGGAAGCATCATTGCTGTGGAAAAAATAGT
GCATATTGCCAATGACTTGTTCCTTCAAGAACAGAAAACCCTTGGCGCAGATGATA
CCATGTTGGCAAAGGATCCCGCATCTGGCATCTGTACTCTTCTGTATGACAGTGCAC
CCAGTGGCAGGTTTGGCACCATGACCTACCTCTCCAAGGCAGCCGCCACCTACGTG
CAGGAGTTCCTGCCCCACAGCATCTGTGCCATGCAATGAGGGCTTTGGTTCCTGGCT
TCTGGGAGCCTTTTGACAGCTGGTCCCTGCCTCGGTTGATTGTGCATGGAACTAAAA
TGTTATTGCCTAATCACTCCAACCCTGCCCCTTTCTGTCCCATCCTTCCCAAGAAGAG
AGAACTTTTTCGATAAACTAACTACTGTAGAAGAAGTGAACACTTACCTGGAGGCT
CACCTTGCAGAACCAGTGACAATCTTATGAGTATAATGAACACTCAGCCAGGCCTG
TCATGATTGGCTTTATTTCTTTCATCATTCATAAAAGTTTGCATGTGTTTTTATTCTCT
AGATCTGTTACCAATATAGTTTTCTAACTCCTGTTTGGGGAGCAAGTGTTAATAATA
ACTTATTCCTAAAGCTTGGTTTTTCTTTTTTGTGATTTTATTGTGTATATGAGTGGTG
GGTACTTTGGGACATTATTTCAAATGAGTAAACTCTAAATTGTTAAATATTTATGCT
GCTATAAATTAGAATTACCTATTCTACGTGCCTTTTCTTAGGAAAAGAGAGTACTGG
ATCATGGTTTTCTTCCTTACTTACAGGTTGCTTCACACTGAGTGATAATTTAAAATTG
TTTCTATGTAGAATCATTGTCTCTACTGTTGCCCTCTCTGTGTCTGCACACATAATGC
CTCTCTCACTTTGGTGAAATATCAAAAAAATGCAAGTACAGCATGCATATTTAAGA
ATTTCTGAACTAACACTTGAAATCACATTTTATAAAGATTGTTGGTACTCAGAGTTG
TATTTGCAGCAGAAAATGCAGAATAACCCAGAAATGGAGCCCGGAGCCCAATTCCA
GAGAGGAAGGGAAAGAAGAGACGATGCCTGGACAATGTCTTATACATAGGGAACA
GAGAGGGAATCAGAGGTGGCTTAGTTCCCTGAAGACAGCCTTATGCGCTTAATCCC
TGCCTTTGAAAAATGGGCCAATACACAACACAGACAATAATTTGAAGCGTCTGTAT
TCAGCAAAGATAAATACAGCAGTCCAGTCTATAGGTGCTCTACTTAGCATTCTTTTC
ATGTGCTCCTGGTCTCTGTCTCACTCACCTACTTTCTACTACATCCAACTCAACTCCC
TGACACCCTATCCCCCCATCTATTCTTAAACTCTTTCATATATATTTTCACTTCCTAA
TTCTAGTGTACATATCCCTGTATATCATGGTATCTGTGGGCATATGTCTGTTTCTTAT
AAAGACACATATTTGTGTGTGTATGTGTGTGTGTGTGTGTGTGTGTGTATAAGCATA
CATGGGAATTTTTCTGAATGTAAGCAGTTTTGATGTGTCTGTATATCAATCTGTATCT
ACATTTGATGGACTGAGTAAGAATGTTGCATGTGAGAGAAGGGATGTGACTGTGGA
CTCAGATTGACACACACAGATTGGTGGAAGAATAGCTTCAGTCCTCTTCCTTCATCC
CATATGTAGGTAGCCTTGCTTGACAAATGTTTCTTCTAACTCTCCTTCTTCTGAATTG
TGCAGAGACTCTGTTAACTTCCCTTTTTGCACTTAGATAACTTCCTACTTCAACCCCC
TCCTTTATCTTGGATTTCTAAAGTTTTTCCCCAATATTGGGAATGGTGAGGGTGGGG
ATGAGAGACTCTCTGAACAATTCTTGCAGAACCAGTGACAATCTTATGAATATAAT
GAATAAGTAAGCATATAGTAGGGTGATAGAATACAGATAGTAGAAACAAAGAGAG
GACAGCAAACCTTTTCAGCACTTCTCTCAGAGCAAATGGAAACACACAGTAAGTAG
CTTTCTGACTACATTATTTTCTGGTCACTTTTAAAGAAAGTTTACAACCTGTTCTGAA
ACTATTTGTCTTTTCACTGGTTGTAAGTGTACCCCAATCTCAGGGAGTATATCTGTA
GTGCCACAGGCAAAAGATCCACTCCTTCCCACACTCATTTGCCTGAACTTACTCGAA
GGGCTGCATTTCTCTGAGTTTACGAAATTGTGTCATTATGGTCCCCATACAGTGGTA
TTTAACTTTTAAAGCAACTTTTAAGAAAACTCGACTTGTTTTTTGTTCATTTTAAGTG
TGTGGTACTAAAAAGACATGTTAGACTTTTTTTAAAAAAGCACTTATGTTTTGAAAA
TAGAATAAATAATAAGAATTTCCAATTAAATCATGTCTGGTGCCAATGTGCAAAAC
TTCA (SEQ ID NO: 12).
In an aspect, a disclosed PHKA1 can comprise the following sequence or a fragment
thereof:
ATGAGGAGCCGGAGTAACTCCGGGGTCCGGCTGGACGGCTACGCTCGACTGGTGCA
ACAGACCATCCTGTGCCATCAGAATCCAGTGACTGGCTTGCTTCCAGCCAGCTATGA
TCAGAAAGATGCTTGGGTCCGAGATAATGTGTACAGCATCTTGGCTGTGTGGGGTTT
GGGCCTGGCCTATCGGAAGAATGCAGACCGGGATGAGGATAAGGCAAAGGCCTAT
GAATTGGAGCAGAGTGTAGTGAAGCTGATGAGAGGACTACTGCACTGCATGATCAG
ACAGGTGGATAAAGTAGAATCCTTCAAATATAGTCAGAGTACTAAGGATAGCCTCC
ATGCAAAGTACAACACCAAAACCTGTGCCACTGTAGTGGGTGATGATCAATGGGGA
CACCTGCAGTTGGATGCTACCTCTGTGTACCTGCTCTTCTTAGCCCAAATGACTGCC
TCAGGACTCCATATCATCCACAGCCTAGATGAAGTCAATTTCATACAGAACCTTGTG
TTTTACATTGAAGCTGCATATAAAACTGCTGACTTCGGGATATGGGAACGTGGAGA
CAAGACCAACCAAGGGATCTCAGAGTTGAATGCCAGTTCAGTTGGAATGGCAAAGG
CAGCCCTGGAAGCATTAGATGAACTGGATCTGTTTGGTGTGAAAGGTGGGCCTCAA
TCAGTTATCCATGTCCTGGCTGATGAAGTACAGCACTGCCAGTCTATCCTAAATTCA
CTACTGCCCCGTGCTTCAACATCAAAAGAGGTTGATGCTAGTCTACTCTCAGTGGTT
TCCTTCCCTGCCTTTGCAGTAGAGGATAGCCAGTTGGTGGAGCTCACAAAACAGGA
AATCATCACCAAGCTTCAGGGTCGTTATGGTTGCTGTCGCTTTCTACGAGATGGATA
TAAAACTCCTAAAGAGGATCCCAATCGTCTGTACTATGAACCAGCTGAGCTGAAGC
TATTTGAAAACATTGAGTGTGAATGGCCATTGTTCTGGACATACTTTATTCTTGATG
GGGTCTTCAGTGGCAATGCAGAACAGGTTCAAGAATATAAAGAGGCTCTTGAAGCA
GTCCTCATCAAGGGCAAAAATGGAGTCCCACTTCTGCCAGAGCTGTACAGTGTTCCT
CCTGACAGGGTCGATGAAGAATATCAGAATCCTCACACTGTGGACCGAGTCCCCAT
GGGGAAATTGCCTCACATGTGGGGTCAGTCTCTATACATTTTAGGAAGCTTGATGGC
AGAGGGATTTTTAGCCCCTGGAGAAATTGATCCCCTGAATCGCAGGTTTTCTACTGT
ACCGAAGCCCGATGTTGTGGTTCAAGTCTCCATTCTAGCTGAAACAGAAGAAATCA
AGACCATTTTGAAGGACAAGGGAATTTACGTGGAGACCATTGCTGAGGTATACCCC
ATCAGAGTACAACCAGCTCGTATTCTCAGCCACATTTATTCCAGCCTAGGATGCAAC
AATAGAATGAAACTCAGTGGACGACCCTACAGACACATGGGAGTGCTTGGAACTTC
AAAACTCTATGACATTCGGAAAACTATCTTTACTTTCACTCCACAGTTTATAGACCA
GCAACAGTTCTACCTGGCTCTGGACAACAAGATGATAGTGGAAATGCTTAGAACAG
ACCTCTCCTACCTCTGTAGCCGCTGGCGGATGACAGGCCAGCCCACCATCACCTTCC
CCATCTCACACAGCATGCTTGATGAAGATGGAACAAGCTTGAATTCAAGTATCCTG
GCAGCACTCCGAAAAATGCAAGATGGGTATTTTGGTGGGGCAAGGGTTCAAACAGG
TAAATTGTCAGAGTTTTTGACAACATCTTGTTGCACACACTTGAGCTTCATGGACCC
TGGACCTGAGGGTAAGCTGTACAGTGAAGATTATGATGACAACTATGATTACCTGG
AATCTGGCAACTGGATGAATGATTATGATTCAACCAGTCATGCTCGCTGTGGTGATG
AAGTTGCTCGTTATTTAGATCACCTTTTGGCGCACACTGCTCCCCATCCTAAACTAG
CCCCTACCTCACAGAAGGGAGGGCTAGATCGGTTCCAAGCTGCTGTGCAAACAACC
TGCGACTTAATGTCCTTGGTGACCAAGGCCAAGGAACTGCATGTACAGAATGTTCA
CATGTATCTTCCTACGAAGTTATTTCAGGCTTCCCGGCCTTCATTCAACTTACTTGAT
TCACCTCATCCCCGACAGGAGAACCAGGTTCCCTCTGTTCGTGTAGAAATACATCTT
CCTAGAGACCAGTCTGGGGAGGTGGACTTTAAAGCACTGGTTTTACAGTTGAAGGA
GACCTCAAGCTTACAGGAACAAGCTGATATCCTCTATATGCTGTATACTATGAAAG
GACCTGACTGGAACACTGAATTGTATAATGAACGGAGTGCTACAGTGAGAGAGCTT
CTTACCGAGCTGTATGGCAAAGTGGGAGAAATTCGTCACTGGGGCCTGATCCGATA
CATTTCTGGGATCTTAAGGAAGAAAGTGGAAGCACTTGATGAGGCCTGCACAGACC
TTCTCTCCCACCAGAAACATTTGACAGTAGGACTTCCTCCAGAACCTCGAGAAAAG
ACTATCTCTGCACCTCTGCCCTATGAGGCGCTCACTCAGCTGATAGATGAAGCCAGT
GAAGGGGATATGAGCATTTCAATCCTTACACAGGAAATAATGGTATATCTAGCCAT
GTATATGCGAACCCAGCCTGGCCTCTTTGCTGAAATGTTTCGACTTCGAATTGGTCT
GATCATACAAGTTATGGCAACAGAACTGGCCCACTCCCTTCGATGCTCAGCTGAGG
AAGCCACAGAGGGCCTGATGAATCTCAGTCCTTCGGCCATGAAGAATCTCCTGCAT
CACATTCTCAGCGGCAAGGAGTTTGGAGTGGAACGAAGCGTTCGTCCCACTGATTC
AAATGTCAGTCCTGCTATTTCTATCCACGAGATTGGTGCTGTTGGAGCAACCAAAAC
AGAACGAACTGGGATCATGCAGTTAAAAAGTGAGATAAAGCAGGTGGAATTTCGTA
GACTGTCAATCTCAGCTGAGAGTCAGTCACCTGGAACCTCTATGACTCCAAGTAGTG
GGTCCTTTCCTAGTGCATATGATCAGCAGTCATCTAAAGATAGTCGTCAAGGTCAAT
GGCAACGCCGAAGAAGGCTGGATGGGGCACTGAATAGAGTTCCAGTTGGATTTTAT
CAGAAAGTATGGAAAGTTTTGCAGAAGTGTCACGGACTTTCTGTTGAAGGGTTTGTC
CTTCCTTCCTCTACCACTAGAGAGATGACTCCAGGTGAGATTAAATTCTCTGTTCAT
GTGGAGTCTGTCCTGAATCGTGTACCTCAGCCAGAGTACCGTCAGCTGCTGGTTGAA
GCCATCCTTGTCCTCACCATGCTGGCAGATATTGAAATTCATAGCATCGGAAGCATC
ATTGCTGTGGAAAAAATAGTGCATATTGCCAATGACTTGTTCCTTCAAGAACAGAA
AACCCTTGGCGCAGATGATACCATGTTGGCAAAGGATCCCGCATCTGGCATCTGTA
CTCTTCTGTATGACAGTGCACCCAGTGGCAGGTTTGGCACCATGACCTACCTCTCCA
AGGCAGCCGCCACCTACGTGCAGGAGTTCCTGCCCCACAGCATCTGTGCCATGCAA
TGA (SEQ ID NO: 13).
In an aspect, a disclosed PHKA1 can comprise the following sequence or a fragment
thereof:
GCCTCCCGCTCGCGTCTCCCGCAAGGTGGAGGCGGCGCAGCGGTCGTTGACCTGTT
GGCGGCGGCCGGGCGGGAGGCTGTGGGCCGCGGCGGTCTCCCGGGGTTGAGGGGA
GGATGCAGGAGTCACACGCGGAGGCCGGACTGGACTAAGGGGGCAGGAGCGGCGC
CTGGGGCGCTGCGGCGGGGCTCTAGACCGCGGGAAGCCAGTGCTAGCGCTGGCGCG
TGGTGGGCCCCTGAGGCCGCCGGAGTCCGGAGAGGTACGCGGAATCCGCGGCGCCA
GGCCTGAGCGGTGGGAGGGCTCTGCGGGGCCTGGTGTTCAGGCGTCCCACCACGAG
GGTGGAGCAGCGTTGGATACTTGTTCCTTAGGGACCGAAGGCTCCGGTGGCACCCG
GGCTATTTCTCAGAGGACAATTAGTAACGTGTCGCCATGAGGAGCCGGAGTAACTC
CGGGGTCCGGCTGGACGGCTACGCTCGACTGGTGCAACAGACCATCCTGTGCCATC
AGAATCCAGTGACTGGCTTGCTTCCAGCCAGCTATGATCAGAAAGATGCTTGGGTC
CGAGATAATGTGTACAGCATCTTGGCTGTGTGGGGTTTGGGCCTGGCCTATCGGAA
GAATGCAGACCGGGATGAGGATAAGGCAAAGGCCTATGAATTGGAGCAGAGTGTA
GTGAAGCTGATGAGAGGACTACTGCACTGCATGATCAGACAGGTGGATAAAGTAGA
ATCCTTCAAATATAGTCAGAGTACTAAGGATAGCCTCCATGCAAAGTACAACACCA
AAACCTGTGCCACTGTAGTGGGTGATGATCAATGGGGACACCTGCAGTTGGATGCT
ACCTCTGTGTACCTGCTCTTCTTAGCCCAAATGACTGCCTCAGGACTCCATATCATC
CACAGCCTAGATGAAGTCAATTTCATACAGAACCTTGTGTTTTACATTGAAGCTGCA
TATAAAACTGCTGACTTCGGGATATGGGAACGTGGAGACAAGACCAACCAAGGGAT
CTCAGAGTTGAATGCCAGTTCAGTTGGAATGGCAAAGGCAGCCCTGGAAGCATTAG
ATGAACTGGATCTGTTTGGTGTGAAAGGTGGGCCTCAATCAGTTATCCATGTCCTGG
CTGATGAAGTACAGCACTGCCAGTCTATCCTAAATTCACTACTGCCCCGTGCTTCAA
CATCAAAAGAGGTTGATGCTAGTCTACTCTCAGTGGTTTCCTTCCCTGCCTTTGCAG
TAGAGGATAGCCAGTTGGTGGAGCTCACAAAACAGGAAATCATCACCAAGCTTCAG
GGTCGTTATGGTTGCTGTCGCTTTCTACGAGATGGATATAAAACTCCTAAAGAGGAT
CCCAATCGTCTGTACTATGAACCAGCTGAGCTGAAGCTATTTGAAAACATTGAGTGT
GAATGGCCATTGTTCTGGACATACTTTATTCTTGATGGGGTCTTCAGTGGCAATGCA
GAACAGGTTCAAGAATATAAAGAGGCTCTTGAAGCAGTCCTCATCAAGGGCAAAAA
TGGAGTCCCACTTCTGCCAGAGCTGTACAGTGTTCCTCCTGACAGGGTCGATGAAGA
ATATCAGAATCCTCACACTGTGGACCGAGTCCCCATGGGGAAATTGCCTCACATGT
GGGGTCAGTCTCTATACATTTTAGGAAGCTTGATGGCAGAGGGATTTTTAGCCCCTG
GAGAAATTGATCCCCTGAATCGCAGGTTTTCTACTGTACCGAAGCCCGATGTTGTGG
TTCAAGTCTCCATTCTAGCTGAAACAGAAGAAATCAAGACCATTTTGAAGGACAAG
GGAATTTACGTGGAGACCATTGCTGAGGTATACCCCATCAGAGTACAACCAGCTCG
TATTCTCAGCCACATTTATTCCAGCCTAGGATGCAACAATAGAATGAAACTCAGTGG
ACGACCCTACAGACACATGGGAGTGCTTGGAACTTCAAAACTCTATGACATTCGGA
AAACTATCTTTACTTTCACTCCACAGTTTATAGACCAGCAACAGTTCTACCTGGCTC
TGGACAACAAGATGATAGTGGAAATGCTTAGAACAGACCTCTCCTACCTCTGTAGC
CGCTGGCGGATGACAGGCCAGCCCACCATCACCTTCCCCATCTCACACAGCATGCTT
GATGAAGATGGAACAAGCTTGAATTCAAGTATCCTGGCAGCACTCCGAAAAATGCA
AGATGGGTATTTTGGTGGGGCAAGGGTTCAAACAGGTAAATTGTCAGAGTTTTTGA
CAACATCTTGTTGCACACACTTGAGCTTCATGGACCCTGGACCTGAGGGTAAGCTGT
ACAGTGAAGATTATGATGACAACTATGATTACCTGGAATCTGGCAACTGGATGAAT
GATTATGATTCAACCAGTCATGCTCGCTGTGGTGATGAAGTTGCTCGTTATTTAGAT
CACCTTTTGGCGCACACTGCTCCCCATCCTAAACTAGCCCCTACCTCACAGAAGGGA
GGGCTAGATCGGTTCCAAGCTGCTGTGCAAACAACCTGCGACTTAATGTCCTTGGTG
ACCAAGGCCAAGGAACTGCATGTACAGAATGTTCACATGTATCTTCCTACGAAGTT
ATTTCAGGCTTCCCGGCCTTCATTCAACTTACTTGATTCACCTCATCCCCGACAGGA
GAACCAGGTTCCCTCTGTTCGTGTAGAAATACATCTTCCTAGAGACCAGTCTGGGGA
GGTGGACTTTAAAGCACTGGTTTTACAGTTGAAGGAGACCTCAAGCTTACAGGAAC
AAGCTGATATCCTCTATATGCTGTATACTATGAAAGGACCTGACTGGAACACTGAAT
TGTATAATGAACGGAGTGCTACAGTGAGAGAGCTTCTTACCGAGCTGTATGGCAAA
GTGGGAGAAATTCGTCACTGGGGCCTGATCCGATACATTTCTGGGATCTTAAGGAA
GAAAGTGGAAGCACTTGATGAGGCCTGCACAGACCTTCTCTCCCACCAGAAACATT
TGACAGTAGGACTTCCTCCAGAACCTCGAGAAAAGACTATCTCTGCACCTCTGCCCT
ATGAGGCGCTCACTCAGCTGATAGATGAAGCCAGTGAAGGGGATATGAGCATTTCA
ATCCTTACACAGGAAATAATGGTATATCTAGCCATGTATATGCGAACCCAGCCTGG
CCTCTTTGCTGAAATGTTTCGACTTCGAATTGGTCTGATCATACAAGTTATGGCAAC
AGAACTGGCCCACTCCCTTCGATGCTCAGCTGAGGAAGCCACAGAGGGCCTGATGA
ATCTCAGTCCTTCGGCCATGAAGAATCTCCTGCATCACATTCTCAGCGGCAAGGAGT
TTGGAGTGGAACGAAGCGTTCGTCCCACTGATTCAAATGTCAGTCCTGCTATTTCTA
TCCACGAGATTGGTGCTGTTGGAGCAACCAAAACAGAACGAACTGGGATCATGCAG
TTAAAAAGTGAGATAAAGCAGTCACCTGGAACCTCTATGACTCCAAGTAGTGGGTC
CTTTCCTAGTGCATATGATCAGCAGTCATCTAAAGATAGTCGTCAAGGTCAATGGCA
ACGCCGAAGAAGGCTGGATGGGGCACTGAATAGAGTTCCAGTTGGATTTTATCAGA
AAGTATGGAAAGTTTTGCAGAAGTGTCACGGACTTTCTGTTGAAGGGTTTGTCCTTC
CTTCCTCTACCACTAGAGAGATGACTCCAGGTGAGATTAAATTCTCTGTTCATGTGG
AGTCTGTCCTGAATCGTGTACCTCAGCCAGAGTACCGTCAGCTGCTGGTTGAAGCCA
TCCTTGTCCTCACCATGCTGGCAGATATTGAAATTCATAGCATCGGAAGCATCATTG
CTGTGGAAAAAATAGTGCATATTGCCAATGACTTGTTCCTTCAAGAACAGAAAACC
CTTGGCGCAGATGATACCATGTTGGCAAAGGATCCCGCATCTGGCATCTGTACTCTT
CTGTATGACAGTGCACCCAGTGGCAGGTTTGGCACCATGACCTACCTCTCCAAGGC
AGCCGCCACCTACGTGCAGGAGTTCCTGCCCCACAGCATCTGTGCCATGCAATGAG
GGCTTTGGTTCCTGGCTTCTGGGAGCCTTTTGACAGCTGGTCCCTGCCTCGGTTGATT
GTGCATGGAACTAAAATGTTATTGCCTAATCACTCCAACCCTGCCCCTTTCTGTCCC
ATCCTTCCCAAGAAGAGAGAACTTTTTCGATAAACTAACTACTGTAGAAGAAGTGA
ACACTTACCTGGAGGCTCACCTTGCAGAACCAGTGACAATCTTATGAGTATAATGA
ACACTCAGCCAGGCCTGTCATGATTGGCTTTATTTCTTTCATCATTCATAAAAGTTTG
CATGTGTTTTTATTCTCTAGATCTGTTACCAATATAGTTTTCTAACTCCTGTTTGGGG
AGCAAGTGTTAATAATAACTTATTCCTAAAGCTTGGTTTTTCTTTTTTGTGATTTTAT
TGTGTATATGAGTGGTGGGTACTTTGGGACATTATTTCAAATGAGTAAACTCTAAAT
TGTTAAATATTTATGCTGCTATAAATTAGAATTACCTATTCTACGTGCCTTTTCTTAG
GAAAAGAGAGTACTGGATCATGGTTTTCTTCCTTACTTACAGGTTGCTTCACACTGA
GTGATAATTTAAAATTGTTTCTATGTAGAATCATTGTCTCTACTGTTGCCCTCTCTGT
GTCTGCACACATAATGCCTCTCTCACTTTGGTGAAATATCAAAAAAATGCAAGTACA
GCATGCATATTTAAGAATTTCTGAACTAACACTTGAAATCACATTTTATAAAGATTG
TTGGTACTCAGAGTTGTATTTGCAGCAGAAAATGCAGAATAACCCAGAAATGGAGC
CCGGAGCCCAATTCCAGAGAGGAAGGGAAAGAAGAGACGATGCCTGGACAATGTC
TTATACATAGGGAACAGAGAGGGAATCAGAGGTGGCTTAGTTCCCTGAAGACAGCC
TTATGCGCTTAATCCCTGCCTTTGAAAAATGGGCCAATACACAACACAGACAATAA
TTTGAAGCGTCTGTATTCAGCAAAGATAAATACAGCAGTCCAGTCTATAGGTGCTCT
ACTTAGCATTCTTTTCATGTGCTCCTGGTCTCTGTCTCACTCACCTACTTTCTACTAC
ATCCAACTCAACTCCCTGACACCCTATCCCCCCATCTATTCTTAAACTCTTTCATATA
TATTTTCACTTCCTAATTCTAGTGTACATATCCCTGTATATCATGGTATCTGTGGGCA
TATGTCTGTTTCTTATAAAGACACATATTTGTGTGTGTATGTGTGTGTGTGTGTGTGT
GTGTGTATAAGCATACATGGGAATTTTTCTGAATGTAAGCAGTTTTGATGTGTCTGT
ATATCAATCTGTATCTACATTTGATGGACTGAGTAAGAATGTTGCATGTGAGAGAA
GGGATGTGACTGTGGACTCAGATTGACACACACAGATTGGTGGAAGAATAGCTTCA
GTCCTCTTCCTTCATCCCATATGTAGGTAGCCTTGCTTGACAAATGTTTCTTCTAACT
CTCCTTCTTCTGAATTGTGCAGAGACTCTGTTAACTTCCCTTTTTGCACTTAGATAAC
TTCCTACTTCAACCCCCTCCTTTATCTTGGATTTCTAAAGTTTTTCCCCAATATTGGG
AATGGTGAGGGTGGGGATGAGAGACTCTCTGAACAATTCTTGCAGAACCAGTGACA
ATCTTATGAATATAATGAATAAGTAAGCATATAGTAGGGTGATAGAATACAGATAG
TAGAAACAAAGAGAGGACAGCAAACCTTTTCAGCACTTCTCTCAGAGCAAATGGAA
ACACACAGTAAGTAGCTTTCTGACTACATTATTTTCTGGTCACTTTTAAAGAAAGTT
TACAACCTGTTCTGAAACTATTTGTCTTTTCACTGGTTGTAAGTGTACCCCAATCTCA
GGGAGTATATCTGTAGTGCCACAGGCAAAAGATCCACTCCTTCCCACACTCATTTGC
CTGAACTTACTCGAAGGGCTGCATTTCTCTGAGTTTACGAAATTGTGTCATTATGGT
CCCCATACAGTGGTATTTAACTTTTAAAGCAACTTTTAAGAAAACTCGACTTGTTTT
TTGTTCATTTTAAGTGTGTGGTACTAAAAAGACATGTTAGACTTTTTTTAAAAAAGC
ACTTATGTTTTGAAAATAGAATAAATAATAAGAATTTCCAATTAAATCATGTCTGGT
GCCAATGTGCAAAACTTCA (SEQ ID NO: 14).
In an aspect, a disclosed PHKA2 can comprise the following sequence or a fragment
thereof:
GCTTTCCAACCGGACTTTGGGGCTAGCGTTTAGAAAAGTCTCGACCTCCTCCGGCCC
GGTCCCATCCCAAGAACCGACTAAGGCTGTGAGTGTCCGGGAACCAGACCCGCTTG
GAGGCCACAGCCCCGACGTCCCGCGCCCACGCGGCAGATCGGGCGCTGCGGCCTGG
GAGCCTCGGGGAGATGCGGAGCAGGAGCAATTCCGGGGTCCGCTTGGACGGGTAC
GCGCGGCTGGTGCAGCAAACCATCCTGTGTTACCAGAATCCCGTCACGGGGCTGCT
GTCAGCCAGCCATGAGCAGAAGGATGCCTGGGTGCGGGATAACATCTACAGTATCC
TGGCCGTGTGGGGCCTGGGCATGGCCTACCGTAAGAATGCAGACCGCGATGAGGAC
AAGGCCAAGGCCTACGAGCTGGAGCAGAACGTGGTGAAGCTGATGCGAGGTCTTCT
CCAGTGCATGATGAGACAGGTGGCCAAAGTGGAGAAGTTCAAACACACTCAGAGC
ACCAAGGACAGCCTGCACGCCAAGTACAACACCGCCACCTGTGGCACGGTGGTGGG
CGACGACCAGTGGGGCCACCTCCAGGTGGATGCCACCTCTCTCTTCCTCCTGTTCCT
GGCCCAGATGACCGCCTCAGGCTTACGTATCATTTTCACTCTCGATGAGGTGGCCTT
CATACAGAATCTTGTCTTTTACATAGAAGCTGCATATAAAGTCGCTGATTATGGAAT
GTGGGAGCGTGGAGATAAGACTAATCAGGGCATCCCGGAATTGAATGCAAGCTCCG
TAGGAATGGCCAAGGCAGCTCTTGAGGCAATTGATGAACTGGACCTTTTTGGAGCC
CATGGAGGACGCAAGTCAGTGATTCATGTTCTGCCAGATGAGGTCGAGCACTGCCA
GTCTATTCTGTTCTCCATGCTGCCAAGAGCGTCGACATCTAAAGAAATTGATGCTGG
ACTTCTTTCCATTATTTCCTTCCCGGCCTTTGCAGTGGAAGATGTAAACCTTGTAAAT
GTGACCAAAAATGAAATTATTTCTAAGCTCCAGGGGCGTTATGGATGCTGTCGCTTC
CTTCGAGATGGTTATAAAACTCCAAGAGAGGACCCTAATCGACTGCATTATGACCC
TGCTGAACTCAAGCTCTTCGAAAACATTGAATGTGAGTGGCCTGTGTTTTGGACATA
TTTTATAATAGATGGAGTCTTCAGTGGTGATGCTGTTCAGGTCCAAGAATACCGAGA
GGCCCTGGAGGGAATACTCATCAGAGGCAAGAATGGGATCCGCCTGGTGCCTGAAC
TCTACGCTGTCCCGCCTAACAAGGTAGATGAAGAGTACAAGAATCCTCACACAGTA
GACCGAGTTCCTATGGGGAAGGTGCCTCATCTGTGGGGCCAATCCTTGTACATCCTC
AGCTCGCTGTTGGCAGAGGGATTCCTTGCCGCTGGTGAAATCGATCCCTTAAATAGA
AGATTTTCCACTTCAGTCAAACCTGATGTTGTAGTACAAGTTACTGTTTTGGCAGAA
AACAATCACATTAAGGACTTATTGAGGAAACACGGGGTGAACGTCCAGAGTATCGC
GGACATTCATCCAATTCAAGTCCAGCCGGGCCGGATTCTTAGTCACATATATGCCAA
GCTTGGACGGAATAAGAATATGAATTTGAGTGGGCGACCGTATCGACATATTGGTG
TCCTTGGAACCTCTAAACTATATGTGATTAGGAACCAAATCTTTACTTTTACACCCC
AGTTCACCGACCAGCATCACTTCTACCTGGCCCTCGACAATGAGATGATCGTGGAG
ATGCTAAGGATCGAGCTGGCCTACCTGTGCACCTGCTGGAGGATGACGGGCAGACC
CACACTCACCTTCCCCATCAGTCGCACCATGCTCACAAATGATGGCTCAGACATTCA
TTCTGCTGTGCTCTCCACAATTAGAAAACTAGAGGATGGATATTTTGGAGGAGCCA
GAGTAAAATTAGGGAACCTTTCGGAATTTCTCACCACATCGTTCTACACATATCTGA
CTTTTCTGGATCCAGACTGTGATGAGAAGTTGTTTGACAATGCCAGCGAAGGGACTT
TCAGTCCTGATAGTGATTCAGATTTGGTAGGATATCTGGAAGACACCTGTAATCAAG
AAAGCCAAGACGAACTTGACCATTATATCAACCACCTTCTGCAAAGCACATCGTTG
AGGTCCTATCTGCCTCCTCTTTGTAAGAACACAGAAGACCGCCATGTCTTCAGTGCT
ATCCACTCCACGCGGGACATACTTTCTGTGATGGCAAAAGCAAAGGGTTTGGAAGT
TCCATTTGTTCCCATGACTTTGCCGACTAAAGTTCTAAGTGCCCACCGTAAATCACT
GAATCTTGTTGATTCTCCTCAGCCACTCCTAGAAAAGGTTCCTGAAAGTGACTTTCA
GTGGCCCAGAGATGACCATGGTGACGTGGACTGTGAGAAGCTGGTTGAGCAGCTAA
AAGATTGTTCGAACCTACAGGACCAAGCAGACATTCTGTACATTCTTTATGTCATAA
AGGGTCCCAGCTGGGACACAAATCTCTCTGGACAGCACGGGGTCACCGTTCAAAAC
CTTCTTGGTGAGCTCTATGGGAAAGCCGGCTTGAACCAGGAGTGGGGTCTGATTCG
CTACATCTCAGGCCTTCTCAGGAAGAAAGTGGAGGTCCTGGCTGAGGCCTGCACAG
ACCTGCTTTCGCACCAGAAGCAGCTCACCGTGGGCCTGCCGCCCGAGCCCCGGGAG
AAGATCATCTCTGCGCCCCTTCCCCCAGAGGAGCTCACAAAACTCATCTACGAGGC
CAGTGGGCAGGACATCAGCATTGCCGTCCTCACGCAGGAGATTGTGGTTTACCTGG
CCATGTATGTCAGGGCGCAGCCCAGCCTCTTTGTGGAGATGCTGAGACTCCGGATTG
GACTGATCATTCAGGTGATGGCCACGGAGCTGGCACGGAGCCTGAACTGCTCAGGA
GAAGAGGCTTCTGAAAGTTTGATGAACCTCAGCCCTTTCGATATGAAAAATCTCCTG
CACCATATTCTAAGTGGGAAAGAGTTTGGCGTTGAAAGAAGTGTGCGCCCTATCCA
CTCCTCCACATCCAGCCCTACCATCTCCATCCACGAGGTGGGCCATACCGGAGTCAC
CAAAACTGAGAGGAGTGGCATTAACAGACTGAGGAGTGAAATGAAACAGATGACT
AGGCGGTTTAGTGCTGATGAACAGTTCTTTTCTGTGGGCCAGGCCGCGTCCAGCAGT
GCGCATTCCTCCAAGTCTGCGAGGTCCAGCACCCCATCCTCGCCCACTGGCACGTCA
TCCTCAGACTCGGGAGGACATCACATCGGCTGGGGTGAGCGGCAGGGCCAGTGGCT
GCGCAGGAGAAGGCTGGATGGGGCCATCAACAGGGTCCCCGTGGGATTCTACCAGA
GGGTGTGGAAGATCCTCCAGAAGTGCCACGGTCTCTCCATCGATGGTTATGTCCTCC
CATCCTCGACGACCCGAGAGATGACCCCGCATGAGATCAAGTTTGCTGTCCATGTC
GAATCGGTGCTGAACCGCGTGCCGCAGCCCGAGTACCGGCAGCTGCTGGTGGAAGC
CATCATGGTGCTGACGCTGCTCTCGGACACGGAGATGACCAGCATCGGGGGCATCA
TCCACGTGGACCAGATCGTGCAGATGGCCAGTCAGCTGTTCTTGCAGGACCAGGTG
TCAATTGGTGCCATGGACACCCTGGAGAAAGACCAAGCCACAGGAATCTGCCACTT
CTTTTATGACAGCGCTCCGAGTGGGGCTTATGGGACGATGACCTACCTAACAAGAG
CAGTGGCTTCTTATTTGCAGGAATTGTTGCCCAATTCGGGCTGCCAGATGCAATAGG
GTCTCACCTGGAAACATGATCACACTCTCAATCTGTCACGTGCCCCCTAGCCTTACT
GGGAACCTTCTGTCCCCCAAGATCCCCTGTGCTATCAGGAAAGCATGTCCCATCAGA
AACACTCTCGGGGGGCAATGGTAGCACTCACCCTGAAACTGATGTATGTTAAAGCC
ACAGAGATAGAGCTGAGGAGTCCTGTGTTCCCCCGCAAGGAGCACCCCGGGATCAT
TTTCTAGGTTCATTTCTCTGGAACATTTGCTGTAGCATCTGGTCTCACGGACTCTGAG
GAGGAATTGGAAATTGGTCTCTTTTGAGTGCAGAGGGAACTGAGACGCCAGCTTAA
ATTGGCTCTTGCAGAGAGTTACAGAAATAGTTTCGATGAGCTAGTGACACATCCTA
AAGATGCAAAGATCCTCCTGGCGGCAGTAGCCTTGACAAGGGCCACCTCTTCACAG
GATGCAGTCTGTCTGTGCACCAAACTCTTCACCAAATAGAACACTTGTGTCTCTCTG
TGGAATGGGGGTTTTCTTGTGCCTTGCTTGCTTTCATAGCCTTCCATTTTATTGGCAT
GGCTGCCTTGATGTAACATAATTCTCTGTCCCCAAGATTTAGAAAATTCCTCTTCGTT
CACCTTGGCTCATGGTCTTCCAGGGTTTTTATCCTGGCTGTCTATGAACTAGGGTTTT
CTCCTGCCTTAGGAAAAATACTGCATCTTCTGGAATCTAGAAAAAAAAAAAAAAAA
AGACAAGGCTCCTCTTATAGCCAGGCAAGCAGTTGTTTAGGGCTAGACCTGTTGTCC
CCGCTCGCGTGGGGTGAACGCCCCGCAGTGGATACTGTCCTCCTGTGCTCTGGGCGA
GGCGTCATAGGGTGAGCACAAGTCAGCAGTGCCATGGAAGTGAACAGGCATGTCCC
CGCAGAAACAATGCTTCCCTCTGAAAAATAGAGCCAGGCTAGGAACCTGTACCCTC
TTGGTAGAAGCACTTTGGTCTTCACTCTCTTGGGTGGGTGCAGTTGGAGGCACCGAT
TGGAGGAGAGCAAGGTTGAGTCAGGTGTCAGGGTCTGGCAAATCTTTTGCCAATGC
CCTAGCAATGGAGTTTCTCTCCGGGCATTGTCTTACCTTGGGGGAAGGAAACAAAA
CCAATGGGAAAACAGTTTCTGTAAATAATAAACCTTGAAAGACAAATGCTCA (SEQ
ID NO: 15).
In an aspect, a disclosed PHKA2 can comprise the following sequence or a fragment
thereof:
ATGCGGAGCAGGAGCAATTCCGGGGTCCGCTTGGACGGGTACGCGCGGCTGGTGCA
GCAAACCATCCTGTGTTACCAGAATCCCGTCACGGGGCTGCTGTCAGCCAGCCATG
AGCAGAAGGATGCCTGGGTGCGGGATAACATCTACAGTATCCTGGCCGTGTGGGGC
CTGGGCATGGCCTACCGTAAGAATGCAGACCGCGATGAGGACAAGGCCAAGGCCT
ACGAGCTGGAGCAGAACGTGGTGAAGCTGATGCGAGGTCTTCTCCAGTGCATGATG
AGACAGGTGGCCAAAGTGGAGAAGTTCAAACACACTCAGAGCACCAAGGACAGCC
TGCACGCCAAGTACAACACCGCCACCTGTGGCACGGTGGTGGGCGACGACCAGTGG
GGCCACCTCCAGGTGGATGCCACCTCTCTCTTCCTCCTGTTCCTGGCCCAGATGACC
GCCTCAGGCTTACGTATCATTTTCACTCTCGATGAGGTGGCCTTCATACAGAATCTT
GTCTTTTACATAGAAGCTGCATATAAAGTCGCTGATTATGGAATGTGGGAGCGTGG
AGATAAGACTAATCAGGGCATCCCGGAATTGAATGCAAGCTCCGTAGGAATGGCCA
AGGCAGCTCTTGAGGCAATTGATGAACTGGACCTTTTTGGAGCCCATGGAGGACGC
AAGTCAGTGATTCATGTTCTGCCAGATGAGGTCGAGCACTGCCAGTCTATTCTGTTC
TCCATGCTGCCAAGAGCGTCGACATCTAAAGAAATTGATGCTGGACTTCTTTCCATT
ATTTCCTTCCCGGCCTTTGCAGTGGAAGATGTAAACCTTGTAAATGTGACCAAAAAT
GAAATTATTTCTAAGCTCCAGGGGCGTTATGGATGCTGTCGCTTCCTTCGAGATGGT
TATAAAACTCCAAGAGAGGACCCTAATCGACTGCATTATGACCCTGCTGAACTCAA
GCTCTTCGAAAACATTGAATGTGAGTGGCCTGTGTTTTGGACATATTTTATAATAGA
TGGAGTCTTCAGTGGTGATGCTGTTCAGGTCCAAGAATACCGAGAGGCCCTGGAGG
GAATACTCATCAGAGGCAAGAATGGGATCCGCCTGGTGCCTGAACTCTACGCTGTC
CCGCCTAACAAGGTAGATGAAGAGTACAAGAATCCTCACACAGTAGACCGAGTTCC
TATGGGGAAGGTGCCTCATCTGTGGGGCCAATCCTTGTACATCCTCAGCTCGCTGTT
GGCAGAGGGATTCCTTGCCGCTGGTGAAATCGATCCCTTAAATAGAAGATTTTCCAC
TTCAGTCAAACCTGATGTTGTAGTACAAGTTACTGTTTTGGCAGAAAACAATCACAT
TAAGGACTTATTGAGGAAACACGGGGTGAACGTCCAGAGTATCGCGGACATTCATC
CAATTCAAGTCCAGCCGGGCCGGATTCTTAGTCACATATATGCCAAGCTTGGACGG
AATAAGAATATGAATTTGAGTGGGCGACCGTATCGACATATTGGTGTCCTTGGAAC
CTCTAAACTATATGTGATTAGGAACCAAATCTTTACTTTTACACCCCAGTTCACCGA
CCAGCATCACTTCTACCTGGCCCTCGACAATGAGATGATCGTGGAGATGCTAAGGA
TCGAGCTGGCCTACCTGTGCACCTGCTGGAGGATGACGGGCAGACCCACACTCACC
TTCCCCATCAGTCGCACCATGCTCACAAATGATGGCTCAGACATTCATTCTGCTGTG
CTCTCCACAATTAGAAAACTAGAGGATGGATATTTTGGAGGAGCCAGAGTAAAATT
AGGGAACCTTTCGGAATTTCTCACCACATCGTTCTACACATATCTGACTTTTCTGGA
TCCAGACTGTGATGAGAAGTTGTTTGACAATGCCAGCGAAGGGACTTTCAGTCCTG
ATAGTGATTCAGATTTGGTAGGATATCTGGAAGACACCTGTAATCAAGAAAGCCAA
GACGAACTTGACCATTATATCAACCACCTTCTGCAAAGCACATCGTTGAGGTCCTAT
CTGCCTCCTCTTTGTAAGAACACAGAAGACCGCCATGTCTTCAGTGCTATCCACTCC
ACGCGGGACATACTTTCTGTGATGGCAAAAGCAAAGGGTTTGGAAGTTCCATTTGTT
CCCATGACTTTGCCGACTAAAGTTCTAAGTGCCCACCGTAAATCACTGAATCTTGTT
GATTCTCCTCAGCCACTCCTAGAAAAGGTTCCTGAAAGTGACTTTCAGTGGCCCAGA
GATGACCATGGTGACGTGGACTGTGAGAAGCTGGTTGAGCAGCTAAAAGATTGTTC
GAACCTACAGGACCAAGCAGACATTCTGTACATTCTTTATGTCATAAAGGGTCCCA
GCTGGGACACAAATCTCTCTGGACAGCACGGGGTCACCGTTCAAAACCTTCTTGGT
GAGCTCTATGGGAAAGCCGGCTTGAACCAGGAGTGGGGTCTGATTCGCTACATCTC
AGGCCTTCTCAGGAAGAAAGTGGAGGTCCTGGCTGAGGCCTGCACAGACCTGCTTT
CGCACCAGAAGCAGCTCACCGTGGGCCTGCCGCCCGAGCCCCGGGAGAAGATCATC
TCTGCGCCCCTTCCCCCAGAGGAGCTCACAAAACTCATCTACGAGGCCAGTGGGCA
GGACATCAGCATTGCCGTCCTCACGCAGGAGATTGTGGTTTACCTGGCCATGTATGT
CAGGGCGCAGCCCAGCCTCTTTGTGGAGATGCTGAGACTCCGGATTGGACTGATCA
TTCAGGTGATGGCCACGGAGCTGGCACGGAGCCTGAACTGCTCAGGAGAAGAGGCT
TCTGAAAGTTTGATGAACCTCAGCCCTTTCGATATGAAAAATCTCCTGCACCATATT
CTAAGTGGGAAAGAGTTTGGCGTTGAAAGAAGTGTGCGCCCTATCCACTCCTCCAC
ATCCAGCCCTACCATCTCCATCCACGAGGTGGGCCATACCGGAGTCACCAAAACTG
AGAGGAGTGGCATTAACAGACTGAGGAGTGAAATGAAACAGATGACTAGGCGGTT
TAGTGCTGATGAACAGTTCTTTTCTGTGGGCCAGGCCGCGTCCAGCAGTGCGCATTC
CTCCAAGTCTGCGAGGTCCAGCACCCCATCCTCGCCCACTGGCACGTCATCCTCAGA
CTCGGGAGGACATCACATCGGCTGGGGTGAGCGGCAGGGCCAGTGGCTGCGCAGG
AGAAGGCTGGATGGGGCCATCAACAGGGTCCCCGTGGGATTCTACCAGAGGGTGTG
GAAGATCCTCCAGAAGTGCCACGGTCTCTCCATCGATGGTTATGTCCTCCCATCCTC
GACGACCCGAGAGATGACCCCGCATGAGATCAAGTTTGCTGTCCATGTCGAATCGG
TGCTGAACCGCGTGCCGCAGCCCGAGTACCGGCAGCTGCTGGTGGAAGCCATCATG
GTGCTGACGCTGCTCTCGGACACGGAGATGACCAGCATCGGGGGCATCATCCACGT
GGACCAGATCGTGCAGATGGCCAGTCAGCTGTTCTTGCAGGACCAGGTGTCAATTG
GTGCCATGGACACCCTGGAGAAAGACCAAGCCACAGGAATCTGCCACTTCTTTTAT
GACAGCGCTCCGAGTGGGGCTTATGGGACGATGACCTACCTAACAAGAGCAGTGGC
TTCTTATTTGCAGGAATTGTTGCCCAATTCGGGCTGCCAGATGCAATAG (SEQ ID
NO: 16).
In an aspect, a disclosed PHKB can comprise the following sequence or a fragment thereof:
ATGGCGGGGGCGGCGGGACTCACGGCAGAAGTGAGCTGGAAGGTCTTGGAGCGAA
GAGCTCGGACCAAGCGCTCAGGCTCAGTTTATGAACCTCTTAAAAGCATTAATCTTC
CAAGACCTGATAATGAAACTCTCTGGGATAAGTTGGACCATTATTACAGAATTGTC
AAGTCAACATTGCTGCTGTATCAAAGTCCAACTACCGGTCTCTTTCCCACTAAAACA
TGCGGTGGTGACCAGAAGGCCAAGATCCAGGACAGCCTATACTGCGCTGCTGGGGC
CTGGGCTTTGGCTCTTGCATACAGGCGAATTGATGATGACAAGGGAAGGACCCATG
AGCTGGAGCACTCAGCTATAAAATGCATGAGAGGAATTCTCTACTGCTATATGCGT
CAGGCCGATAAGGTCCAGCAGTTTAAGCAGGATCCACGCCCAACAACATGTCTTCA
CTCTGTTTTCAATGTGCATACAGGAGATGAGTTGCTTTCCTATGAGGAATATGGTCA
TCTTCAGATAAATGCAGTGTCACTTTATCTCCTTTACCTTGTGGAAATGATTTCCTCA
GGACTCCAGATTATCTACAACACTGATGAGGTCTCTTTTATTCAAAACCTTGTATTTT
GTGTGGAAAGAGTTTACCGTGTGCCTGACTTTGGTGTCTGGGAAAGAGGAAGCAAA
TATAATAATGGCAGCACAGAGCTACATTCGAGCTCGGTTGGTTTAGCAAAAGCAGC
TCTAGAAGCAATTAATGGATTCAACCTTTTTGGCAACCAGGGCTGTTCGTGGTCAGT
TATATTTGTGGATCTCGATGCTCACAATCGCAACAGGCAAACTTTGTGCTCGCTGTT
ACCCAGAGAATCAAGATCACATAATACAGATGCTGCCCTGCTCCCCTGCATCAGTT
ATCCTGCATTTGCCCTGGATGATGAAGTTCTTTTTAGCCAGACACTTGATAAAGTGG
TTAGAAAATTAAAAGGAAAATATGGATTTAAACGTTTCTTGAGAGATGGGTATAGA
ACATCATTGGAAGATCCCAACAGATGCTACTACAAGCCAGCTGAAATTAAGCTATT
TGATGGCATTGAATGTGAATTTCCCATATTTTTCCTTTATATGATGATTGATGGAGTT
TTTAGAGGCAATCCTAAGCAAGTACAGGAATATCAGGATCTTTTGACTCCAGTACTT
CATCATACCACAGAAGGATATCCTGTTGTACCAAAGTACTATTATGTGCCAGCTGAC
TTTGTAGAATATGAAAAAAATAACCCTGGTAGTCAAAAACGATTTCCTAGCAACTG
TGGCCGTGATGGAAAACTGTTTCTTTGGGGACAAGCACTTTATATCATCGCAAAACT
CCTGGCTGATGAACTTATTAGTCCTAAAGACATTGATCCTGTCCAGCGCTATGTCCC
ACTAAAGGATCAACGTAACGTGAGCATGAGGTTTTCCAATCAGGGCCCACTGGAAA
ATGACTTGGTAGTTCATGTGGCACTTATAGCAGAAAGCCAAAGACTTCAAGTTTTTC
TGAACACATATGGTATTCAAACTCAAACTCCTCAACAAGTAGAACCCATTCAGATA
TGGCCTCAGCAGGAGCTTGTGAAAGCTTATTTGCAGCTGGGTATCAATGAAAAGTT
AGGACTCTCTGGAAGGCCAGACAGGCCCATTGGCTGCCTCGGGACATCAAAGATTT
ATCGCATTCTAGGAAAGACTGTGGTTTGTTACCCGATTATTTTCGACCTAAGTGATT
TCTACATGTCTCAGGATGTTTTCCTGCTGATAGATGACATAAAGAATGCGCTGCAGT
TCATTAAACAATATTGGAAAATGCATGGACGTCCACTTTTCCTTGTTCTCATCCGGG
AAGACAATATAAGAGGTAGCCGGTTCAACCCCATATTAGATATGCTGGCAGCCCTT
AAAAAAGGAATAATTGGAGGAGTCAAAGTTCATGTGGATCGTCTACAGACACTAAT
ATCTGGAGCTGTGGTAGAACAACTTGATTTCCTACGAATCAGTGACACAGAAGAGC
TTCCAGAATTTAAGAGTTTTGAGGAACTAGAACCTCCCAAACATTCAAAAGTCAAA
CGGCAAAGCAGCACCCCTAGTGCTCCTGAACTGGGACAGCAGCCGGATGTCAACAT
TAGTGAATGGAAGGACAAACCCACCCACGAAATTCTTCAAAAACTGAATGATTGCA
GTTGTCTGGCTAGCCAAGCCATCCTGCTGGGTATACTGCTCAAAAGAGAAGGCCCC
AACTTCATCACAAAGGAAGGTACCGTTTCTGATCACATTGAGAGAGTCTATAGAAG
AGCTGGCAGCCAAAAACTTTGGTTGGCGGTGCGCTACGGGGCTGCATTTACCCAGA
AATTTTCTTCCTCTATAGCCCCACACATTACTACTTTTCTGGTACATGGGAAACAGG
TAACTCTGGGTGCCTTTGGGCATGAAGAAGAAGTTATCTCTAATCCTTTGTCTCCAA
GAGTGATTCAAAACATCATCTATTATAAGTGTAACACCCATGATGAGAGGGAAGCG
GTCATTCAGCAAGAACTGGTCATCCATATTGGCTGGATCATCTCCAATAACCCTGAG
TTATTCAGTGGCATGCTGAAAATACGAATCGGGTGGATCATCCATGCCATGGAGTA
TGAACTTCAGATCCGTGGCGGAGACAAGCCAGCCTTGGACTTGTATCAGCTGTCAC
CTAGTGAAGTTAAACAGCTTCTGCTGGATATTCTGCAGCCTCAACAGAATGGAAGA
TGTTGGCTGAACAGGCGTCAGATCGATGGGTCTTTGAATAGAACTCCCACCGGGTTC
TATGACCGAGTGTGGCAGATTCTGGAGCGCACGCCCAATGGGATCATTGTTGCTGG
GAAGCATTTGCCTCAGCAACCAACCCTGTCAGATATGACCATGTATGAGATGAATTT
CTCTCTCCTTGTTGAAGACACGTTGGGAAATATTGACCAGCCACAGTACAGACAGA
TCGTTGTAGAGTTACTTATGGTTGTATCCATTGTACTGGAAAGAAACCCCGAGCTAG
AATTTCAAGACAAAGTAGATCTAGACAGACTGGTCAAAGAAGCATTTAATGAATTT
CAAAAAGATCAGAGTCGGCTAAAGGAAATTGAAAAACAAGATGACATGACTTCCTT
TTACAACACTCCTCCCCTGGGAAAAAGAGGAACATGCAGCTATTTGACAAAGGCGG
TGATGAATCTGCTGCTGGAAGGAGAAGTCAAGCCAAACAATGATGACCCGTGTCTG
ATTAGCTAG (SEQ ID NO: 17).

In an aspect, a disclosed CALM1 can comprise the sequence set forth in SEQ ID NO:60 or a fragment thereof. In an aspect, a disclosed CALM2 can comprise the sequence set forth in SEQ ID NO:61 or a fragment thereof. In an aspect, a disclosed CALM3 can comprise the sequence set forth in SEQ ID NO:62 or a fragment thereof. In an aspect, a disclosed CALM can comprise a sequence having about 30%, about 40%, about 50%, about 60%, about 70%, about 80%, about 85%, about 90%, about 95%, or greater than 95% identity than the sequence set forth in SEQ ID NO:60, SEQ ID NO:61, or SEQ ID NO:62.

In an aspect, a disclosed PHKG2 can comprise the following sequence or a fragment
thereof:
ATGACGCTGGACGTGGGGCCGGAGGATGAGCTGCCCGACTGGGCCGCCGCCAAAG
AGTTTTACCAGAAGTACGACCCTAAGGACGTCATCGGCAGAGGAGTGAGCTCTGTG
GTCCGCCGTTGTGTTCATCGAGCTACTGGCCACGAGTTTGCGGTGAAGATTATGGAA
GTGACAGCTGAGCGGCTGAGTCCTGAGCAGCTGGAGGAGGTGCGGGAAGCCACAC
GGCGAGAGACACACATCCTTCGCCAGGTCGCCGGCCACCCCCACATCATCACCCTC
ATCGATTCCTACGAGTCTTCTAGCTTCATGTTCCTGGTGTTTGACCTGATGCGGAAG
GGAGAGCTGTTTGACTATCTCACAGAGAAGGTGGCCCTCTCTGAAAAGGAAACCAG
GTCCATCATGCGGTCTCTGCTGGAAGCAGTGAGCTTTCTCCATGCCAACAACATTGT
GCATCGAGATCTGAAGCCCGAGAATATTCTCCTAGATGACAATATGCAGATCCGAC
TTTCAGATTTCGGGTTCTCCTGCCACTTGGAACCTGGCGAGAAGCTTCGAGAGTTGT
GTGGGACCCCAGGGTATCTAGCGCCAGAGATCCTTAAATGCTCCATGGATGAAACC
CACCCAGGCTATGGCAAGGAGGTCGACCTCTGGGCCTGTGGGGTGATCTTGTTCAC
ACTCCTGGCTGGCTCGCCACCCTTCTGGCACCGGCGGCAGATCCTGATGTTACGCAT
GATCATGGAGGGCCAGTACCAGTTCAGTTCCCCCGAGTGGGATGACCGTTCCAGCA
CTGTCAAAGACCTGATCTCCAGGCTGCTGCAGGTGGATCCTGAGGCACGCCTGACA
GCTGAGCAGGCCCTACAGCACCCCTTCTTTGAGCGTTGTGAAGGCAGCCAACCCTG
GAACCTCACCCCCCGCCAGCGGTTCCGGGTGGCAGTGTGGACAGTGCTGGCTGCTG
GACGAGTGGCCCTAAGCACCCATCGTGTACGGCCACTGACCAAGAATGCACTGTTG
AGGGACCCTTATGCGCTGCGGTCAGTGCGGCACCTCATCGACAACTGTGCCTTCCGG
CTCTACGGGCACTGGATAAGGAAGCAGTGGATTGGAAAGCTGATGGCTTGTGTATG
A (SEQ ID NO: 18).
In an aspect, a disclosed PHKG2 can comprise the following sequence or a fragment
thereof:
ATGACACTTGATGTGGGCCCTGAAGATGAATTGCCTGACTGGGCTGCTGCCAAAGA
ATTTTACCAAAAATATGACCCTAAAGATGTGATTGGAAGGGGAGTTTCTAGTGTTGT
GAGAAGGTGTGTCCACAGGGCTACAGGGCATGAATTTGCAGTCAAGATTATGGAAG
TCACTGCTGAAAGGTTGTCCCCTGAGCAGCTGGAGGAAGTTAGGGAAGCAACTAGG
AGAGAGACACATATACTTAGACAAGTTGCAGGTCACCCTCACATTATTACTCTGATT
GATAGTTATGAGTCTTCCAGCTTCATGTTCCTTGTGTTTGACCTCATGAGGAAGGGG
GAGTTGTTTGACTATCTCACAGAAAAAGTGGCCCTGAGTGAAAAGGAGACAAGGTC
CATAATGAGGAGTCTCCTGGAAGCTGTGAGCTTCCTTCATGCTAACAATATAGTCCA
TAGAGATCTGAAACCTGAGAATATCCTTCTGGATGACAATATGCAGATCAGATTGA
GTGATTTTGGCTTCAGCTGTCATCTGGAGCCTGGTGAGAAGCTTAGGGAACTCTGTG
GCACACCAGGGTATTTGGCACCTGAAATCCTGAAATGTAGCATGGATGAGACCCAC
CCTGGCTATGGTAAAGAGGTTGACCTTTGGGCTTGTGGAGTAATACTTTTCACCCTC
CTTGCTGGGTCACCACCTTTCTGGCATAGAAGGCAGATTCTGATGCTGAGGATGATT
ATGGAGGGACAATATCAGTTCTCAAGTCCTGAGTGGGATGATAGATCCTCAACTGT
CAAGGATCTTATCTCAAGGCTTCTTCAAGTGGATCCAGAGGCTAGACTCACAGCTG
AACAAGCTCTCCAACACCCTTTTTTTGAAAGGTGTGAAGGTTCTCAGCCTTGGAACC
TGACTCCTAGACAGAGGTTCAGGGTGGCTGTGTGGACTGTGCTTGCAGCTGGTAGG
GTAGCACTTAGCACACACAGGGTTAGGCCACTGACCAAAAATGCTCTTTTGAGAGA
TCCCTATGCTCTGAGGTCTGTTAGGCACCTCATCGACAATTGTGCTTTCAGGCTCTAT
GGTCATTGGATAAGAAAACAGTGGATTGGCAAGCTGATGGCTTGCGTATGA (SEQ ID
NO: 19).
In an aspect, a disclosed PHKG2 can comprise the following sequence or a fragment
thereof:
ATGACTTTGGACGTGGGCCCTGAAGATGAGCTCCCTGACTGGGCTGCAGCTAAAGA
GTTCTACCAGAAATATGACCCTAAAGATGTGATTGGTAGAGGAGTCAGTTCTGTTGT
GAGAAGATGTGTACACAGGGCTACTGGGCATGAGTTTGCCGTTAAGATCATGGAAG
TGACAGCTGAGAGGTTGTCCCCAGAGCAACTGGAAGAGGTGAGAGAAGCAACAAG
AAGGGAGACCCATATTCTTAGGCAGGTAGCTGGACATCCCCATATCATCACCTTGAT
AGATAGTTATGAATCCAGTTCTTTTATGTTCCTGGTGTTTGATTTGATGAGGAAGGG
GGAACTGTTTGATTATCTGACTGAAAAAGTGGCACTTAGTGAGAAAGAAACTAGAT
CTATAATGAGGTCCCTGCTTGAAGCTGTGAGTTTTCTCCATGCCAATAATATAGTAC
ACAGGGACCTCAAACCAGAGAATATCCTCCTTGACGATAATATGCAGATCAGGCTT
AGTGATTTTGGCTTTTCATGTCACCTTGAGCCAGGAGAGAAACTTAGGGAACTTTGT
GGCACACCTGGATATCTGGCTCCAGAGATATTGAAGTGTAGCATGGATGAGACTCA
TCCAGGGTATGGCAAGGAGGTGGACTTGTGGGCCTGTGGTGTGATACTCTTCACTCT
GCTTGCTGGGAGTCCTCCTTTCTGGCATAGGAGACAGATTTTGATGCTCAGGATGAT
CATGGAAGGGCAGTATCAGTTTAGCTCTCCTGAATGGGATGATAGAAGTAGCACCG
TTAAGGATCTTATTTCCAGATTGTTGCAAGTGGACCCTGAGGCTAGACTGACAGCTG
AACAGGCACTGCAGCATCCATTTTTTGAGAGATGCGAAGGTTCCCAGCCCTGGAAT
CTCACCCCTAGACAAAGGTTCAGGGTGGCAGTCTGGACTGTATTGGCTGCAGGTAG
GGTGGCCCTTTCCACACACAGAGTCAGGCCCTTGACCAAGAATGCTCTTCTGAGGG
ACCCTTATGCTCTCAGATCTGTGAGGCACCTCATAGATAACTGTGCCTTCAGGTTGT
ATGGCCATTGGATAAGGAAGCAATGGATTGGTAAGCTGATGGCCTGTGTTTGA (SEQ
ID NO: 20).
In an aspect, a disclosed PYGL can comprise the following sequence or a fragment thereof:
ACCCCTGCCCGGCAGCCCAGCGCCTCCGGCCGCACTTCCAGCTCTCTGCGCAGCCCG
CCGCGCAGCCCGCCGCCCCAGCCATGGCGAAGCCCCTGACGGACCAGGAGAAGCG
GCGGCAGATCAGCATCCGCGGCATCGTGGGCGTGGAGAACGTGGCAGAGCTGAAG
AAGAGTTTCAACCGGCACCTGCACTTCACGCTGGTCAAGGACCGCAACGTGGCCAC
CACCCGCGACTACTACTTCGCGCTGGCGCACACGGTGCGCGACCACCTGGTGGGGC
GCTGGATCCGCACGCAGCAGCACTACTACGACAAGTGCCCCAAGCTTGGATTGGAT
ATAGAAGAGTTAGAAGAAATTGAAGAAGATGCTGGACTTGGCAATGGTGGTCTTGG
GAGACTTGCTGCCTGCTTCTTGGATTCCATGGCAACCCTGGGACTTGCAGCCTATGG
ATACGGCATTCGGTATGAATATGGGATTTTCAATCAGAAGATCCGAGATGGATGGC
AGGTAGAAGAAGCAGATGATTGGCTCAGATATGGAAACCCTTGGGAGAAGTCCCGC
CCAGAATTCATGCTGCCTGTGCACTTCTATGGAAAAGTAGAACACACCAACACCGG
GACCAAGTGGATTGACACTCAAGTGGTCCTGGCTCTGCCATATGACACCCCCGTGCC
CGGCTACATGAATAACACTGTCAACACCATGCGCCTCTGGTCTGCTCGGGCACCAA
ATGACTTTAACCTCAGAGACTTTAATGTTGGAGACTACATTCAGGCTGTGCTGGACC
GAAACCTGGCCGAGAACATCTCCCGGGTCCTCTATCCCAATGACAATTTTTTTGAAG
GGAAGGAGCTAAGATTGAAGCAGGAATACTTTGTGGTGGCTGCAACCTTGCAAGAT
ATCATCCGCCGTTTCAAAGCCTCCAAGTTTGGCTCCACCCGTGGTGCAGGAACTGTG
TTTGATGCCTTCCCGGATCAGGTGGCCATCCAGCTGAATGACACTCACCCTGCACTC
GCGATCCCTGAGCTGATGAGGATTTTTGTGGATATTGAAAAACTGCCCTGGTCCAAG
GCATGGGAGCTCACCCAGAAGACCTTCGCCTACACCAACCACACAGTGCTCCCGGA
AGCCCTGGAGCGCTGGCCCGTGGACCTGGTGGAGAAGCTGCTCCCTCGACATTTGG
AAATCATTTATGAGATAAATCAGAAGCATTTAGATAGAATTGTGGCCTTGTTTCCTA
AAGATGTGGACCGTCTGAGAAGGATGTCTCTGATAGAAGAGGAAGGAAGCAAAAG
GATCAACATGGCCCATCTCTGCATTGTCGGTTCCCATGCTGTGAATGGCGTGGCTAA
AATCCACTCAGACATCGTGAAGACTAAAGTATTCAAGGACTTCAGTGAGCTAGAAC
CTGACAAGTTTCAGAATAAAACCAATGGGATCACTCCAAGGCGCTGGCTCCTACTC
TGCAACCCAGGACTTGCAGAGCTCATAGCAGAGAAAATTGGAGAAGACTATGTGAA
AGACCTGAGCCAGCTGACGAAGCTCCACAGCTTCCTGGGTGATGATGTCTTCCTCCG
GGAACTCGCCAAGGTGAAGCAGGAGAATAAGCTGAAGTTTTCTCAGTTCCTGGAGA
CGGAGTACAAAGTGAAGATCAACCCATCCTCCATGTTTGATGTCCAGGTGAAGAGG
ATACATGAGTACAAGCGACAGCTCTTGAACTGTCTGCATGTGATCACGATGTACAA
CCGCATTAAGAAAGACCCTAAGAAGTTATTCGTGCCAAGGACAGTTATCATTGGTG
GTAAAGCTGCCCCAGGATATCACATGGCCAAAATGATCATAAAGCTGATCACTTCA
GTGGCAGATGTGGTGAACAATGACCCTATGGTTGGAAGCAAGTTGAAAGTCATCTT
CTTGGAGAACTACAGAGTATCTCTTGCTGAAAAAGTCATTCCAGCCACAGATCTGTC
AGAGCAGATTTCCACTGCAGGCACCGAAGCCTCGGGGACAGGCAATATGAAGTTCA
TGCTAAATGGGGCCCTAACTATCGGGACCATGGATGGGGCCAATGTGGAAATGGCA
GAAGAAGCTGGGGAAGAGAACCTGTTCATCTTTGGCATGAGGATAGATGATGTGGC
TGCTTTGGACAAGAAAGGGTACGAGGCAAAAGAATACTATGAGGCACTTCCAGAGC
TGAAGCTGGTCATTGATCAAATTGACAATGGCTTTTTTTCTCCCAAGCAGCCTGACC
TCTTCAAAGATATCATCAACATGCTATTTTATCATGACAGGTTTAAAGTCTTTGCAG
ACTACGAAGCCTATGTCAAGTGTCAAGATAAAGTGAGTCAGCTGTACATGAATCCA
AAGGCCTGGAACACAATGGTACTCAAAAACATAGCTGCCTCGGGGAAATTCTCCAG
TGACCGAACAATTAAAGAATATGCCCAAAACATCTGGAACGTGGAACCTTCAGATC
TAAAGATTTCTCTATCCAATGAATCTAACAAAGTCAATGGAAATTGAACTCTAGAAT
TGTCTCTAGAAAACATAGCTTCTTACTGAACTTGAACATTTTTACAACATTCACTGG
TTTTTGTTTTGTTAGCTAATAATCTATAATAGTTGAGTATCTCTGGGAATGGGGAGG
GAAATTATATGTAATAGAGCTTAAAAATAAAGTGTCAATTTCCAAGGGCTA (SEQ ID
NO: 21).
In an aspect, a disclosed PYGL can comprise the following sequence or a fragment thereof:
ACCCCTGCCCGGCAGCCCAGCGCCTCCGGCCGCACTTCCAGCTCTCTGCGCAGCCCG
CCGCGCAGCCCGCCGCCCCAGCCATGGCGAAGCCCCTGACGGACCAGGAGAAGCG
GCGGCAGATCAGCATCCGCGGCATCGTGGGCGTGGAGAACGTGGCAGAGCTGAAG
AAGAGTTTCAACCGGCACCTGCACTTCACGCTGGTCAAGGACCGCAACGTGGCCAC
CACCCGCGACTACTACTTCGCGCTGGCGCACACGGTGCGCGACCACCTGGTGGGGC
GCTGGATCCGCACGCAGCAGCACTACTACGACAAGTGCCCCAAGAGGGTATATTAC
CTCTCTCTGGAATTTTACATGGGCCGAACATTACAGAACACCATGATCAACCTCGGT
CTGCAAAATGCCTGTGATGAGGCCATTTACCAGCTTGGATTGGATATAGAAGAGTT
AGAAGAAATTGAAGAAGATGCTGGACTTGGCAATGGTGGTCTTGGGAGACTTGCTG
CCTGCTTCTTGGATTCCATGGCAACCCTGGGACTTGCAGCCTATGGATACGGCATTC
GGTATGAATATGGGATTTTCAATCAGAAGATCCGAGATGGATGGCAGGTAGAAGAA
GCAGATGATTGGCTCAGATATGGAAACCCTTGGGAGAAGTCCCGCCCAGAATTCAT
GCTGCCTGTGCACTTCTATGGAAAAGTAGAACACACCAACACCGGGACCAAGTGGA
TTGACACTCAAGTGGTCCTGGCTCTGCCATATGACACCCCCGTGCCCGGCTACATGA
ATAACACTGTCAACACCATGCGCCTCTGGTCTGCTCGGGCACCAAATGACTTTAACC
TCAGAGACTTTAATGTTGGAGACTACATTCAGGCTGTGCTGGACCGAAACCTGGCC
GAGAACATCTCCCGGGTCCTCTATCCCAATGACAATTTTTTTGAAGGGAAGGAGCTA
AGATTGAAGCAGGAATACTTTGTGGTGGCTGCAACCTTGCAAGATATCATCCGCCG
TTTCAAAGCCTCCAAGTTTGGCTCCACCCGTGGTGCAGGAACTGTGTTTGATGCCTT
CCCGGATCAGGTGGCCATCCAGCTGAATGACACTCACCCTGCACTCGCGATCCCTG
AGCTGATGAGGATTTTTGTGGATATTGAAAAACTGCCCTGGTCCAAGGCATGGGAG
CTCACCCAGAAGACCTTCGCCTACACCAACCACACAGTGCTCCCGGAAGCCCTGGA
GCGCTGGCCCGTGGACCTGGTGGAGAAGCTGCTCCCTCGACATTTGGAAATCATTTA
TGAGATAAATCAGAAGCATTTAGATAGAATTGTGGCCTTGTTTCCTAAAGATGTGG
ACCGTCTGAGAAGGATGTCTCTGATAGAAGAGGAAGGAAGCAAAAGGATCAACAT
GGCCCATCTCTGCATTGTCGGTTCCCATGCTGTGAATGGCGTGGCTAAAATCCACTC
AGACATCGTGAAGACTAAAGTATTCAAGGACTTCAGTGAGCTAGAACCTGACAAGT
TTCAGAATAAAACCAATGGGATCACTCCAAGGCGCTGGCTCCTACTCTGCAACCCA
GGACTTGCAGAGCTCATAGCAGAGAAAATTGGAGAAGACTATGTGAAAGACCTGA
GCCAGCTGACGAAGCTCCACAGCTTCCTGGGTGATGATGTCTTCCTCCGGGAACTCG
CCAAGGTGAAGCAGGAGAATAAGCTGAAGTTTTCTCAGTTCCTGGAGACGGAGTAC
AAAGTGAAGATCAACCCATCCTCCATGTTTGATGTCCAGGTGAAGAGGATACATGA
GTACAAGCGACAGCTCTTGAACTGTCTGCATGTGATCACGATGTACAACCGCATTA
AGAAAGACCCTAAGAAGTTATTCGTGCCAAGGACAGTTATCATTGGTGGTAAAGCT
GCCCCAGGATATCACATGGCCAAAATGATCATAAAGCTGATCACTTCAGTGGCAGA
TGTGGTGAACAATGACCCTATGGTTGGAAGCAAGTTGAAAGTCATCTTCTTGGAGA
ACTACAGAGTATCTCTTGCTGAAAAAGTCATTCCAGCCACAGATCTGTCAGAGCAG
ATTTCCACTGCAGGCACCGAAGCCTCGGGGACAGGCAATATGAAGTTCATGCTAAA
TGGGGCCCTAACTATCGGGACCATGGATGGGGCCAATGTGGAAATGGCAGAAGAA
GCTGGGGAAGAGAACCTGTTCATCTTTGGCATGAGGATAGATGATGTGGCTGCTTT
GGACAAGAAAGGGTACGAGGCAAAAGAATACTATGAGGCACTTCCAGAGCTGAAG
CTGGTCATTGATCAAATTGACAATGGCTTTTTTTCTCCCAAGCAGCCTGACCTCTTCA
AAGATATCATCAACATGCTATTTTATCATGACAGGTTTAAAGTCTTTGCAGACTACG
AAGCCTATGTCAAGTGTCAAGATAAAGTGAGTCAGCTGTACATGAATCCAAAGGCC
TGGAACACAATGGTACTCAAAAACATAGCTGCCTCGGGGAAATTCTCCAGTGACCG
AACAATTAAAGAATATGCCCAAAACATCTGGAACGTGGAACCTTCAGATCTAAAGA
TTTCTCTATCCAATGAATCTAACAAAGTCAATGGAAATTGAACTCTAGAATTGTCTC
TAGAAAACATAGCTTCTTACTGAACTTGAACATTTTTACAACATTCACTGGTTTTTGT
TTTGTTAGCTAATAATCTATAATAGTTGAGTATCTCTGGGAATGGGGAGGGAAATTA
TATGTAATAGAGCTTAAAAATAAAGTGTCAATTTCCAAGGGCTA (SEQ ID NO: 22).
In an aspect, a disclosed Cas9 can comprise the following sequence or a fragment thereof:
ATGAAAAGGAATTATATCTTAGGATTAGATATCGGAATTACATCAGTGGGTTATGG
AATTATTGATTATGAAACTAGAGATGTCATAGATGCGGGCGTACGTTTATTTAAAGA
GGCTAATGTTGAAAATAATGAAGGACGACGATCAAAAAGAGGTGCCAGAAGGCTT
AAGAGGCGTCGTAGACATAGAATACAAAGAGTAAAGAAACTTTTATTTGATTACAA
TTTGTTGACAGATCATAGTGAGCTAAGTGGAATCAATCCTTACGAGGCGCGCGTAA
AGGGATTAAGTCAAAAATTAAGTGAAGAGGAATTTTCTGCGGCATTGCTACATTTA
GCAAAGCGTAGAGGTGTACATAATGTTAATGAAGTGGAAGAAGATACAGGTAATG
AATTATCCACTAAAGAACAAATTTCAAGAAATAGTAAAGCGTTAGAAGAGAAGTAT
GTTGCAGAATTACAGTTGGAACGTTTGAAAAAAGACGGTGAAGTGAGAGGTTCGAT
TAACCGTTTCAAAACATCTGACTATGTAAAAGAAGCAAAGCAGTTATTAAAAGTAC
AAAAAGCATATCATCAACTTGATCAATCATTTATAGACACTTATATTGATTTATTGG
AAACAAGAAGAACATATTATGAGGGACCAGGTGAAGGTAGCCCATTTGGATGGAA
AGATATTAAAGAATGGTATGAAATGTTAATGGGACATTGTACGTATTTCCCAGAAG
AATTACGTAGTGTGAAATATGCCTATAATGCTGATTTATATAATGCGCTGAATGATT
TGAACAACTTGGTTATTACACGAGATGAGAATGAGAAGCTAGAGTATTATGAAAAA
TTCCAAATTATCGAGAATGTCTTTAAACAAAAGAAAAAGCCGACGCTTAAACAAAT
TGCGAAGGAAATCTTGGTGAATGAAGAAGACATCAAAGGCTATCGTGTCACAAGTA
CAGGTAAACCAGAATTTACAAACTTGAAAGTTTATCACGATATCAAAGATATTACA
GCAAGAAAAGAAATTATCGAGAATGCAGAGCTACTCGATCAAATAGCTAAAATATT
AACTATTTACCAGTCATCAGAAGATATACAAGAAGAATTAACAAACCTAAATTCAG
AATTGACACAAGAAGAGATTGAACAAATTTCAAACTTGAAAGGTTATACAGGAACT
CATAACCTTTCACTAAAGGCAATAAATTTAATATTAGACGAATTGTGGCATACGAA
CGATAATCAAATAGCTATTTTCAATCGTTTGAAACTTGTACCTAAAAAGGTAGATTT
AAGCCAACAAAAAGAAATTCCTACTACTTTAGTTGATGATTTTATACTGTCTCCAGT
AGTGAAACGTTCATTTATACAATCTATTAAAGTTATTAACGCTATTATTAAAAAATA
CGGTTTGCCAAATGATATTATTATTGAACTTGCGAGAGAAAAGAATTCTAAAGATG
CACAAAAAATGATTAATGAAATGCAGAAGAGAAATCGTCAAACGAATGAACGTAT
TGAGGAAATTATAAGAACGACAGGTAAAGAAAATGCTAAATATTTAATTGAAAAA
ATTAAGCTGCACGATATGCAAGAAGGGAAATGTTTATACTCGTTAGAAGCAATCCC
TCTAGAAGATTTACTTAATAATCCATTCAATTACGAAGTAGACCATATCATTCCACG
TTCTGTTTCTTTCGATAACTCTTTCAATAATAAAGTGTTGGTGAAACAAGAAGAAAA
TAGTAAAAAAGGTAATAGAACGCCATTTCAATATTTAAGTTCTTCAGATTCTAAAAT
AAGTTATGAGACATTCAAAAAGCATATTTTAAATCTTGCTAAAGGCAAAGGTAGAA
TCTCTAAGACGAAAAAAGAATATTTGTTAGAAGAACGAGATATCAATCGCTTCAGT
GTCCAAAAAGATTTTATTAACCGTAACTTAGTAGATACACGCTATGCGACAAGAGG
GTTAATGAACTTATTAAGATCTTATTTTAGAGTGAATAACTTAGATGTCAAAGTGAA
ATCGATTAATGGCGGATTCACAAGTTTCTTAAGAAGGAAATGGAAGTTCAAAAAAG
AAAGAAATAAGGGCTACAAACACCATGCTGAAGATGCACTGATTATTGCGAACGCT
GATTTTATTTTCAAAGAATGGAAAAAACTAGATAAAGCTAAAAAAGTGATGGAAAA
TCAAATGTTTGAAGAAAAGCAAGCTGAAAGTATGCCTGAAATTGAGACTGAGCAAG
AGTATAAAGAAATTTTTATAACGCCTCATCAAATTAAACATATTAAGGATTTTAAAG
ATTATAAATATTCACATAGAGTTGATAAAAAGCCGAATAGAGAGTTAATAAATGAT
ACATTATATTCTACGAGAAAAGATGACAAGGGTAATACATTAATCGTTAATAACTT
AAATGGTTTATACGATAAAGATAATGATAAATTGAAAAAATTAATTAATAAATCAC
CTGAAAAATTATTGATGTATCATCATGATCCACAAACATATCAAAAATTAAAATTG
ATCATGGAACAATATGGCGATGAGAAAAATCCGCTTTATAAATATTATGAAGAAAC
AGGCAATTACTTAACAAAATATAGTAAAAAAGATAACGGACCAGTCATCAAAAAA
ATTAAATATTATGGTAACAAGCTAAATGCGCATTTAGATATTACGGATGATTATCCA
AATAGCAGAAATAAAGTAGTAAAACTTTCATTAAAACCATATCGCTTTGATGTTTAT
TTAGATAATGGGGTATATAAATTTGTGACAGTTAAAAATTTAGATGTTATCAAAAA
AGAAAACTACTATGAAGTTAATTCAAAGTGTTATGAAGAAGCAAAAAAACTGAAG
AAAATTAGTAATCAAGCAGAATTTATCGCAAGTTTTTACAATAATGACTTGATTAAG
ATTAACGGAGAATTATATAGAGTCATAGGTGTAAATAATGATCTACTTAACAGAAT
TGAAGTAAATATGATAGACATCACATATAGAGAATATTTAGAGAACATGAATGATA
AAAGACCACCTAGAATAATTAAAACAATAGCAAGCAAAACACAATCTATTAAAAA
GTATTCTACAGATATTCTAGGCAATCTTTATGAAGTGAAGAGTAAAAAGCATCCTCA
AATCATAAAGAAAGGATGA (SEQ ID NO: 31).
In an aspect, a disclosed GAA can comprise the following sequence or a fragment thereof:
CTCGAGCAACCATGGGAGTGAGGCACCCGCCCTGCTCCCACCGGCTCCTGGCCGTC
TGCGCCCTCGTGTCCTTGGCAACCGCTGCACTCCTGGGGCACATCCTACTCCATGAT
TTCCTGCTGGTTCCCCGAGAGCTGAGTGGCTCCTCCCCAGTCCTGGAGGAGACTCAC
CCAGCTCACCAGCAGGGAGCCAGCAGACCAGGGCCCCGGGATGCCCAGGCACACC
CCGGCCGTCCCAGAGCAGTGCCCACACAGTGCGACGTCCCCCCCAACAGCCGCTTC
GATTGCGCCCCTGACAAGGCCATCACCCAGGAACAGTGCGAGGCCCGCGGCTGCTG
CTACATCCCTGCAAAGCAGGGGCTGCAGGGAGCCCAGATGGGGCAGCCCTGGTGCT
TCTTCCCACCCAGCTACCCCAGCTACAAGCTGGAGAACCTGAGCTCCTCTGAAATGG
GCTACACGGCCACCCTGACCCGTACCACCCCCACCTTCTTCCCCAAGGACATCCTGA
CCCTGCGGCTGGACGTGATGATGGAGACTGAGAACCGCCTCCACTTCACGATCAAA
GATCCAGCTAACAGGCGCTACGAGGTGCCCTTGGAGACCCCGCGTGTCCACAGCCG
GGCACCGTCCCCACTCTACAGCGTGGAGTTCTCTGAGGAGCCCTTCGGGGTGATCGT
GCACCGGCAGCTGGACGGCCGCGTGCTGCTGAACACGACGGTGGCGCCCCTGTTCT
TTGCGGACCAGTTCCTTCAGCTGTCCACCTCGCTGCCCTCGCAGTATATCACAGGCC
TCGCCGAGCACCTCAGTCCCCTGATGCTCAGCACCAGCTGGACCAGGATCACCCTGT
GGAACCGGGACCTTGCGCCCACGCCCGGTGCGAACCTCTACGGGTCTCACCCTTTCT
ACCTGGCGCTGGAGGACGGCGGGTCGGCACACGGGGTGTTCCTGCTAAACAGCAAT
GCCATGGATGTGGTCCTGCAGCCGAGCCCTGCCCTTAGCTGGAGGTCGACAGGTGG
GATCCTGGATGTCTACATCTTCCTGGGCCCAGAGCCCAAGAGCGTGGTGCAGCAGT
ACCTGGACGTTGTGGGATACCCGTTCATGCCGCCATACTGGGGCCTGGGCTTCCACC
TGTGCCGCTGGGGCTACTCCTCCACCGCTATCACCCGCCAGGTGGTGGAGAACATG
ACCAGGGCCCACTTCCCCCTGGACGTCCAATGGAACGACCTGGACTACATGGACTC
CCGGAGGGACTTCACGTTCAACAAGGATGGCTTCCGGGACTTCCCGGCCATGGTGC
AGGAGCTGCACCAGGGCGGCCGGCGCTACATGATGATCGTGGATCCTGCCATCAGC
AGCTCGGGCCCTGCCGGGAGCTACAGGCCCTACGACGAGGGTCTGCGGAGGGGGGT
TTTCATCACCAACGAGACCGGCCAGCCGCTGATTGGGAAGGTATGGCCCGGGTCCA
CTGCCTTCCCCGACTTCACCAACCCCACAGCCCTGGCCTGGTGGGAGGACATGGTG
GCTGAGTTCCATGACCAGGTGCCCTTCGACGGCATGTGGATTGACATGAACGAGCC
TTCCAACTTCATCAGGGGCTCTGAGGACGGCTGCCCCAACAATGAGCTGGAGAACC
CACCCTACGTGCCTGGGGTGGTTGGGGGGACCCTCCAGGCGGCCACCATCTGTGCC
TCCAGCCACCAGTTTCTCTCCACACACTACAACCTGCACAACCTCTACGGCCTGACC
GAAGCCATCGCCTCCCACAGGGCGCTGGTGAAGGCTCGGGGGACACGCCCATTTGT
GATCTCCCGCTCGACCTTTGCTGGCCACGGCCGATACGCCGGCCACTGGACGGGGG
ACGTGTGGAGCTCCTGGGAGCAGCTCGCCTCCTCCGTGCCAGAAATCCTGCAGTTTA
ACCTGCTGGGGGTGCCTCTGGTCGGGGCCGACGTCTGCGGCTTCCTGGGCAACACCT
CAGAGGAGCTGTGTGTGCGCTGGACCCAGCTGGGGGCCTTCTACCCCTTCATGCGG
AACCACAACAGCCTGCTCAGTCTGCCCCAGGAGCCGTACAGCTTCAGCGAGCCGGC
CCAGCAGGCCATGAGGAAGGCCCTCACCCTGCGCTACGCACTCCTCCCCCACCTCTA
CACGCTGTTCCACCAGGCCCACGTCGCGGGGGAGACCGTGGCCCGGCCCCTCTTCCT
GGAGTTCCCCAAGGACTCTAGCACCTGGACTGTGGACCACCAGCTCCTGTGGGGGG
AGGCCCTGCTCATCACCCCAGTGCTCCAGGCCGGGAAGGCCGAAGTGACTGGCTAC
TTCCCCTTGGGCACATGGTACGACCTGCAGACGGTGCCAATAGAGGCCCTTGGCAG
CCTCCCACCCCCACCTGCAGCTCCCCGTGAGCCAGCCATCCACAGCGAGGGGCAGT
GGGTGACGCTGCCGGCCCCCCTGGACACCATCAACGTCCACCTCCGGGCTGGGTAC
ATCATCCCCCTGCAGGGCCCTGGCCTCACAACCACAGAGTCCCGCCAGCAGCCCAT
GGCCCTGGCTGTGGCCCTGACCAAGGGTGGAGAGGCCCGAGGGGAGCTGTTCTGGG
ACGATGGAGAGAGCCTGGAAGTGCTGGAGCGAGGGGCCTACACACAGGTCATCTTC
CTGGCCAGGAATAACACGATCGTGAATGAGCTGGTACGTGTGACCAGTGAGGGAGC
TGGCCTGCAGCTGCAGAAGGTGACTGTCCTGGGCGTGGCCACGGCGCCCCAGCAGG
TCCTCTCCAACGGTGTCCCTGTCTCCAACTTCACCTACAGCCCCGACACCAAGGTCC
TGGACATCTGTGTCTCGCTGTTGATGGGAGAGCAGTTTCTCGTCAGCTGGTGTTAAA
CTCGAG (SEQ ID NO: 39).
In an aspect, a disclosed GAA can comprise the following sequence or a fragment thereof:
GCGCCTGCGCGGGAGGCCGCGTCACGTGACCCACCGCGGCCCCGCCCCGCGACGAG
CTCCCGCCGGTCACGTGACCCGCCTCTGCGCGCCCCCGGGCACGACCCCGGAGTCTC
CGCGGGCGGCCAGGGCGCGCGTGCGCGGAGGTGAGCCGGGCCGGGGCTGCGGGGC
TTCCCTGAGCGCGGGCCGGGTCGGTGGGGCGGTCGGCTGCCCGCGCCGGCCTCTCA
GTTGGGAAAGCTGAGGTTGTCGCCGGGGCCGCGGGTGGAGGTCGGGGATGAGGCA
GCAGGTAGGACAGTGACCTCGGTGACGCGAAGGACCCCGGCCACCTCTAGGTTCTC
CTCGTCCGCCCGTTGTTCAGCGAGGGAGGCTCTGGGCCTGCCGCAGCTGACGGGGA
AACTGAGGCACGGAGCGGGCCTGTAGGAGCTGTCCAGGCCATCTCCAACCATGGGA
GTGAGGCACCCGCCCTGCTCCCACCGGCTCCTGGCCGTCTGCGCCCTCGTGTCCTTG
GCAACCGCTGCACTCCTGGGGCACATCCTACTCCATGATTTCCTGCTGGTTCCCCGA
GAGCTGAGTGGCTCCTCCCCAGTCCTGGAGGAGACTCACCCAGCTCACCAGCAGGG
AGCCAGCAGACCAGGGCCCCGGGATGCCCAGGCACACCCCGGCCGTCCCAGAGCA
GTGCCCACACAGTGCGACGTCCCCCCCAACAGCCGCTTCGATTGCGCCCCTGACAA
GGCCATCACCCAGGAACAGTGCGAGGCCCGCGGCTGCTGCTACATCCCTGCAAAGC
AGGGGCTGCAGGGAGCCCAGATGGGGCAGCCCTGGTGCTTCTTCCCACCCAGCTAC
CCCAGCTACAAGCTGGAGAACCTGAGCTCCTCTGAAATGGGCTACACGGCCACCCT
GACCCGTACCACCCCCACCTTCTTCCCCAAGGACATCCTGACCCTGCGGCTGGACGT
GATGATGGAGACTGAGAACCGCCTCCACTTCACGATCAAAGATCCAGCTAACAGGC
GCTACGAGGTGCCCTTGGAGACCCCGCGTGTCCACAGCCGGGCACCGTCCCCACTC
TACAGCGTGGAGTTCTCCGAGGAGCCCTTCGGGGTGATCGTGCACCGGCAGCTGGA
CGGCCGCGTGCTGCTGAACACGACGGTGGCGCCCCTGTTCTTTGCGGACCAGTTCCT
TCAGCTGTCCACCTCGCTGCCCTCGCAGTATATCACAGGCCTCGCCGAGCACCTCAG
TCCCCTGATGCTCAGCACCAGCTGGACCAGGATCACCCTGTGGAACCGGGACCTTG
CGCCCACGCCCGGTGCGAACCTCTACGGGTCTCACCCTTTCTACCTGGCGCTGGAGG
ACGGCGGGTCGGCACACGGGGTGTTCCTGCTAAACAGCAATGCCATGGATGTGGTC
CTGCAGCCGAGCCCTGCCCTTAGCTGGAGGTCGACAGGTGGGATCCTGGATGTCTA
CATCTTCCTGGGCCCAGAGCCCAAGAGCGTGGTGCAGCAGTACCTGGACGTTGTGG
GATACCCGTTCATGCCGCCATACTGGGGCCTGGGCTTCCACCTGTGCCGCTGGGGCT
ACTCCTCCACCGCTATCACCCGCCAGGTGGTGGAGAACATGACCAGGGCCCACTTC
CCCCTGGACGTCCAATGGAACGACCTGGACTACATGGACTCCCGGAGGGACTTCAC
GTTCAACAAGGATGGCTTCCGGGACTTCCCGGCCATGGTGCAGGAGCTGCACCAGG
GCGGCCGGCGCTACATGATGATCGTGGATCCTGCCATCAGCAGCTCGGGCCCTGCC
GGGAGCTACAGGCCCTACGACGAGGGTCTGCGGAGGGGGGTTTTCATCACCAACGA
GACCGGCCAGCCGCTGATTGGGAAGGTATGGCCCGGGTCCACTGCCTTCCCCGACT
TCACCAACCCCACAGCCCTGGCCTGGTGGGAGGACATGGTGGCTGAGTTCCATGAC
CAGGTGCCCTTCGACGGCATGTGGATTGACATGAACGAGCCTTCCAACTTCATCAG
AGGCTCTGAGGACGGCTGCCCCAACAATGAGCTGGAGAACCCACCCTACGTGCCTG
GGGTGGTTGGGGGGACCCTCCAGGCGGCCACCATCTGTGCCTCCAGCCACCAGTTT
CTCTCCACACACTACAACCTGCACAACCTCTACGGCCTGACCGAAGCCATCGCCTCC
CACAGGGCGCTGGTGAAGGCTCGGGGGACACGCCCATTTGTGATCTCCCGCTCGAC
CTTTGCTGGCCACGGCCGATACGCCGGCCACTGGACGGGGGACGTGTGGAGCTCCT
GGGAGCAGCTCGCCTCCTCCGTGCCAGAAATCCTGCAGTTTAACCTGCTGGGGGTG
CCTCTGGTCGGGGCCGACGTCTGCGGCTTCCTGGGCAACACCTCAGAGGAGCTGTG
TGTGCGCTGGACCCAGCTGGGGGCCTTCTACCCCTTCATGCGGAACCACAACAGCCT
GCTCAGTCTGCCCCAGGAGCCGTACAGCTTCAGCGAGCCGGCCCAGCAGGCCATGA
GGAAGGCCCTCACCCTGCGCTACGCACTCCTCCCCCACCTCTACACACTGTTCCACC
AGGCCCACGTCGCGGGGGAGACCGTGGCCCGGCCCCTCTTCCTGGAGTTCCCCAAG
GACTCTAGCACCTGGACTGTGGACCACCAGCTCCTGTGGGGGGAGGCCCTGCTCAT
CACCCCAGTGCTCCAGGCCGGGAAGGCCGAAGTGACTGGCTACTTCCCCTTGGGCA
CATGGTACGACCTGCAGACGGTGCCAATAGAGGCCCTTGGCAGCCTCCCACCCCCA
CCTGCAGCTCCCCGTGAGCCAGCCATCCACAGCGAGGGGCAGTGGGTGACGCTGCC
GGCCCCCCTGGACACCATCAACGTCCACCTCCGGGCTGGGTACATCATCCCCCTGCA
GGGCCCTGGCCTCACAACCACAGAGTCCCGCCAGCAGCCCATGGCCCTGGCTGTGG
CCCTGACCAAGGGTGGAGAGGCCCGAGGGGAGCTGTTCTGGGACGATGGAGAGAG
CCTGGAAGTGCTGGAGCGAGGGGCCTACACACAGGTCATCTTCCTGGCCAGGAATA
ACACGATCGTGAATGAGCTGGTACGTGTGACCAGTGAGGGAGCTGGCCTGCAGCTG
CAGAAGGTGACTGTCCTGGGCGTGGCCACGGCGCCCCAGCAGGTCCTCTCCAACGG
TGTCCCTGTCTCCAACTTCACCTACAGCCCCGACACCAAGGTCCTGGACATCTGTGT
CTCGCTGTTGATGGGAGAGCAGTTTCTCGTCAGCTGGTGTTAGCCGGGCGGAGTGTG
TTAGTCTCTCCAGAGGGAGGCTGGTTCCCCAGGGAAGCAGAGCCTGTGTGCGGGCA
GCAGCTGTGTGCGGGCCTGGGGGTTGCATGTGTCACCTGGAGCTGGGCACTAACCA
TTCCAAGCCGCCGCATCGCTTGTTTCCACCTCCTGGGCCGGGGCTCTGGCCCCCAAC
GTGTCTAGGAGAGCTTTCTCCCTAGATCGCACTGTGGGCCGGGGCCTGGAGGGCTG
CTCTGTGTTAATAAGATTGTAAGGTTTGCCCTCCTCACCTGTTGCCGGCATGCGGGT
AGTATTAGCCACCCCCCTCCATCTGTTCCCAGCACCGGAGAAGGGGGTGCTCAGGT
GGAGGTGTGGGGTATGCACCTGAGCTCCTGCTTCGCGCCTGCTGCTCTGCCCCAACG
CGACCGCTTCCCGGCTGCCCAGAGGGCTGGATGCCTGCCGGTCCCCGAGCAAGCCT
GGGAACTCAGGAAAATTCACAGGACTTGGGAGATTCTAAATCTTAAGTGCAATTAT
TTTAATAAAAGGGGCATTTGGAATC (SEQ ID NO: 40).
In an aspect, a disclosed LSP promoter can have the following sequence or a fragment
thereof:
GAGTTAATTTTTAAAAAGCAGTCAAAAGTCCAAGTGGCCCTTGCGAGCATTTACTCT
CTCTGTTTGCTCTGGTTAATAATCTCAGGAGCACAAAATTCCTTACTAGTCCTAGAA
GTTAATTTTTAAAAAGCAGTCAAAAGTCCAAGTCCAAGTGGCCCTTGCGAGCATTTA
CTCTCTCTGTTTGCTCTGGTTAATAATCTCAGGAGCACAAACATTCCTTACTAGTTCT
AGAGCGGCCGCCAGTGTGCTGGAATTCGGCTTTTTTAGGGCTGGAAGCTACCTTTGA
CATCATTTCCTCTGCGAATGCATGTATAATTTCTACAGAACCTATTAGAAAGGATCA
CCCAGCCTCTGCTTTTGTACAACTTTCCCTTAAAAAATGCCAATTCCACTGCTGTTTG
GCCCAATAGTGAGAACTTTTTCCTGCTGCCTCTTGGTGCTTTTGCCTATGGCCCCTAT
TCTGCCTGCTGAAGACACTCTTGCCAGCATGGACTTAAACCCCTCCAGCTCTGACAA
TCCTCTTTCTCTTTTGTTTTACATGAAGGGTCTGGCAGCCAAAGCAATCACTCAAAG
GTTCAAACCTTATCATTTTTTGCTTTGTTCCTCTTGGCCTTGGTTTTGTACATCAGCTT
TGAAAATACCATCCCAGGGTTAATGCTGGGGTTAATTTATAACTAAGAGTGCTCTAG
TTTTGCAATACAGGACATGCTATAAAAATGGAAAGATGTTGCTTTCTGAGAGATCA
GCTTACATGT. (SEQ ID NO: 56)

### 2. Vectors

Disclosed herein is a vector comprising a disclosed isolated nucleic acid molecule. Disclosed herein is a vector comprising a disclosed isolated nucleic acid molecule encoding one or more disclosed encoded polypeptides. Disclosed herein is a vector comprising a disclosed isolated nucleic acid molecule encoding one or more disclosed encoded polypeptides, wherein the nucleic acid sequence is CpG depleted and codon-optimized for expression in a human cell. Disclosed herein is a vector comprising an isolated nucleic acid molecule comprising a nucleic acid sequence encoding a polypeptide capable of preventing glycogen accumulation and/or degrading accumulated glycogen. Disclosed herein is a vector comprising an isolated nucleic acid molecule comprising a nucleic acid sequence encoding a polypeptide capable of preventing glycogen accumulation and/or degrading accumulated glycogen, wherein the nucleic acid sequence is CpG depleted and codon-optimized for expression in a human cell. Disclosed herein is a vector comprising an isolated nucleic acid molecule comprising a nucleic acid sequence encoding a polypeptide capable of reducing or inhibiting the expression level and/or activity level of glycogen synthase. Disclosed herein is a vector comprising an isolated nucleic acid molecule comprising a nucleic acid sequence encoding a polypeptide capable of reducing or inhibiting the expression level and/or activity level of glycogen synthase, wherein the nucleic acid sequence is CpG depleted and codon-optimized for expression in a human cell. Disclosed herein is a vector comprising an isolated nucleic acid molecule comprising a nucleic acid sequence encoding a polypeptide capable of preventing glycogen accumulation and/or degrading accumulated glycogen and a nucleic acid sequence encoding a polypeptide capable of reducing or inhibiting the expression level and/or activity level of glycogen synthase.

Disclosed herein is a vector comprising an isolated nucleic acid molecule, wherein the isolated nucleic acid sequence encodes human PhK al, PhK α2, PhK β, PhK δ, PhK γ2, and/or glycogen phosphorylase. Disclosed herein is a vector comprising an isolated nucleic acid molecule, wherein the isolated nucleic acid sequence encodes human PhK al, PhK α2, PhK β, PhK δ, PhK γ2, and/or glycogen phosphorylase, wherein the isolated nucleic acid sequence is CpG-depleted and codon-optimized for expression in a human cell. Disclosed herein is a vector comprising an isolated nucleic acid molecule, wherein the isolated nucleic acid sequence encodes human PhK al, PhK α2, PhK 3, PhK δ, PhK γ2, and/or glycogen phosphorylase. Disclosed herein is a vector comprising an isolated nucleic acid molecule, wherein the isolated nucleic acid sequence encodes human PhK al, PhK α2, PhK, PhK δ, PhK γ2, and/or glycogen phosphorylase, wherein the isolated nucleic acid sequence is CpG-depleted and codon-optimized for expression in a human cell. Disclosed herein is a vector comprising an isolated nucleic acid sequence having the sequence set forth in any one of SEQ ID NO:44-SEQ ID NO:55. Disclosed herein is a vector comprising an isolated nucleic acid sequence having at least 30%, at least 40%, at least 50%, at least 60%, at least 70%, at least 80%, or at least 90% identity to the sequence set forth in any one of SEQ ID NO:44-SEQ ID NO:55. Disclosed herein is a vector comprising an isolated nucleic acid sequence having at least 30%, at least 40%, at least 50%, at least 60%, at least 70%, at least 80%, or at least 90% identity to the sequence set forth in any one of SEQ ID NO:44-SEQ ID NO:55.

Disclosed herein is a vector comprising an isolated nucleic acid molecule, wherein the isolated nucleic acid sequence encodes alpha glucosidase or GAA. Disclosed herein is a vector comprising an isolated nucleic acid molecule, wherein the isolated nucleic acid sequence encodes alpha glucosidase or GAA, wherein the isolated nucleic acid sequence is CpG-depleted and codon-optimized for expression in a human cell.

Disclosed herein is a vector comprising an isolated nucleic acid molecule comprising a nucleic acid sequence encoding a polypeptide capable of restoring one or more aspects of the glycogen metabolic pathway. For example, in an aspect, a disclosed isolated vector can restore the balance of glycogen synthesis and degradation. Disclosed herein is a vector comprising an isolated nucleic acid molecule comprising a nucleic acid sequence encoding a polypeptide capable restoring one or more aspects of the glycogen metabolic pathway, wherein the nucleic acid sequence is CpG depleted and codon-optimized for expression in a human cell. Disclosed herein is a vector comprising an isolated nucleic acid molecule comprising a nucleic acid sequence encoding a polypeptide capable of restoring one or more aspects of the glycogen metabolic pathway, and a nucleic acid sequence encoding a polypeptide capable of reducing the expression level and/or activity level of glycogen synthase. Disclosed herein is a vector comprising an isolated nucleic acid molecule comprising a nucleic acid sequence encoding a polypeptide capable of restoring one or more aspects of the glycogenolysis pathway. Disclosed herein is a vector comprising an isolated nucleic acid molecule comprising a nucleic acid sequence encoding a polypeptide capable restoring one or more aspects of the glycogenolysis metabolic pathway, wherein the nucleic acid sequence is CpG depleted and codon-optimized for expression in a human cell.

Disclosed herein is a vector comprising an isolated nucleic acid molecule comprising a nucleic acid sequence encoding a polypeptide capable of restoring one or more aspects of the glycogenolysis metabolic pathway, and a nucleic acid sequence encoding a polypeptide capable of reducing the expression level and/or activity level of glycogen synthase and restoring the balance of glycogen synthesis to glycogen breakdown.

Disclosed herein is vectoring comprising an isolated nucleic acid molecule comprising a nucleic acid sequence encoding a polypeptide capable of restoring the balance of glycogen metabolism comprising administering to a subject having GSD IX and/or GSD VI a therapeutically effective amount of a disclosed isolated nucleic acid molecule, a disclosed vector, a disclosed pharmaceutical formulation, or a combination thereof, wherein glycogen metabolism comprises glycogen synthesis and breakdown.

Disclosed herein is a vector comprising an isolated nucleic acid molecule comprising a nucleic acid sequence encoding a polypeptide capable of restoring one or more aspects of the glycogenesis metabolic pathway. Disclosed herein is a vector comprising an isolated nucleic acid molecule comprising a nucleic acid sequence encoding a polypeptide capable restoring one or more aspects of the glycogenesis metabolic pathway, wherein the nucleic acid sequence is CpG depleted and codon-optimized for expression in a human cell. Disclosed herein is a vector comprising an isolated nucleic acid molecule comprising a nucleic acid sequence encoding a polypeptide capable of restoring one or more aspects of the glycogenesis metabolic pathway, and a nucleic acid sequence encoding a polypeptide capable of reducing the expression level and/or activity level of glycogen synthase.

Disclosed herein is a vector comprising an isolated nucleic acid molecule comprising a nucleic acid sequence encoding a polypeptide capable of restoring PhK subunit activity and/or functionality. Disclosed herein is a vector comprising an isolated nucleic acid molecule comprising a nucleic acid sequence encoding a polypeptide capable restoring PhK subunit activity and/or functionality, wherein the nucleic acid sequence is CpG depleted and codon-optimized for expression in a human cell. Disclosed herein is a vector comprising an isolated nucleic acid molecule comprising a nucleic acid sequence encoding a polypeptide capable of restoring PhK subunit activity and/or functionality, and a nucleic acid sequence encoding a polypeptide capable of reducing the expression level and/or activity level of glycogen synthase.

In an aspect, a disclosed vector can restore the functionality and/or structural integrity of the PhK complex. For example, in an aspect, by restoring the functionality and/or structural integrity of a subunit (such as α2, δ, β, and γ2), then the functionality and/or structural integrity of the PhK complex (the heterotetramer) can be restored.

In an aspect, a therapeutically effective amount of disclosed vector can be delivered via intravenous (IV) administration and can comprise a range of about 1×10¹⁰ vg/kg to about 2×10¹⁴ vg/kg. In an aspect, for example, a disclosed vector can be administered at a dose of about 1×10¹¹ to about 8×10¹³ vg/kg or about 1×10¹² to about 8×10¹³ vg/kg. In an aspect, a disclosed vector can be administered at a dose of about 1×10¹³ to about 6×10¹³ vg/kg. In an aspect, a disclosed vector can be administered at a dose of at least about 1×10¹⁰, at least about 5×10¹⁰, at least about 1×10¹¹, at least about 5×10¹¹, at least about 1×10¹², at least about 5×10¹², at least about 1×10¹³, at least about 5×10¹³, or at least about 1×10¹⁴ vg/kg. In an aspect, a disclosed vector can be administered at a dose of no more than about 1×10¹⁰, no more than about 5×10¹⁰, no more than about 1×10¹¹, no more than about 5×10¹¹, no more than about 1×10¹², no more than about 5×10¹², no more than about 1×10¹³, no more than about 5×10¹³, or no more than about 1×10¹⁴ vg/kg. In an aspect, a disclosed vector can be administered at a dose of about 1×10¹² vg/kg. In an aspect, a disclosed vector can be administered at a dose of about 1×10¹¹ vg/kg. In an aspect, a disclosed vector can be administered in a single dose, or in multiple doses (such as 2, 3, 4, 5, 6, 7, 8, 9 or 10 doses) as needed for the desired therapeutic results.

In an aspect, “CpG-free” can mean completely free of CpGs or partially free of CpGs. In an aspect, “CpG-free” can mean “CpG-depleted”. In an aspect, “CpG-depleted” can mean “CpG-free”. In an aspect, “CpG-depleted” can mean completely depleted of CpGs or partially depleted of CpGs. In an aspect, “CpG-free” can mean “CpG-optimized” for a desired and/or ideal expression level. CpG depletion and/or optimization is known to the skilled person in the art.

In an aspect, a disclosed nucleic acid sequence can have a coding sequence that is less than about 4.5 kilobases.

In an aspect, a disclosed vector can be a viral vector or a non-viral vector. In an aspect, a disclosed non-viral vector can be a polymer-based vector, a peptide-based vector, a lipid nanoparticle, a solid lipid nanoparticle, or a cationic lipid-based vector. In an aspect, a disclosed viral vector can be an adenovirus vector, an AAV vector, a herpes simplex virus vector, a retrovirus vector, a lentivirus vector, and alphavirus vector, a flavivirus vector, a rhabdovirus vector, a measles virus vector, a Newcastle disease viral vector, a poxvirus vector, or a picornavirus vector.

In an aspect, a disclosed viral vector can be an adeno-associated virus (AAV) vector In an aspect, a disclosed AAV vector can include naturally isolated serotypes including, but not limited to, AAV1, AAV2, AAV3 (including 3a and 3b), AAV4, AAV5, AAV6, AAV7, AAV8, AAVrh8, AAV9, AAV10, AAVrh10, AAV11, AAV12, AAV13, AAVrh39, AAVrh43, AAVcy.7 as well as bovine AAV, caprine AAV, canine AAV, equine AAV, ovine AAV, avian AAV, primate AAV, non-primate AAV, and any other virus classified by the International Committee on Taxonomy of Viruses (ICTV) as an AAV. In an aspect, an AAV capsid can be a chimera either created by capsid evolution or by rational capsid engineering from a naturally isolated AAV variants to capture desirable serotype features such as enhanced or specific tissue tropism and/or a host immune response escape. Naturally isolated AAV variants include, but not limited to, AAV-DJ, AAV-HAE1, AAV-HAE2, AAVM41, AAV-1829, AAV2 Y/F, AAV2 T/V, AAV2i8, AAV2.5, AAV9.45, AAV9.61, AAV-B1, AAV-AS, AAV9.45A-String (e.g., AAV9.45-AS), AAV9.45Angiopep, AAV9.47-Angiopep, and AAV9.47-AS, AAV-PHP.B, AAV-PHP.eB, AAV-PHP.S, AAV-F, AAVcc.47, and AAVcc.81. In an aspect, a disclosed AAV vector can be AAV-Rh74 or a related variant (e.g., capsid variants like RHM4-1). In an aspect, a disclosed AAV vector can be AAV8. In an aspect, a disclosed AAV vector can be AAVhum.8. In an aspect, a disclosed AAV vector can be a self-complementary AAV as disclosed herein.

In an aspect, a disclosed vector can comprise a liver-specific promoter operably linked to the isolated nucleic acid molecule. In an aspect, a disclosed liver-specific promoter can be the thyroxin binding globulin (TBG) promoter, the α1-microglobulin/bikunin enhancer/thyroid hormone-binding globulin promoter, the human albumin (hALB) promoter, the thyroid hormone-binding globulin promoter, the thyroxin binding globulin promoter, the α-1-anti-trypsin promoter, the bovine albumin (bAlb) promoter, the murine albumin (mAlb) promoter, the human α1-antitrypsin (hAAT) promoter, the ApoEhAAT promoter comprising the ApoE enhancer and the hAAT promoter, the transthyretin (TTR) promoter, the liver fatty acid binding protein promoter, the hepatitis B virus (HBV) promoter, the DC172 promoter consisting of the hAAT promoter and the α1-microglobulin enhancer, the DC190 promoter containing the human albumin promoter and the prothrombin enhancer, or any one of other natural and synthetic liver-specific promoter.

In an aspect, a disclosed liver-specific promoter can comprise any liver-specific promoter known to the art. In an aspect, a liver-specific promoter can comprise about 845-bp and comprise the thyroid hormone-binding globulin promoter sequences (2382 to 13), two copies of α1-microglobulinybikunin enhancer sequences (22,804 through 22,704), and a 71-bp leader sequence as described by Ill C R, et al. (1997). In an aspect, a disclosed liver-specific promoter can comprise the sequence set forth in SEQ ID NO:56. In an aspect, a disclosed liver-specific promoter can comprise a sequence having at least 40%, 50%, 60%, 70%, 80%, 85%, 86%, 87%, 88%, 89%, 90%, 91%, 92%, 93%, 94%, 95%, 96%, 97%, 98%, or 99% identity to the sequence set forth in SEQ ID NO:56. In an aspect, a disclosed liver-specific promoter can comprise a sequence having at least 40%-60%, at least 60%-80%, at least 80%-90%, or at least 90%-100% identity to the sequence set forth in SEQ ID NO:56.

In an aspect, a disclosed ubiquitous promoter can be a CMV enhancer/chicken β-actin promoter (CB promoter).

In an aspect, a disclosed promoter can be a promoter/enhancer. In an aspect, a disclosed promoter can be an endogenous promoter. In an aspect, a disclosed endogenous promoter can be an endogenous promoter/enhancer. In an aspect, a disclosed endogenous promoter or a disclosed endogenous promoter/enhancer can generally be obtained from a non-coding region upstream of a transcription initiation site of a gene of interest (such as, for example, a disclosed phosphorylase kinase or phosphorylase kinase subunit (e.g., PhK α2, PhK β, PhK γ2, PhK δ) or some other enzyme involved in the glycogen metabolic pathway (PYGL)). In an aspect, a disclosed endogenous promoter or a disclosed endogenous promoter/enhancer can be used for constitutive and efficient expression of a disclosed gene (e.g., a nucleic acid sequence encoding a polypeptide capable of preventing glycogen accumulation and/or degrading accumulated glycogen).

In an aspect, a disclosed endogenous promoter or a disclosed endogenous promoter/enhancer can be used for constitutive and efficient expression of a phosphorylase kinase or a phosphorylase kinase subunit. In an aspect, a disclosed endogenous promoter or a disclosed endogenous promoter/enhancer can comprise the promoter for the gene encoding the phosphorylase kinase regulatory subunit alpha 2 (PhK α2) (SEQ ID NO:63), the phosphorylase kinase regulatory subunit beta (PhK β) (SEQ ID NO:64), the phosphorylase kinase catalytic subunit gamma 2 (PhK γ2) (SEQ ID NO:65), the phosphorylase kinase regulatory subunit delta (PhK δ), or glycogen phosphorylase (PYGL) (SEQ ID NO:66). For example, in an aspect, when an encoded polypeptide comprises the PhK α2 subunit, the disclosed endogenous promoter or a disclosed endogenous promoter/enhancer can comprise the promoter or promoter/enhancer for the gene encoding the PhK α2 subunit. Similarly, when an encoded polypeptide comprises the PhK R subunit, the disclosed endogenous promoter or a disclosed endogenous promoter/enhancer can comprise the promoter or promoter/enhancer for the gene encoding the PhK β subunit. For example, in an aspect, a disclosed endogenous promoter or a disclosed endogenous promoter/enhancer can comprise the sequence set forth in SEQ ID NO:63-SEQ ID NO:66 or a fragment thereof. For example, in an aspect, a disclosed endogenous promoter or a disclosed endogenous promoter/enhancer can comprise a sequence having at least 40%, at least 50%, at least 60%, at least 70%, at least 80%, at least 90%, at least 95%, or more than 95% identity to the sequence set forth in SEQ ID NO:63-SEQ ID NO:66 or a fragment thereof.

In an aspect, a disclosed nucleic acid sequence can comprise the sequence for a phosphorylase kinase or a subunit of a phosphorylase kinase (e.g., PHKA1, PHKA2, PHKB, CALM1, CALM2, CALM3, and/or PHKG2). In an aspect, a disclosed nucleic acid sequence can comprise the sequence for glycogen phosphorylase liver form (e.g., PYGL). In an aspect, a disclosed nucleic acid can comprise the sequence set forth in any one of SEQ ID NO:12-SEQ ID NO:22 or SEQ ID NO:60-SEQ ID NO:62. In an aspect, a disclosed encoded polypeptide can comprise a sequence having at least 30%, at least 40%, at least 50%, at least 60%, at least 70%, at least 80%, or at least 90% identity to the sequence set forth in any one of SEQ ID NO:12-SEQ ID NO:22 or SEQ ID NO:60-SEQ ID NO:62.

In an aspect, a disclosed encoded polypeptide can comprise the sequence set forth in any one of SEQ ID NO:01-SEQ ID NO:11 or SEQ ID NO:57-SEQ ID NO:59. In an aspect, a disclosed encoded polypeptide can comprise a sequence having at least 40-59%, at least 50-69%, or at least 80-99% identity to the sequence set forth in SEQ ID NO:01-SEQ ID NO:11 or SEQ ID NO:57-SEQ ID NO:59.

In an aspect, a disclosed viral vector can comprise an isolated nucleic acid molecule comprising the tissue specific promoter, the CpG-depleted and codon-optimized nucleic acid sequence encoding the polypeptide, and a polyadenylation sequence.

Disclosed herein is an AAV vector comprising a disclosed isolated nucleic acid molecule.

Disclosed herein is a vector comprising a disclosed isolated nucleic acid molecule encoding one or more disclosed encoded polypeptides. Disclosed herein is an AAV vector comprising a disclosed isolated nucleic acid molecule encoding one or more disclosed encoded polypeptides, wherein the nucleic acid sequence is CpG depleted and codon-optimized for expression in a human cell. Disclosed herein is an AAV vector comprising an isolated nucleic acid molecule comprising a nucleic acid sequence encoding a polypeptide capable of preventing glycogen accumulation and/or degrading accumulated glycogen. Disclosed herein is an AAV vector comprising an isolated nucleic acid molecule comprising a nucleic acid sequence encoding a polypeptide capable of preventing glycogen accumulation and/or degrading accumulated glycogen, wherein the nucleic acid sequence is CpG depleted and codon-optimized for expression in a human cell. Disclosed herein is an AAV vector comprising an isolated nucleic acid molecule comprising a nucleic acid sequence encoding a polypeptide capable of reducing or inhibiting the expression level and/or activity level of glycogen synthase. Disclosed herein is an AAV vector comprising an isolated nucleic acid molecule comprising a nucleic acid sequence encoding a polypeptide capable of preventing glycogen accumulation and/or degrading accumulated glycogen and a nucleic acid sequence encoding a polypeptide capable of reducing or inhibiting the expression level and/or activity level of glycogen synthase. Disclosed herein is an AAV vector comprising an isolated nucleic acid molecule, wherein the isolated nucleic acid sequence encodes human glycogen phosphorylase kinase subunit α1, glycogen phosphorylase kinase subunit α2, glycogen phosphorylase kinase subunit β, glycogen phosphorylase kinase subunit δ, glycogen phosphorylase kinase subunit γ2, and/or glycogen phosphorylase. Disclosed herein is an AAV vector comprising an isolated nucleic acid molecule, wherein the isolated nucleic acid sequence encodes human glycogen phosphorylase kinase subunit α1, glycogen phosphorylase kinase subunit α2, glycogen phosphorylase kinase subunit β, glycogen phosphorylase kinase subunit δ, glycogen phosphorylase kinase subunit γ2, and/or glycogen phosphorylase, wherein the isolated nucleic acid sequence is CpG-depleted and codon-optimized for expression in a human cell.

Disclosed herein is an AAV vector comprising an isolated nucleic acid molecule, wherein the isolated nucleic acid sequence encodes human PHKA1, PHKA2, PHKB, CALM1, CALM2, CALM3, PHKG2, and/or PGYL. Disclosed herein is an AAV vector comprising an isolated nucleic acid molecule, wherein the isolated nucleic acid sequence encodes human PHKA1, PHKA2, PHKB, CALM1, CALM2, CALM3, PHKG2, and/or PGYL, wherein the isolated nucleic acid sequence is CpG-depleted and codon-optimized for expression in a human cell.

Disclosed herein is an AAV vector comprising an isolated nucleic acid molecule, wherein the isolated nucleic acid sequence encodes alpha glucosidase or GAA. Disclosed herein is an AAV vector comprising an isolated nucleic acid molecule, wherein the isolated nucleic acid sequence encodes alpha glucosidase or GAA, wherein the isolated nucleic acid sequence is CpG-depleted and codon-optimized for expression in a human cell.

In an aspect, the nucleic acid sequence can have a coding sequence that is less than about 4.5 kilobases.

In an aspect, a disclosed nucleic acid sequence can comprise the sequence for a phosphorylase kinase or a subunit of a phosphorylase kinase (e.g., PHKA1, PHKA2, PHKB, CALM1, CALM2, CALM3, and/or PHKG2). In an aspect, a disclosed nucleic acid sequence can comprise the sequence for glycogen phosphorylase liver form (e.g., PYGL). In an aspect, a disclosed nucleic acid can comprise the sequence set forth in any one of SEQ ID NO:12-SEQ ID NO:22 or SEQ ID NO:60-SEQ ID NO:62. In an aspect, a disclosed nucleic acid sequence can comprise a sequence having at least 30%, at least 40%, at least 50%, at least 60%, at least 70%, at least 80%, or at least 90% identity to the sequence set forth in any one of SEQ ID NO:12-SEQ ID NO:22 or SEQ ID NO:60-SEQ ID NO:62.

In an aspect, a disclosed encoded polypeptide can comprise the sequence set forth in any one of SEQ ID NO:01-SEQ ID NO:11 or SEQ ID NO:57-SEQ ID NO:59. In an aspect, a disclosed encoded polypeptide can comprise a sequence having at least 40-59%, at least 50-69%, or at least 80-99% identity to the sequence set forth in SEQ ID NO:01-SEQ ID NO:11 or SEQ ID NO:57-SEQ ID NO:59.

In an aspect, a disclosed viral vector can comprise an isolated nucleic acid molecule comprising the tissue specific promoter, the CpG-depleted and codon-optimized nucleic acid sequence encoding the polypeptide, and a polyadenylation sequence.

In an aspect, a disclosed AAV vector can include naturally isolated serotypes including, but not limited to, AAV1, AAV2, AAV3 (including 3a and 3b), AAV4, AAV5, AAV6, AAV7, AAV8, AAVrh8, AAV9, AAV10, AAVrh10, AAV11, AAV12, AAV13, AAVrh39, AAVrh43, AAVcy.7 as well as bovine AAV, caprine AAV, canine AAV, equine AAV, ovine AAV, avian AAV, primate AAV, non-primate AAV, and any other virus classified by the International Committee on Taxonomy of Viruses (ICTV) as an AAV. In an aspect, an AAV capsid can be a chimera either created by capsid evolution or by rational capsid engineering from a naturally isolated AAV variants to capture desirable serotype features such as enhanced or specific tissue tropism and/or a host immune response escape. Naturally isolated AAV variants include, but not limited to, AAV-DJ, AAV-HAE1, AAV-HAE2, AAVM41, AAV-1829, AAV2 Y/F, AAV2 TN, AAV2i8, AAV2.5, AAV9.45, AAV9.61, AAV-B1, AAV-AS, AAV9.45A-String (e.g., AAV9.45-AS), AAV9.45Angiopep, AAV9.47-Angiopep, and AAV9.47-AS, AAV-PHP.B, AAV-PHP.eB, AAV-PHP.S, AAV-F, AAVcc.47, and AAVcc.81. In an aspect, a disclosed AAV vector can be AAV-Rh74 or a related variant (e.g., capsid variants such as RHM4-1). In an aspect, a disclosed AAV vector can be AAV8. In an aspect, a disclosed AAV vector can be AAVhum.8. In an aspect, a disclosed AAV vector can be a self-complementary AAV as disclosed herein.

In an aspect, a disclosed AAV vector can comprise a liver-specific promoter operably linked to the isolated nucleic acid molecule. In an aspect, a disclosed liver-specific promoter can be the thyroxin binding globulin (TBG) promoter, the α1-microglobulin/bikunin enhancer/thyroid hormone-binding globulin promoter, the human albumin (hALB) promoter, the thyroid hormone-binding globulin promoter, the thyroxin binding globulin promoter, the α-1-anti-trypsin promoter, the bovine albumin (bAlb) promoter, the murine albumin (mAlb) promoter, the human α1-antitrypsin (hAAT) promoter, the ApoEhAAT promoter comprising the ApoE enhancer and the hAAT promoter, the transthyretin (TTR) promoter, the liver fatty acid binding protein promoter, the hepatitis B virus (HBV) promoter, the DC172 promoter consisting of the hAAT promoter and the α1-microglobulin enhancer, the DC190 promoter containing the human albumin promoter and the prothrombin enhancer, or any one of other natural and synthetic liver-specific promoter.

In an aspect, a disclosed liver-specific promoter can comprise any liver-specific promoter known to the art. In an aspect, a liver specific promoter can comprise about 845-bp and comprise the thyroid hormone-binding globulin promoter sequences (2382 to 13), two copies of α1-microglobulinybikunin enhancer sequences (22,804 through 22,704), and a 71-bp leader sequence as described by Ill C R, et al. (1997). In an aspect, a disclosed liver-specific promoter can comprise the sequence set forth in SEQ ID NO:56. In an aspect, a disclosed liver-specific promoter can comprise a sequence having at least 40%, 50%, 60%, 70%, 80%, 85%, 86%, 87%, 88%, 89%, 90%, 91%, 92%, 93%, 94%, 95%, 96%, 97%, 98%, or 99% identity to the sequence set forth in SEQ ID NO:56. In an aspect, a disclosed liver-specific promoter can comprise a sequence having at least 40%-60%, at least 60%-80%, at least 80%-90%, or at least 90%-100% identity to the sequence set forth in SEQ ID NO:56.

In an aspect, a disclosed ubiquitous promoter can be a CMV enhancer/chicken β-actin promoter (CB promoter).

In an aspect, a disclosed promoter can be a promoter/enhancer. In an aspect, a disclosed promoter can be an endogenous promoter. In an aspect, a disclosed endogenous promoter can be an endogenous promoter/enhancer. In an aspect, a disclosed endogenous promoter or a disclosed endogenous promoter/enhancer can generally be obtained from a non-coding region upstream of a transcription initiation site of a gene of interest (such as, for example, a disclosed phosphorylase kinase or phosphorylase kinase subunit (e.g., PhK α2, PhK β, PhK γ2, PhK δ) or some other enzyme involved in the glycogen metabolic pathway (PYGL)). In an aspect, a disclosed endogenous promoter or a disclosed endogenous promoter/enhancer can be used for constitutive and efficient expression of a disclosed gene (e.g., a nucleic acid sequence encoding a polypeptide capable of preventing glycogen accumulation and/or degrading accumulated glycogen).

In an aspect, a disclosed endogenous promoter or a disclosed endogenous promoter/enhancer can be used for constitutive and efficient expression of a phosphorylase kinase or a phosphorylase kinase subunit. In an aspect, a disclosed endogenous promoter or a disclosed endogenous promoter/enhancer can comprise the promoter for the gene encoding the phosphorylase kinase regulatory subunit alpha 2 (PhK α2) (SEQ ID NO:63), the phosphorylase kinase regulatory subunit beta (PhK β) (SEQ ID NO:64), the phosphorylase kinase catalytic subunit gamma 2 (PhK γ2) (SEQ ID NO:65), the phosphorylase kinase regulatory subunit delta (PhK δ), or glycogen phosphorylase (PYGL) (SEQ ID NO:66). For example, in an aspect, when an encoded polypeptide comprises the PhK α2 subunit, the disclosed endogenous promoter or a disclosed endogenous promoter/enhancer can comprise the promoter or promoter/enhancer for the gene encoding the PhK α2 subunit. Similarly, when an encoded polypeptide comprises the PhK R subunit, the disclosed endogenous promoter or a disclosed endogenous promoter/enhancer can comprise the promoter or promoter/enhancer for the gene encoding the PhK β subunit. For example, in an aspect, a disclosed endogenous promoter or a disclosed endogenous promoter/enhancer can comprise the sequence set forth in SEQ ID NO:63-SEQ ID NO:66 or a fragment thereof. For example, in an aspect, a disclosed endogenous promoter or a disclosed endogenous promoter/enhancer can comprise a sequence having at least 40%, at least 50%, at least 60%, at least 70%, at least 80%, at least 90%, at least 95%, or more than 95% identity to the sequence set forth in SEQ ID NO:63-SEQ ID NO:66 or a fragment thereof.

### 3. Formulations

Disclosed herein is a pharmaceutical formulation comprising a disclosed vector and/or or a disclosed isolated nucleic acid molecule. Disclosed herein is a pharmaceutical formulation comprising an isolated nucleic acid molecule comprising a nucleic acid sequence encoding a polypeptide capable of preventing glycogen accumulation and/or degrading accumulated glycogen and pharmaceutically acceptable carrier. Disclosed herein is a pharmaceutical formulation comprising a vector comprising an isolated nucleic acid molecule comprising a nucleic acid sequence encoding a polypeptide capable of preventing glycogen accumulation and/or degrading accumulated glycogen and pharmaceutically acceptable carrier.

Disclosed herein is a pharmaceutical formulation comprising an isolated nucleic acid molecule comprising a nucleic acid sequence encoding a polypeptide capable of preventing glycogen accumulation and/or degrading accumulated glycogen, and pharmaceutically acceptable carrier, wherein the encoded polypeptide comprises human glycogen phosphorylase kinase subunit α1, glycogen phosphorylase kinase subunit α2, glycogen phosphorylase kinase subunit β, glycogen phosphorylase kinase subunit δ, glycogen phosphorylase kinase subunit γ2, and/or glycogen phosphorylase. Disclosed herein is a pharmaceutical formulation comprising a vector comprising an isolated nucleic acid molecule comprising a nucleic acid sequence encoding a polypeptide capable of preventing glycogen accumulation and/or degrading accumulated glycogen, and pharmaceutically acceptable carrier, wherein the encoded polypeptide comprises human glycogen phosphorylase kinase subunit α1, glycogen phosphorylase kinase subunit α2, glycogen phosphorylase kinase subunit β, glycogen phosphorylase kinase subunit δ, glycogen phosphorylase kinase subunit γ2, and/or glycogen phosphorylase.

Disclosed herein is a pharmaceutical formulation comprising a vector comprising an isolated nucleic acid molecule comprising a nucleic acid sequence encoding a polypeptide capable of preventing glycogen accumulation and/or degrading accumulated glycogen, wherein the nucleic acid sequence is CpG depleted and codon-optimized for expression in a human in a pharmaceutically acceptable carrier. Disclosed herein is a pharmaceutical formulation comprising a vector comprising an isolated nucleic acid molecule comprising a nucleic acid sequence encoding a polypeptide capable of preventing glycogen accumulation and/or degrading accumulated glycogen, wherein the nucleic acid sequence is CpG depleted and codon-optimized for expression in a human in a pharmaceutically acceptable carrier, and wherein the encoded polypeptide comprises human glycogen phosphorylase kinase subunit α1, glycogen phosphorylase kinase subunit α2, glycogen phosphorylase kinase subunit β, glycogen phosphorylase kinase subunit δ, glycogen phosphorylase kinase subunit γ2, and/or glycogen phosphorylase.

Disclosed herein is a pharmaceutical formulation comprising an isolated nucleic acid molecule comprising a nucleic acid sequence encoding a polypeptide capable of restoring one or more aspects of the glycogen metabolic pathway. Disclosed herein is a pharmaceutical formulation comprising an isolated nucleic acid molecule comprising a nucleic acid sequence encoding a polypeptide capable restoring one or more aspects of the glycogen metabolic pathway, wherein the nucleic acid sequence is CpG depleted and codon-optimized for expression in a human cell. Disclosed herein is a pharmaceutical formulation comprising an isolated nucleic acid molecule comprising a nucleic acid sequence encoding a polypeptide capable of restoring one or more aspects of the glycogen metabolic pathway, and a nucleic acid sequence encoding a polypeptide capable of reducing the expression level and/or activity level of glycogen synthase.

Disclosed herein is a pharmaceutical formulation comprising an isolated nucleic acid molecule comprising a nucleic acid sequence encoding a polypeptide capable of restoring one or more aspects of the glycogenolysis metabolic pathway. Disclosed herein is a pharmaceutical formulation comprising an isolated nucleic acid molecule comprising a nucleic acid sequence encoding a polypeptide capable restoring one or more aspects of the glycogenolysis metabolic pathway, wherein the nucleic acid sequence is CpG depleted and codon-optimized for expression in a human cell. Disclosed herein is a pharmaceutical formulation comprising an isolated nucleic acid molecule comprising a nucleic acid sequence encoding a polypeptide capable of restoring one or more aspects of the glycogenolysis metabolic pathway, and a nucleic acid sequence encoding a polypeptide capable of reducing the expression level and/or activity level of glycogen synthase. For example, in an aspect, a disclosed pharmaceutical formulation can restore the balance of glycogen synthesis and degradation.

Disclosed herein is a pharmaceutical formulation comprising an isolated nucleic acid molecule comprising a nucleic acid sequence encoding a polypeptide capable of restoring one or more aspects of the glycogenesis metabolic pathway. Disclosed herein is a pharmaceutical formulation comprising an isolated nucleic acid molecule comprising a nucleic acid sequence encoding a polypeptide capable restoring one or more aspects of the glycogenesis metabolic pathway, wherein the nucleic acid sequence is CpG depleted and codon-optimized for expression in a human cell. Disclosed herein is a pharmaceutical formulation comprising an isolated nucleic acid molecule comprising a nucleic acid sequence encoding a polypeptide capable of restoring one or more aspects of the glycogenesis metabolic pathway, and a nucleic acid sequence encoding a polypeptide capable of reducing the expression level and/or activity level of glycogen synthase.

Disclosed herein is a pharmaceutical formulation comprising an isolated nucleic acid molecule comprising a nucleic acid sequence encoding a polypeptide capable of restoring PhK subunit activity and/or functionality. Disclosed herein is a pharmaceutical formulation comprising an isolated nucleic acid molecule comprising a nucleic acid sequence encoding a polypeptide capable restoring PhK subunit activity and/or functionality, wherein the nucleic acid sequence is CpG depleted and codon-optimized for expression in a human cell. Disclosed herein is a pharmaceutical formulation comprising an isolated nucleic acid molecule comprising a nucleic acid sequence encoding a polypeptide capable of restoring PhK subunit activity and/or functionality, and a nucleic acid sequence encoding a polypeptide capable of reducing the expression level and/or activity level of glycogen synthase.

Disclosed herein is a pharmaceutical formulation comprising a vector comprising an isolated nucleic acid molecule comprising a nucleic acid sequence encoding a polypeptide capable of restoring one or more aspects of the glycogen metabolic pathway. Disclosed herein is a pharmaceutical formulation comprising a vector comprising an isolated nucleic acid molecule comprising a nucleic acid sequence encoding a polypeptide capable restoring one or more aspects of the glycogen metabolic pathway, wherein the nucleic acid sequence is CpG depleted and codon-optimized for expression in a human cell. Disclosed herein is a pharmaceutical formulation comprising a vector comprising an isolated nucleic acid molecule comprising a nucleic acid sequence encoding a polypeptide capable of restoring one or more aspects of the glycogen metabolic pathway, and a nucleic acid sequence encoding a polypeptide capable of reducing the expression level and/or activity level of glycogen synthase.

Disclosed herein is a pharmaceutical formulation comprising a vector comprising an isolated nucleic acid molecule comprising a nucleic acid sequence encoding a polypeptide capable of restoring one or more aspects of the glycogenolysis metabolic pathway. Disclosed herein is a pharmaceutical formulation comprising a vector comprising an isolated nucleic acid molecule comprising a nucleic acid sequence encoding a polypeptide capable restoring one or more aspects of the glycogenolysis metabolic pathway, wherein the nucleic acid sequence is CpG depleted and codon-optimized for expression in a human cell. Disclosed herein is a pharmaceutical formulation comprising a vector comprising an isolated nucleic acid molecule comprising a nucleic acid sequence encoding a polypeptide capable of restoring one or more aspects of the glycogenolysis metabolic pathway, and a nucleic acid sequence encoding a polypeptide capable of reducing the expression level and/or activity level of glycogen synthase.

Disclosed herein is a pharmaceutical formulation comprising a vector comprising an isolated nucleic acid molecule comprising a nucleic acid sequence encoding a polypeptide capable of restoring one or more aspects of the glycogenesis metabolic pathway. Disclosed herein is a pharmaceutical formulation comprising a vector comprising an isolated nucleic acid molecule comprising a nucleic acid sequence encoding a polypeptide capable restoring one or more aspects of the glycogenesis metabolic pathway, wherein the nucleic acid sequence is CpG depleted and codon-optimized for expression in a human cell. Disclosed herein is a pharmaceutical formulation comprising a vector comprising an isolated nucleic acid molecule comprising a nucleic acid sequence encoding a polypeptide capable of restoring one or more aspects of the glycogenesis metabolic pathway, and a nucleic acid sequence encoding a polypeptide capable of reducing the expression level and/or activity level of glycogen synthase.

Disclosed herein is a pharmaceutical formulation comprising an isolated nucleic acid molecule comprising a vector comprising a nucleic acid sequence encoding a polypeptide capable of restoring PhK subunit activity and/or functionality. Disclosed herein is a pharmaceutical formulation comprising a vector comprising an isolated nucleic acid molecule comprising a nucleic acid sequence encoding a polypeptide capable restoring PhK subunit activity and/or functionality, wherein the nucleic acid sequence is CpG depleted and codon-optimized for expression in a human cell. Disclosed herein is a pharmaceutical formulation comprising a vector comprising an isolated nucleic acid molecule comprising a nucleic acid sequence encoding a polypeptide capable of restoring PhK subunit activity and/or functionality, and a nucleic acid sequence encoding a polypeptide capable of reducing the expression level and/or activity level of glycogen synthase.

In an aspect, a disclosed pharmaceutical formulation can restore the functionality and/or structural integrity of the PhK complex. For example, in an aspect, by restoring the functionality and/or structural integrity of a subunit (such as α2, δ, β, and γ2), then the functionality and/or structural integrity of the PhK complex (the heterotetramer) can be restored.

In an aspect, a disclosed formulation can comprise (i) one or more active agents, (ii) biologically active agents, (iii) one or more pharmaceutically active agents, (iv) one or more immune-based therapeutic agents, (v) one or more clinically approved agents, or (vi) a combination thereof. In an aspect, a disclosed composition can comprise one or more proteasome inhibitors. In an aspect, a disclosed composition can comprise one or more immunosuppressives or immunosuppressive agents. In an aspect, an immunosuppressive agent can be anti-thymocyte globulin (ATG), cyclosporine (CSP), mycophenolate mofetil (MMF), or a combination thereof. In an aspect, a disclosed formulation can comprise an anaplerotic agent (such as, for example, C7 compounds like triheptanoin or MCT).

In an aspect, a disclosed method can comprise reducing the expression level, activity level, or both of glycogen synthase. In an aspect, a glycogen synthase can be GYS1 (muscle glycogen synthase) or GYS2 (liver glycogen synthase) or both. In an aspect, a disclosed GYS can be GYS2. In an aspect, a disclosed formulation can comprise a RNA therapeutic. A RNA therapeutic can comprise RNA-mediated interference (RNAi) and/or antisense oligonucleotides (ASO). In an aspect, a disclosed RNA therapeutic can be directed at GYS1, GYS2, or both. In an aspect, a disclosed RNA therapeutic can be directed at GYS2.

A disclosed RNA therapeutic can comprise therapy delivered via LNPs. In an aspect, a disclosed formulation can comprise an enzyme or enzyme precursor for enzyme replacement therapy (ERT).

In an aspect, a disclosed formulation can comprise a disclosed small molecule. In an aspect, a disclosed small molecule can inhibit and/or reduce the expression level and/or the activity level of glycogen synthase. In an aspect, a disclosed small molecule can, for example, inhibit glycogen synthase (i.e., GYS1 and/or GYS2) in a cell or a subject to reduce glycogen synthesis and/or glycogen accumulation in cells and tissues (e.g., skeletal muscle, lung tissue, liver tissue, brain tissue, or any other tissue having glycogen accumulation) when PHKA1, PHKA2, PHKB, PHKG2, CALM1, CALM2, CALM3, GAA, and/or GBE activity and/or expression levels are reduced (e.g., SRT). In an aspect, a disclosed small molecule can be guaiacol. In an aspect, a disclosed formulation can comprise an inhibitor of phosphorylation. For example, a disclosed formulation can comprise a modulator of the enzyme activity of GYS1 whereby the modulator acts through inhibitory phosphorylation (e.g., reduced phosphorylation of GYS1 kinase AMPK). For example, a disclosed formulation can comprise a modulator of the enzyme activity of GYS2.

### 4. Plasmids

Disclosed herein is a plasmid comprising one or more disclosed isolated nucleic acids and one or more disclosed promoters.

Disclosed herein is a plasmid comprising a nucleic acid sequence encoding a liver specific promoter (LSP) and hPHKG2. In an aspect, a plasmid comprising a nucleic acid sequence encoding a liver specific promoter (LSP) and hPHKG2 can comprise the sequence set forth in SEQ ID NO:48 or a sequence having at least 30%, at least 40%, at least 50%, at least 60%, at least 70%, at least 80%, or at least 90% identity to the sequence set forth in SEQ ID NO:48 or a sequence having at least 40-59%, at least 50-69%, or at least 80-99% identity to the sequence set forth in SEQ ID NO:48.

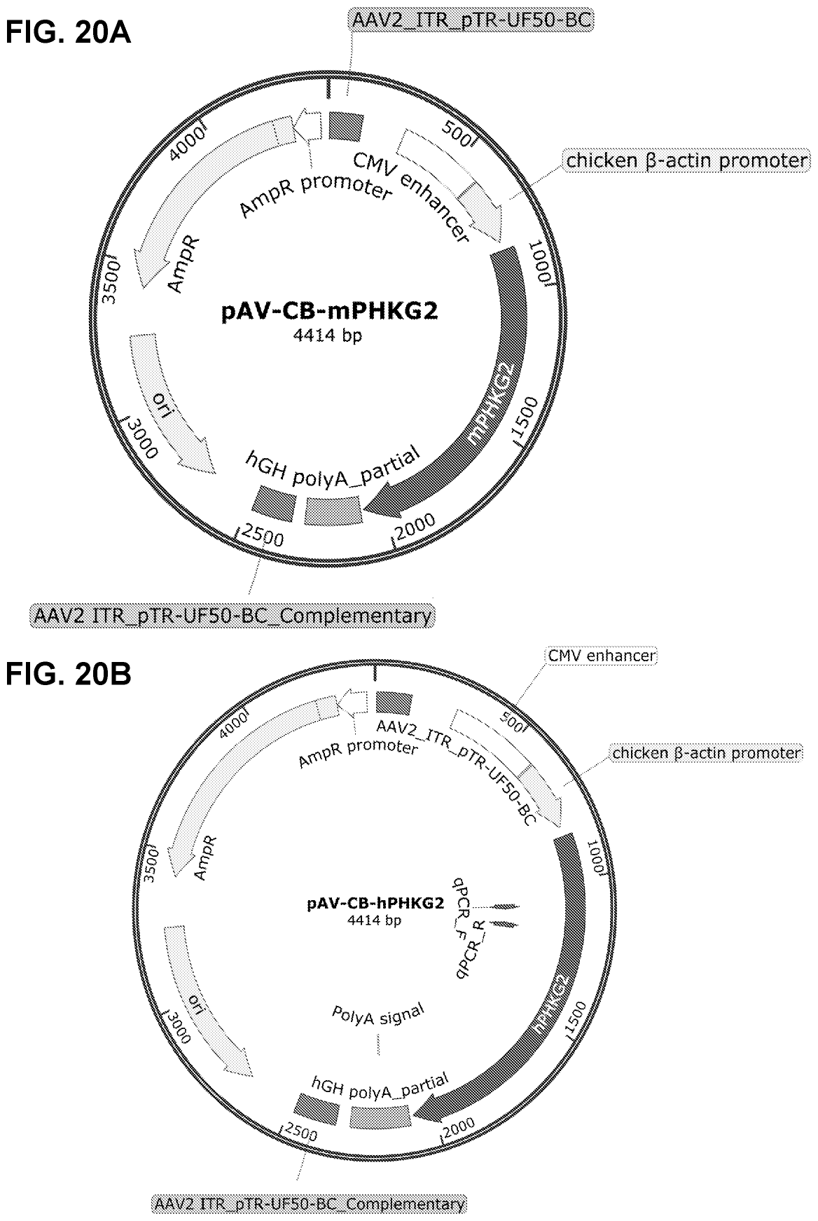

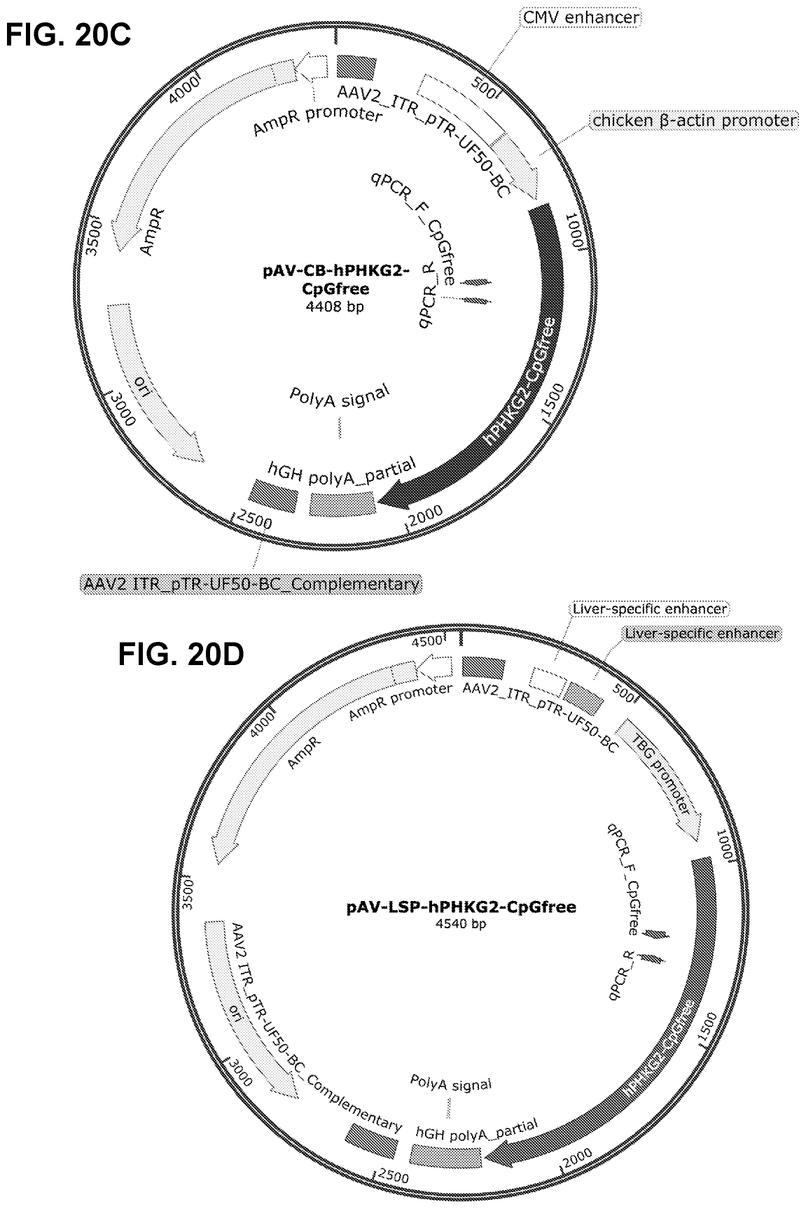

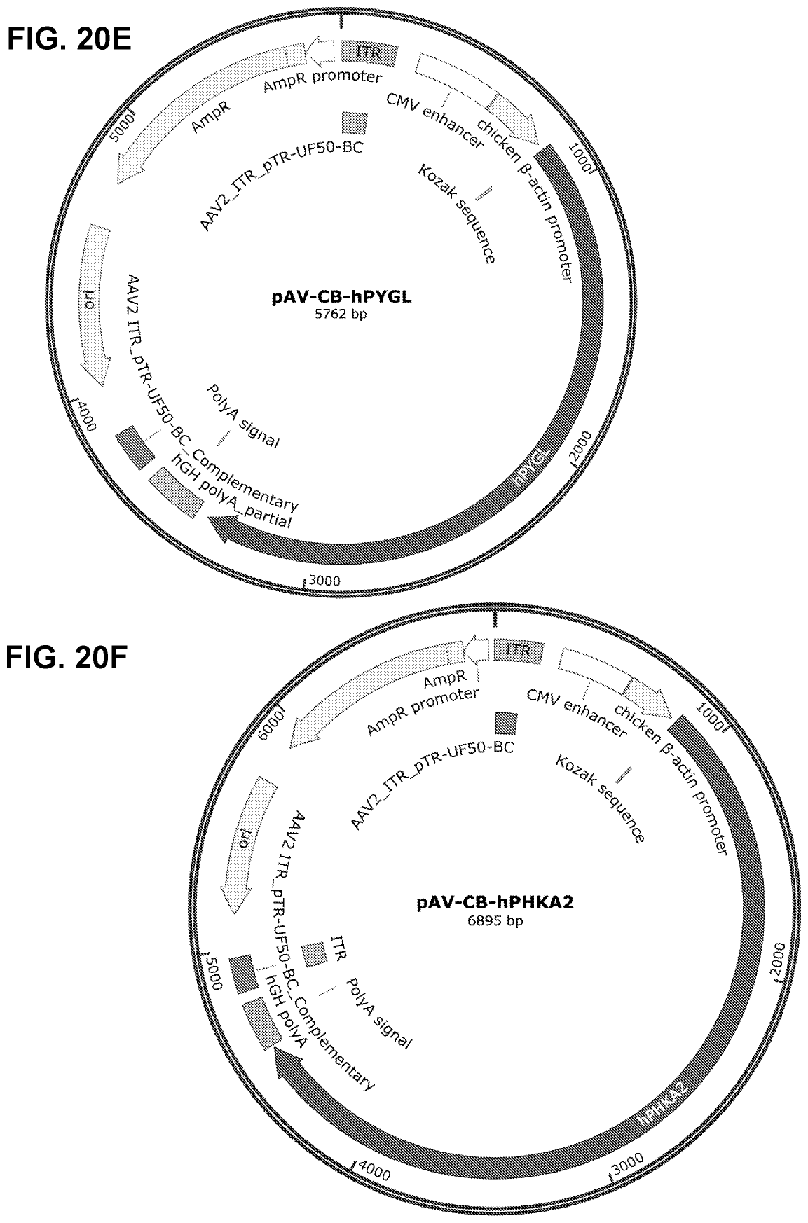

Disclosed herein is a plasmid comprising a nucleic acid sequence encoding a liver specific promoter (LSP) and hPHKG2^{CpG-depleted}. In an aspect, a plasmid comprising a nucleic acid sequence encoding a LSP and hPHKG2^{CpG-depleted} can comprise the sequence set forth in SEQ ID NO:49 or a sequence having at least 30%, at least 40%, at least 50%, at least 60%, at least 70%, at least 80%, or at least 90% identity to the sequence set forth in SEQ ID NO:49 or a sequence having at least 40-59%, at least 50-69%, or at least 80-99% identity to the sequence set forth in SEQ ID NO:49. In an aspect, a plasmid comprising a nucleic acid sequence encoding a LSP and hPHKG2^{CpG-depleted} can comprise the plasmid depicted in FIG. 20D.

Disclosed herein is a plasmid comprising a nucleic acid sequence encoding a CMV enhancer/chicken β-actin (CB) promoter and hPHKG2. In an aspect, a plasmid comprising a nucleic acid sequence encoding a CB promoter and hPHKG2 can comprise the sequence set forth in SEQ ID NO:50 or a sequence having at least 30%, at least 40%, at least 50%, at least 60%, at least 70%, at least 80%, or at least 90% identity to the sequence set forth in SEQ ID NO:50 or a sequence having at least 40-59%, at least 50-69%, or at least 80-99% identity to the sequence set forth in SEQ ID NO:50. In an aspect, a plasmid comprising a nucleic acid sequence encoding a CB promoter and hPHKG2 can comprise the plasmid depicted in FIG. 20B.

Disclosed herein is a plasmid comprising a nucleic acid sequence encoding a CMV enhancer/chicken β-actin (CB) promoter and hPHKG2^{CpG-depleted}. In an aspect, a plasmid comprising a nucleic acid sequence encoding a CB promoter and hPHKG2^{CpG-depleted} can comprise the sequence set forth in SEQ ID NO:51 or a sequence having at least 30%, at least 40%, at least 50%, at least 60%, at least 70%, at least 80%, or at least 90% identity to the sequence set forth in SEQ ID NO:51 or a sequence having at least 40-59%, at least 50-69%, or at least 80-99% identity to the sequence set forth in SEQ ID NO:51. In an aspect, a plasmid comprising a nucleic acid sequence encoding a CB promoter and hPHKG2^{CpG-depleted} can comprise the plasmid depicted in FIG. 20C.

Disclosed herein is a plasmid comprising a nucleic acid sequence encoding a liver specific promoter (LSP) and hPHKA2. In an aspect, a plasmid comprising a nucleic acid sequence encoding a LSP and hPHKA2 can comprise the sequence set forth in SEQ ID NO:44 or a sequence having at least 30%, at least 40%, at least 50%, at least 60%, at least 70%, at least 80%, or at least 90% identity to the sequence set forth in SEQ ID NO:44 or a sequence having at least 40-59%, at least 50-69%, or at least 80-99% identity to the sequence set forth in SEQ ID NO:44.

Disclosed herein is a plasmid comprising a nucleic acid sequence encoding a liver specific promoter (LSP) and hPHKA2^{CpG-depleted}. In an aspect, a plasmid comprising a nucleic acid sequence encoding a LSP and hPHKA2^{CpG-depleted} can comprise the sequence set forth in SEQ ID NO:45 or a sequence having at least 30%, at least 40%, at least 50%, at least 60%, at least 70%, at least 80%, or at least 90% identity to the sequence set forth in SEQ ID NO:45 or a sequence having at least 40-59%, at least 50-69%, or at least 80-99% identity to the sequence set forth in SEQ ID NO:45.

Disclosed herein is a plasmid comprising a nucleic acid sequence encoding a CMV enhancer/chicken β-actin (CB) promoter and hPHKA2. In an aspect, a plasmid comprising a nucleic acid sequence encoding a CB promoter and hPHKA2 can comprise the sequence set forth in SEQ ID NO:46 or a sequence having at least 30%, at least 40%, at least 50%, at least 60%, at least 70%, at least 80%, or at least 90% identity to the sequence set forth in SEQ ID NO:46 or a sequence having at least 40-59%, at least 50-69%, or at least 80-99% identity to the sequence set forth in SEQ ID NO:46. In an aspect, a plasmid comprising a nucleic acid sequence encoding a CB promoter and CpG-depleted hPHKA2 can comprise the plasmid depicted in FIG. 20F.

Disclosed herein is a plasmid comprising a nucleic acid sequence encoding a CMV enhancer/chicken β-actin (CB) promoter and hPHKA2^{CpG-depleted}. In an aspect, a plasmid comprising a nucleic acid sequence encoding a CB promoter and hPHKA2^{CpG-depleted} can comprise the sequence set forth in SEQ ID NO:47 or a sequence having at least 30%, at least 40%, at least 50%, at least 60%, at least 70%, at least 80%, or at least 90% identity to the sequence set forth in SEQ ID NO:47 or a sequence having at least 40-59%, at least 50-69%, or at least 80-99% identity to the sequence set forth in SEQ ID NO:47.

Disclosed herein is a plasmid comprising a nucleic acid sequence encoding a liver specific promoter (LSP) and hPYGL. In an aspect, a plasmid comprising a nucleic acid sequence encoding a LSP and hPYGL can comprise the sequence set forth in SEQ ID NO:52 or a sequence having at least 30%, at least 40%, at least 50%, at least 60%, at least 70%, at least 80%, or at least 90% identity to the sequence set forth in SEQ ID NO:52 or a sequence having at least 40-59%, at least 50-69%, or at least 80-99% identity to the sequence set forth in SEQ ID NO:52.

Disclosed herein is a plasmid comprising a nucleic acid sequence encoding a liver specific promoter (LSP) and hPYGL^{CpG-depleted}. In an aspect, a plasmid comprising a nucleic acid sequence encoding a LSP and hPYGL^{CpG-depleted} can comprise the sequence set forth in SEQ ID NO:53 or a sequence having at least 30%, at least 40%, at least 50%, at least 60%, at least 70%, at least 80%, or at least 90% identity to the sequence set forth in SEQ ID NO:53 or a sequence having at least 40-59%, at least 50-69%, or at least 80-99% identity to the sequence set forth in SEQ ID NO:53.

Disclosed herein is a plasmid comprising a nucleic acid sequence encoding a CMV enhancer/chicken β-actin (CB) promoter and hPYGL. In an aspect, a plasmid comprising a nucleic acid sequence encoding a CB promoter and hPYGL can comprise the sequence set forth in SEQ ID NO:54 or a sequence having at least 30%, at least 40%, at least 50%, at least 60%, at least 70%, at least 80%, or at least 90% identity to the sequence set forth in SEQ ID NO:54 or a sequence having at least 40-59%, at least 50-69%, or at least 80-99% identity to the sequence set forth in SEQ ID NO:54. In an aspect, a plasmid comprising a nucleic acid sequence encoding a CB promoter and hPYGL can comprise the plasmid depicted in FIG. 20E.

Disclosed herein is a plasmid comprising a nucleic acid sequence encoding a CMV enhancer/chicken β-actin (CB) promoter and hPYGL^{CpG-depleted}. In an aspect, a plasmid comprising a nucleic acid sequence encoding a CB promoter and hPYGL^{CpG-depleted} can comprise the sequence set forth in SEQ ID NO:55 or a sequence having at least 30%, at least 40%, at least 50%, at least 60%, at least 70%, at least 80%, or at least 90% identity to the sequence set forth in SEQ ID NO:55 or a sequence having at least 40-59%, at least 50-69%, or at least 80-99% identity to the sequence set forth in SEQ ID NO:55.

Disclosed herein is a plasmid comprising a nucleic acid sequence encoding a liver specific promoter (LSP) and hPHKB. In an aspect, a plasmid comprising a nucleic acid sequence encoding a LSP and hPHKB can comprise the sequence set forth in SEQ ID NO:67 or a sequence having at least 30%, at least 40%, at least 50%, at least 60%, at least 70%, at least 80%, or at least 90% identity to the sequence set forth in SEQ ID NO:67 or a sequence having at least 40-59%, at least 50-69%, or at least 80-99% identity to the sequence set forth in SEQ ID NO:67.

Disclosed herein is a plasmid comprising a nucleic acid sequence encoding a liver specific promoter (LSP) and hPHKB^{CpG-depleted}. In an aspect, a plasmid comprising a nucleic acid sequence encoding a LSP and hPHKB^{CpG-depleted} can comprise the sequence set forth in SEQ ID NO:68 or a sequence having at least 30%, at least 40%, at least 50%, at least 60%, at least 70%, at least 80%, or at least 90% identity to the sequence set forth in SEQ ID NO:68 or a sequence having at least 40-59%, at least 50-69%, or at least 80-99% identity to the sequence set forth in SEQ ID NO:68.

Disclosed herein is a plasmid comprising a nucleic acid sequence encoding a CMV enhancer/chicken β-actin (CB) promoter and hPHKB. In an aspect, a plasmid comprising a nucleic acid sequence encoding a CB promoter and hPHKB can comprise the sequence set forth in SEQ ID NO:69 or a sequence having at least 30%, at least 40%, at least 50%, at least 60%, at least 70%, at least 80%, or at least 90% identity to the sequence set forth in SEQ ID NO:69 or a sequence having at least 40-59%, at least 50-69%, or at least 80-99% identity to the sequence set forth in SEQ ID NO:69.

Disclosed herein is a plasmid comprising a nucleic acid sequence encoding a CMV enhancer/chicken β-actin (CB) promoter and hPHKB^{CpG-depleted}. In an aspect, a plasmid comprising a nucleic acid sequence encoding a CB promoter and hPHKB^{CpG-depleted} Can comprise the sequence set forth in SEQ ID NO:70 or a sequence having at least 30%, at least 40%, at least 50%, at least 60%, at least 70%, at least 80%, or at least 90% identity to the sequence set forth in SEQ ID NO:70 or a sequence having at least 40-59%, at least 50-69%, or at least 80-99% identity to the sequence set forth in SEQ ID NO:70.

### 5. Cells

Disclosed herein are cells comprising a disclosed isolated nucleic acid molecule, a disclosed vector, and/or a disclosed plasmid. Cells are known to the art. In an aspect, a disclosed cell can comprise the plasmid set forth in any one of FIG. 20A-FIG. 20E. In an aspect, a disclosed cell can comprise the plasmid set forth in any one of SEQ ID NO:44-SEQ ID NO:55 or SEQ ID NO:67-SEQ ID NO:70.

### 6. Animals

Disclosed herein are animals treated with one or more disclosed isolated nucleic acid molecules, disclosed vectors, disclosed pharmaceutical formulations, and/or disclosed plasmids. Cells are known to the art. In an aspect, a disclosed animal has been treated with a vector comprising the plasmid set forth in any one of SEQ ID NO:44-SEQ ID NO:55 or SEQ ID NO:67-SEQ ID NO:70.

### D. Methods of Treating and/or Preventing GSD IX and/or GSD VI Disease Progression

### 1. Vector Based Methods

Disclosed herein is a method of treating and/or preventing GSD IX al, GSD IX α2, GSD IX β, GSD IX δ, GSD IX γ2, and/or GSD VI disease progression comprising administering to a subject in need thereof a therapeutically effective amount of a disclosed vector comprising a disclosed isolated nucleic acid molecule, a disclosed pharmaceutical formulation, or a combination thereof. Disclosed herein is a method of treating and/or preventing disease progression comprising administering to a subject having GSD IX and/or GSD VI a therapeutically effective amount of a disclosed vector comprising a disclosed isolated nucleic acid molecule, a disclosed pharmaceutical formulation, or a combination thereof.

Disclosed herein is a method of treating and/or preventing GSD IX al, GSD IX α2, GSD IX β, GSD IX δ, GSD IX γ2, and/or GSD VI disease progression comprising administering to a subject in need thereof a therapeutically effective amount of a disclosed isolated nucleic acid molecule, a disclosed vector, and/or a disclosed pharmaceutical formulation, and administering to the subject an RNA therapeutic to reduce or inhibit the expression level and/or activity level of glycogen synthase, wherein the RNA therapeutic comprises RNAi or antisense oligonucleotides or wherein the RNA therapeutic comprises miRNA. Disclosed herein is a method of treating and/or preventing disease progression comprising administering to a subject having GSD IX and/or GSD VI a therapeutically effective amount of a disclosed isolated nucleic acid molecule, a disclosed vector, and/or a disclosed pharmaceutical formulation, and administering to the subject an RNA therapeutic to reduce or inhibit the expression level and/or activity level of glycogen synthase, wherein the RNA therapeutic comprises RNAi or antisense oligonucleotides or wherein the RNA therapeutic comprises miRNA.

Disclosed herein is a method of treating and/or preventing GSD IX al, GSD IX α2, GSD IX β, GSD IX δ, GSD IX γ2, and/or GSD VI disease progression comprising administering to a subject in need thereof a therapeutically effective amount of a disclosed isolated nucleic acid molecule, a disclosed vector, and/or a disclosed pharmaceutical formulation, and administering to the subject a small molecule to reduce or inhibit the expression level and/or activity level of glycogen synthase, wherein the small molecule targets GYS1 and/or GYS2, transcription of GYS1 and/or GYS2, and/or translation of GYS1 and/or GYS2. Disclosed herein is a method of treating and/or preventing disease progression comprising administering to a subject having GSD IX and/or GSD VI a therapeutically effective amount of a disclosed isolated nucleic acid molecule, a disclosed vector, and/or a disclosed pharmaceutical formulation, and administering to the subject a small molecule to reduce or inhibit the expression level and/or activity level of glycogen synthase, wherein the small molecule targets GYS1 and/or GYS2, transcription of GYS1 and/or GYS2, and/or translation of GYS1 and/or GYS2.

Disclosed herein is a method of treating and/or preventing GSD IX al, GSD IX α2, GSD IX β, GSD IX δ, GSD IX γ2, and/or GSD VI disease progression comprising administering to a subject in need thereof a therapeutically effective amount of a disclosed isolated nucleic acid molecule, a disclosed vector, and/or a disclosed pharmaceutical formulation, and using a gene editing system to reduce or inhibit the expression level and/or activity level of glycogen synthase, wherein the gene editing system comprises a Cas9 enzyme sequence (or a derivative thereof) and a guide RNA (gRNA). Disclosed herein is a method of treating and/or preventing disease progression comprising administering to a subject having GSD IX and/or GSD VI a therapeutically effective amount of a disclosed isolated nucleic acid molecule, a disclosed vector, and/or a disclosed pharmaceutical formulation, and using a gene editing system to reduce or inhibit the expression level and/or activity level of glycogen synthase, wherein the gene editing system comprises a Cas9 enzyme sequence (or a derivative thereof) and a guide RNA (gRNA).

Disclosed herein is a method of treating and/or preventing GSD IX al, GSD IX α2, GSD IX β, GSD IX δ, GSD IX γ2, and/or GSD VI disease progression comprising administering to a subject in need thereof a therapeutically effective amount of a disclosed isolated nucleic acid molecule, a disclosed vector, and/or a disclosed pharmaceutical formulation, and reducing or inhibiting the expression level and/or activity level of glycogen synthase, wherein reducing or inhibiting the expression level and/or activity level of glycogen synthase comprises any means known to and practiced by the art and/or disclosed herein. Disclosed herein is a method of treating and/or preventing disease progression comprising administering to a subject having GSD IX and/or GSD VI a therapeutically effective amount of a disclosed isolated nucleic acid molecule, a disclosed vector, and/or a disclosed pharmaceutical formulation, and reducing or inhibiting the expression level and/or activity level of glycogen synthase, wherein reducing or inhibiting the expression level and/or activity level of glycogen synthase comprises any means known to and practiced by the art and/or disclosed herein.

Disclosed herein is a method of treating and/or preventing GSD IX al, GSD IX α2, GSD IX β, GSD IX δ, GSD IX γ2, and/or GSD VI disease progression comprising administering to a subject in need thereof a therapeutically effective amount of the vector comprising an isolated nucleic acid molecule comprising a nucleic acid sequence encoding a polypeptide capable of preventing glycogen accumulation and/or degrading accumulated glycogen, wherein the nucleic acid sequence is CpG depleted and codon-optimized for expression in a human cell, and preventing glycogen accumulation and/or degrading accumulated glycogen in the subject. Disclosed herein is a method of treating and/or preventing disease progression comprising administering to a subject having GSD IX and/or GSD VI a therapeutically effective amount of the vector comprising an isolated nucleic acid molecule comprising a nucleic acid sequence encoding a polypeptide capable of preventing glycogen accumulation and/or degrading accumulated glycogen, wherein the nucleic acid sequence is CpG depleted and codon-optimized for expression in a human cell, and preventing glycogen accumulation and/or degrading accumulated glycogen in the subject.

Disclosed herein is a method of treating and/or preventing GSD IX al, GSD IX α2, GSD IX β, GSD IX δ, GSD IX γ2, and/or GSD VI disease progression comprising preventing glycogen accumulation and/or degrading accumulated glycogen in a subject in need thereof by administering to the subject a therapeutically effective amount of a vector comprising an isolated nucleic acid molecule comprising a nucleic acid sequence encoding a polypeptide capable of preventing glycogen accumulation and/or degrading accumulated glycogen, wherein the nucleic acid sequence is CpG depleted and codon-optimized for expression in a human cell. Disclosed herein is a method of treating and/or preventing disease progression comprising preventing glycogen accumulation and/or degrading accumulated glycogen in a subject having GSD IX and/or GSD VI by administering to the subject a therapeutically effective amount of a vector comprising an isolated nucleic acid molecule comprising a nucleic acid sequence encoding a polypeptide capable of preventing glycogen accumulation and/or degrading accumulated glycogen, wherein the nucleic acid sequence is CpG depleted and codon-optimized for expression in a human cell.

Disclosed herein is a method of treating and/or preventing GSD IX al, GSD IX α2, GSD IX β, GSD IX δ, GSD IX γ2, and/or GSD VI disease progression comprising administering to a subject in need thereof a therapeutically effective amount of a vector comprising an isolated nucleic acid molecule comprising a nucleic acid sequence encoding a polypeptide capable of preventing glycogen accumulation and/or degrading accumulated glycogen, wherein the nucleic acid sequence is CpG depleted and codon-optimized for expression in a human cell, thereby preventing glycogen accumulation and/or degrading accumulated glycogen in the subject. Disclosed herein is a method of treating and/or preventing disease progression comprising administering to a subject having GSD IX and/or GSD VI a therapeutically effective amount of a vector comprising an isolated nucleic acid molecule comprising a nucleic acid sequence encoding a polypeptide capable of preventing glycogen accumulation and/or degrading accumulated glycogen, wherein the nucleic acid sequence is CpG depleted and codon-optimized for expression in a human cell, thereby preventing glycogen accumulation and/or degrading accumulated glycogen in the subject.

Disclosed herein is a method of preventing glycogen accumulation and/or degrading accumulated glycogen comprising administering to a subject having GSD IX al, GSD IX α2, GSD IX β, GSD IX δ, GSD IX γ2, and/or GSD VI disease progression a therapeutically effective amount of a vector comprising an isolated nucleic acid molecule comprising a nucleic acid sequence encoding a polypeptide capable of preventing glycogen accumulation and/or degrading accumulated glycogen, wherein the nucleic acid sequence is CpG depleted and codon-optimized for expression in a human cell. Disclosed herein is a method of treating and/or preventing disease progression comprising administering to a subject having GSD IX and/or GSD VI a therapeutically effective amount of a vector comprising an isolated nucleic acid molecule comprising a nucleic acid sequence encoding a polypeptide capable of preventing glycogen accumulation and/or degrading accumulated glycogen, wherein the nucleic acid sequence is CpG depleted and codon-optimized for expression in a human cell.

Disclosed herein is a method of treating and/or preventing GSD VI and/or GSD IX disease progression comprising administering to a subject in need thereof a therapeutically effective amount of a disclosed vector comprising a disclosed isolated nucleic acid molecule, a disclosed pharmaceutical formulation, or a combination thereof, and reducing the expression level and/or activity level of glycogen synthase.

Disclosed herein is a method of treating and/or preventing GSD IX al, GSD IX α2, GSD IX β, GSD IX δ, GSD IX γ2, and/or GSD VI disease progression comprising administering to a subject in need thereof a therapeutically effective amount of a disclosed vector comprising a disclosed isolated nucleic acid molecule, a disclosed pharmaceutical formulation, or a combination thereof, and reducing the expression level and/or activity level of glycogen synthase Disclosed herein is a method of treating and/or preventing disease progression comprising administering to a subject having GSD IX and/or GSD VI a therapeutically effective amount of a disclosed vector comprising a disclosed isolated nucleic acid molecule, a disclosed pharmaceutical formulation, or a combination thereof, and reducing the expression level and/or activity level of glycogen synthase.

Disclosed herein is a method of treating and/or preventing GSD IX al, GSD IX α2, GSD IX β, GSD IX δ, GSD IX γ2, and/or GSD VI disease progression comprising administering to a subject in need thereof a therapeutically effective amount of the vector comprising an isolated nucleic acid molecule comprising a nucleic acid sequence encoding a polypeptide capable of preventing glycogen accumulation and/or degrading accumulated glycogen, wherein the nucleic acid sequence is CpG depleted and codon-optimized for expression in a human cell, reducing the expression level and/or activity level of glycogen synthase, and preventing glycogen accumulation and/or degrading accumulated glycogen in the subject. Disclosed herein is a method of treating and/or preventing disease progression comprising administering to a subject having GSD IX and/or GSD VI a therapeutically effective amount of the vector comprising an isolated nucleic acid molecule comprising a nucleic acid sequence encoding a polypeptide capable of preventing glycogen accumulation and/or degrading accumulated glycogen, wherein the nucleic acid sequence is CpG depleted and codon-optimized for expression in a human cell, reducing the expression level and/or activity level of glycogen synthase, and preventing glycogen accumulation and/or degrading accumulated glycogen in the subject.

Disclosed herein is a method of treating and/or preventing GSD IX al, GSD IX α2, GSD IX β, GSD IX δ, GSD IX γ2, and/or GSD VI disease progression comprising preventing glycogen accumulation and/or degrading accumulated glycogen in a subject in need thereof by administering to the subject a therapeutically effective amount of a vector comprising an isolated nucleic acid molecule comprising a nucleic acid sequence encoding a polypeptide capable of preventing glycogen accumulation and/or degrading accumulated glycogen, wherein the nucleic acid sequence is CpG depleted and codon-optimized for expression in a human cell, and reducing the expression level and/or activity level of glycogen synthase. Disclosed herein is a method of treating and/or preventing disease progression comprising administering to a subject having GSD IX and/or GSD VI a therapeutically effective amount of a vector comprising an isolated nucleic acid molecule comprising a nucleic acid sequence encoding a polypeptide capable of preventing glycogen accumulation and/or degrading accumulated glycogen, wherein the nucleic acid sequence is CpG depleted and codon-optimized for expression in a human cell, and reducing the expression level and/or activity level of glycogen synthase.

Disclosed herein is a method of treating and/or preventing GSD IX al, GSD IX α2, GSD IX β, GSD IX δ, GSD IX γ2, and/or GSD VI disease progression comprising administering to a subject in need thereof a therapeutically effective amount of a vector comprising an isolated nucleic acid molecule comprising a nucleic acid sequence encoding a polypeptide capable of preventing glycogen accumulation and/or degrading accumulated glycogen, wherein the nucleic acid sequence is CpG depleted and codon-optimized for expression in a human cell, reducing the expression level and/or activity level of glycogen synthase, thereby preventing glycogen accumulation and/or degrading accumulated glycogen in the subject. Disclosed herein is a method of treating and/or preventing disease progression comprising administering to a subject having GSD IX and/or GSD VI a therapeutically effective amount of a vector comprising an isolated nucleic acid molecule comprising a nucleic acid sequence encoding a polypeptide capable of preventing glycogen accumulation and/or degrading accumulated glycogen, wherein the nucleic acid sequence is CpG depleted and codon-optimized for expression in a human cell, reducing the expression level and/or activity level of glycogen synthase, thereby preventing glycogen accumulation and/or degrading accumulated glycogen in the subject.

Disclosed herein is a method of preventing glycogen accumulation and/or degrading accumulated glycogen comprising administering to a subject having GSD IX al, GSD IX α2, GSD IX β, GSD IX δ, GSD IX γ2, and/or GSD VI disease progression a therapeutically effective amount of a vector comprising an isolated nucleic acid molecule comprising a nucleic acid sequence encoding a polypeptide capable of preventing glycogen accumulation and/or degrading accumulated glycogen, wherein the nucleic acid sequence is CpG depleted and codon-optimized for expression in a human cell, and reducing the expression level and/or activity level of glycogen synthase. Disclosed herein is a method of preventing glycogen accumulation and/or degrading accumulated glycogen comprising administering to a subject having GSD IX and/or GSD VI a therapeutically effective amount of a vector comprising an isolated nucleic acid molecule comprising a nucleic acid sequence encoding a polypeptide capable of preventing glycogen accumulation and/or degrading accumulated glycogen, wherein the nucleic acid sequence is CpG depleted and codon-optimized for expression in a human cell, and reducing the expression level and/or activity level of glycogen synthase.

In an aspect, a disclosed method can restore the balance of glycogen metabolism, wherein glycogen metabolism comprises glycogen synthesis and breakdown. In an aspect, a disclosed method can restore the functionality and/or structural integrity of the PhK complex. For example, in an aspect, by restoring the functionality and/or structural integrity of a subunit (such as α2, δ, β, and γ2), then the functionality and/or structural integrity of the PhK complex (the heterotetramer) can be restored.

In an aspect, a subject having GSD IX can have a defect in and/or a missing phosphorylase kinase regulatory subunit alpha 2 (PhK α2) subunit, phosphorylase kinase regulatory subunit beta (PhK β) subunit, phosphorylase kinase catalytic subunit gamma 2 (PhK γ2) subunit, phosphorylase kinase regulatory subunit delta (PhK δ) subunit, or any combination thereof. In an aspect, a subject having GSD VI can have a defect in and/or a missing PYGL.

In an aspect of a disclosed method, reducing the expression level and/or activity level of glycogen synthase can comprise SRT, siRNA-based therapies, shRNA-based therapies, antisense therapies, gene-editing therapies, therapies using one or more small molecules or peptide drugs, and/or therapies using a gene editing system.

an aspect, a disclosed method can correct a deficiency in the PhK complex, can restore the functionality of the PhK complex, or both. In an aspect, a disclosed method can correct or restore one or more aspects of the glycogen synthesis pathway or the glycogenolysis pathway or both. In an aspect, a disclosed method can correct, restore, supplement, and/or replenish the enzymatic activity of one or more glycogen phosphorylase kinases and/or glycogen phosphorylases. In an aspect, a disclosed method can correct, restore, supplement, and/or replenish the enzymatic activity of one or more human glycogen phosphorylase kinase subunit α1, glycogen phosphorylase kinase subunit α2, glycogen phosphorylase kinase subunit β, glycogen phosphorylase kinase subunit δ, glycogen phosphorylase kinase subunit γ2, and/or glycogen phosphorylase.

In an aspect, a disclosed method can restore one or more aspects of the glycogen metabolic pathway, restore one or more aspects of the glycogenolysis metabolic pathway, can restore one or more aspects of the glycogenesis metabolic pathway, can restore PhK subunit activity and/or functionality, can restore PYGL activity and/or functionality, or any combination thereof. For example, in an aspect, a disclosed method can restore the balance of glycogen synthesis and degradation. In an aspect, a disclosed method can restore the balance of glycogen metabolism, wherein glycogen metabolism comprises glycogen synthesis and breakdown. In an aspect, a disclosed method can restore the functionality and/or structural integrity of the PhK complex. For example, in an aspect, by restoring the functionality and/or structural integrity of a subunit (such as α2, δ, β, and γ2), then the functionality and/or structural integrity of the PhK complex (the heterotetramer) can be restored. In an aspect, restoring one or more aspects of a disclosed metabolic pathway can comprise restoring the activity and/or functionality of one or more enzymes identified in FIG. 1. In an aspect, restoring PhK subunit activity and/or functionality can comprise restoring the activity and/or functionality of the al subunit, the α2 subunit, the R subunit, the γ2 subunit, or any combination thereof.

In an aspect, restoring PhK activity and/or functionality or restoring PYGL activity and/or functionality can be a 10%, 20%, 30%, 40%, 50%, 60%, 70%, 80%, 90%, 100%, or any amount of restoration when compared to a pre-existing level such as, for example, a pre-treatment level. In an aspect, the amount of restoration can be 10-20%, 20-30%, 30-40%, 40-50%, 50-60%, 60-70%, 70-80%, 80-90%, or 90-100% more than a pre-existing level such as, for example, a pre-treatment level. In an aspect, restoration can be measured against a control level (e.g., a level in a subject not having a GSD). In an aspect, restoration can be a partial or incomplete restoration. In an aspect, restoration can be complete or near complete restoration such that the level of expression, activity and/or functionality is similar to that of a wild-type or control level.

In an aspect, a disclosed method can comprise restoring one or more aspects of cellular homeostasis and/or cellular functionality and/or metabolic dysregulation. In an aspect, restoring one or more aspects of cellular homeostasis and/or cellular functionality can comprise one or more of the following: (i) correcting cell starvation in one or more cell types (such as, for example, liver cells and muscle cells); (ii) normalizing aspects of the autophagy pathway (such as, for example, correcting, preventing, reducing, and/or ameliorating autophagy); (iii) improving, enhancing, restoring, and/or preserving mitochondrial functionality and/or structural integrity; (iv) improving, enhancing, restoring, and/or preserving organelle functionality and/or structural integrity; (v) preventing, slowing, and/or eliminating hypoglycemia, ketosis, and/or other liver abnormalities; (vi) correcting liver enzyme dysregulation; (vii) reversing, inhibiting, preventing, stabilizing, and/or slowing the rate of progression of the multi-systemic manifestations of GSD IX; (viii) reversing, inhibiting, preventing, stabilizing, and/or slowing the rate of progression of liver disease including fibrosis, cirrhosis, hepatic adenomas, and/or liver hepatocellular carcinoma, or (ix) any combination thereof. In an aspect, restoring one or more aspects of cellular homeostasis can comprise improving, enhancing, restoring, and/or preserving one or more aspects of cellular structural and/or functional integrity.

In an aspect of a disclosed method, techniques to monitor, measure, and/or assess the restoring one or more aspects of cellular homeostasis and/or cellular functionality can comprise qualitative (or subjective) means as well as quantitative (or objective) means. These means are known to the skilled person. For example, hypoglycemia can be sporadically and/or continuously measured and monitored. Methods and techniques for measuring and monitoring hypoglycemia are known to the skilled person and include, but not limited to, by continuous glucose monitoring (CGM) methods and capillary blood glucose sticks.

In an aspect, the nucleic acid sequence can have a coding sequence that is less than about 4.5 kilobases.

In an aspect, a disclosed vector can be a viral vector or a non-viral vector. In an aspect, a disclosed non-viral vector can be a polymer-based vector, a peptide-based vector, a lipid nanoparticle, a solid lipid nanoparticle, or a cationic lipid-based vector.

In an aspect, a disclosed viral vector can be an adenovirus vector, an AAV vector, a herpes simplex virus vector, a retrovirus vector, a lentivirus vector, and alphavirus vector, a flavivirus vector, a rhabdovirus vector, a measles virus vector, a Newcastle disease viral vector, a poxvirus vector, or a picornavirus vector.

In an aspect, a disclosed AAV vector can include naturally isolated serotypes including, but not limited to, AAV1, AAV2, AAV3 (including 3a and 3b), AAV4, AAV5, AAV6, AAV7, AAV8, AAVrh8, AAV9, AAV10, AAVrh10, AAV11, AAV12, AAV13, AAVrh39, AAVrh43, AAVcy.7 as well as bovine AAV, caprine AAV, canine AAV, equine AAV, ovine AAV, avian AAV, primate AAV, non-primate AAV, and any other virus classified by the International Committee on Taxonomy of Viruses (ICTV) as an AAV. In an aspect, an AAV capsid can be a chimera either created by capsid evolution or by rational capsid engineering from a naturally isolated AAV variants to capture desirable serotype features such as enhanced or specific tissue tropism and/or a host immune response escape. Naturally isolated AAV variants include, but are not limited to, AAV-DJ, AAV-HAE1, AAV-HAE2, AAVM41, AAV-1829, AAV2 Y/F, AAV2 T/V, AAV2i8, AAV2.5, AAV9.45, AAV9.61, AAV-B1, AAV-AS, AAV9.45A-String (e.g., AAV9.45-AS), AAV9.45Angiopep, AAV9.47-Angiopep, and AAV9.47-AS, AAV-PHP.B, AAV-PHP.eB, AAV-PHP.S, AAV-F, AAVcc.47, and AAVcc.81. In an aspect, a disclosed AAV vector can be AAV-Rh74 or a related variant (e.g., capsid variants such as RHM4-1). In an aspect, a disclosed AAV vector can be AAV8. In an aspect, a disclosed AAV vector can be AAVhum.8. In an aspect, a disclosed AAV vector can be a self-complementary AAV as disclosed herein.

In an aspect, a disclosed vector can comprise a liver-specific promoter operably linked to the isolated nucleic acid molecule. In an aspect, a disclosed liver-specific promoter can be the thyroxin binding globulin (TBG) promoter, the α1-microglobulin/bikunin enhancer/thyroid hormone-binding globulin promoter, the human albumin (hALB) promoter, the thyroid hormone-binding globulin promoter, the α-1-anti-trypsin promoter, the bovine albumin (bAlb) promoter, the murine albumin (mAlb) promoter, the human α1-antitrypsin (hAAT) promoter, the ApoEhAAT promoter comprising the ApoE enhancer and the hAAT promoter, the transthyretin (TTR) promoter, the liver fatty acid binding protein promoter, the hepatitis B virus (HBV) promoter, the DC172 promoter comprising the hAAT promoter and the α1-microglobulin enhancer, the DC190 promoter comprising the human albumin promoter and the prothrombin enhancer, or any other natural or synthetic liver-specific promoter.

In an aspect, a liver specific promoter can comprise about 845-bp and comprise the thyroid hormone-binding globulin promoter sequences (2382 to 13), two copies of α1-microglobulinybikunin enhancer sequences (22,804 through 22,704), and a 71-bp leader sequence as described by Ill C R, et al. (1997).

In an aspect, a disclosed liver-specific promoter can comprise any liver-specific promoter known to the art. In an aspect, a liver specific promoter can comprise about 845-bp and comprise the thyroid hormone-binding globulin promoter sequences (2382 to 13), two copies of α1-microglobulinybikunin enhancer sequences (22,804 through 22,704), and a 71-bp leader sequence as described by Ill C R, et al. (1997). In an aspect, a disclosed liver-specific promoter can comprise the sequence set forth in SEQ ID NO:56. In an aspect, a disclosed liver-specific promoter can comprise a sequence having at least 40%, 50%, 60%, 70%, 80%, 85%, 86%, 87%, 88%, 89%, 90%, 91%, 92%, 93%, 94%, 95%, 96%, 97%, 98%, or 99% identity to the sequence set forth in SEQ ID NO:56. In an aspect, a disclosed liver-specific promoter can comprise a sequence having at least 40%-60%, at least 60%-80%, at least 80%-90%, or at least 90%-100% identity to the sequence set forth in SEQ ID NO:56.

In an aspect, a disclosed promoter can be a promoter/enhancer. In an aspect, a disclosed promoter can be an endogenous promoter. In an aspect, a disclosed endogenous promoter can be an endogenous promoter/enhancer. In an aspect, a disclosed endogenous promoter or a disclosed endogenous promoter/enhancer can generally be obtained from a non-coding region upstream of a transcription initiation site of a gene of interest (such as, for example, a disclosed phosphorylase kinase or phosphorylase kinase subunit (e.g., PhK α2, PhK β, PhK γ2, PhK δ) or some other enzyme involved in the glycogen metabolic pathway (PYGL)). In an aspect, a disclosed endogenous promoter or a disclosed endogenous promoter/enhancer can be used for constitutive and efficient expression of a disclosed gene (e.g., a nucleic acid sequence encoding a polypeptide capable of preventing glycogen accumulation and/or degrading accumulated glycogen).

In an aspect, a disclosed endogenous promoter or a disclosed endogenous promoter/enhancer can be used for constitutive and efficient expression of a phosphorylase kinase or a phosphorylase kinase subunit. In an aspect, a disclosed endogenous promoter or a disclosed endogenous promoter/enhancer can comprise the promoter for the gene encoding the phosphorylase kinase regulatory subunit alpha 2 (PhK α2) (SEQ ID NO:63), the phosphorylase kinase regulatory subunit beta (PhK β) (SEQ ID NO:64), the phosphorylase kinase catalytic subunit gamma 2 (PhK γ2) (SEQ ID NO:65), the phosphorylase kinase regulatory subunit delta (PhK δ), or glycogen phosphorylase (PYGL) (SEQ ID NO:66). For example, in an aspect, when an encoded polypeptide comprises the PhK α2 subunit, the disclosed endogenous promoter or a disclosed endogenous promoter/enhancer can comprise the promoter or promoter/enhancer for the gene encoding the PhK α2 subunit. Similarly, when an encoded polypeptide comprises the PhK R subunit, the disclosed endogenous promoter or a disclosed endogenous promoter/enhancer can comprise the promoter or promoter/enhancer for the gene encoding the PhK β subunit. For example, in an aspect, a disclosed endogenous promoter or a disclosed endogenous promoter/enhancer can comprise the sequence set forth in SEQ ID NO:63-SEQ ID NO:66 or a fragment thereof. For example, in an aspect, a disclosed endogenous promoter or a disclosed endogenous promoter/enhancer can comprise a sequence having at least 40%, at least 50%, at least 60%, at least 70%, at least 80%, at least 90%, at least 95%, or more than 95% identity to the sequence set forth in SEQ ID NO:63-SEQ ID NO:66 or a fragment thereof.

In an aspect, a disclosed encoded polypeptide can degrade glycogen. In an aspect, a disclosed encoded polypeptide can be a phosphorylase kinase. In an aspect, an encoded polypeptide can be a phosphorylase. In an aspect, a disclosed encoded polypeptide can be derived from a human or a non-human source.

In an aspect, a disclosed encoded polypeptide can comprise the sequence set forth in SEQ ID NO:01-SEQ ID NO: 11 or SEQ ID NO:57-SEQ ID NO:59. In an aspect, a disclosed encoded polypeptide can comprise a sequence having at least 30%, at least 40%, at least 50%, at least 60%, at least 70%, at least 80%, or at least 90% identity to the sequence set forth in SEQ ID NO:01-SEQ ID NO:11 or SEQ ID NO:57-SEQ ID NO:59. In an aspect, a disclosed nucleic acid sequence can comprise the sequence set forth in SEQ ID NO:12-SEQ ID NO:22 or SEQ ID NO:60-SEQ ID NO:62. In an aspect, a disclosed nucleic acid sequence can comprise a sequence having at least 40-59%, at least 50-69%, or at least 80-99% identity to the sequence set forth in SEQ ID NO: 12-SEQ ID NO:22 or SEQ ID NO:60-SEQ ID NO:62.

In an aspect, a disclosed vector can comprise an isolated nucleic acid molecule comprising the liver-specific promoter, the CpG-depleted and codon-optimized nucleic acid sequence encoding the polypeptide, and a polyadenylation signal.

In an aspect, a subject can be a human subject. In an aspect, a disclosed vector can be delivered to the subject's liver.

In an aspect, a disclosed vector can be administered via intravenous, intraarterial, intramuscular, intraperitoneal, subcutaneous, intra-CSF, intrathecal, intraventricular, intrahepatic, hepatic intra-arterial, hepatic portal vein (HPV), or in utero administration. In an aspect, a disclosed vector can be administered via intra-CSF administration in combination with a disclosed nucleic acid molecule, a disclosed vector, and/or a disclosed pharmaceutical formulation. In an aspect, a disclosed vector can be administered via intra-CSF administration in combination with RNAi, antisense oligonucleotides, miRNA, one or more small molecules, one or more therapeutic agents, one or more proteasome inhibitors, one or more immune modulators, and/or a gene editing system. In an aspect, a disclosed vector can be administered via LNP administration. In an aspect, a subject can be a human subject. In an aspect, a disclosed vector can be delivered to the subject's liver, heart, skeletal muscle, smooth muscle, CNS, PNS, or a combination thereof. In an aspect, a disclosed vector can be concurrently and/or serially administered to a subject via multiple routes of administration. For example, in an aspect, administering a disclosed vector can comprise intravenous administration and intra-cistern magna (ICM) administration. In an aspect, administering a disclosed vector can comprise IV administration and intrathecal (ITH) administration.

In an aspect, a therapeutically effective amount of disclosed vector can be delivered via intravenous (IV) administration and can comprise a range of about 1×10¹⁰ vg/kg to about 2×10¹⁴ vg/kg. In an aspect, for example, a disclosed vector can be administered at a dose of about 1×10¹¹ to about 8×10¹³ vg/kg or about 1×10¹² to about 8×10¹³ vg/kg. In an aspect, a disclosed vector can be administered at a dose of about 1×10¹³ to about 6×10¹³ vg/kg. In an aspect, a disclosed vector can be administered at a dose of at least about 1×10¹⁰, at least about 5×10¹⁰, at least about 1×10¹¹, at least about 5×10¹¹, at least about 1×10¹², at least about 5×10¹², at least about 1×10¹³, at least about 5×10¹³, or at least about 1×10¹⁴ vg/kg. In an aspect, a disclosed vector can be administered at a dose of no more than about 1×10¹⁰, no more than about 5×10¹⁰, no more than about 1×10¹¹, no more than about 5×10¹¹, no more than about 1×10¹², no more than about 5×10¹², no more than about 1×10¹³, no more than about 5×10¹³, or no more than about 1×10¹⁴ vg/kg. In an aspect, a disclosed vector can be administered at a dose of about 1×10¹² vg/kg. In an aspect, a disclosed vector can be administered at a dose of about 1×10¹¹ vg/kg.

In an aspect, a disclosed vector can be administered in a single dose, or in multiple doses (such as 2, 3, 4, 5, 6, 7, 8, 9 or 10 doses) as needed for the desired therapeutic results.

In an aspect, a disclosed method can further comprise administering to the subject a therapeutically effective amount of a therapeutic agent. A therapeutic agent can be any disclosed agent that effects a desired clinical outcome.

In an aspect, a disclosed method can further comprise monitoring the subject for adverse effects. In an aspect, in the absence of adverse effects, the method can further comprise continuing to treat the subject. In an aspect, in the presence of adverse effects, the method can further comprise modifying the treating step.

In an aspect, a disclosed method can further comprise reducing glycogen levels by administering a glycogen synthase inhibitor (e.g., RNAi, ASO, etc.) to the subject, or modifying the subject's diet, for example, by using cornstarch or another slow release starch to prevent hypoglycemia, or modifying the subject's diet, for example, by consuming a high amount of protein, fat, or other anaplerotic agents (such as, for example, C7 compounds like triheptanoin or MCT), exercise or a combination thereof.

In an aspect, a disclosed method can comprise reducing the expression level, activity level, or both of glycogen synthase. In an aspect, a glycogen synthase can be GYS1 (muscle glycogen synthase) or GYS2 (liver glycogen synthase) or both. In an aspect, a disclosed GYS can be GYS2. In an aspect, a disclosed formulation can comprise a RNA therapeutic. A RNA therapeutic can comprise RNA-mediated interference (RNAi) and/or antisense oligonucleotides (ASO). In an aspect, a disclosed RNA therapeutic can be directed at GYS1, GYS2, or both. In an aspect, a disclosed RNA therapeutic can be directed at GYS2.

In an aspect of a disclosed method, reducing the expression level and/or activity level of glycogen synthase can comprise SRT. For example, in an aspect, SRT can comprise inhibiting glycogen synthase (i.e., GYS1 and/or GYS2) in a cell or a subject to reduce glycogen synthesis and/or glycogen accumulation in cells and tissues (e.g., skeletal muscle, lung tissue, liver tissue, brain tissue, or any other tissue having glycogen accumulation) when GAA and/or GBE activity and/or expression levels are reduced. In an aspect, SRT can comprise siRNA-based therapies, shRNA-based therapies, antisense therapies, gene-editing therapies, and therapies using one or more small molecules or peptide drugs.

In an aspect of a disclosed method, reducing the expression level and/or activity level of glycogen synthase can comprise administering a small molecule. In an aspect, a disclosed small molecule can reduce activity and/or expression of GYS1 in view of the reduced activity and/or expression level of GAA, GBE, or one or more other enzymes in the metabolic pathways of glycogen synthesis and breakdown. In an aspect, a disclosed small molecule can traverse the blood-brain-barrier. In an aspect, a disclosed small molecule can be guaiacol.

In an aspect, a disclosed method can comprise restoring glucose homeostasis. In an aspect of a disclosed method, techniques to monitor, measure, and/or assess the restoring glucose homeostasis can comprise qualitative (or subjective) means as well as quantitative (or objective) means. These means are known to the skilled person. For example, hypoglycemia can be sporadically and/or continuously measured and monitored. Methods and techniques for measuring and monitoring hypoglycemia are known to the skilled person and include, but not limited to, by continuous glucose monitoring (CGM) methods and capillary blood glucose sticks.

In an aspect, a disclosed method can comprise restoring one or more aspects of cellular homeostasis and/or cellular functionality and/or metabolic dysregulation. In an aspect, restoring one or more aspects of cellular homeostasis and/or cellular functionality can comprise one or more of the following: (i) correcting cell starvation in one or more cell types (such as, for example, liver cells and muscle cells); (ii) normalizing aspects of the autophagy pathway (such as, for example, correcting, preventing, reducing, and/or ameliorating autophagy); (iii) improving, enhancing, restoring, and/or preserving mitochondrial functionality and/or structural integrity; (iv) improving, enhancing, restoring, and/or preserving organelle functionality and/or structural integrity; (v) preventing, slowing, and/or eliminating hypoglycemia, ketosis, and/or other liver abnormalities; (vi) correcting liver enzyme dysregulation; (vii) reversing, inhibiting, preventing, stabilizing, and/or slowing the rate of progression of the multi-systemic manifestations of GSD IX; (viii) reversing, inhibiting, preventing, stabilizing, and/or slowing the rate of progression of liver disease including fibrosis, cirrhosis, hepatic adenomas, and/or liver hepatocellular carcinoma, or (ix) any combination thereof. In an aspect, restoring one or more aspects of cellular homeostasis can comprise improving, enhancing, restoring, and/or preserving one or more aspects of cellular structural and/or functional integrity.

In an aspect, a disclosed method of reducing the expression level and/or activity level of glycogen synthase can comprise using a gene editing system. In an aspect, a gene editing system can comprise CRISPR/Cas9, or can comprise zinc finger nucleases (ZFNs), transcription activator-like effector nucleases (TALENs), and/or homing endonucleases.

In an aspect, a disclosed method can further comprise gene editing one or more relevant genes (such as, for example, genes in the glycogenolysis pathway), wherein editing includes but is not limited to single gene knockout, loss of function screening of multiple genes at one, gene knockin, or a combination thereof.

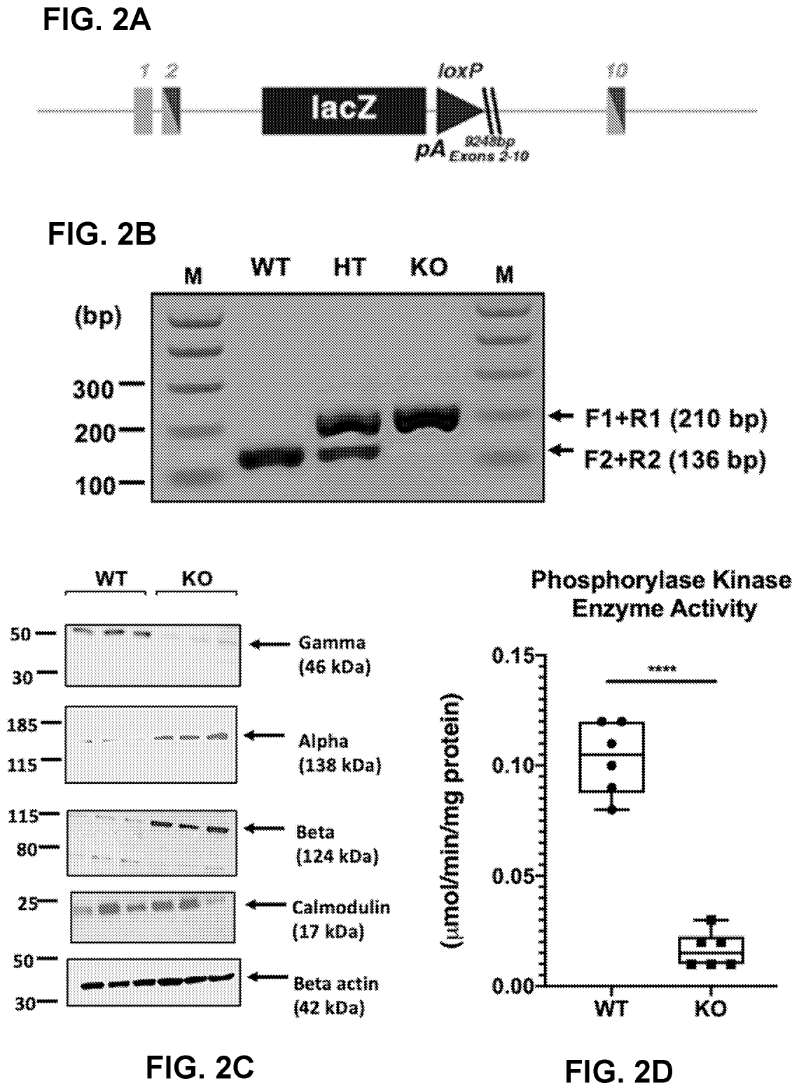

In an aspect, a disclosed method can further comprise administering to the subject a therapeutically effective amount of an agent that can correct one or more aspects of a dysregulated glycogen metabolic pathway, such as glycogen synthesis or glycogenolysis. In an aspect, such an agent can comprise an enzyme for enzyme replacement therapy. In an aspect, a disclosed enzyme can replace a mutated or dysfunction or nonexistent product of the GAA, GBE, PHKA1, PHKA2, PHKB, CALM1, CALM2, CALM3, PHKG2, and/or PYGL gene (FIG. 1 and FIG. 2). In an aspect, a disclosed enzyme can replace any enzyme in a dysregulated or dysfunctional glycogen metabolic pathway, such as those identified in FIG. 1 and FIG. 2. In an aspect, a disclosed method can comprise replacing one or more enzymes in a dysregulated or dysfunctional glycogen metabolic pathway.

As known to the art, glycogen synthesis and breakdown is regulated according to the energy state of the cell determined by the ratio of ATP to ADP. When glucose is abundant the amount of ATP is higher and that of AMP is low so that AMPK remains unphosphorylated and inactive. However, when glucose concentrations are low, ATP production decreases, while ATP is converted to ADP and AMP by cellular processes that use ATP as an energy source. Higher concentrations of ADP and AMP activate AMPK, or more specifically, its a subunit (AMPKα). Active AMPKα triggers catabolic metabolism, which prevents the synthesis of glycogen, lipids, and most proteins while activating glycogen breakdown, oxidative phosphorylation, and mitochondrial biogenesis.

In an aspect, a disclosed method can comprise administering an oligonucleotide therapeutic agent. A disclosed oligonucleotide therapeutic agent can comprise a single-stranded or double-stranded DNA, iRNA, shRNA, siRNA, mRNA, non-coding RNA (ncRNA), an antisense molecule, miRNA, a morpholino, a peptide-nucleic acid (PNA), or an analog or conjugate thereof. In an aspect, a disclosed oligonucleotide therapeutic agent can be an ASO or an RNAi. In an aspect, a disclosed oligonucleotide therapeutic agent can comprise one or more modifications at any position applicable.

In an aspect, a disclosed oligonucleotide therapeutic agent can comprise a CRISPR-based endonuclease. In an aspect, a disclosed endonuclease can be Cas9. In an aspect, a disclosed Cas9 can be from Staphylococcus aureus or Streptococcus pyogenes. Cas9 can have the amino acid sequence set forth in SEQ ID NO:32, SEQ ID NO:33, or a fragment thereof. In an aspect, a disclosed Cas9 can have a sequence having at least 75%, at least 80%, at least 85%, at least 90%, or at least 95% identity to the amino acid sequence set forth in SEQ ID NO:32, SEQ ID NO:33, or a fragment thereof. In an aspect, a disclosed nucleic acid sequence for Cas9 can comprise the sequence set forth in SEQ ID NO:31 or a fragment thereof. In an aspect, a disclosed nucleic acid sequence for Cas9 can comprise a sequence having at least 80%, at least 85%, at least 90%, or at least 95% identity to the sequence set forth in SEQ ID NO:31 or a fragment thereof.

In an aspect, a disclosed method can further comprise administering one or more immune modulators. In an aspect, a disclosed immune modulator can be methotrexate, rituximab, intravenous gamma globulin, or bortezomib, or a combination thereof. In an aspect, a disclosed immune modulator can be bortezomib or SVP-Rapamycin. In an aspect, a disclosed immune modulator can be Tacrolimus. In an aspect, a disclosed immune modulator such as methotrexate can be administered at a transient low to high dose. In an aspect, a disclosed immune modulator can be administered at a dose of about 0.1 mg/kg body weight to about 0.6 mg/kg body weight. In an aspect, a disclosed immune modulator can be administered at a dose of about 0.4 mg/kg body weight. In an aspect, a disclosed immune modulator can be administered at about a daily dose of 0.4 mg/kg body weight for 3 to 5 or greater cycles, with up to three days per cycle. In an aspect, a disclosed immune modulator can be administered at about a daily dose of 0.4 mg/kg body weight for a minimum of 3 cycles, with three days per cycle. In an aspect, a person skilled in the art can determine the appropriate number of cycles. In an aspect, a disclosed immune modulator can be administered as many times as necessary to achieve a desired clinical effect.

In an aspect, a disclosed immune modulator can be administered orally about one hour before a disclosed therapeutic agent. In an aspect, a disclosed immune modulator can be administered subcutaneously about 15 minutes before a disclosed therapeutic agent. In an aspect, a disclosed immune modulator can be administered concurrently with a disclosed therapeutic agent. In an aspect, a disclosed immune modulator can be administered orally about one hour or a few days before a disclosed isolated nucleic acid molecule, a disclosed vector, a disclosed pharmaceutical formulation, or a combination thereof. In an aspect, a disclosed immune modulator can be administered subcutaneously about 15 minutes before or a few days before a disclosed isolated nucleic acid molecule, a disclosed vector, a disclosed pharmaceutical formulation, or a combination thereof. In an aspect, a disclosed immune modulator can be administered concurrently with a disclosed isolated nucleic acid molecule, a disclosed vector, a disclosed pharmaceutical formulation, or a combination thereof.

In an aspect, a disclosed method can further comprise administering one or more proteasome inhibitors (e.g., bortezomib, carfilzomib, marizomib, ixazomib, and oprozomib). In an aspect, a proteasome inhibitor can be an agent that acts on plasma cells (e.g., daratumumab). In an aspect, an agent that acts on a plasma cell can be melphalan hydrochloride, melphalan, pamidronate disodium, carmustine, carfilzomib, carmustine, cyclophosphamide, daratumumab, doxorubicin hydrochloride liposome, doxorubicin hydrochloride liposome, elotuzumab, melphalan hydrochloride, panobinostat, ixazomib citrate, carfilzomib, lenalidomide, melphalan, melphalan hydrochloride, plerixafor, ixazomib citrate, pamidronate disodium, panobinostat, plerixafor, pomalidomide, pomalidomide, lenalidomide, selinexor, thalidomide, thalidomide, bortezomib, selinexor, zoledronic acid, or zoledronic acid.

In an aspect, a disclosed method can further comprise administering one or more proteasome inhibitors or agents that act on plasma cells prior to administering a disclosed isolated nucleic acid molecule, a disclosed vector, or a disclosed pharmaceutical formulation. In an aspect, a disclosed method can comprise administering one or more proteasome inhibitors or one or more agents that act on plasma cells concurrently with administering a disclosed isolated nucleic acid molecule, a disclosed vector, or a disclosed pharmaceutical formulation. In an aspect, a disclosed method can comprise administering one or more proteasome inhibitors or one or more agents that act on plasma cells subsequent to administering a disclosed isolated nucleic acid molecule, a disclosed vector, or a disclosed pharmaceutical formulation. In an aspect, a disclosed method can further comprise administering one or more proteasome inhibitors more than 1 time. In an aspect, a disclosed method can comprise administering one or more proteasome inhibitors repeatedly over time.

In an aspect, a disclosed method can further comprise administering one or more immunosuppressive agents. In an aspect, an immunosuppressive agent can be, but is not limited to, azathioprine, methotrexate, sirolimus, anti-thymocyte globulin (ATG), cyclosporine (CSP), mycophenolate mofetil (MMF), steroids, or a combination thereof. In an aspect, a disclosed method can comprise administering one or more immunosuppressive agents more than 1 time. In an aspect, a disclosed method can comprise administering one or more one or more immunosuppressive agents repeatedly over time. In an aspect, a disclosed method can comprise administering a compound that targets or alters antigen presentation or humoral or cell mediated or innate immune responses.

In an aspect, a disclosed method can further comprise administering a compound that exerts a therapeutic effect against B cells and/or a compound that targets or alters antigen presentation or humoral or cell mediated immune response. In an aspect, a disclosed compound can be rituximab, methotrexate, intravenous gamma globulin, anti CD4 antibody, anti CD2, an anti-FcRN antibody, a BTK inhibitor, an anti-IGF1R antibody, a CD19 antibody (e.g., inebilizumab), an anti-IL6 antibody (e.g., tocilizumab), an antibody to CD40, an IL2 mutein, or a combination thereof. Also disclosed herein are Treg infusions that can be administered as a way to help with immune tolerance (e.g., antigen specific Treg cells to AAV).

In an aspect, a disclosed method can further comprise administering lipid nanoparticles (LNPs). In an aspect, LNPs can be organ-targeted. In an aspect, LNPs can be liver-targeted or testes-targeted. For example, in an aspect, mRNA therapy with LNP encapsulation for systemic delivery to hepatocytes has the potential to restore metabolic enzymatic activity for one or more glycogen storage diseases such as GSD IX al, GSD IX α2, GSD IX β, GSD IX δ, GSD IX γ2, and/or GSD VI. In an aspect, the mRNA therapy focuses on human glycogen phosphorylase kinase subunit α1, glycogen phosphorylase kinase subunit α2, glycogen phosphorylase kinase subunit β, glycogen phosphorylase kinase subunit γ2, and/or glycogen phosphorylase. In an aspect, the mRNA therapy focuses on one or more genes in the glycogen synthesis and/or glycogenolysis pathway (FIG. 1 and FIG. 2).

In an aspect, a disclosed method can further comprise treating a subject that has developed or is likely to develop neutralizing antibodies (ABs) to a disclosed vector, capsid, and/or transgene. In an aspect, treating a subject that has developed or is likely to develop neutralizing antibodies can comprise plasmapheresis and immunosuppression. In an aspect, a disclosed method can comprise using immunosuppression to decrease the T cell, B cell, and/or plasma cell population, decrease the innate immune response, inflammatory response, and antibody levels in general. In an aspect, a disclosed method can comprise administering an IgG-degrading agent that depletes pre-existing neutralizing antibodies. In an aspect, a disclosed method can comprise administering to the subject IdeS or IdeZ, rapamycin, and/or SVP-Rapamycin. In an aspect, a disclosed method can comprise administering Tacrolimus. In an aspect, a disclosed IgG-degrading agent is bacteria-derived IdeS or IdeZ.

In an aspect, a disclosed method can further comprise administering the subject a disclosed RNA therapeutic.

In an aspect, a disclosed method can further comprise administering to the subject an effective amount an isolated nucleic acid encoding a protein that is deficient or absent in the subject. In an aspect, a disclosed encoded protein can comprise a recombinant human protein such as, for example, recombinant alpha-glucosidase (GAA). In an aspect, a disclosed GAA can comprise the amino acid sequence set forth in SEQ ID NO:34, SEQ ID NO:35, SEQ ID NO:36, SEQ ID NO:37, SEQ ID NO:38, or a fragment thereof. In an aspect, a disclosed GAA can comprise a sequence having at least 30%, at least 40%, at least 50%, at least 60%, at least 70%, at least 80%, or at least 90% identity to the amino acid sequence set forth in SEQ ID NO:34, SEQ ID NO:35, SEQ ID NO:36, SEQ ID NO:37, SEQ ID NO:38, or a fragment thereof. In an aspect, a disclosed GAA can be Myozyme or Lumizyme. In an aspect of a disclosed method, a disclosed isolated nucleic acid sequence for GAA can comprise the sequence set forth in SEQ ID NO:39, SEQ ID NO:40, or a fragment thereof. In an aspect, a disclosed isolated nucleic acid sequence for GAA can comprise a sequence having at least 30%, at least 40%, at least 50%, at least 60%, at least 70%, at least 80%, or at least 90% identity to the nucleotide sequence set forth in SEQ ID NO:39, SEQ ID NO:40, or a fragment thereof. In an aspect, a disclosed isolated nucleic acid encoding a recombinant protein such as, for example, GAA, can be present in a disclosed viral vector including, for example, an AAV vector or a self-complementary AAV vector. In an aspect, a disclosed immune modulator and a disclosed therapeutic agent can be concurrently administered. In an aspect, a disclosed composition comprising GAA or a disclosed vector comprising a disclosed isolated nucleic acid molecule encoding GAA can be administered prior to, concurrent with, or after the administration of a disclosed vector comprising a disclosed isolated nucleic acid molecule comprising a nucleic acid sequence encoding a polypeptide capable of preventing glycogen accumulation and/or degrading accumulated glycogen.

In an aspect, a disclosed method can comprise modifying one or more of the disclosed steps.

### 2. Nucleic Acid Based Methods

Disclosed herein is a method of treating and/or preventing GSD IX al, GSD IX α2, GSD IX β, GSD IX δ, GSD IX γ2, and/or GSD VI disease progression comprising administering to a subject in need thereof a disclosed isolated nucleic acid molecule. Disclosed herein is a method of treating and/or preventing disease progression comprising administering to a subject having GSD IX and/or GSD VI a disclosed isolated nucleic acid molecule.

Disclosed herein is a method of treating and/or preventing GSD IX al, GSD IX α2, GSD IX β, GSD IX δ, GSD IX γ2, and/or GSD VI disease progression comprising administering to a subject in need thereof an isolated nucleic acid molecule comprising a nucleic acid sequence encoding a polypeptide, wherein the nucleic acid sequence is CpG-depleted and codon-optimized for expression in a human cell, and preventing glycogen accumulation and/or degrading accumulated a glycogen in the subject. Disclosed herein is a method of treating and/or preventing disease progression comprising administering to a subject having GSD IX and/or GSD VI an isolated nucleic acid molecule comprising a nucleic acid sequence encoding a polypeptide, wherein the nucleic acid sequence is CpG-depleted and codon-optimized for expression in a human cell, wherein glycogen accumulation is prevented and/or accumulated glycogen is degraded in the subject.

Disclosed herein is a method of treating and/or preventing GSD IX al, GSD IX α2, GSD IX β, GSD IX δ, GSD IX γ2, and/or GSD VI disease progression comprising preventing glycogen accumulation and/or degrading accumulated glycogen in a subject in need thereof by administering to the subject an isolated nucleic acid molecule comprising a nucleic acid sequence encoding a polypeptide, wherein the nucleic acid sequence is CpG-depleted and codon-optimized for expression in a human cell. Disclosed herein is a method of treating and/or preventing disease progression comprising preventing glycogen accumulation and/or degrading accumulated glycogen in a subject having GSD IX and/or GSD VI by administering to the subject an isolated nucleic acid molecule comprising a nucleic acid sequence encoding a polypeptide, wherein the nucleic acid sequence is CpG-depleted and codon-optimized for expression in a human cell.

Disclosed herein is a method of treating and/or preventing GSD IX al, GSD IX α2, GSD IX β, GSD IX δ, GSD IX γ2, and/or GSD VI disease progression comprising administering to a subject in need thereof an isolated nucleic acid molecule comprising a nucleic acid sequence encoding a polypeptide, wherein the nucleic acid sequence is CpG-depleted and codon-optimized for expression in a human cell, thereby preventing glycogen accumulation and/or degrading accumulated glycogen in the subject. Disclosed herein is a method of treating and/or preventing disease progression comprising administering to a subject having GSD IX and/or GSD VI an isolated nucleic acid molecule comprising a nucleic acid sequence encoding a polypeptide, wherein the nucleic acid sequence is CpG-depleted and codon-optimized for expression in a human cell, wherein glycogen accumulation is prevented and/or accumulated glycogen is degraded in the subject.

Disclosed herein is a method of preventing glycogen accumulation and/or degrading accumulated glycogen comprising administering to a subject having GSD IX al, GSD IX α2, GSD IX β, GSD IX δ, GSD IX γ2, and/or GSD VI an isolated nucleic acid molecule comprising a nucleic acid sequence encoding a polypeptide, wherein the nucleic acid sequence is CpG-depleted and codon-optimized for expression in a human cell. Disclosed herein is a method of preventing glycogen accumulation and/or degrading accumulated glycogen comprising administering to a subject having GSD IX and/or GSD VI an isolated nucleic acid molecule comprising a nucleic acid sequence encoding a polypeptide, wherein the nucleic acid sequence is CpG-depleted and codon-optimized for expression in a human cell.

Disclosed herein is a method of treating and/or reducing liver disease comprising administering to a subject having GSD IX al, GSD IX α2, GSD IX β, GSD IX δ, GSD IX γ2, and/or GSD VI an isolated nucleic acid molecule comprising a nucleic acid sequence encoding a polypeptide, wherein the nucleic acid sequence is CpG-depleted and codon-optimized for expression in a human, and preventing glycogen accumulation and/or degrading accumulated a glycogen in the subject. Disclosed herein is a method of treating and/or reducing liver disease comprising administering to a subject having GSD IX and/or GSD VI an isolated nucleic acid molecule comprising a nucleic acid sequence encoding a polypeptide, wherein the nucleic acid sequence is CpG-depleted and codon-optimized for expression in a human, wherein glycogen accumulation is prevented and/or accumulated glycogen is degraded in the subject.

Disclosed herein is a method of treating and/or reducing liver disease, comprising: preventing glycogen accumulation and/or degrading accumulated glycogen in a subject having GSD IX al, GSD IX α2, GSD IX β, GSD IX δ, GSD IX γ2, and/or GSD VI by administering to the subject an isolated nucleic acid molecule comprising a nucleic acid sequence encoding a polypeptide, wherein the nucleic acid sequence is CpG-depleted and codon-optimized for expression in a human. Disclosed herein is a method of treating and/or reducing liver disease, comprising: preventing glycogen accumulation and/or degrading accumulated glycogen in a subject having GSD IX and/or GSD VI by administering to the subject an isolated nucleic acid molecule comprising a nucleic acid sequence encoding a polypeptide, wherein the nucleic acid sequence is CpG-depleted and codon-optimized for expression in a human.

Disclosed herein is a method of treating and/or reducing liver disease, comprising: administering to a subject having GSD IX al, GSD IX α2, GSD IX β, GSD IX δ, GSD IX γ2, and/or GSD VI an isolated nucleic acid molecule comprising a nucleic acid sequence encoding a polypeptide, wherein the nucleic acid sequence is CpG-depleted and codon-optimized for expression in a human, thereby preventing glycogen accumulation and/or degrading accumulated glycogen in the subject. Disclosed herein is a method of treating and/or reducing liver disease, comprising: administering to a subject having GSD IX and/or GSD VI an isolated nucleic acid molecule comprising a nucleic acid sequence encoding a polypeptide, wherein the nucleic acid sequence is CpG-depleted and codon-optimized for expression in a human, wherein glycogen accumulation is prevented and/or accumulated glycogen is degraded in the subject.

Disclosed herein is a method of treating and/or preventing GSD IX al, GSD IX α2, GSD IX β, GSD IX δ, GSD IX γ2, and/or GSD VI disease progression comprising administering to a subject in need thereof a disclosed isolated nucleic acid molecule, and reducing the expression level and/or activity level of glycogen synthase. Disclosed herein is a method of treating and/or preventing disease progression comprising administering to a subject having GSD IX and/or GSD VI a disclosed isolated nucleic acid molecule, and reducing the expression level and/or activity level of glycogen synthase.

Disclosed herein is a method of treating and/or preventing GSD IX al, GSD IX α2, GSD IX β, GSD IX δ, GSD IX γ2, and/or GSD VI disease progression comprising administering to a subject in need thereof an isolated nucleic acid molecule comprising a nucleic acid sequence encoding a polypeptide, wherein the nucleic acid sequence is CpG-depleted and codon-optimized for expression in a human cell, reducing the expression level and/or activity level of glycogen synthase, and preventing glycogen accumulation and/or degrading accumulated a glycogen in the subject. Disclosed herein is a method of treating and/or preventing disease progression comprising administering to a subject having GSD IX and/or GSD VI an isolated nucleic acid molecule comprising a nucleic acid sequence encoding a polypeptide, wherein the nucleic acid sequence is CpG-depleted and codon-optimized for expression in a human cell, reducing the expression level and/or activity level of glycogen synthase, wherein glycogen accumulation is prevented and/or accumulated glycogen is degraded in the subject.

Disclosed herein is a method of treating and/or preventing GSD IX al, GSD IX α2, GSD IX β, GSD IX δ, GSD IX γ2, and/or GSD VI disease progression comprising preventing glycogen accumulation and/or degrading accumulated glycogen in a subject in need thereof by administering to the subject an isolated nucleic acid molecule comprising a nucleic acid sequence encoding a polypeptide, wherein the nucleic acid sequence is CpG-depleted and codon-optimized for expression in a human cell, and reducing the expression level and/or activity level of glycogen synthase. Disclosed herein is a method of treating and/or preventing disease progression comprising preventing glycogen accumulation and/or degrading accumulated glycogen in a subject having GSD IX and/or GSD VI by administering to the subject an isolated nucleic acid molecule comprising a nucleic acid sequence encoding a polypeptide, wherein the nucleic acid sequence is CpG-depleted and codon-optimized for expression in a human cell, and reducing the expression level and/or activity level of glycogen synthase.

Disclosed herein is a method of treating and/or preventing GSD IX al, GSD IX α2, GSD IX β, GSD IX δ, GSD IX γ2, and/or GSD VI disease progression comprising administering to a subject in need thereof an isolated nucleic acid molecule comprising a nucleic acid sequence encoding a polypeptide, wherein the nucleic acid sequence is CpG-depleted and codon-optimized for expression in a human cell, and reducing the expression level and/or activity level of glycogen synthase, thereby preventing glycogen accumulation and/or degrading accumulated glycogen in the subject. Disclosed herein is a method of treating and/or preventing disease progression comprising administering to a subject having GSD IX and/or GSD VI an isolated nucleic acid molecule comprising a nucleic acid sequence encoding a polypeptide, wherein the nucleic acid sequence is CpG-depleted and codon-optimized for expression in a human cell, and reducing the expression level and/or activity level of glycogen synthase, wherein glycogen accumulation is prevented and/or accumulated glycogen is degraded in the subject.

Disclosed herein is a method of preventing glycogen accumulation and/or degrading accumulated glycogen comprising administering to a subject having GSD IX al, GSD IX α2, GSD IX β, GSD IX δ, GSD IX γ2, and/or GSD VI an isolated nucleic acid molecule comprising a nucleic acid sequence encoding a polypeptide, wherein the nucleic acid sequence is CpG-depleted and codon-optimized for expression in a human cell, and reducing the expression level and/or activity level of glycogen synthase. Disclosed herein is a method of preventing glycogen accumulation and/or degrading accumulated glycogen comprising administering to a subject having GSD IX and/or GSD VI an isolated nucleic acid molecule comprising a nucleic acid sequence encoding a polypeptide, wherein the nucleic acid sequence is CpG-depleted and codon-optimized for expression in a human cell, and reducing the expression level and/or activity level of glycogen synthase.

Disclosed herein is a method of treating and/or reducing liver disease comprising administering to a subject having GSD IX al, GSD IX α2, GSD IX β, GSD IX δ, GSD IX γ2, and/or GSD VI an isolated nucleic acid molecule comprising a nucleic acid sequence encoding a polypeptide, wherein the nucleic acid sequence is CpG-depleted and codon-optimized for expression in a human, reducing the expression level and/or activity level of glycogen synthase, and preventing glycogen accumulation and/or degrading accumulated a glycogen in the subject. Disclosed herein is a method of treating and/or reducing liver disease comprising administering to a subject having GSD IX and/or GSD VI an isolated nucleic acid molecule comprising a nucleic acid sequence encoding a polypeptide, wherein the nucleic acid sequence is CpG-depleted and codon-optimized for expression in a human, reducing the expression level and/or activity level of glycogen synthase, wherein glycogen accumulation is prevented and/or accumulated glycogen is degraded in the subject.

Disclosed herein is a method of treating and/or reducing liver disease comprising preventing glycogen accumulation and/or degrading accumulated glycogen in a subject having GSD IX al, GSD IX α2, GSD IX β, GSD IX δ, GSD IX γ2, and/or GSD VI by administering to the subject an isolated nucleic acid molecule comprising a nucleic acid sequence encoding a polypeptide, wherein the nucleic acid sequence is CpG-depleted and codon-optimized for expression in a human, and reducing the expression level and/or activity level of glycogen synthase. Disclosed herein is a method of treating and/or reducing liver disease comprising preventing glycogen accumulation and/or degrading accumulated in a subject having GSD IX and/or GSD VI by administering to the subject an isolated nucleic acid molecule comprising a nucleic acid sequence encoding a polypeptide, wherein the nucleic acid sequence is CpG-depleted and codon-optimized for expression in a human, and reducing the expression level and/or activity level of glycogen synthase.

Disclosed herein is a method of treating and/or reducing liver disease, comprising: administering to a subject having GSD IX al, GSD IX α2, GSD IX β, GSD IX δ, GSD IX γ2, and/or GSD VI an isolated nucleic acid molecule comprising a nucleic acid sequence encoding a polypeptide, wherein the nucleic acid sequence is CpG-depleted and codon-optimized for expression in a human, and reducing the expression level and/or activity level of glycogen synthase, thereby preventing glycogen accumulation and/or degrading accumulated glycogen in the subject. Disclosed herein is a method of treating and/or reducing liver disease, comprising: administering to a subject having GSD IX and/or GSD VI an isolated nucleic acid molecule comprising a nucleic acid sequence encoding a polypeptide, wherein the nucleic acid sequence is CpG-depleted and codon-optimized for expression in a human, and reducing the expression level and/or activity level of glycogen synthase, wherein glycogen accumulation is prevented and/or accumulated glycogen is degraded in the subject.

In an aspect, a disclosed method can restore one or more aspects of the glycogen metabolic pathway, restore one or more aspects of the glycogenolysis metabolic pathway, can restore one or more aspects of the glycogenesis metabolic pathway, can restore PhK subunit activity and/or functionality, can restore PYGL activity and/or functionality, or any combination thereof. In an aspect, restoring one or more aspects of a disclosed metabolic pathway can comprise restoring the activity and/or functionality of one or more enzymes identified in FIG. 1. In an aspect, restoring PhK subunit activity and/or functionality can comprise restoring the activity and/or functionality of the al subunit, the α2 subunit, the β subunit, the γ2 subunit, or any combination thereof. For example, in an aspect, a disclosed method can restore the balance of glycogen metabolism, wherein glycogen metabolism comprises glycogen synthesis and breakdown. In an aspect, a disclosed method can restore the functionality and/or structural integrity of the PhK complex. For example, in an aspect, by restoring the functionality and/or structural integrity of a subunit (such as α2, δ, β, and γ2), then the functionality and/or structural integrity of the PhK complex (the heterotetramer) can be restored.

In an aspect, restoring PhK activity and/or functionality or restoring PYGL activity and/or functionality can be a 10%, 20%, 30%, 40%, 50%, 60%, 70%, 80%, 90%, 100%, or any amount of restoration when compared to a pre-existing level such as, for example, a pre-treatment level. In an aspect, the amount of restoration can be 10-20%, 20-30%, 30-40%, 40-50%, 50-60%, 60-70%, 70-80%, 80-90%, or 90-100% more than a pre-existing level such as, for example, a pre-treatment level. In an aspect, restoration can be measured against a control level (e.g., a level in a subject not having a GSD). In an aspect, restoration can be a partial or incomplete restoration. In an aspect, restoration can be complete or near complete restoration such that the level of expression, activity and/or functionality is similar to that of a wild-type or control level.

In an aspect, a disclosed method can comprise restoring one or more aspects of cellular homeostasis and/or cellular functionality and/or metabolic dysregulation. In an aspect, restoring one or more aspects of cellular homeostasis and/or cellular functionality can comprise one or more of the following: (i) correcting cell starvation in one or more cell types (such as, for example, liver cells and muscle cells); (ii) normalizing aspects of the autophagy pathway (such as, for example, correcting, preventing, reducing, and/or ameliorating autophagy); (iii) improving, enhancing, restoring, and/or preserving mitochondrial functionality and/or structural integrity; (iv) improving, enhancing, restoring, and/or preserving organelle functionality and/or structural integrity; (v) preventing, slowing, and/or eliminating hypoglycemia, ketosis, and/or other liver abnormalities; (vi) correcting liver enzyme dysregulation; (vii) reversing, inhibiting, preventing, stabilizing, and/or slowing the rate of progression of the multi-systemic manifestations of GSD IX; (viii) reversing, inhibiting, preventing, stabilizing, and/or slowing the rate of progression of liver disease including fibrosis, cirrhosis, hepatic adenomas, and/or liver hepatocellular carcinoma, or (ix) any combination thereof. In an aspect, restoring one or more aspects of cellular homeostasis can comprise improving, enhancing, restoring, and/or preserving one or more aspects of cellular structural and/or functional integrity.

In an aspect of a disclosed method, techniques to monitor, measure, and/or assess the restoring one or more aspects of cellular homeostasis and/or cellular functionality can comprise qualitative (or subjective) means as well as quantitative (or objective) means. These means are known to the skilled person.

In an aspect, the nucleic acid sequence can have a coding sequence that is less than about 4.5 kilobases.

In an aspect, a disclosed isolated nucleic acid molecule can be present in a vector. In an aspect, a disclosed vector can be a viral vector or a non-viral vector. In an aspect, a disclosed non-viral vector can be a polymer-based vector, a peptide-based vector, a lipid nanoparticle, a solid lipid nanoparticle, or a cationic lipid based vector.

In an aspect, a disclosed viral vector can be an adenovirus vector, an AAV vector, a herpes simplex virus vector, a retrovirus vector, a lentivirus vector, and alphavirus vector, a flavivirus vector, a rhabdovirus vector, a measles virus vector, a Newcastle disease viral vector, a poxvirus vector, or a picornavirus vector.

In an aspect, a disclosed AAV vector can include naturally isolated serotypes including, but not limited to, AAV1, AAV2, AAV3 (including 3a and 3b), AAV4, AAV5, AAV6, AAV7, AAV8, AAVrh8, AAV9, AAV10, AAVrh10, AAV11, AAV12, AAV13, AAVrh39, AAVrh43, AAVcy.7 as well as bovine AAV, caprine AAV, canine AAV, equine AAV, ovine AAV, avian AAV, primate AAV, non-primate AAV, and any other virus classified by the International Committee on Taxonomy of Viruses (ICTV) as an AAV. In an aspect, an AAV capsid can be a chimera either created by capsid evolution or by rational capsid engineering from a naturally isolated AAV variants to capture desirable serotype features such as enhanced or specific tissue tropism and/or a host immune response escape. Naturally isolated AAV variants include, but are not limited to, AAV-DJ, AAV-HAE1, AAV-HAE2, AAVM41, AAV-1829, AAV2 Y/F, AAV2 T/V, AAV2i8, AAV2.5, AAV9.45, AAV9.61, AAV-B1, AAV-AS, AAV9.45A-String (e.g., AAV9.45-AS), AAV9.45Angiopep, AAV9.47-Angiopep, and AAV9.47-AS, AAV-PHP.B, AAV-PHP.eB, AAV-PHP.S, AAV-F, AAVcc.47, and AAVcc.81. In an aspect, a disclosed AAV vector can be AAV-Rh74 or a related variant (e.g., capsid variants such as RHM4-1). In an aspect, a disclosed AAV vector can be AAV8. In an aspect, a disclosed AAV vector can be AAVhum.8. In an aspect, a disclosed AAV vector can be a self-complementary AAV as disclosed herein.

In an aspect, a disclosed vector can comprise a liver-specific promoter operably linked to the isolated nucleic acid molecule. In an aspect, a disclosed liver-specific promoter can be the thyroxin binding globulin (TBG) promoter, the α1-microglobulin/bikunin enhancer/thyroid hormone-binding globulin promoter, the human albumin (hALB) promoter, the thyroid hormone-binding globulin promoter, the thyroxin binding globulin promoter, the α-1-anti-trypsin promoter, the bovine albumin (bAlb) promoter, the murine albumin (mAlb) promoter, the human α1-antitrypsin (hAAT) promoter, the ApoEhAAT promoter comprising the ApoE enhancer and the hAAT promoter, the transthyretin (TTR) promoter, the liver fatty acid binding protein promoter, the hepatitis B virus (HBV) promoter, the DC172 promoter consisting of the hAAT promoter and the α1-microglobulin enhancer, the DC190 promoter containing the human albumin promoter and the prothrombin enhancer, or any one of other natural and synthetic liver-specific promoter.

In an aspect, a disclosed liver-specific promoter can comprise any liver-specific promoter known to the art. In an aspect, a liver specific promoter can comprise about 845-bp and comprise the thyroid hormone-binding globulin promoter sequences (2382 to 13), two copies of α1-microglobulinybikunin enhancer sequences (22,804 through 22,704), and a 71-bp leader sequence (Ill C R, et al. (1997) Blood Coagul Fibrinolysis. 8 Suppl 2:S23-S30). In an aspect, a disclosed liver-specific promoter can comprise the sequence set forth in SEQ ID NO:56. In an aspect, a disclosed liver-specific promoter can comprise a sequence having at least 40%, 50%, 60%, 70%, 80%, 85%, 86%, 87%, 88%, 89%, 90%, 91%, 92%, 93%, 94%, 95%, 96%, 97%, 98%, or 99% identity to the sequence set forth in SEQ ID NO:56. In an aspect, a disclosed liver-specific promoter can comprise a sequence having at least 40%-60%, at least 60%-80%, at least 80%-90%, or at least 90%-100% identity to the sequence set forth in SEQ ID NO:56.

In an aspect, a disclosed promoter can be a promoter/enhancer. In an aspect, a disclosed promoter can be an endogenous promoter. In an aspect, a disclosed endogenous promoter can be an endogenous promoter/enhancer. In an aspect, a disclosed endogenous promoter or a disclosed endogenous promoter/enhancer can generally be obtained from a non-coding region upstream of a transcription initiation site of a gene of interest (such as, for example, a disclosed phosphorylase kinase or phosphorylase kinase subunit (e.g., PhK α2, PhK β, PhK γ2, PhK δ) or some other enzyme involved in the glycogen metabolic pathway (PYGL)). In an aspect, a disclosed endogenous promoter or a disclosed endogenous promoter/enhancer can be used for constitutive and efficient expression of a disclosed gene (e.g., a nucleic acid sequence encoding a polypeptide capable of preventing glycogen accumulation and/or degrading accumulated glycogen).

In an aspect, a disclosed endogenous promoter or a disclosed endogenous promoter/enhancer can be used for constitutive and efficient expression of a phosphorylase kinase or a phosphorylase kinase subunit. In an aspect, a disclosed endogenous promoter or a disclosed endogenous promoter/enhancer can comprise the promoter for the gene encoding the phosphorylase kinase regulatory subunit alpha 2 (PhK α2) (SEQ ID NO:63), the phosphorylase kinase regulatory subunit beta (PhK β) (SEQ ID NO:64), the phosphorylase kinase catalytic subunit gamma 2 (PhK γ2) (SEQ ID NO:65), the phosphorylase kinase regulatory subunit delta (PhK δ), or glycogen phosphorylase (PYGL) (SEQ ID NO:66). For example, in an aspect, when an encoded polypeptide comprises the PhK α2 subunit, the disclosed endogenous promoter or a disclosed endogenous promoter/enhancer can comprise the promoter or promoter/enhancer for the gene encoding the PhK α2 subunit. Similarly, when an encoded polypeptide comprises the PhK R subunit, the disclosed endogenous promoter or a disclosed endogenous promoter/enhancer can comprise the promoter or promoter/enhancer for the gene encoding the PhK β subunit. For example, in an aspect, a disclosed endogenous promoter or a disclosed endogenous promoter/enhancer can comprise the sequence set forth in SEQ ID NO:63-SEQ ID NO:66 or a fragment thereof. For example, in an aspect, a disclosed endogenous promoter or a disclosed endogenous promoter/enhancer can comprise a sequence having at least 40%, at least 50%, at least 60%, at least 70%, at least 80%, at least 90%, at least 95%, or more than 95% identity to the sequence set forth in SEQ ID NO:63-SEQ ID NO:66 or a fragment thereof.

In an aspect, a disclosed ubiquitous promoter can be a CMV enhancer/chicken β-actin promoter (CB promoter).

In an aspect, a disclosed encoded polypeptide can degrade glycogen and/or prevent glycogen accumulation. In an aspect, a disclosed encoded polypeptide can comprise a glycogen phosphorylase kinase or a glycogen phosphorylase liver. In an aspect, a disclosed encoded polypeptide can be derived from a human or a non-human source. In an aspect, a disclosed encoded polypeptide can comprise the sequence set forth in any one of SEQ ID NO:01-SEQ ID NO:11 or SEQ ID NO:57-SEQ ID NO:59. In an aspect, a disclosed encoded polypeptide can comprise a sequence having at least 40-59%, at least 50-69%, or at least 80-99% identity to the sequence set forth in any one of SEQ ID NO:01-SEQ ID NO: 11 or SEQ ID NO:57-SEQ ID NO:59.

In an aspect, a disclosed nucleic acid sequence can comprise the sequence for a glycogen phosphorylase kinase or a subunit thereof or a glycogen phosphorylase. In an aspect, a disclosed nucleic acid sequence can comprise the sequence set forth in any one of SEQ ID NO:12-SEQ ID NO:22 or SEQ ID NO:60-SEQ ID NO:62. In an aspect, a disclosed nucleic acid sequence can comprise a sequence having at least 30%, at least 40%, at least 50%, at least 60%, at least 70%, at least 80%, or at least 90% identity to the sequence set forth in any one of SEQ ID NO:12-SEQ ID NO:22 or SEQ ID NO:60-SEQ ID NO:62.

In an aspect, a disclosed viral vector can comprise an isolated nucleic acid molecule comprising the liver-specific promoter, the CpG-depleted and codon-optimized nucleic acid sequence encoding the polypeptide, and a polyadenylation signal.

In an aspect, a subject can be a human subject. In an aspect, a disclosed vector or a disclosed isolated nucleic acid molecule can be delivered to the subject's liver, heart, skeletal muscle, smooth muscle, CNS, PNS, or a combination thereof. In an aspect, a disclosed vector can be delivered to the subject's liver. In an aspect, a disclosed vector can be concurrently and/or serially administered to a subject via multiple routes of administration. For example, in an aspect, administering a disclosed vector can comprise intravenous administration and intra-cistem magna (ICM) administration. In an aspect, administering a disclosed vector can comprise IV administration and intrathecal (ITH) administration.

In an aspect, a therapeutically effective amount of disclosed vector can be delivered via intravenous (IV) administration and can comprise a range of about 1×10¹⁰ vg/kg to about 2×10¹⁴ vg/kg. In an aspect, for example, a disclosed vector can be administered at a dose of about 1×10¹¹ to about 8×10¹³ vg/kg or about 1×10¹² to about 8×10¹³ vg/kg. In an aspect, a disclosed vector can be administered at a dose of about 1×10¹³ to about 6×10¹³ vg/kg. In an aspect, a disclosed vector can be administered at a dose of at least about 1×10¹⁰, at least about 5×10¹⁰, at least about 1×10¹¹, at least about 5×10¹¹, at least about 1×10¹², at least about 5×10¹², at least about 1×10¹³, at least about 5×10¹³, or at least about 1×10¹⁴ vg/kg. In an aspect, a disclosed vector can be administered at a dose of no more than about 1×10¹⁰, no more than about 5×10¹⁰, no more than about 1×10¹¹, no more than about 5×10¹¹, no more than about 1×10¹², no more than about 5×10¹², no more than about 1×10¹³, no more than about 5×10¹³, or no more than about 1×10¹⁴ vg/kg. In an aspect, a disclosed vector can be administered at a dose of about 1×10¹² vg/kg. In an aspect, a disclosed vector can be administered at a dose of about 1×10¹¹ vg/kg.

In an aspect, a disclosed vector can be administered in a single dose, or in multiple doses (such as 2, 3, 4, 5, 6, 7, 8, 9 or 10 doses) as needed for the desired therapeutic results.

In an aspect, a disclosed vector can be administered via intravenous, intraarterial, intramuscular, intraperitoneal, subcutaneous, intra-CSF, intrathecal, intraventricular, intrahepatic, hepatic intra-arterial, hepatic portal vein (HPV), or in utero administration. In an aspect, a disclosed isolated nucleic acid molecule can be administered via intra-CSF administration in combination with a disclosed nucleic acid molecule, a disclosed vector, and/or a disclosed pharmaceutical formulation. In an aspect, a disclosed isolated nucleic acid molecule can be administered via intra-CSF administration in combination with RNAi, antisense oligonucleotides, miRNA, one or more small molecules, one or more therapeutic agents, one or more proteasome inhibitors, one or more immune modulators, and/or a gene editing system. In an aspect, a disclosed isolated nucleic acid molecule can be administered via LNP administration. In an aspect, a subject can be a human subject. In an aspect, disclosed isolated nucleic acid molecule can be delivered to the subject's liver, heart, skeletal muscle, smooth muscle, CNS, PNS, or a combination thereof. In an aspect, administration can comprise a combination of routes. In an aspect, a disclosed method can further comprise administering to the subject a therapeutically effective amount of a therapeutic agent. A therapeutic agent can be any disclosed agent that effects a desired clinical outcome.

In an aspect, a disclosed method can further comprise monitoring the subject for adverse effects. In an aspect, in the absence of adverse effects, the method can further comprise continuing to treat the subject. In an aspect, in the presence of adverse effects, the method can further comprise modifying the treating step. For example, hypoglycemia can be sporadically and/or continuously measured and monitored. Methods and techniques for measuring and monitoring hypoglycemia are known to the skilled person and include, but not limited to, by continuous glucose monitoring (CGM) methods and capillary blood glucose sticks.

In an aspect, a disclosed method can further comprise reducing glycogen levels by administering a glycogen synthase inhibitor (e.g., RNAi, ASO, etc.) to the subject, or modifying the subject's diet, for example, by using cornstarch or another slow release starch to prevent hypoglycemia, or modifying the subject's diet, for example, by consuming a high amount of protein, fat, or other anaplerotic agents (such as, for example, C7 compounds like triheptanoin or MCT), exercise or a combination thereof. In an aspect, a disclosed method can comprise reducing the expression level, activity level, or both of glycogen synthase. In an aspect, a glycogen synthase can be GYS1 (muscle glycogen synthase) or GYS2 (liver glycogen synthase) or both.

In an aspect, a disclosed method can comprise gene editing one or more relevant genes (such as, for example, genes in the glycogenolysis pathway), wherein editing includes but is not limited to single gene knockout, loss of function screening of multiple genes at one, gene knockin, or a combination thereof.

In an aspect, a disclosed method can further comprise administering to the subject a therapeutically effective amount of an agent that can correct one or more aspects of a dysregulated glycogen metabolic pathway, such as glycogen synthesis or glycogenolysis. In an aspect, such an agent can comprise an enzyme for enzyme replacement therapy. In an aspect, a disclosed enzyme can replace a mutated or dysfunction or nonexistence product of the PHKG2 gene, or a combination thereof. In an aspect, a disclosed enzyme can replace any enzyme in a dysregulated or dysfunctional glycogen metabolic pathway. In an aspect, a disclosed method can further comprise replacing one or more enzymes in a dysregulated or dysfunctional glycogen metabolic pathway.

In an aspect, a disclosed method can further comprise administering one or more immune modulators. In an aspect, a disclosed immune modulator can be methotrexate, rituximab, intravenous gamma globulin, or bortezomib, or a combination thereof. In an aspect, a disclosed immune modulator can be bortezomib or SVP-Rapamycin. In an aspect, a disclosed immune modulator can be Tacrolimus.

In an aspect, a disclosed immune modulator such as methotrexate can be administered at a transient low to high dose. In an aspect, a disclosed immune modulator can be administered at a dose of about 0.1 mg/kg body weight to about 0.6 mg/kg body weight. In an aspect, a disclosed immune modulator can be administered at a dose of about 0.4 mg/kg body weight. In an aspect, a disclosed immune modulator can be administered at about a daily dose of 0.4 mg/kg body weight for 3 to 5 or greater cycles, with up to three days per cycle. In an aspect, a disclosed immune modulator can be administered at about a daily dose of 0.4 mg/kg body weight for a minimum of 3 cycles, with three days per cycle. In an aspect, a person skilled in the art can determine the appropriate number of cycles. In an aspect, a disclosed immune modulator can be administered as many times as necessary to achieve a desired clinical effect.

In an aspect, a disclosed immune modulator can be administered orally about one hour before a disclosed therapeutic agent. In an aspect, a disclosed immune modulator can be administered subcutaneously about 15 minutes before a disclosed therapeutic agent. In an aspect, a disclosed immune modulator can be administered concurrently with a disclosed therapeutic agent. In an aspect, a disclosed immune modulator can be administered orally about one hour or a few days before a disclosed isolated nucleic acid molecule, a disclosed vector, a disclosed pharmaceutical formulation, or a combination thereof. In an aspect, a disclosed immune modulator can be administered subcutaneously about 15 minutes before or a few days before a disclosed isolated nucleic acid molecule, a disclosed vector, a disclosed pharmaceutical formulation, or a combination thereof. In an aspect, a disclosed immune modulator can be administered concurrently with a disclosed isolated nucleic acid molecule, a disclosed vector, a disclosed pharmaceutical formulation, or a combination thereof.

In an aspect, a disclosed method can further comprise administering one or more proteasome inhibitors (e.g., bortezomib, carfilzomib, marizomib, ixazomib, and oprozomib). In an aspect, a proteasome inhibitor can be an agent that acts on plasma cells (e.g., daratumumab). In an aspect, an agent that acts on a plasma cell can be melphalan hydrochloride, melphalan, pamidronate disodium, carmustine, carfilzomib, carmustine, cyclophosphamide, daratumumab, doxorubicin hydrochloride liposome, doxorubicin hydrochloride liposome, elotuzumab, melphalan hydrochloride, panobinostat, ixazomib citrate, carfilzomib, lenalidomide, melphalan, melphalan hydrochloride, plerixafor, ixazomib citrate, pamidronate disodium, panobinostat, plerixafor, pomalidomide, pomalidomide, lenalidomide, selinexor, thalidomide, thalidomide, bortezomib, selinexor, zoledronic acid, or zoledronic acid.

In an aspect, a disclosed method can further comprise administering one or more proteasome inhibitors or agents that act on plasma cells prior to administering a disclosed isolated nucleic acid molecule, a disclosed vector, or a disclosed pharmaceutical formulation. In an aspect, a disclosed method can comprise administering one or more proteasome inhibitors or one or more agents that act on plasma cells concurrently with administering a disclosed isolated nucleic acid molecule, a disclosed vector, or a disclosed pharmaceutical formulation. In an aspect, a disclosed method can comprise administering one or more proteasome inhibitors or one or more agents that act on plasma cells subsequent to administering a disclosed isolated nucleic acid molecule, a disclosed vector, or a disclosed pharmaceutical formulation. In an aspect, a disclosed method can further comprise administering one or more proteasome inhibitors more than 1 time. In an aspect, a disclosed method can further comprise administering one or more proteasome inhibitors repeatedly over time.

In an aspect, a disclosed method can comprise administering one or more immunosuppressive agents. In an aspect, an immunosuppressive agent can be, but is not limited to, azathioprine, methotrexate, sirolimus, anti-thymocyte globulin (ATG), cyclosporine (CSP), mycophenolate mofetil (MMF), steroids, or a combination thereof. In an aspect, a disclosed method can comprise administering one or more immunosuppressive agents more than 1 time. In an aspect, a disclosed method can comprise administering one or more one or more immunosuppressive agents repeatedly over time. In an aspect, a disclosed method can comprise administering a compound that targets or alters antigen presentation or humoral or cell mediated or innate immune responses.

In an aspect, a disclosed method can comprise administering a compound that exerts a therapeutic effect against B cells and/or a compound that targets or alters antigen presentation or humoral or cell mediated immune response. In an aspect, a disclosed compound can be rituximab, methotrexate, intravenous gamma globulin, anti CD4 antibody, anti CD2, an anti-FcRN antibody, a BTK inhibitor, an anti-IGF1R antibody, a CD19 antibody (e.g., inebilizumab), an anti-IL6 antibody (e.g., tocilizumab), an antibody to CD40, an IL2 mutein, or a combination thereof. Also disclosed herein are Treg infusions that can be administered as a way to help with immune tolerance (e.g., antigen specific Treg cells to AAV).

In an aspect, a disclosed method can further comprise administering lipid nanoparticles (LNPs). In an aspect, LNPs can be organ targeted. In an aspect, LNPs can be liver-targeted or testes-targeted. For example, in an aspect, mRNA therapy with lipid nanoparticle encapsulation for systemic delivery to hepatocytes has the potential to restore metabolic enzymatic activity for one or more glycogen storage diseases such as GSD IX al, GSD IX α2, GSD IX β, GSD IX δ, GSD IX γ2, and/or GSD VI. In an aspect, the mRNA therapy focuses on phosphorylase kinase catalytic subunit alpha 1 (PHKA1), phosphorylase kinase catalytic subunit alpha 2 (PHKA2), phosphorylase kinase catalytic subunit beta (PHKB), glycogen phosphorylase kinase subunit δ (CALM1, CALM2, CALM3), phosphorylase kinase catalytic subunit gamma 2 (PHKG2), and/or glycogen phosphorylase, liver form (PGYL). In an aspect, the mRNA therapy focuses on one or more genes in the glycogenolysis pathway (such as those identified in FIG. 1).

In an aspect, a disclosed method can further comprise treating a subject that has developed or is likely to develop neutralizing antibodies (ABs) to a disclosed vector, capsid, and/or transgene. In an aspect, treating a subject that has developed or is likely to develop neutralizing antibodies can comprise plasmapheresis and immunosuppression. In an aspect, a disclosed method can comprise using immunosuppression to decrease the T cell, B cell, and/or plasma cell population, decrease the innate immune response, inflammatory response, and antibody levels in general. In an aspect, a disclosed method can comprise administering an IgG-degrading agent that depletes pre-existing neutralizing antibodies. In an aspect, a disclosed method can comprise administering to the subject IdeS or IdeZ, rapamycin, and/or SVP-Rapamycin. In an aspect, a disclosed method can comprise administering Tacrolimus. In an aspect, a disclosed IgG-degrading agent is bacteria-derived IdeS or IdeZ. In an aspect, a disclosed method can further comprise administering the subject a disclosed RNA therapeutic.

In an aspect, a disclosed method can further comprise administering to the subject an effective amount an isolated nucleic acid encoding a protein that is deficient or absent in the subject. In an aspect, a disclosed encoded protein can comprise a recombinant human protein such as, for example, recombinant alpha glucosidase (GAA). In an aspect, a disclosed GAA can comprise the amino acid sequence set forth in SEQ ID NO:34, SEQ ID NO:35, SEQ ID NO:36, SEQ ID NO:37, SEQ ID NO:38, or a fragment thereof. In an aspect, a disclosed GAA can comprise a sequence having at least 30%, at least 40%, at least 50%, at least 60%, at least 70%, at least 80%, or at least 90% identity to the sequence set forth in SEQ ID NO:34, SEQ ID NO:35, SEQ ID NO:36, SEQ ID NO:37, SEQ ID NO:38, or a fragment thereof. In an aspect, a disclosed GAA can be Myozyme or Lumizyme. In an aspect of a disclosed method, a disclosed isolated nucleic acid sequence for GAA can comprise the sequence set forth in SEQ ID NO:39, SEQ ID NO:40, or a fragment thereof. In an aspect, a disclosed isolated nucleic acid sequence for GAA can comprise a sequence having at least 30%, at least 40%, at least 50%, at least 60%, at least 70%, at least 80%, or at least 90% identity to the sequence set forth in SEQ ID NO:39, SEQ ID NO:40, or a fragment thereof. In an aspect, a disclosed isolated nucleic acid encoding a recombinant protein such as, for example, GAA, can be present in a disclosed viral vector. In an aspect, a disclosed isolated nucleic acid encoding a recombinant protein such as, for example, GAA, can be present in a disclosed viral vector including, for example, an AAV vector or a self-complementary AAV vector. In an aspect, a disclosed immune modulator and a disclosed therapeutic agent can be concurrently administered. In an aspect, a disclosed composition comprising GAA or a disclosed vector comprising a disclosed isolated nucleic acid molecule encoding GAA can be administered prior to, concurrent with, or after the administration of a disclosed isolated nucleic acid molecule comprising a nucleic acid sequence encoding a polypeptide capable of preventing glycogen accumulation and/or degrading accumulated glycogen.

In an aspect, a disclosed method can further comprise modifying one or more of the disclosed steps.

### 3. RNA Therapeutic Based Methods

Disclosed herein is a method of treating and/or preventing GSD IX al, GSD IX α2, GSD IX β, GSD IX δ, GSD IX γ2, and/or GSD VI disease progression comprising administering to a subject in need thereof a therapeutically effective amount of an RNA therapeutic. Disclosed herein is a method of treating and/or preventing disease progression comprising administering to a subject having GSD IX and/or GSD VI a therapeutically effective amount of an RNA therapeutic.

Disclosed herein is a method of preventing glycogen accumulation and/or degrading accumulated glycogen comprising administering to a subject having GSD IX al, GSD IX α2, GSD IX β, GSD IX δ, GSD IX γ2, and/or GSD VI a therapeutically effective amount of an RNA therapeutic. Disclosed herein is a method of preventing glycogen accumulation and/or degrading accumulated glycogen comprising administering to a subject having GSD IX and/or GSD VI a therapeutically effective amount of an RNA therapeutic.

Disclosed herein is a method of treating and/or reducing liver disease comprising administering to a subject having GSD IX al, GSD IX α2, GSD IX β, GSD IX δ, GSD IX γ2, and/or GSD VI a therapeutically effective amount of an RNA therapeutic. Disclosed herein is a method of treating and/or reducing liver disease comprising administering to a subject having GSD IX and/or GSD VI a therapeutically effective amount of an RNA therapeutic.

Disclosed herein is a method of treating and/or preventing GSD IX al, GSD IX α2, GSD IX β, GSD IX δ, GSD IX γ2, and/or GSD VI disease progression comprising administering to a subject in need thereof a therapeutically effective amount of an RNA therapeutic, and reducing the expression level and/or activity level of glycogen synthase. Disclosed herein is a method of treating and/or preventing disease progression comprising administering to a subject having GSD IX and/or GSD VI to a subject having GSD IX and/or GSD VI a therapeutically effective amount of an RNA therapeutic, and reducing the expression level and/or activity level of glycogen synthase.

Disclosed herein is a method of preventing glycogen accumulation and/or degrading accumulated glycogen comprising administering to a subject having GSD IX al, GSD IX α2, GSD IX β, GSD IX δ, GSD IX γ2, and/or GSD VI a therapeutically effective amount of an RNA therapeutic, and reducing the expression level and/or activity level of glycogen synthase. Disclosed herein is a method of preventing glycogen accumulation and/or degrading accumulated glycogen comprising administering to a subject having GSD IX and/or GSD VI a therapeutically effective amount of an RNA therapeutic, and reducing the expression level and/or activity level of glycogen synthase.

Disclosed herein is a method of treating and/or reducing liver disease comprising administering to a subject having GSD IX al, GSD IX α2, GSD IX β, GSD IX δ, GSD IX γ2, and/or GSD VI a therapeutically effective amount of an RNA therapeutic, and reducing the expression level and/or activity level of glycogen synthase. Disclosed herein is a method of treating and/or reducing liver disease comprising administering to a subject having GSD IX and/or GSD VI a therapeutically effective amount of an RNA therapeutic, and reducing the expression level and/or activity level of glycogen synthase.

In an aspect, a disclosed method comprising administering an RNA therapeutic can correct a deficiency in the PhK complex, can restore the functionality of the PhK complex, or both. In an aspect, a disclosed method comprising administering an RNA therapeutic can correct or restore one or more aspect of the glycogen synthesis pathway or the glycogenolysis pathway or both (such as those identified in FIG. 1). In an aspect, a disclosed method can correct, restore, supplement, and/or replenish the enzymatic activity of one or more glycogen phosphorylase kinases and/or glycogen phosphorylases. In an aspect, a disclosed method can correct, restore, supplement, and/or replenish the enzymatic activity of one or more of human glycogen phosphorylase kinase subunit α1, glycogen phosphorylase kinase subunit α2, glycogen phosphorylase kinase subunit β, glycogen phosphorylase kinase subunit δ, glycogen phosphorylase kinase subunit γ2, and/or glycogen phosphorylase.

In an aspect, a disclosed method can restore one or more aspects of the glycogen metabolic pathway, restore one or more aspects of the glycogenolysis metabolic pathway, can restore one or more aspects of the glycogenesis metabolic pathway, can restore PhK subunit activity and/or functionality, can restore PYGL activity and/or functionality, or any combination thereof. For example, in an aspect, a disclosed method can restore the balance of glycogen synthesis and degradation. In an aspect, a disclosed method can restore the balance of glycogen metabolism, wherein glycogen metabolism comprises glycogen synthesis and breakdown. In an aspect, a disclosed method can restore the functionality and/or structural integrity of the PhK complex. For example, in an aspect, by restoring the functionality and/or structural integrity of a subunit (such as α2, δ, β, and γ2), then the functionality and/or structural integrity of the PhK complex (the heterotetramer) can be restored. In an aspect, restoring one or more aspects of a disclosed metabolic pathway can comprise restoring the activity and/or functionality of one or more enzymes identified in FIG. 1. In an aspect, restoring PhK subunit activity and/or functionality can comprise restoring the activity and/or functionality of the al subunit, the α2 subunit, the R subunit, the γ2 subunit, or any combination thereof.

In an aspect, restoring PhK activity and/or functionality or restoring PYGL activity and/or functionality can be a 10%, 20%, 30%, 40%, 50%, 60%, 70%, 80%, 90%, 100%, or any amount of restoration when compared to a pre-existing level such as, for example, a pre-treatment level. In an aspect, the amount of restoration can be 10-20%, 20-30%, 30-40%, 40-50%, 50-60%, 60-70%, 70-80%, 80-90%, or 90-100% more than a pre-existing level such as, for example, a pre-treatment level. In an aspect, restoration can be measured against a control level (e.g., a level in a subject not having a GSD). In an aspect, restoration can be a partial or incomplete restoration. In an aspect, restoration can be complete or near complete restoration such that the level of expression, activity and/or functionality is similar to that of a wild-type or control level.

In an aspect, a disclosed method can comprise restoring one or more aspects of cellular homeostasis and/or cellular functionality and/or metabolic dysregulation. In an aspect, restoring one or more aspects of cellular homeostasis and/or cellular functionality can comprise one or more of the following: (i) correcting cell starvation in one or more cell types (such as, for example, liver cells and muscle cells); (ii) normalizing aspects of the autophagy pathway (such as, for example, correcting, preventing, reducing, and/or ameliorating autophagy); (iii) improving, enhancing, restoring, and/or preserving mitochondrial functionality and/or structural integrity; (iv) improving, enhancing, restoring, and/or preserving organelle functionality and/or structural integrity; (v) preventing, slowing, and/or eliminating hypoglycemia, ketosis, and/or other liver abnormalities; (vi) correcting liver enzyme dysregulation; (vii) reversing, inhibiting, preventing, stabilizing, and/or slowing the rate of progression of the multi-systemic manifestations of GSD IX; (viii) reversing, inhibiting, preventing, stabilizing, and/or slowing the rate of progression of liver disease including fibrosis, cirrhosis, hepatic adenomas, and/or liver hepatocellular carcinoma, or (ix) any combination thereof. In an aspect, restoring one or more aspects of cellular homeostasis can comprise improving, enhancing, restoring, and/or preserving one or more aspects of cellular structural and/or functional integrity.

In an aspect of a disclosed method, techniques to monitor, measure, and/or assess the restoring one or more aspects of cellular homeostasis and/or cellular functionality can comprise qualitative (or subjective) means as well as quantitative (or objective) means. These means are known to the skilled person.

In an aspect, a disclosed mRNA molecule can be translated in one or more targeted cells to produce functional versions of the one or more genes in the glycogen synthesis pathway or glycogenolysis pathway.

In an aspect, a disclosed RNA therapeutic can comprise oligonucleotides to target RNA. In an aspect, a disclosed RNA therapeutic can comprise RNA interference (RNAi). In an aspect, a disclosed RNA therapeutic can comprise antisense oligonucleotides (ASOs).

In an aspect, a disclosed RNA therapeutic can inhibit mRNA translation of one or more genes in the glycogen synthesis or glycogenolysis pathways. In an aspect, the one or more genes in the glycogen synthesis or glycogenolysis pathways can be phosphorylase kinase catalytic subunit gamma 2 (PHKG2), phosphorylase a, phosphorylase b, a debranching enzyme, a branching enzyme, an acid alpha-glucosidase enzyme, or glucose-6-phosphatase, or a combination thereof. In an aspect, a disclosed RNA therapeutic can inhibit mRNA translation of liver glycogen synthase (GYS1) or muscle glycogen synthase (GYS2).

In an aspect, a disclosed RNA therapeutic can comprise an mRNA molecule encoding one or more genes in the glycogen synthesis or glycogenolysis pathways. In an aspect, the encoded gene can be a wild-type gene or a non-mutated gene. In an aspect, the one or more genes in the glycogen synthesis or glycogenolysis pathways can be phosphorylase kinase regulatory subunit alpha 1 (PHKA1), phosphorylase kinase regulatory subunit alpha 2 (PHKA2), phosphorylase kinase regulatory subunit beta (PHKB), glycogen phosphorylase kinase regulatory subunit delta (CALM1, CALM2, CALM3), phosphorylase kinase catalytic subunit gamma 2 (PHKG2), glycogen phosphorylase (PYGL), phosphorylase a, phosphorylase b, a debranching enzyme, a branching enzyme, an acid alpha-glucosidase enzyme, or glucose-6-phosphatase, or a combination thereof.

In an aspect, delivery of a disclosed RNA therapeutic can be via lipoplex-mediated transfection, polyplex-mediated transfection, polypeptide-mediated delivery, virus-mediated delivery, cell-mediated delivery, or exosome-mediated delivery, or a combination thereof.

In an aspect, a disclosed RNA therapeutic can be administered via lipid nanoparticles (LNPs). In an aspect, LNPs can be organ targeted. In an aspect, the LNPs can be targeted to a subject's liver, heart, skeletal muscle, smooth muscle, CNS, PNS, or a combination thereof. In an aspect, LNPs can be liver-targeted or testes-targeted. For example, in an aspect, a disclosed RNA therapeutic can be administered via LNP encapsulation to a subject's liver.

In an aspect, a disclosed mRNA molecule can comprise at least one modification that confers increased or enhanced stability to the nucleic acid, including, for example, improved resistance to nuclease digestion in vivo. As used herein, the terms “stable” and “stability” can refer to increased or enhanced resistance of the disclosed nucleic acid molecules (including mRNA molecules) to degradation by, for example, nucleases (i.e., endonucleases or exonucleases). Increased stability can comprise less sensitivity to hydrolysis or other destruction by endogenous enzymes (e.g., endonucleases or exonucleases) or conditions within the target cell or tissue, thereby increasing or enhancing the residence of such mRNA in the target cell, tissue, subject, and/or cytoplasm. Increased stability can also comprise the improved half-life of a nucleic acid relative to its naturally occurring, unmodified counterpart (e.g. the wild-type version of the mRNA). Other stability increasing measures include modifications to the mRNA that improve or enhance translation of mRNA nucleic acids, including for example, the inclusion of sequences that function in the initiation of protein translation (e.g., the Kozak consensus sequence). In an aspect, a disclosed mRNA has undergone a chemical or biological modification to render it more stable. Exemplary modifications to an mRNA include the depletion of a base (e.g., by deletion or by the substitution of one nucleotide for another) or modification of a base, for example, the chemical modification of a base.

In an aspect, a disclosed method comprising administering an RNA therapeutic can further comprise administering to the subject a therapeutically effective amount of a therapeutic agent.

In an aspect, a disclosed method comprising administering an RNA therapeutic can further comprise reducing glycogen levels by administering a glycogen synthase inhibitor (e.g., RNAi, ASO, etc.) to the subject, or modifying the subject's diet, for example, by using cornstarch or another slow release starch to prevent hypoglycemia, or modifying the subject's diet, for example, by consuming a high amount of protein, fat, or other anaplerotic agents (such as, for example, C7 compounds like triheptanoin), exercise or a combination thereof.

In an aspect, a disclosed method can comprise reducing the expression level, activity level, or both of glycogen synthase. In an aspect, a glycogen synthase can be GYS1 (muscle glycogen synthase) or GYS2 (liver glycogen synthase) or both. In an aspect, a disclosed GYS can be GYS2. In an aspect, a disclosed formulation can comprise a RNA therapeutic. A RNA therapeutic can comprise RNA-mediated interference (RNAi) and/or antisense oligonucleotides (ASO). In an aspect, a disclosed RNA therapeutic can be directed at GYS1, GYS2, or both. In an aspect, a disclosed RNA therapeutic can be directed at GYS2.

In an aspect, a disclosed method comprising administering an RNA therapeutic can further comprise gene editing one or more relevant genes (such as, for example, genes in the glycogenolysis pathway), wherein editing includes but is not limited to single gene knockout, loss of function screening of multiple genes at one, gene knockin, or a combination thereof.

In an aspect, a disclosed method comprising administering an RNA therapeutic can further comprise administering to the subject a therapeutically effective amount of an agent that can correct one or more aspects of a dysregulated glycogen metabolic pathway, such as glycogen synthesis or glycogenolysis. In an aspect, such an agent can comprise an enzyme for enzyme replacement therapy. In an aspect, a disclosed enzyme can replace a mutated or dysfunction or nonexistence product of the PHKG2 gene, or a combination thereof. In an aspect, a disclosed enzyme can replace any enzyme in a dysregulated or dysfunctional glycogen metabolic pathway.

In an aspect, a disclosed method comprising administering an RNA therapeutic can further comprise administering one or more immune modulators. In an aspect, a disclosed immune modulator can be methotrexate, rituximab, intravenous gamma globulin, or bortezomib, or a combination thereof. In an aspect, a disclosed immune modulator can be bortezomib or SVP-Rapamycin. In an aspect, a disclosed immune modulator can be Tacrolimus.

In an aspect, a disclosed immune modulator such as methotrexate can be administered at a transient low to high dose. In an aspect, a disclosed immune modulator can be administered at a dose of about 0.1 mg/kg body weight to about 0.6 mg/kg body weight. In an aspect, a disclosed immune modulator can be administered at a dose of about 0.4 mg/kg body weight. In an aspect, a disclosed immune modulator can be administered at about a daily dose of 0.4 mg/kg body weight for 3 to 5 or greater cycles, with up to three days per cycle. In an aspect, a disclosed immune modulator can be administered at about a daily dose of 0.4 mg/kg body weight for a minimum of 3 cycles, with three days per cycle. In an aspect, a person skilled in the art can determine the appropriate number of cycles. In an aspect, a disclosed immune modulator can be administered as many times as necessary to achieve a desired clinical effect.

In an aspect, a disclosed immune modulator can be administered orally about one hour before a disclosed therapeutic agent. In an aspect, a disclosed immune modulator can be administered subcutaneously about 15 minutes before a disclosed therapeutic agent. In an aspect, a disclosed immune modulator can be administered concurrently with a disclosed therapeutic agent. In an aspect, a disclosed immune modulator can be administered orally about one hour or a few days before a disclosed isolated nucleic acid molecule, a disclosed vector, a disclosed pharmaceutical formulation, or a combination thereof. In an aspect, a disclosed immune modulator can be administered subcutaneously about 15 minutes before or a few days before a disclosed isolated nucleic acid molecule, a disclosed vector, a disclosed pharmaceutical formulation, or a combination thereof. In an aspect, a disclosed immune modulator can be administered concurrently with a disclosed isolated nucleic acid molecule, a disclosed vector, a disclosed pharmaceutical formulation, or a combination thereof.

In an aspect, a disclosed method comprising administering an RNA therapeutic can further comprise administering one or more proteasome inhibitors (e.g., bortezomib, carfilzomib, marizomib, ixazomib, and oprozomib). In an aspect, a proteasome inhibitor can be an agent that acts on plasma cells (e.g., daratumumab). In an aspect, an agent that acts on a plasma cell can be melphalan hydrochloride, melphalan, pamidronate disodium, carmustine, carfilzomib, carmustine, cyclophosphamide, daratumumab, doxorubicin hydrochloride liposome, doxorubicin hydrochloride liposome, elotuzumab, melphalan hydrochloride, panobinostat, ixazomib citrate, carfilzomib, lenalidomide, melphalan, melphalan hydrochloride, plerixafor, ixazomib citrate, pamidronate disodium, panobinostat, plerixafor, pomalidomide, pomalidomide, lenalidomide, selinexor, thalidomide, thalidomide, bortezomib, selinexor, zoledronic acid, or zoledronic acid.

In an aspect, a disclosed method comprising administering an RNA therapeutic can further comprise administering one or more proteasome inhibitors or agents that act on plasma cells prior to administering a disclosed isolated nucleic acid molecule, a disclosed vector, or a disclosed pharmaceutical formulation. In an aspect, a disclosed method can comprise administering one or more proteasome inhibitors or one or more agents that act on plasma cells concurrently with administering a disclosed isolated nucleic acid molecule, a disclosed vector, or a disclosed pharmaceutical formulation. In an aspect, a disclosed method can comprise administering one or more proteasome inhibitors or one or more agents that act on plasma cells subsequent to administering a disclosed isolated nucleic acid molecule, a disclosed vector, or a disclosed pharmaceutical formulation. In an aspect, a disclosed method comprising administering an RNA therapeutic can further comprise administering one or more proteasome inhibitors more than 1 time. In an aspect, a disclosed method can comprise administering one or more proteasome inhibitors repeatedly over time.

In an aspect, a disclosed method can further comprise administering one or more immunosuppressive agents. In an aspect, an immunosuppressive agent can be, but is not limited to, azathioprine, methotrexate, sirolimus, anti-thymocyte globulin (ATG), cyclosporine (CSP), mycophenolate mofetil (MMF), steroids, or a combination thereof. In an aspect, a disclosed method can further comprise administering one or more immunosuppressive agents more than 1 time. In an aspect, a disclosed method can further comprise administering one or more one or more immunosuppressive agents repeatedly over time. In an aspect, a disclosed method can comprise administering a compound that targets or alters antigen presentation or humoral or cell mediated or innate immune responses.

In an aspect, a disclosed method comprising administering an RNA therapeutic can further comprise administering a compound that exerts a therapeutic effect against B cells and/or a compound that targets or alters antigen presentation or humoral or cell mediated immune response. In an aspect, a disclosed compound can be rituximab, methotrexate, intravenous gamma globulin, anti CD4 antibody, anti CD2, an anti-FcRN antibody, a BTK inhibitor, an anti-IGFTR antibody, a CD19 antibody (e.g., inebilizumab), an anti-IL6 antibody (e.g., tocilizumab), an antibody to CD40, an IL2 mutein, or a combination thereof. Also disclosed herein are Treg infusions that can be administered as a way to help with immune tolerance (e.g., antigen specific Treg cells to AAV).

In an aspect, a disclosed method comprising administering an RNA therapeutic can further comprise treating a subject that has developed or is likely to develop neutralizing antibodies (ABs) to a disclosed vector, capsid, and/or transgene. In an aspect, treating a subject that has developed or is likely to develop neutralizing antibodies can comprise plasmapheresis and immunosuppression. In an aspect, a disclosed method can comprise using immunosuppression to decrease the T cell, B cell, and/or plasma cell population, decrease the innate immune response, inflammatory response, and antibody levels in general. In an aspect, a disclosed method can comprise administering an IgG-degrading agent that depletes pre-existing neutralizing antibodies. In an aspect, a disclosed method can comprise administering to the subject IdeS or IdeZ, rapamycin, and/or SVP-Rapamycin. In an aspect, a disclosed method can comprise administering Tacrolimus. In an aspect, a disclosed IgG-degrading agent is bacteria-derived IdeS or IdeZ.

In an aspect, a disclosed method comprising administering an RNA therapeutic can further comprise administering one or more disclosed nucleic acid molecules, disclosed vectors, or disclosed formulations.

In an aspect, a disclosed method comprising administering an RNA therapeutic can comprise of or more of the disclosed steps.

In an aspect, a disclosed method can further comprise administering to the subject an effective amount an isolated nucleic acid encoding a protein that is deficient or absent in the subject. In an aspect, a disclosed encoded protein can comprise a recombinant human protein such as, for example, recombinant alpha-glucosidase (GAA). In an aspect, a disclosed GAA can comprise the amino acid sequence set forth in SEQ ID NO:34, SEQ ID NO:35, SEQ ID NO:36, SEQ ID NO:37, SEQ ID NO:38, or a fragment thereof. In an aspect, a disclosed GAA can comprise a sequence having at least 30%, at least 40%, at least 50%, at least 60%, at least 70%, at least 80%, or at least 90% identity to the sequence set forth in SEQ ID NO:34, SEQ ID NO:35, SEQ ID NO:36, SEQ ID NO:37, SEQ ID NO:38, or a fragment thereof. In an aspect, a disclosed GAA can be Myozyme or Lumizyme. In an aspect of a disclosed method, a disclosed isolated nucleic acid sequence for GAA can comprise the sequence set forth in SEQ ID NO:39, SEQ ID NO:40, or a fragment thereof. In an aspect, a disclosed isolated nucleic acid sequence for GAA can comprise a sequence having at least 30%, at least 40%, at least 50%, at least 60%, at least 70%, at least 80%, or at least 90% identity to the sequence set forth in SEQ ID NO:39, SEQ ID NO:40, or a fragment thereof. In an aspect, a disclosed isolated nucleic acid sequence encoding a recombinant protein such as, for example, GAA, can be present in a disclosed viral vector. In an aspect, a disclosed isolated nucleic acid encoding a recombinant protein such as, for example, GAA, can be present in a disclosed viral vector including, for example, an AAV vector or a self-complementary AAV vector. In an aspect, a disclosed immune modulator and a disclosed therapeutic agent can be concurrently administered. In an aspect, a disclosed composition comprising GAA or a disclosed vector comprising a disclosed isolated nucleic acid molecule encoding GAA can be administered prior to, concurrent with, or after the administration of a disclosed RNA therapeutic.

### E. Agents

### 1. Biologically Active Agents

As used herein, the term “biologically active agent” or “biologic active agent” or “bioactive agent” means an agent that is capable of providing a local or systemic biological, physiological, or therapeutic effect in the biological system to which it is applied. For example, the bioactive agent can act to control infection or inflammation, enhance cell growth and tissue regeneration, control tumor growth, act as an analgesic, promote anti-cell attachment, and enhance bone growth, among other functions. Other suitable bioactive agents can include anti-viral agents, vaccines, hormones, antibodies (including active antibody fragments sFv, Fv, and Fab fragments), aptamers, peptide mimetics, functional nucleic acids, therapeutic proteins, peptides, or nucleic acids. Other bioactive agents include prodrugs, which are agents that are not biologically active when administered but, upon administration to a subject are converted to bioactive agents through metabolism or some other mechanism. Additionally, any of the compositions of the invention can contain combinations of two or more bioactive agents. It is understood that a biologically active agent can be used in connection with administration to various subjects, for example, to humans (i.e., medical administration) or to animals (i.e., veterinary administration). As used herein, the recitation of a biologically active agent inherently encompasses the pharmaceutically acceptable salts thereof.

### 2. Pharmaceutically Active Agents

As used herein, the term “pharmaceutically active agent” includes a “drug” or a “vaccine” and means a molecule, group of molecules, complex or substance administered to an organism for diagnostic, therapeutic, preventative medical, or veterinary purposes. This term includes externally and internally administered topical, localized and systemic human and animal pharmaceuticals, treatments, remedies, nutraceuticals, cosmeceuticals, biologicals, devices, diagnostics and contraceptives, including preparations useful in clinical and veterinary screening, prevention, prophylaxis, healing, wellness, detection, imaging, diagnosis, therapy, surgery, monitoring, cosmetics, prosthetics, forensics and the like. This term may also be used in reference to agriceutical, workplace, military, industrial and environmental therapeutics or remedies comprising selected molecules or selected nucleic acid sequences capable of recognizing cellular receptors, membrane receptors, hormone receptors, therapeutic receptors, microbes, viruses or selected targets comprising or capable of contacting plants, animals and/or humans. This term can also specifically include nucleic acids and compounds comprising nucleic acids that produce a bioactive effect, for example deoxyribonucleic acid (DNA) or ribonucleic acid (RNA). Pharmaceutically active agents include the herein disclosed categories and specific examples. It is not intended that the category be limited by the specific examples. Those of ordinary skill in the art will recognize also numerous other compounds that fall within the categories and that are useful according to the invention. Examples include a radiosensitizer, the combination of a radiosensitizer and a chemotherapeutic, a steroid, a xanthine, a beta-2-agonist bronchodilator, an anti-inflammatory agent, an analgesic agent, a calcium antagonist, an angiotensin-converting enzyme inhibitors, abeta-blocker, acentrally active alpha-agonist, an alpha-1-antagonist, carbonic anhydrase inhibitors, prostaglandin analogs, a combination of an alpha agonist and a beta blocker, a combination of a carbonic anhydrase inhibitor and a beta blocker, an anticholinergic/antispasmodic agent, a vasopressin analogue, an antiarrhythmic agent, an antiparkinsonian agent, an antiangina/antihypertensive agent, an anticoagulant agent, an antiplatelet agent, a sedative, an ansiolytic agent, a peptidic agent, a biopolymeric agent, an antineoplastic agent, a laxative, an antidiarrheal agent, an antimicrobial agent, an antifungal agent, or a vaccine. In a further aspect, the pharmaceutically active agent can be coumarin, albumin, bromolidine, steroids such as betamethasone, dexamethasone, methylprednisolone, prednisolone, prednisone, triamcinolone, budesonide, hydrocortisone, and pharmaceutically acceptable hydrocortisone derivatives; xanthines such as theophylline and doxophylline; beta-2-agonist bronchodilators such as salbutamol, fenterol, clenbuterol, bambuterol, salmeterol, fenoterol; antiinflammatory agents, including antiasthmatic anti-inflammatory agents, antiarthritis antiinflammatory agents, and non-steroidal antiinflammatory agents, examples of which include but are not limited to sulfides, mesalamine, budesonide, salazopyrin, diclofenac, pharmaceutically acceptable diclofenac salts, nimesulide, naproxene, acetominophen, ibuprofen, ketoprofen and piroxicam; analgesic agents such as salicylates; calcium channel blockers such as nifedipine, amlodipine, and nicardipine; angiotensin-converting enzyme inhibitors such as captopril, benazepril hydrochloride, fosinopril sodium, trandolapril, ramipril, lisinopril, enalapril, quinapril hydrochloride, and moexipril hydrochloride; beta-blockers (i.e., beta adrenergic blocking agents) such as sotalol hydrochloride, timolol maleate, timol hemihydrate, levobunolol hydrochloride, esmolol hydrochloride, carteolol, propanolol hydrochloride, betaxolol hydrochloride, penbutolol sulfate, metoprolol tartrate, metoprolol succinate, acebutolol hydrochloride, atenolol, pindolol, and bisoprolol fumarate; centrally active alpha-2-agonists (i.e., alpha adrenergic receptor agonist) such as clonidine, brimonidine tartrate, and apraclonidine hydrochloride; alpha-1-antagonists such as doxazosin and prazosin; anticholinergic/antispasmodic agents such as dicyclomine hydrochloride, scopolamine hydrobromide, glycopyrrolate, clidinium bromide, flavoxate, and oxybutynin; vasopressin analogues such as vasopressin and desmopressin; prostaglandin analogs such as latanoprost, travoprost, and bimatoprost; cholinergics (i.e., acetylcholine receptor agonists) such as pilocarpine hydrochloride and carbachol; glutamate receptor agonists such as the N-methyl D-aspartate receptor agonist memantine; anti-Vascular endothelial growth factor (VEGF) aptamers such as pegaptanib; anti-VEGF antibodies (including but not limited to anti-VEGF-A antibodies) such as ranibizumab and bevacizumab; carbonic anhydrase inhibitors such as methazolamide, brinzolamide, dorzolamide hydrochloride, and acetazolamide; antiarrhythmic agents such as quinidine, lidocaine, tocainide hydrochloride, mexiletine hydrochloride, digoxin, verapamil hydrochloride, propafenone hydrochloride, flecaimide acetate, procainamide hydrochloride, moricizine hydrochloride, and diisopyramide phosphate; antiparkinsonian agents, such as dopamine, L-Dopa/Carbidopa, selegiline, dihydroergocryptine, pergolide, lisuride, apomorphine, and bromocryptine; antiangina agents and antihypertensive agents such as isosorbide mononitrate, isosorbide dinitrate, propranolol, atenolol and verapamil; anticoagulant and antiplatelet agents such as coumadin, warfarin, acetylsalicylic acid, and ticlopidine; sedatives such as benzodiazapines and barbiturates; ansiolytic agents such as lorazepam, bromazepam, and diazepam; peptidic and biopolymeric agents such as calcitonin, leuprolide and other LHRH agonists, hirudin, cyclosporin, insulin, somatostatin, protirelin, interferon, desmopressin, somatotropin, thymopentin, pidotimod, erythropoietin, interleukins, melatonin, granulocyte/macrophage-CSF, and heparin; antineoplastic agents such as etoposide, etoposide phosphate, cyclophosphamide, methotrexate, 5-fluorouracil, vincristine, doxorubicin, cisplatin, hydroxyurea, leucovorin calcium, tamoxifen, flutamide, asparaginase, altretamine, mitotane, and procarbazine hydrochloride; laxatives such as senna concentrate, casanthranol, bisacodyl, and sodium picosulphate; antidiarrheal agents such as difenoxine hydrochloride, loperamide hydrochloride, furazolidone, diphenoxylate hydrochloride, and microorganisms; vaccines such as bacterial and viral vaccines; antimicrobial agents such as penicillins, cephalosporins, and macrolides, antifungal agents such as imidazolic and triazolic derivatives; and nucleic acids such as DNA sequences encoding for biological proteins, and antisense oligonucleotides. It is understood that a pharmaceutically active agent can be used in connection with administration to various subjects, for example, to humans (i.e., medical administration) or to animals (i.e., veterinary administration). As used herein, the recitation of a pharmaceutically active agent inherently encompasses the pharmaceutically acceptable salts thereof.

### 3. Anti-Bacterial Agents

As used herein, anti-bacterial agents are known to the art. For example, the art generally recognizes several categories of anti-bacterial agents including (1) penicillins, (2) cephalosporins, (3) quinolones, (4) aminoglycosides, (5) monobactams, (6) carbapenems, (7) macrolides, and (8) other agents. For example, as used herein, an anti-bacterial agent can comprise Afenide, Amikacin, Amoxicillin, Ampicillin, Arsphenamine, Augmentin, Azithromycin, Azlocillin, Aztreonam, Bacampicillin, Bacitracin, Balofloxacin, Besifloxacin, Capreomycin, Carbacephem (loracarbef), Carbenicillin, Cefacetrile (cephacetrile), Cefaclomezine, Cefaclor, Cefadroxil (cefadroxyl), Cefalexin (cephalexin), Cefaloglycin (cephaloglycin), Cefalonium (cephalonium), Cefaloram, Cefaloridine (cephaloradine), Cefalotin (cephalothin), Cefamandole, Cefaparole, Cefapirin (cephapirin), Cefatrizine, Cefazaflur, Cefazedone, Cefazolin (cephazolin), Cefcanel, Cefcapene, Cefclidine, Cefdaloxime, Cefdinir, Cefditoren, Cefedrolor, Cefempidone, Cefepime, Cefetamet, Cefetrizole, Cefivitril, Cefixime, Cefluprenam, Cefmatilen, Cefmenoxime, Cefmepidium, Cefmetazole, Cefodizime, Cefonicid, Cefoperazone, Cefoselis, Cefotaxime, Cefotetan, Cefovecin, Cefoxazole, Cefoxitin, Cefozopran, Cefpimizole, Cefpirome, Cefpodoxime, Cefprozil (cefproxil), Cefquinome, Cefradine (cephradine), Cefrotil, Cefroxadine, Cefsumide, Ceftaroline, Ceftazidime, Ceftazidime/Avibactam, Cefteram, Ceftezole, Ceftibuten, Ceftiofur, Ceftiolene, Ceftioxide, Ceftizoxime, Ceftobiprole, Ceftriaxone, Cefuracetime, Cefuroxime, Cefuzonam, Cephalexin, Chloramphenicol, Chlorhexidine, Ciprofloxacin, Clarithromycin, Clavulanic Acid, Clinafloxacin, Clindamycin, Cloxacillin, Colimycin, Colistimethate, Colistin, Crysticillin, Cycloserine 2, Demeclocycline, Dicloxacillin, Dirithromycin, Doripenem, Doxycycline, Efprozil, Enoxacin, Ertapenem, Erythromycin, Ethambutol, Flucloxacillin, Flumequine, Fosfomycin, Furazolidone, Gatifloxacin, Geldanamycin, Gemifloxacin, Gentamicin, Glycopeptides, Grepafloxacin, Herbimycin, Imipenem, Isoniazid, Kanamycin, Levofloxacin, Lincomycin, Linezolid, Lipoglycopeptides, Lomefloxacin, Meropenem, Meticillin, Metronidazole, Mezlocillin, Minocycline, Mitomycin, Moxifloxacin, Mupirocin, Nadifloxacin, Nafcillin, Nalidixic Acid, Neomycin, Netilmicin, Nitrofurantoin, Norfloxacin, Ofloxacin, Oxacillin, Oxazolidinones, Oxolinic Acid, Oxytetracycline, Oxytetracycline, Paromomycin, Pazufloxacin, Pefloxacin, Penicillin G, Penicillin V, Pipemidic Acid, Piperacillin, Piromidic Acid, Pivampicillin, Pivmecillinam, Platensimycin, Polymyxin B, Pristinamycin, Prontosil, Prulifloxacin, Pvampicillin, Pyrazinamide, Quinupristin/dalfopristin, Rifabutin, Rifalazil, Rifampin, Rifamycin, Rifapentine, Rosoxacin, Roxithromycin, Rufloxacin, Sitafloxacin, Sparfloxacin, Spectinomycin, Spiramycin, Streptomycin, Sulbactam, Sulfacetamide, Sulfamethizole, Sulfamethoxazole, Sulfanilimide, Sulfisoxazole, Sulphonamides, Sultamicillin, Teicoplanin, Telavancin, Telithromycin, Temafloxacin, Tetracycline, Thiamphenicol, Ticarcillin, Tigecycline, Tinidazole, Tobramycin, Tosufloxacin, Trimethoprim, Trimethoprim-Sulfamethoxazole, Troleandomycin, Trovafloxacin, Tuberactinomycin, Vancomycin, Viomycin, or pharmaceutically acceptable salts thereof (e.g., such as, for example, chloride, bromide, iodide, and periodate), or a combination thereof. As used herein, the recitation of an anti-bacterial agent inherently encompasses the pharmaceutically acceptable salts thereof.

### 4. Anti-Fungal Agents

Anti-fungal agents are known to the art. The art generally recognizes several categories of anti-fungal agents including (1) azoles (imidazoles), (2) antimetabolites, (3) allylamines, (4) morpholine, (5) glucan synthesis inhibitors (echinocandins), (6) polyenes, (7) benoxaaborale; (8) other antifungal/onychomycosis agents, and (9) new classes of antifungal/onychomycosis agents. For example, as used herein, an anti-fungal agent can comprise Abafungin, Albaconazole, Amorolfin, Amphotericin B, Anidulafungin, Bifonazole, Butenafine, Butoconazole, Candicidin, Caspofungin, Ciclopirox, Clotrimazole, Econazole, Fenticonazole, Filipin, Fluconazole, Flucytosine, Griseofulvin, Haloprogin, Hamycin, Isavuconazole, Isoconazole, Itraconazole, Ketoconazole, Micafungin, Miconazole, Naftifine, Natamycin, Nystatin, Omoconazole, Oxiconazole, Polygodial, Posaconazole, Ravuconazole, Rimocidin, Sertaconazole, Sulconazole, Terbinafine, Terconazole, Tioconazole, Tolnaftate, Undecylenic Acid, Voriconazole, or pharmaceutically acceptable salts thereof, or a combination thereof. In an aspect, an anti-fungal agent can be an azole. Azoles include, but are not limited to, the following: clotrimazole, econazole, fluconazole, itraconazole, ketoconazole, miconazole, oxiconazole, sulconazole, and voriconazole. As used herein, the recitation of an anti-fungal agent inherently encompasses the pharmaceutically acceptable salts thereof.

### 5. Anti-Viral Agents

Anti-viral agents are known to the art. As used herein, for example, an anti-viral can comprise Abacavir, Acyclovir (Aciclovir), Adefovir, Amantadine, Ampligen, Amprenavir (Agenerase), Umifenovir (Arbidol), Atazanavir, Atripla, Baloxavir marboxil (Xofluza), Biktarvy, Boceprevir, Bulevirtide, Cidofovir, Cobicistat (Tybost), Combivir, Daclatasvir (Daklinza), Darunavir, Delavirdine, Descovy, Didanosine, Docosanol, Dolutegravir, Doravirine (Pifeltro), Edoxudine, Efavirenz, Elvitegravir, Emtricitabine, Enfuvirtide, Entecavir, Etravirine (Intelence), Famciclovir, Fomivirsen, Fosamprenavir, Foscamet, Ganciclovir (Cytovene), Ibacitabine, Ibalizumab (Trogarzo), Idoxuridine, Imiquimod, Imunovir, Indinavir, Lamivudine, Letermovir (Prevymis), Lopinavir, Loviride, Maraviroc, Methisazone, Moroxydine, Nelfinavir, Nevirapine, Nexavir (formerly Kutapressin), Nitazoxanide, Norvir, Oseltamivir (Tamiflu), Penciclovir, Peramivir, Penciclovir, Peramivir (Rapivab), Pleconaril, Podophyllotoxin, Raltegravir, Remdesivir, Ribavirin, Rilpivirine (Edurant), Rilpivirine, Rimantadine, Ritonavir, Saquinavir, Simeprevir (Olysio), Sofosbuvir, Stavudine, Taribavirin (Viramidine), Telaprevir, Telbivudine (Tyzeka), Tenofovir alafenamide, Tenofovir disoproxil, Tenofovir, Tipranavir, Trifluridine, Trizivir, Tromantadine, Truvada, Umifenovirk, Valaciclovir, Valganciclovir (Valtrex), Vicriviroc, Vidarabine, Zalcitabine, Zanamivir (Relenza), Zidovudine, and combinations thereof. As used herein, the recitation of any anti-viral agent inherently encompasses the pharmaceutically acceptable salts thereof.

### 6. Corticosteroids

Corticosteroids are well-known in the art. Corticosteroids mimic the effects of hormones that the body produces naturally in your adrenal glands. Corticosteroids can suppress inflammation and can reduce the signs and symptoms of inflammatory conditions (e.g., arthritis and asthma). Corticosteroids can also suppress the immune system. Corticosteroids can act on a number of different cells (e.g., mast cells, neutrophils, macrophages and lymphocytes) and a number of different mediators (e.g., histamine, leukotriene, and cytokine subtypes).

Steroids include, but are not limited to, the following: triamcinolone and its derivatives (e.g., diacetate, hexacetonide, and acetonide), betamethasone and its derivatives (e.g., dipropionate, benzoate, sodium phosphate, acetate, and valerate), dexamethasone and its derivatives (e.g., dipropionate and valerate), flunisolide, prednisone and its derivatives (e.g., acetate), prednisolone and its derivatives (e.g., acetate, sodium phosphate, and tebutate), methylprednisolone and its derivatives (e.g., acetate and sodium succinate), fluocinolone and its derivatives (e.g., acetonide), diflorasone and its derivatives (e.g., diacetate), halcinonide, desoximetasone (desoxymethasone), diflucortolone and its derivatives (e.g., valerate), flucloronide (fluclorolone acetonide), fluocinonide, fluocortolone, fluprednidene and its derivatives (e.g., acetate), flurandrenolide (flurandrenolone), clobetasol and its derivatives (e.g., propionate), clobetasone and its derivatives (e.g., butyrate), alclometasone, flumethasone and its derivatives (e.g., pivalate), fluocortolone and its derivatives (e.g., hexanoate), amcinonide, beclometasone and its derivatives (e.g., dipropionate), fluticasone and its derivatives (e.g., propionate), difluprednate, prednicarbate, flurandrenolide, mometasone, and desonide. As used herein, the recitation of a corticosteroid inherently encompasses the pharmaceutically acceptable salts thereof.

### 7. Analgesics

The compositions of the present disclosure can also be used in combination therapies with opioids and other analgesics, including narcotic analgesics, Mu receptor antagonists, Kappa receptor antagonists, non-narcotic (i.e., non-addictive) analgesics, monoamine uptake inhibitors, adenosine regulating agents, cannabinoid derivatives, Substance P antagonists, neurokinin-1 receptor antagonists and sodium channel blockers, among others. Preferred combination therapies comprise a composition useful in methods described herein with one or more compounds selected from aceclofenac, acemetacin, .alpha.-acetamidocaproic acid, acetaminophen, acetaminosalol, acetanilide, acetylsalicylic acid (aspirin), S-adenosylmethionine, alclofenac, alfentanil, allylprodine, alminoprofen, aloxiprin, alphaprodine, aluminum bis (acetylsalicylate), amfenac, aminochlorthenoxazin, 3-amino-4-hydroxybutyric acid, 2-atnino-4-picoline, aminopropylon, aminopyrine, amixetrine, ammonium salicylate, ampiroxicam, amtolmetin guacil, anileridine, antipyrine, antipyrine salicylate, antrafenine, apazone, bendazac, benorylate, benoxaprofen, benzpiperylon, benzydamine, benzylmorphine, bermoprofen, bezitramide, .alpha.-bisabolol, bromfenac, p-bromoacetanilide, 5-bromosalicylic acid acetate, bromosaligenin, bucetin, bucloxic acid, bucolome, bufexamac, bumadizon, buprenorphine, butacetin, butibufen, butophanol, calcium acetylsalicylate, carbamazepine, carbiphene, carprofen, carsalam, chlorobutanol, chlorthenoxazin, choline salicylate, cinchophen, cinmetacin, ciramadol, clidanac, clometacin, clonitazene, clonixin, clopirac, clove, codeine, codeine methyl bromide, codeine phosphate, codeine sulfate, cropropamide, crotethamide, desomorphine, dexoxadrol, dextromoramide, dezocine, diampromide, diclofenac sodium, difenamizole, difenpiramide, diflunisal, dihydrocodeine, dihydrocodeinone enol acetate, dihydromorphine, dihydroxyalutninum acetylsalicylate, dimenoxadol, dimepheptanol, dimethylthiambutene, dioxaphetyl butyrate, dipipanone, diprocetyl, dipyrone, ditazol, droxicam, emorfazone, enfenamic acid, epirizole, eptazocine, etersalate, ethenzamide, ethoheptazine, ethoxazene, ethylmethylthiambutene, ethylmorphine, etodolac, etofenamate, etonitazene, eugenol, felbinac, fenbufen, fenclozic acid, fendosal, fenoprofen, fentanyl, fentiazac, fepradinol, feprazone, floctafenine, flufenamic acid, flunoxaprofen, fluoresone, flupirtine, fluproquazone, flurbiprofen, fosfosal, gentisic acid, glafenine, glucametacin, glycol salicylate, guaiazulene, hydrocodone, hydromorphone, hydroxypethidine, ibufenac, ibuprofen, ibuproxam, imidazole salicylate, indomethacin, indoprofen, isofezolac, isoladol, isomethadone, isonixin, isoxepac, isoxicam, ketobemidone, ketoprofen, ketorolac, p-lactophenetide, lefetamine, levorphanol, lofentanil, lonazolac, lomoxicam, loxoprofen, lysine acetylsalicylate, magnesium acetylsalicylate, meclofenamic acid, mefenamic acid, meperidine, meptazinol, mesalamine, metazocine, methadone hydrochloride, methotrimeprazine, metiazinic acid, metofoline, metopon, mofebutazone, mofezolac, morazone, morphine, morphine hydrochloride, morphine sulfate, morpholine salicylate, myrophine, nabumetone, nalbuphine, 1-naphthyl salicylate, naproxen, narceine, nefopam, nicomorphine, nifenazone, niflumic acid, nimesulide, 5′-nitro-2′-propoxyacetanilide, norlevorphanol, normethadone, normorphine, norpipanone, olsalazine, opium, oxaceprol, oxametacine, oxaprozin, oxycodone, oxymorphone, oxyphenbutazone, papaveretum, paranyline, parsalmide, pentazocine, perisoxal, phenacetin, phenadoxone, phenazocine, phenazopyridine hydrochloride, phenocoll, phenoperidine, phenopyrazone, phenyl acetylsalicylate, phenylbutazone, phenyl salicylate, phenyramidol, piketoprofen, piminodine, pipebuzone, piperylone, piprofen, pirazolac, piritramide, piroxicam, pranoprofen, proglumetacin, proheptazine, promedol, propacetamol, propiram, propoxyphene, propyphenazone, proquazone, protizinic acid, ramifenazone, remifentanil, rimazolium metilsulfate, salacetamide, salicin, salicylamide, salicylamide o-acetic acid, salicylsulfuric acid, salsalte, salverine, simetride, sodium salicylate, sufentanil, sulfasalazine, sulindac, superoxide dismutase, suprofen, suxibuzone, talniflumate, tenidap, tenoxicam, terofenamate, tetrandrine, thiazolinobutazone, tiaprofenic acid, tiaramide, tilidine, tinoridine, tolfenamic acid, tolmetin, tramadol, tropesin, viminol, xenbucin, ximoprofen, zaltoprofen and zomepirac. Analgesics are well known in the art. See, for example, The Merck Index, 12th Edition (1996), Therapeutic Category and Biological Activity Index, and the lists provided under “Analgesic”, “Anti-inflammatory” and “Antipyretic”. As used herein, the recitation of an analgesic inherently encompasses the pharmaceutically acceptable salts thereof.

### 8. Immunostimulants

The term “immunostimulant” is used herein to describe a substance which evokes, increases, and/or prolongs an immune response to an antigen. Immunomodulatory agents modulate the immune system, and, as used herein, immunostimulants are also referred to as immunomodulatory agents, where it is understood that the desired modulation is to stimulate the immune system. There are two main categories of immunostimulants, specific and non-specific. Specific immunostimulants provide antigenic specificity in immune response, such as vaccines or any antigen, and non-specific immunostimulants act irrespective of antigenic specificity to augment immune response of other antigen or stimulate components of the immune system without antigenic specificity, such as adjuvants and non-specific immunostimulators.

Immunostimulants can include, but are not limited to, levamisole, thalidomide, erythema nodosum leprosum, BCG, cytokines such as interleukins or interferons, including recombinant cytokines and interleukin 2 (aldeslukin), 3D-MPL, QS21, CpG ODN 7909, miltefosine, anti-PD-1 or PD-1 targeting drugs, and acid (DCA, a macrophage stimulator), imiquimod and resiquimod (which activate immune cells through the toll-like receptor 7), chlorooxygen compounds such as tetrachlorodecaoxide (TCDO), agonistic CD40 antibodies, soluble CD40L, 4-1BB:4-1BBL agonists, OX40 agonists, TLR agonists, moieties that deplete regulatory T cells, arabinitol-ceramide, glycerol-ceramide, 6-deoxy and 6-sulfono-myo-insitolceramide, iNKT agonists, and TLR agonists. As used herein, the recitation of an immunostimulant inherently encompasses the pharmaceutically acceptable salts thereof.

### 9. Immune-Based Product

As used herein, immune-based products include, but are not limited to, toll-like receptors modulators such as tlr1, tlr2, tlr3, tlr4, tlr5, tlr6, tlr7, tlr8, tlr9, tlr10, tlr11, tlr12, and tlr13; programmed cell death protein 1 (Pd-1) modulators; programmed death-ligand 1 (Pd-L1) modulators; IL-15 agonists; DermaVir; interleukin-7; plaquenil (hydroxychloroquine); proleukin (aldesleukin, IL-2); interferon alfa; interferon alfa-2b; interferon alfa-n3; pegylated interferon alfa; interferon gamma; hydroxyurea; mycophenolate mofetil (MPA) and its ester derivative mycophenolate mofetil (MMF); ribavirin; rintatolimod, polymer polyethyleneimine (PEI); gepon; rintatolimod; IL-12; WF-10; VGV-1; MOR-22; BMS-936559; CYT-107, interleukin-15/Fc fusion protein, normferon, peginterferon alfa-2a, peginterferon alfa-2b, recombinant interleukin-15, RPI-MN, GS-9620, and IR-103. As used herein, the recitation of an immune-based product inherently encompasses the pharmaceutically acceptable salts thereof.

### F. Kits

Disclosed herein is a kit comprising a disclosed isolated nucleic acid molecule, a disclosed vector, a disclosed pharmaceutical formulation, a disclosed RNA therapeutic, or a combination thereof. In an aspect, a kit can comprise a disclosed isolated nucleic acid molecule, a disclosed vector, a disclosed pharmaceutical formulation, a disclosed RNA therapeutic, or a combination thereof, and one or more agents. “Agents” and “Therapeutic Agents” are known to the art and are described supra. In an aspect, the one or more agents can treat, prevent, inhibit, and/or ameliorate one or more comorbidities in a subject. In an aspect, one or more active agents can treat, inhibit, prevent, and/or ameliorate a GSD IX al, GSD IX α2, GSD IX J, GSD IX δ, GSD IX γ2, and/or GSD VI symptom or a GSD IX al, GSD IX α2, GSD IX J, GSD IX δ, GSD IX γ2, and/or GSD VI related complication.

In an aspect, a disclosed kit can comprise at least two components constituting the kit. Together, the components constitute a functional unit for a given purpose (such as, for example, treating a subject diagnosed with or suspected of having GSD IX al, GSD IX α2, GSD IX 3, GSD IX δ, GSD IX γ2, and/or GSD VI). Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation. In an aspect, a kit for use in a disclosed method can comprise one or more containers holding a disclosed isolated nucleic acid molecule, a disclosed vector, a disclosed pharmaceutical formulation, a disclosed RNA therapeutic, or a combination thereof, and a label or package insert with instructions for use. In an aspect, suitable containers include, for example, bottles, vials, syringes, blister pack, etc. The containers can be formed from a variety of materials such as glass or plastic. The container can hold a disclosed isolated nucleic acid molecule, a disclosed vector, a disclosed pharmaceutical formulation, or a combination thereof, and can have a sterile access port (for example the container may be an intravenous solution bag or a vial having a stopper pierceable by a hypodermic injection needle). The label or package insert can indicate that a disclosed isolated nucleic acid molecule, a disclosed vector, a disclosed pharmaceutical formulation, a disclosed RNA therapeutic, or a combination thereof can be used for treating, preventing, inhibiting, and/or ameliorating GSD IX al, GSD IX α2, GSD IX J, GSD IX δ, GSD IX γ2, and/or GSD VI or complications and/or symptoms associated with GSD IX al, GSD IX α2, GSD IX 3, GSD IX δ, GSD IX γ2, and/or GSD VI. A kit can comprise additional components necessary for administration such as, for example, other buffers, diluents, filters, needles, and syringes. In an aspect, one or more active agents can reduce the expression level and/or activity level of glycogen synthase. These one or more active agents include agents for SRT and/or small molecules (e.g., guaiacol).

### VII. EXAMPLES

To date, there have been four animal models described with PhK deficiency. These animals arose spontaneously in nature or were transgenically derived. (Walvoort 1983; Akman et al., 2011). The I-mouse strain was associated with muscle-specific PhK deficiency and an X-linked recessive mutation in the Phkal gene. (Gross et al., 1976; Mefford et al., 2013; Varsanyi et al., 1978; Lyon et al., 1963). The V-mouse strain was associated with partial muscle-specific PhK deficiency and an X-linked dominant mutation in the Phka1 gene. (Varsfinyi, et al., 1980). The University of Connecticut School of Medicine mouse model was associated with liver and muscle PhK deficiency as well as autosomal recessive mutations in the Phkb gene. (Wilson et al., 2018). The only liver specific GSD IX model was identified in an inbred strain of NZR/Mh rats (gsd/gsd) that was discovered four decades ago with liver-specific PhK deficiency, and later identified to have autosomal recessive mutations in the Phkg2 gene. (Maichele A J, et al. (1996) Nat. Genet. 14:337-340; Malthus R, et al. (1980) Biochem. J. 188:99-106; Haynes P, et al. (1983) 42:289-301). However, this rat model has not been studied in publication since 1996. (Maichele A J, et al. (1996) Nat. Genet. 14:337-340). Mice are a preferable model to rats as they are smaller in size, have a shorter gestation period, and generally cost less to maintain. (Bryda E C, et al. (2013) Mo. Med. 110:207-211). Thus, there is an urgent need for a viable GSD IX and GSD VI mouse models.

The Examples that follow are illustrative of specific embodiments of the invention, and various uses thereof. They set forth for explanatory purposes only and are not to be taken as limiting the invention.

### Example 1

### Generation of GSD IX γ2 Mice (Phkg2^{-/-}) and Verification at the DNA and Protein Levels

The investigation of disease progression and evaluation of novel therapeutics relies on the development of robust animal models. (Guénet J L, et al. (2011) Mol. Genet. Genomic Med. 286:1-20; Smithies O, et al. (1993) Trends Genet. 9:112-116; Wine J J, et al. (2002) Methods Mol. Med. 70:31-46). The first mouse model for GSD IX γ2 was created and characterized herein. Heterozygous Phkg2 mutant (Phkg2^{+/-}) mice carrying the Phkg2^{tm1.1} knockout allele (C57BL/6N-Phkg2^{tm1.1(KOMP)Vlcg}/JMmucd, RRID:MMRRC_049060-UCD) were purchased from the Mutant Mouse Resource and Research Center (MMRRC) at University of California at Davis. These mice were then cross-bred to produce homozygous Phkg2 knockout (KO, Phkg2^{-/-}) mice and wild-type (WT, Phkg2^{+/+}) controls. Pieces of tails were collected for genomic DNA extraction to confirm genotype by PCR. Primer pairs GGGAGCAGGGATTGCTACTG (Forward 1) and TTCCTCAGGGTTCCTGTTCTG (Reverse 1) were used for genotyping the KO allele and GGTAAACTGGCTCGGATTAGGG (Forward 2) and TTGACTGTAGCGGCTGATGTTG (Reverse 2) were used for the WT allele. (Valenzuela D M, et al. (2003) Nat. Biotechnol. 21:652-659; Dickinson M E, et al. (2016) Nature. 537:508-514). The primers are summarized in Table 5 provided below.

TABLE 5
Primers for Phkg2^{-/-} and Wild-Type Mice

| Description | Sequence | SEQ ID NO. |
|---|---|---|
| Forward 1 for KO | GGGAGCAGGGATTGCTACTG | SEQ ID NO: 23 |
| Reverse 1 for KO | TTCCTCAGGGTTCCTGTTCTG | SEQ ID NO: 24 |
| Forward 2 for WT | GGTAAACTGGCTCGGATTAGGG | SEQ ID NO: 25 |
| Reverse 2 for WT | TTGACTGTAGCGGCTGATGTTG | SEQ ID NO: 26 |

The Phkg2^{tm1} knockout allele was generated with a targeting vector by replacing critical exons containing the entire PhK coding regions in the Phkg2 allele with a DNA fragment containing the lacZ and LoxP-floxed neomycin expression cassettes. As illustrated in FIG. 2A, the Phkg2^{tm1.1} knockout allele was generated via Cre-mediated excision of the neomycin selection cassette from the parental Phkg2^{tm1.1} knockout allele. As shown in FIG. 2B, genotype was confirmed via genomic DNA extraction, PCR, and gel electrophoresis. The first PCR product at 210 bp in length was amplified from the KO allele using the F1 and R1 primers. The second PCR product at 136 bp in length was amplified via PCR from the WT allele using the F2 and R2 primers. Primers added to genomic DNA of heterozygous mice (HT) generated both the KO and WT PCR products. M indicates the size marker.

### Example 2

### Phkg2^{-/-} Mice Experienced Hepatomegaly

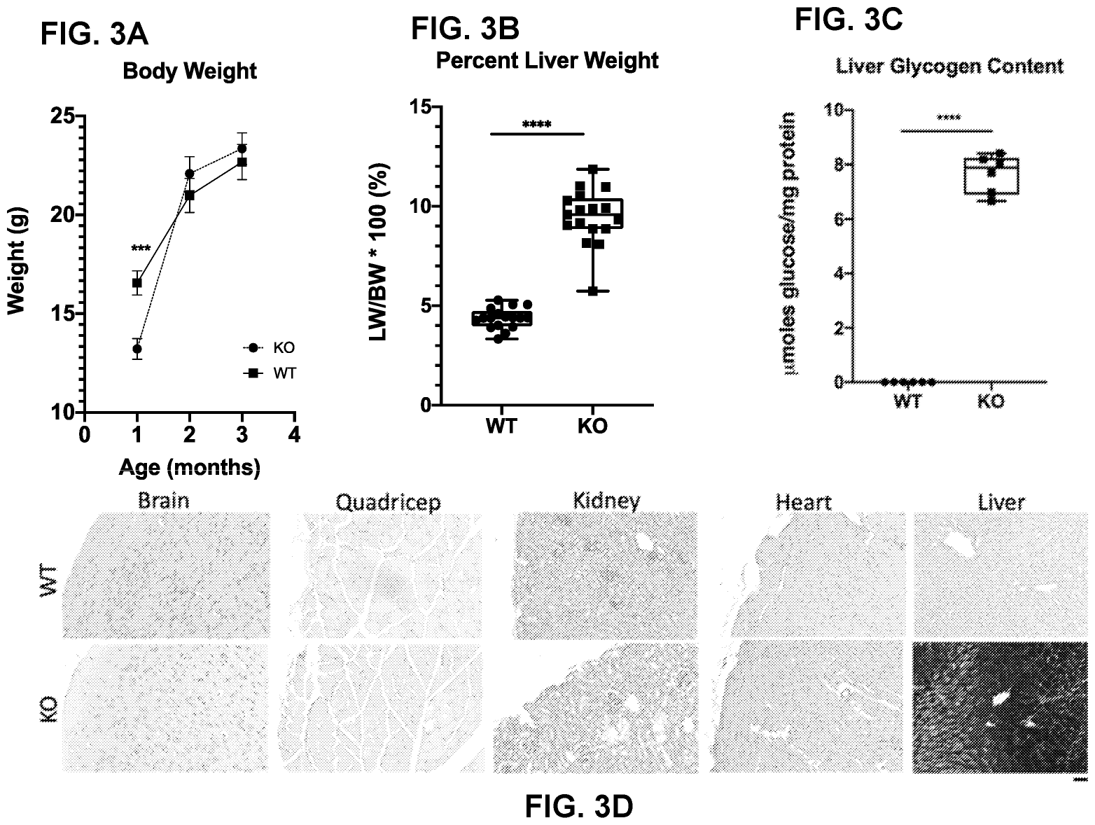

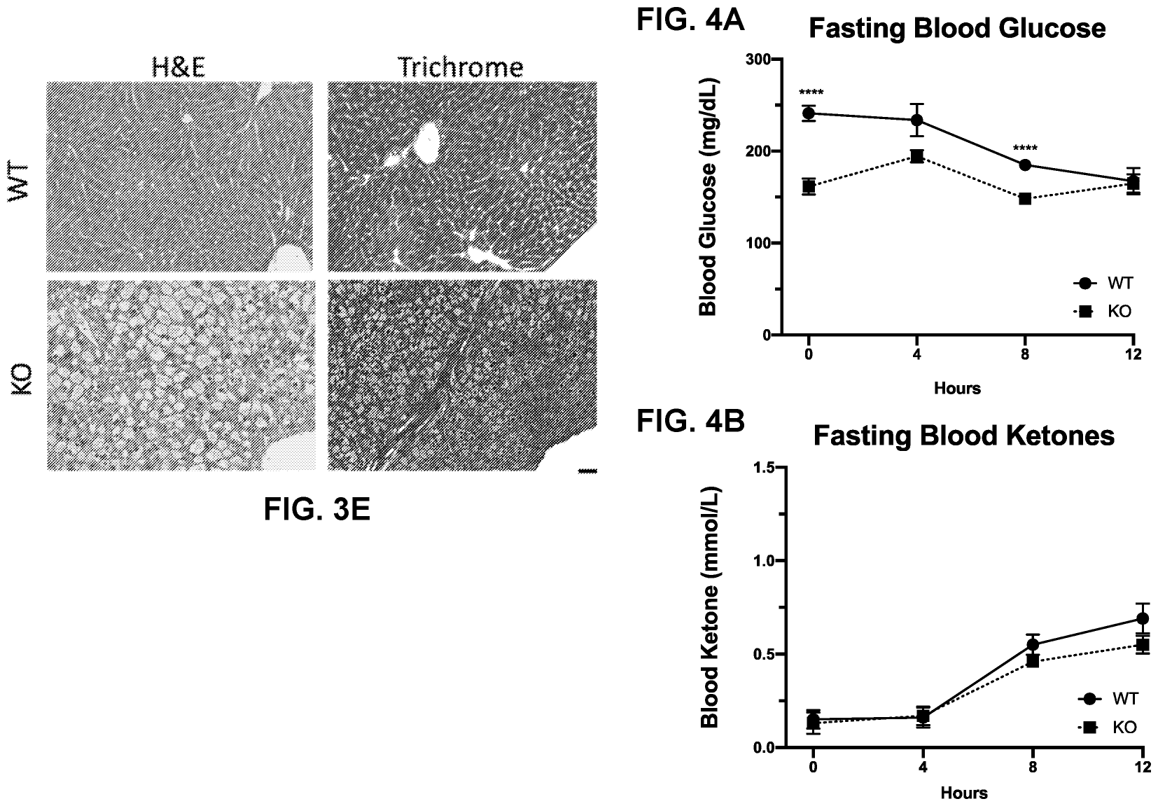

As GSD IX is associated with delayed growth, the body weight and liver weight of the newly generated knockout mice were measured. Specifically, body weight was measured at 1 month, 2 months, and 3 months of age. At 3 months of age, mice were euthanized and dissected. Whole liver was collected and weighed. Liver weight was reported as a percent of body weight using the ratio of Liver weight/Body Weight (LW/BW)*100, a metric for hepatomegaly. A growth curve was developed for total body weight in male and female WT (n=46) and KO mice (n=46). As FIG. 3A shows, KO mice demonstrated significantly lower average body weight at 1 month of age (p<0.001) then did the WT mice. When mice were weaned at 1 month, they were given a constant food source and no longer needed to compete with WT littermates. There was no difference in body weight between KO and WT mice at 2 months or age. Similarly, there was no difference in body weight between KO and WT mice at 3 months of age. Liver weight (LW) was measured and was normalized to overall body weight (BW) as a quantitative metric for enlarged liver or hepatomegaly. FIG. 3B shows that the % liver weight (LW/BW*100) was significantly higher (p<0.0001) in KO mice (n=21) when compared to WT mice (n=17). Min to max demonstrating all points.

### Example 3

### Phkg2^{-/-} Mice Produced Less γ2 Subunit than Wild-Type Mice

As PhK is one of the most upstream enzymes in the glycogenolysis pathway, the production of liver isoforms of the four PhK subunits in GSD IX γ2 Mice (Phkg2^{-/-}) were examined. Tissues were homogenized in cold lysis buffer [PBS containing 1% NP40, 0.5% sodium deoxycholate, 0.1% SDS, and a protease/phosphatase inhibitor cocktail (Cell Signaling Technology, Danvers, MA)] using an electric homogenizer. Tissue lysates were cleared by centrifugation at 18,000 g at 4° C. for 15 minutes. Protein concentrations of the supernatants were measured by BCA protein assay. Equal amounts of protein were loaded and separated on SDS-PAGE gels and transferred to nitrocellulose membranes. The membranes were blocked in 3% BSA/PBST, incubated with primary antibodies overnight at 4° C., washed, incubated with secondary antibodies, washed again, and developed using ECL kit (Bio-rad, Hercules, CA). The images were obtained by the image analyzer (Bio-rad, Hercules, CA). (Lim J A, et al. (2019) Mol. Ther.—Methods Clin. Dev. 12:233-245). The following antibodies were used: anti-PHKG2 (Proteintech, Rosemont, IL), anti-PHKB (Proteintech, Rosemont, IL), anti-PHKA2 (Proteintech, Rosemont, IL), anti-CALM (ThermoFisher Scientific, Waltham, MA), anti-Beta actin (Sigma-Aldrich Co., St. Louis, MO), and anti-rabbit IgG-Peroxidase (Sigma-Aldrich Co., St. Louis, MO).

As shown in FIG. 2C, Western blot was performed for each subunit of the liver PhK enzyme: α2, β3, γ2, and calmodulin δ. Western blot analysis demonstrated a decreased level of γ2 subunit protein in KO mice (n=3) when compared to WT mice (n=3). Western blot analysis also demonstrated that there were increased amounts of α2 and β protein in KO mice when compared with WT mice. There was no difference in the δ (calmodulin) protein in KO mice when compared with WT mice. Beta actin served as the loading control. Phosphorylase kinase (PhK) activity was significantly less in KO mice compared to WT controls. WT n=6, KO n=6. Min to max demonstrating all points. ****p<0.0001.

### Example 4

### PhK Activity was Decreased in Phkg2^{-/-} Mice

To measure PhK enzymatic activity in GSD IX γ2 knockout mice (Phkg2^{-/-}), frozen liver samples were examined using standard spectrophotometric methods by the Duke University Health System Glycogen Storage Disease Laboratory. Enzyme activity was measured indirectly by measuring the amount of glucose released from glycogen substrate using glucose reagent (Infinity™; Cat No. TR15421) from ThermoScientific (Fisher Diagnostics, Middletown, VA, USA). Enzyme activity was expressed as pmol/min/g of liver tissue. (Bali D S, et al. (2014) Mol. Genet. Metab. 111:309-313). FIG. 2D shows that PhK enzyme activity was significantly decreased (p<0.0001) in KO mice (n=6) when compared with WT mice (n=6). Min to max demonstrating all points. ****p<0.0001.

### Example 5

### Phkg2^{-/-} Mice Demonstrated Significant Increases in Liver Glycogen

As continued glycogen accumulation in the liver leads to hepatomegaly and progressive liver fibrosis, histology was performed. Fresh tissue samples were fixed in 10% NBF for 48 hours and post-fixed with 1% periodic acid (PA) in 10% NBF for 48 hours at 4° C. Samples were washed with PBS, dehydrated with ascending grades of alcohol, cleared with xylene, and infiltrated with paraffin. Sections were cut with a microtome and mounted for staining. For Periodic acid-Schiff (PAS), slides were processed based on previously described methodology. (Lim J A, et al. (2019)). In brief, slides were deparaffinized and rehydrated. Slides were oxidized with freshly made 0.5% PA for 5 minutes and rinsed with distilled water for 1 minute. Slides were then stained with Schiff reagent for 15 minutes and washed with tap water for 10 minutes. Slides were counterstained with Hematoxylin, rinsed with tap water, and incubated with bluing reagent for 1 minute. Slides were then dehydrated and mounted. The periodic acid oxidizes glycogen to an aldehyde product which reacts with Schiff reagent to produce a purple stain. As FIG. 3D shows, there was minimal difference in the PAS staining between KO and WT mice in the brain, quadriceps, kidney, and heart. But the PAS staining in the liver was markedly different between the KO mice and WT mice. In FIG. 3D, images were captured at 20× and the scale bar is 50 μm. To verify PAS stain, a glycogen assay was performed on liver lysates from WT and KO mice. Liver tissue from KO mice demonstrate significantly higher glycogen content levels than WT controls. This remarkable increase in PAS staining in the liver of KO mice was also verified with a glycogen content assay. (FIG. 3C, n=6 WT and n=6 KO). Min to max demonstrating all points. ****p<0.0001.

### Example 6

### Phkg2^{-/-} Mice Demonstrated Characteristic GSD Hepatocyte Architectural Changes and Early Perisinusoidal Fibrosis

As GSD IX γ2 is associated with hepatomegaly, fibrosis, and carcinoma, fresh tissue samples were subjected to histology based on previously described methodology. (Cardiff R D, et al. (2014) Cold Spring Harb. Protoc. 2014:655-658). In brief, slides were deparaffinized and rehydrated. Slides were stained with hematoxylin solution for 3 minutes, washed with running water and then stained with Eosin Y solution for 2 minutes to define hepatocyte architecture. Slides were then dehydrated and mounted. For Masson's Trichrome staining, slides were treated using the Sigma-Aldrich Masson's Trichrome Stain Kit No. HT15. Slides were deparaffinized and rehydrated. Slides were incubated in Bouin's solution at room temperature overnight. Slides were washed with running tap water, stained with Weigert's Iron Hematoxylin solution for 5 minutes, washed with running tap water, stained with Biebrich Scarlet-Acid Fuschin for 5 minutes, rinsed with distilled water, stained with phosphotungstic/phosphomolybdic acid for 5 minutes, rinsed with distilled water, stained in Aniline Blue Solution for 5 minutes, rinsed with distilled water, stained in 1% acetic acid for 2 minutes, and rinsed in distilled water. Slides were then dehydrated and mounted. All imaging of slides was performed using a BZ-X710 microscope (Keyence America, Itasca, IL). In FIG. 3E discussed below, the images were captured at 20× and the scale bar is 50 μm.

FIG. 3E shows that KO mice demonstrated heterogeneously enlarged hepatocytes with pale cytoplasm and distinct cell membranes as well as concentrated, lateralized, and pyknotic nuclei. Slides were stained with Hematoxylin & Eosin (H&E) to identify hepatocyte architecture. These hepatocyte architectural changes are characteristic of the changes seen in patients with liver GSDs. (Halaby C A, et al. (2019) Genet. Med. 21:2686-2694). There was no evidence of hepatocyte architectural changes in WT mice. KO mice (bottom panels) demonstrate heterogeneously enlarged hepatocytes with pale cytoplasm and distinct cell membranes as well as lateralized, pyknotic nuclei. Slides were stained with Masson's Trichrome to identify tissue fibrosis. FIG. 3E demonstrated that KO mice showed evidence of early perisinusoidal liver fibrosis, which is seen in patients with liver GSDs. (Halaby C A, et al. (2019) Genet. Med. 21:2686-2694; Degrassi M, et al. (2020) Dig. Liver Dis. 42:459-469). There was no evidence of liver fibrosis in WT mice. Images were captured at 20×. Scale bar is 50 μm.

### Example 7

### Liver Glycogen Content is Significantly Elevated in Phkg2^{-/-} Mice

To measure glycogen accumulation (a hallmark of GSD IX) in the tissues of GSD IX γ2 knockout mice (Phkg2), frozen tissues were homogenized in distilled water (1 mg of tissue/20 μL of water) using an electric homogenizer. Homogenization was followed by sonication for 15 seconds and centrifugation at 18,000 g at 4° C. for 15 minutes. The 1:5 diluted lysates were boiled for 3 minutes to inactivate endogenous enzymes. Samples were incubated with 0.175 U/mL (final concentration in the reaction) of amyloglucosidase (Sigma-Aldrich Co., St. Louis, MO) for 90 minutes at 37° C. The mixtures were boiled for 3 minutes to stop the reaction. 30 μL of the mixtures were incubated with 1 mL of Pointe Scientific Glucose Hexokinase Liquid Reagent (Fisher, Hampton, NH) for 10 minutes at room temperature. Absorbance was measured at 340 nm with a UV-VIS spectrophotometer (Shimadzu UV-1700 PharmaSpec). (Lim J A, et al (2019)). FIG. 3C shows that glycogen content in the liver was significantly elevated (p<0.0001) in KO mice (n=6) when compared with WT mice (n=6). Min to max demonstrating all points. The significant difference in glycogen accumulation specific to the liver of KO mice is a key characteristic of liver GSD IX γ2.

### Example 8

### Phkg2^{-/-} Mice Demonstrated a Significant Decrease in Blood Glucose, an Increase in ALP, AST, ALT, and an Increase in Urine Hex4 Levels

Due to the disruption of the glycogenolysis pathway in GSD IX γ2 mice (Phkg2), blood was sampled via submandibular bleeding. Blood glucose concentration was measured using the AlphaTRAK2 whole blood glucometer and blood ketone level was measured using the Nova Max Glucose Ketone Meter. (Ayala J E, et al. (2010) DMM Dis. Model. Mech. 3:525-534). Additional blood was collected at 3 months of age after 24 hours of fasting via submandibular bleed in a red top collection tube and centrifuged at 2,000 g, 4° C. for 10 minutes to isolate serum. Serum samples were sent out for liver and lipid panel testing by a commercial laboratory (IDEXX Laboratories, Inc.). The liver panel included alkaline phosphatase (ALP), aspartate aminotransferase (AST), alanine aminotransferase (ALT), conjugated bilirubin (CB), unconjugated bilirubin (UCB), total bilirubin (TB), creatine kinase (CK), total protein (TP), albumin (ALB), and gamma-glutamyl transferase (GGT). The lipid panel included total cholesterol (TC), triglycerides, low-density lipoprotein (LDL), and high-density lipoprotein (HDL). Urine glucose tetrasaccharide Glcα1-6Glcα1-4Glcα1-4Glc (Glc₄), a biomarker of glycogen storage diseases also referred to as hexose tetrasaccharide (Hex₄), was evaluated in urine samples collected at 3 months of age after 24 hours of fasting. Samples were stored at −80° C. and shipped on dry ice to the Duke University Health System Biochemical Genetics Laboratory for Hex₄ analysis based on previously described mass spectrometry methods. (Young S P, et al. (2020) JIMD Rep. jmd2.12181; Young S P, et al. (2003) Anal. Biochem. 316:175-180).

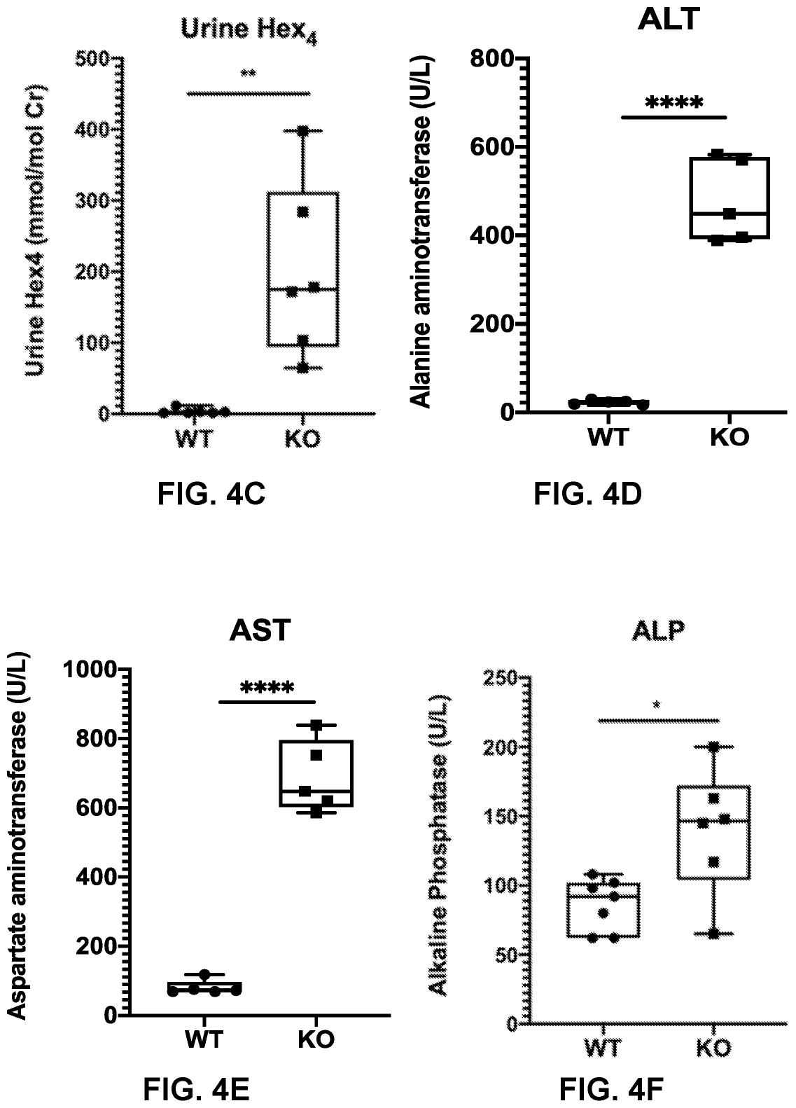

FIG. 4A shows that KO mice demonstrated significant hypoglycemia when compared to WT mice (p<0.05). In other words, blood glucose was significantly lower in KO mice than WT controls. FIG. 4B shows that while ketones varied widely in the KO group (n=7), the ketone values in KO group did not differ significantly when compared to WT mice (n=6). In line with elevated glycogen accumulation seen on PAS stain (FIG. 3D) and glycogen assay (FIG. 3C), FIG. 4C shows there was a significant elevation (p<0.01) in urine Hex₄ in 24 hour fasted KO mice (n=6) when compared to WT mice (n=7). FIG. 4D-FIG. 4F show that liver enzymes including alanine aminotransferase (ALT) (FIG. 4D), aspartate aminotransferase (AST) (FIG. 4E), and ALP (FIG. 4F) were significantly elevated in KO mice (n=6) vs. WT mice (n=6) (ALT (p<0.001), AST (p<0.001), alkaline phosphatase (ALP) (p<0.05)). No significant differences were observed for GGT, TP, Albumin, TB, CB, UCB, TC, triglycerides, LDL, and HDL. All numerical data was evaluated using Prism software version 8 (GraphPad, La Jolla, CA). Statistical analysis of all quantitative data collected was performed using parametric unpaired T-tests to determine the differences between KO and WT groups. One star (*) indicated p value <0.05. Two stars (**) indicated p<0.01. Three stars (***) indicated p<0.001. Four stars (****) indicated p<0.0001. A p value <0.05 was considered a statistically significant difference.

### Example 9

### Summary of Data Presented in Examples 1-8

The disclosed data verified the loss of the Phkg2 gene at the DNA and protein level. At the DNA level, genotyping confirmed that targeted deletion was achieved via insertion of the Velocigene-Regeneron targeted vector into exon 2 of the Phkg2 allele. (See FIG. 2A-FIG. 2B). At the protein level, KO mice demonstrated decreased levels of γ2 subunit protein and decreased PhK enzyme activity when compared to WT mice. (See FIG. 2C-FIG. 2D). This decrease in PhK enzyme activity prevented adequate breakdown of glycogen into glucose, which lead to glycogen accumulation in the liver, the disease defining phenotype of GSD IX γ2. Western blot analysis also demonstrated increased levels of α2 and β subunit proteins. In the Phkg2^{-/-} mouse, glycogen accumulation in the liver manifested as significant hepatomegaly, remarkable liver glycogen accumulation on PAS-stained histology slides (FIG. 3D), and significant liver glycogen on confirmatory glycogen content analysis (FIG. 3C). The Phkg2^{-/-} mouse also demonstrated liver glycogen-associated characteristics. Blood glucose showed significant hypoglycemia in KO mice compared to WT mice. (See FIG. 4A). Blood ketones were found elevated in some KO mice during hypoglycemia, but the differences compared to WT mice were not statistically significant. (See FIG. 4B). Blood liver panel demonstrated significantly elevated ALT (FIG. 4D), AST (FIG. 4E), and ALP (FIG. 4F) levels. Urine Hex₄ was significantly elevated (FIG. 4C). This is the first time that elevated Hex₄ levels, also known as Glc₄, have been associated with liver-only glycogen accumulation in an animal model. Glc₄ has been identified as a robust biomarker in muscle associated glycogen storage diseases such as Pompe (GSD II) (Kishnani P S, et al. (2006) Genet. Med. 8:267-288). In GSD III, Glc₄ can be elevated secondary to glycogen accumulation in liver and/or muscle. (Kishnani P S, et al. (2010) Genet. Med. 12:446-463). Now, for the first time, the data presented herein demonstrate that elevated Glc4 in the Phkg2^{-/-} mouse supports the use of urine Glc₄ as a biomarker for liver-specific GSDs. The disclosed mouse model mirrored the architectural and fibrotic changes seen in GSD IX γ2 patients. (Halaby C A, et al. (2019) Genet. Med. 21:2686-2694; Degrassi M, et al. (2020) Dig. Liver Dis.). H&E-stained liver histology slides from liver GSD IX γ2 patients displayed hepatocytes that were heterogeneously enlarged with distinct cell membrane, pale cytoplasm, and concentrated, pyknotic, and lateralized nuclei. The pale cytoplasm was persistent glycogen accumulation. H&E-stained liver histology slides (left panels) from the disclosed mouse model demonstrated the same characteristic features. (See FIG. 3E). Trichrome stained histology slides (right panels) from liver GSD patients displayed progressive liver fibrosis and damage. Trichrome stained histology slides from the disclosed mouse model at 3 months of age demonstrated early perisinusoidal liver fibrosis, which is a sign of early liver damage secondary to liver glycogen accumulation. (See FIG. 3E). The Phkg2^{-/-} mouse recapitulated the early signs of liver-specific fibrosis seen in patients with liver GSD IX γ2. (Fernandes S A, et al. (2020)). Identification of liver structural changes allows for the description of further liver disease progression. The data described herein characterize—for the first time—a mouse model for liver GSD IX γ2. The data further demonstrated that the model recapitulated the liver-specific glycogen accumulation phenotype of patients with GSD IX γ2.

### Example 10

### Characterization of Aged GSD IX γ2 Mice (Phkg2^{-/-})

To measure the growth of the knockout mice, body weight is measured at 6 months, 7 months, 8 months, 9 months, 10 months, 11 months, and 12 months. Then, at either 6, 9, or 12 months of age, male and female KO mice are fasted for 24 hours and then euthanized for blood, urine, and tissue collection mice. Whole liver is collected and weighed. Liver weight is reported as a percent of body weight using the ratio of Liver weight/Body Weight (LW/BW)*100, a metric for hepatomegaly.

To examine protein levels in those mice euthanized at either 6, 9, or 12 months of age, tissues are homogenized in cold lysis buffer [PBS containing 1% NP40, 0.5% sodium deoxycholate, 0.1% SDS, and a protease/phosphatase inhibitor cocktail (Cell Signaling Technology, Danvers, MA)] using an electric homogenizer. Tissue lysates are cleared by centrifugation at 18,000 g at 4° C. for 15 minutes. Protein concentrations of the supernatants are measured by BCA protein assay. Equal amounts of protein are loaded and separated on SDS-PAGE gels and transferred to nitrocellulose membranes. The membranes are blocked in 3% BSA/PBST, incubated with primary antibodies overnight at 4° C., washed, incubated with secondary antibodies, washed again, and developed using ECL kit (Bio-rad, Hercules, CA). The images are obtained by the image analyzer (Bio-rad, Hercules, CA). (Lim J A, et al (2019)). The following antibodies are used: anti-PHKG2 (Proteintech, Rosemont, IL), anti-PHKB (Proteintech, Rosemont, IL), anti-PHKA2 (Proteintech, Rosemont, IL), anti-CALM (ThermoFisher Scientific, Waltham, MA), anti-Beta actin (Sigma-Aldrich Co., St. Louis, MO), and anti-rabbit IgG-Peroxidase (Sigma-Aldrich Co., St. Louis, MO).

To measure the PhK enzymatic activity in GSD IX γ2 knockout mice (Phkg2^{-/-}), the mice are euthanized at either 6, 9, or 12 months of age, PhK enzyme activity is analyzed in frozen liver samples using standard spectrophotometric methods by the Duke University Health System Glycogen Storage Disease Laboratory. Enzyme activity is measured indirectly by measuring the amount of glucose released from glycogen substrate using glucose reagent (Infinity™; Cat No. TR15421) from ThermoScientific (Fisher Diagnostics, Middletown, VA, USA). Enzyme activity is expressed as μmol/min/g of liver tissue. (Bali D S, et al. (2014) Mol. Genet. Metab. 111:309-313).

To measure glycogen accumulation (a hallmark of GSD IX) in the tissues of GSD IX γ2 knockout mice (Phkg2^{-/-}) euthanized at either 6, 9, or 12 months of age, frozen tissues are homogenized in distilled water (1 mg of tissue/20 μL of water) using an electric homogenizer. Homogenization is followed by sonication for 15 seconds and centrifugation at 18,000 g at 4° C. for 15 minutes. The 1:5 diluted lysates are boiled for 3 minutes to inactivate endogenous enzymes. Samples are incubated with 0.175 U/mL (final concentration in the reaction) of amyloglucosidase (Sigma-Aldrich Co., St. Louis, MO) for 90 minutes at 37° C. The mixtures are boiled for 3 minutes to stop the reaction. 30 μL of the mixtures are incubated with 1 mL of Pointe Scientific Glucose Hexokinase Liquid Reagent (Fisher, Hampton, NH) for 10 minutes at room temperature. Absorbance is measured at 340 nm with a UV-VIS spectrophotometer (Shimadzu UV-1700 PharmaSpec). (Lim J A, et al (2019)).

As continued glycogen accumulation in the liver leads to hepatomegaly and progressive liver fibrosis, histology is performed. For those mice euthanized at either 6, 9, or 12 months of age, fresh tissue samples are fixed in 10% NBF for 48 hours and post-fixed with 1% periodic acid (PA) in 10% NBF for 48 hours at 4° C. Samples are washed with PBS, dehydrated with ascending grades of alcohol, cleared with xylene, and infiltrated with paraffin. Sections are cut with a microtome and mounted for staining.

For Periodic Acid-Schiff (PAS), slides are processed based on previously described methodology. (Lim et al., 2019). In brief, slides are deparaffinized and rehydrated. Slides are oxidized with freshly made 0.5% PA for 5 minutes and rinsed with distilled water for 1 minute. Slides are then stained with Schiff reagent for 15 minutes and washed with tap water for 10 minutes. Slides are counterstained with Hematoxylin, rinsed with tap water, and incubated with bluing reagent for 1 minute. Slides are then dehydrated and mounted. The periodic acid oxidizes glycogen to an aldehyde product which reacts with Schiff reagent to produce a purple stain.

For Hematoxylin and Eosin staining (H&E), slides are treated based on previously described methodology. (Cardiff R D, et al. (2014) Cold Spring Harb. Protoc. 2014:655-658). In brief, slides are deparaffinized and rehydrated. Slides are stained with hematoxylin solution for 3 minutes, washed with running water and then stained with Eosin Y solution for 2 minutes. Slides are then dehydrated and mounted.

For Masson's Trichrome staining, slides are treated using the Sigma-Aldrich Masson's Trichrome Stain Kit No. HT15. Slides were deparaffinized and rehydrated. Slides are incubated in Bouin's solution at room temperature overnight. Slides are washed with running tap water, stained with Weigert's Iron Hematoxylin solution for 5 minutes, washed with running tap water, stained with Biebrich Scarlet-Acid Fuschin for 5 minutes, rinsed with distilled water, stained with phosphotungstic/phosphomolybdic acid for 5 minutes, rinsed with distilled water, stained in Aniline Blue Solution for 5 minutes, rinsed with distilled water, stained in 1% acetic acid for 2 minutes, and rinsed in distilled water. Slides are then dehydrated and mounted. All imaging of slides is performed using a BZ-X710 microscope (Keyence America, Itasca, IL). Stages of progression from glycogen accumulation to liver fibrosis to liver cirrhosis are identified to determine the latest age at which therapy may be able to rescue disease.

Blood is sampled via submandibular bleeding for those mice euthanized at either 6, 9, or 12 months of age. Blood glucose concentration is measured using the AlphaTRAK2 whole blood glucometer and blood ketone level is measured using the Nova Max Glucose Ketone Meter. (Ayala et al., 2010). Additional blood is collected at either 6, 9, or 12 months of age after 24 hours of fasting via submandibular bleed in a red top collection tube and centrifuged at 2,000 g, 4° C. for 10 minutes to isolate serum. Serum samples are sent out for liver and lipid panel testing by a commercial laboratory (IDEXX Laboratories, Inc.). The liver panel includes alkaline phosphatase (ALP), aspartate aminotransferase (AST), alanine aminotransferase (ALT), conjugated bilirubin (CB), unconjugated bilirubin (UCB), total bilirubin (TB), creatine kinase (CK), total protein (TP), albumin (ALB), and gamma-glutamyl transferase (GGT). The lipid panel includes total cholesterol (TC), triglycerides, low-density lipoprotein (LDL), and high-density lipoprotein (HDL). Urine glucose tetrasaccharide Glcα1-6 Glcα1-4Glcα1-4Glc (Glc₄), a biomarker of glycogen storage diseases also referred to as hexose tetrasaccharide (Hex₄), is evaluated in urine samples collected at either 6, 9, or 12 months of age after 24 hours of fasting. Samples are stored at −80° C. and shipped on dry ice to the Duke University Health System Biochemical Genetics Laboratory for Hex₄ analysis based on previously described mass spectrometry methods. (Young S P, et al. (2020) JIMD Rep. jmd2.12181; Young S P, et al. (2003) Anal. Biochem. 316:175-180).

For those mice euthanized at either 6, 9, or 12 months of age, all numerical data is evaluated using Prism software version 8 (GraphPad, La Jolla, CA). Statistical analysis of all quantitative data collected is performed using parametric unpaired T-tests to determine the differences between KO and WT groups. One star (*) indicates p value <0.05. Two stars (**) indicates p<0.01. Three stars (***) indicates p<0.001. Four stars (****) indicates p<0.0001. A p value <0.05 is considered a statistically significant difference.

### Example 11

### Gene Therapy to Reduce Liver Disease in GSD IX γ2 Mouse Model

Viral gene therapy is an ideal therapeutic for the treatment of tissue-specific monogenic diseases like GSD IX γ2. Viral gene therapy capitalizes on a virus's ability to package and transfer genomic material. Current human liver-directed gene therapies utilize an AAV8 capsid (AAV8) based on preclinical efficacy in mice. (Bond J E, et al. (2019) Cell. Immunol. 342:103737; Pipe S, et al. (2019) Mol. Ther.—Methods Clin. Dev. 15:170-178). Recent studies, however, have demonstrated that AAV8 has greater tropism for murine than human hepatocytes. (Lisowski L, et al. (2014) Nature. 506:382-386; Vercauteren K, et al. (2016) Mol. Ther. 24:1042-1049).

Therefore, using an iterative, structure-guided evolutionary approach, the AAV8 capsid surface was re-engineered to yield a new AAV variant called humanized AAV8 capsid (AAVhum.8). (Havlik L P, et al. (2020) J. Virol. 94(19):e00976-20). The transduction efficiency of AAVhum.8 was compared to AAV8 via injection into a humanized mouse model. Humanized mice were retro-orbitally injected with an AAV8 or AAVhum.8 capsid carrying a GFP transgene at a dose of 5×10¹³ vg/kg to compare transduction efficiency in human and murine hepatocytes. FIG. 5A-FIG. 5F shows the liver histology slides from humanized mice injected with AAV8 (FIG. 5A-FIG. 5C) and AAVhum.8 (FIG. 5D-FIG. 5F) capsids carrying GFP transgenes. FIG. 5A shows albumin staining for human hepatocytes in red and FIG. 5B shows GFP expression in green indicating AAV8 transduction. The merged image indicates the AAV8 capsid transduced better in mouse hepatocytes (FIG. 5C). FIG. 5D shows albumin staining for human hepatocytes in red and FIG. 5E shows GFP expression in green indicating AAVhum.8 transduction. The merged image indicates the AAVhum.8 capsid more equally transduced human and mouse hepatocytes (FIG. 5F). FIG. 5G provides a graph quantifying GFP positive cells per field, demonstrating that when compared to AAV8, the AAVhum.8 capsid transduces as well in mice and better in human hepatocytes. These data demonstrate that AAVhum.8 showed a tenfold increase in the total number of transduced human hepatocytes. In terms of percentage of total human hepatocytes transduced, AAV8 transduced an average of 3.39%, while AAVhum.8 transduced an average of 34.4%. (Havlik L P, et al., 2020).

Building upon these data, a new cassette containing a human codon optimized, CpG-depleted PHKG2 gene under the control of the liver-specific thyroxin binding globulin (TBG) promoter flanked by AAV2 ITRs is generated. The cassette is delivered in either an AAV8 capsid (AAV8-LSP-PHKG2) or an AAVhum.8 capsid (AAV.hum8-LSP-PHKG2). The TBG promoter provides liver tissue specificity, evades a T and B cell immunological response to the transgene, and is well-established in clinical trials. (Sawamoto K, et al. (2020) Mol. Ther.—Methods Clin. Dev. 18:50-61). Deletion of CpG elements from the transgene cassette further helps evade an immunological response. (Faust et al., 2013; Xiang et al., 2020). Using the triple plasmid transfection protocol previously described (Grieger J C et al. (2016); Havlik L P, et al. (2020)), AAV vectors are generated. Briefly, following transfection and harvest, virus preparations are purified via iodixonal gradient ultracentrifugation and endotoxin removal. Titers are determined via qPCR compared to a standard.

### Example 12

### Evaluation of AAV8-LSP-PHKG2 and AAV.hum8-LSP-PHKG2 in Phkg2^{-/-} Mice

At 2 months of age, the mice receive either PBS or AAV gene therapy (using AAV8 or AAVhum.8) via intravenous tail vein injection. AAV gene therapy is delivered first at a low dose of 5×10¹² vector genome (vg/kg). This initial dose is based on successful liver targeted AAV8 gene therapy in liver GSD murine and clinical trials. (Koeberl D D, et al. (2006) 13:1281-1289; Cho J H, et al. (2019) J. Inherit. Metab. Dis. 42:459-469; Lim J A, et al. (2019)). Dosage is de-escalated or escalated based on the benefit assessed at 5×10¹² vg/kg. Mice are euthanized at either 3, 6, 9, or 12 months of age. Liver disease burden is quantified using the morphological measurements, blood and urine assays, tissue assays, and histological analysis described herein including at least the measurement of liver:body weight ratio, western blot analysis, PhK enzymatic activity analysis, glycogen content analysis, histology, and blood and urine analyses. AAV vector transduction efficiency is evaluated via biodistribution and transgene expression. Biodistribution in liver tissue lysate is quantified via qPCR with primers specific to the Phkg2 transgene and beta actin as a housekeeping gene. Transgene expression is evaluated at the RNA level via RT-qPCR using primers specific to the Phkg2 transgene and beta actin as a housekeeping gene. Transgene protein levels are evaluated using immunofluorescence and immunohistochemistry using the anti-PHKG2 antibody (Proteintech, Rosemont, IL).

Statistical significance is determined by unpaired two-tailed Student's t-test using Prism software (GraphPad, La Jolla, CA). Data is presented as mean±standard deviation (SD). *p<0.05 is considered statistically significant. Statistical power calculations are performed based on existing quantitative molecular data. The number of groups=2. The desired power of test=0.90. The alpha=0.05. The confidence interval=90%. Calculations indicate that an estimate of 6 mice/group (3 male, 3 female) is appropriate for molecular experiments.

### Example 13

### Generation and Characterization of PHKA2 Transgene Constructs

Several new constructs containing the original human PHKA2 gene as well as human codon optimized, CpG-depleted PHKA2 gene are generated using a pcDNA plasmid obtained from GenScript and various promoters. Table 6 shows the various combinations of constructs having the PHKA2 transgene used in these experiments.

TABLE 6
Combination of PHKA2 Transgene, Promoters, and Cells Lines

| Gene | Promoter | Cell Line |
|---|---|---|
| PHKA2 | Universal | HEK293 |
| PHKA2 | Liver Specific | Huh7 |
| PHKA2 | Endogenous | Huh7 |
| PHKA2 | Liver Specific | HepG2 |
| PHKA2 | Endogenous | HepG2 |
| PHKA2^{CpG-free} | Universal | HEK293 |
| PHKA2^{CpG-free} | Liver Specific | Huh7 |
| PHKA2^{CpG-free} | Endogenous | Huh7 |
| PHKA2^{CpG-free} | Liver Specific | HepG2 |
| PHKA2^{CpG-free} | Endogenous | HepG2 |

First, the original human PHKA2 gene is placed under the control of at least three (3) different promoters—(i) a universal promoter, (ii) a liver-specific promoter (e.g., a thyroxin binding globulin (TBG) promoter flanked by AAV2 ITRs), and (iii) an endogenous promoter. The TBG promoter provides liver tissue specificity, evades a T and B cell immunological response to the transgene, and is well-established in clinical trials. (Sawamoto K, et al. (2020)).

Second, the human codon optimized, CpG-depleted PHKA2 gene is placed under the control of at least three (3) different promoters—(i) a universal promoter, (ii) a liver-specific promoter (e.g., a thyroxin binding globulin (TBG) promoter flanked by AAV2 ITRs), and (iii) a endogenous promoter. Deletion of CpG elements from the transgene cassette further helps evade an immunological response. (Faust et al., 2013; Xiang et al., 2020).

Third, using the triple plasmid transfection protocol previously described (Grieger J C et al. (2016); Havlik L P, et al. (2020)), AAV vectors are generated (including but not limited to AAV8 capsid, AAVhum.8 capsid, or AAV9).

Fourth, following transfection and harvest, virus preparations are purified via iodixonal gradient ultracentrifugation and endotoxin removal as described above. Titers are determined via qPCR compared to a standard as described above. Western blot is performed while beta actin serves as the loading control. Similarly, enzyme activity of is assayed with cell lysates. Enzyme activity is measured indirectly by measuring the amount of glucose released from glycogen substrate using glucose reagent (Infinity™; Cat No. TR15421) from ThermoScientific (Fisher Diagnostics, Middletown, VA, USA

Fifth, at about 2-3 months of age, the mice (wild-type and PHKA2^{-/-}) receive either PBS or AAV gene therapy (using AAV8, AAVhum.8, or AAV9) via intravenous tail vein injection. AAV gene therapy (directed at delivered PHKA2 transgene) is delivered first at a dose between 5×10¹² vector genome (vg/kg) and 1×10¹³ vg/kg. Dosage is de-escalated or escalated based on the benefit assessed at this first dose. Mice are euthanized at either 1, 3, 6, 9, or 12 months of age.

Sixth, liver disease burden is quantified using the morphological measurements, blood and urine assays, tissue assays, and histological analysis described herein including at least the measurement of liver:body weight ratio (e.g., presence of hepatomegaly), western blot analysis, PhK enzymatic activity analysis, glycogen content analysis, histology (including assessment of fibrosis), and blood and urine analyses (e.g., detection of at least urine Hex4, liver enzymes like ALT and AST), and hypoglycemia). Liver weight is reported as a percent of body weight using the ratio of Liver weight/Body Weight (LW/BW)*100, a metric for hepatomegaly. AAV vector transduction efficiency is evaluated via biodistribution and transgene expression. Biodistribution in liver tissue lysate is quantified via qPCR with primers specific to the PHKA2 transgene and beta actin as a housekeeping gene. Transgene expression is evaluated at the RNA level via RT-qPCR using primers specific to the PHKA2 transgene and beta actin as a housekeeping gene as previously described. Transgene protein levels are evaluated using immunofluorescence and immunohistochemistry using the anti-PHKA2 antibody (Proteintech, Rosemont, IL). Following transfection of each of these constructs, expression of the PhK α2 subunit is assessed.

Statistical significance is determined by unpaired two-tailed Student's t-test using Prism software (GraphPad, La Jolla, CA). Data is presented as mean±standard deviation (SD). *p<0.05 is considered statistically significant. Calculations indicate that an estimate of at least 6 mice per group (divided between genders) is appropriate for molecular experiments.

### Example 14

### Generation and Characterization of PHKG2 Transgene Constructs

Several new constructs containing the original human PHKG2 gene as well as human codon optimized, CpG-depleted PHKG2 gene are generated using a pcDNA plasmid obtained from GenScript and various promoters. Table 7 shows the various combinations of constructs having the PHKG2 transgene used in these experiments.

TABLE 7
Combination of PHKG2 Transgene, Promoters, and Cells Lines

| Gene | Promoter | Cell Line |
|---|---|---|
| PHKG2 | Universal | HEK293 |
| PHKG2 | Liver Specific | Huh7 |
| PHKG2 | Endogenous | Huh7 |
| PHKG2 | Liver Specific | HepG2 |
| PHKG2 | Endogenous | HepG2 |
| PHKG2^{CpG-free} | Universal | HEK293 |
| PHKG2^{CpG-free} | Liver Specific | Huh7 |
| PHKG2^{CpG-free} | Endogenous | Huh7 |
| PHKG2^{CpG-free} | Liver Specific | HepG2 |
| PHKG2^{CpG-free} | Endogenous | HepG2 |

First, the original human PHKG2 gene is placed under the control of at least three (3) different promoters—(i) a universal promoter, (ii) a liver-specific promoter (e.g., a thyroxin binding globulin (TBG) promoter flanked by AAV2 ITRs), and (iii) a endogenous promoter. The TBG promoter provides liver tissue specificity, evades a T and B cell immunological response to the transgene, and is well-established in clinical trials. (Sawamoto K, et al. (2020)).

Second, the human codon optimized, CpG-depleted PHKG2 gene is placed under the control of at least three (3) different promoters—(i) a universal promoter, (ii) a liver-specific promoter (e.g., a thyroxin binding globulin (TBG) promoter flanked by AAV2 ITRs), and (iii) a endogenous promoter. Deletion of CpG elements from the transgene cassette further helps evade an immunological response. (Faust et al., 2013; Xiang et al., 2020).

Third, using the triple plasmid transfection protocol previously described (Grieger J C et al. (2016); Havlik L P, et al. (2020)), AAV vectors are generated (including but not limited to AAV8 capsid, AAVhum.8 capsid, or AAV9).

Fourth, following transfection and harvest, virus preparations are purified via iodixonal gradient ultracentrifugation and endotoxin removal. Titers are determined via qPCR compared to a standard. Following transfection of each of these constructs, expression of the PHKG2 subunit is assessed. Western blot is performed while beta actin serves as the loading control. Similarly, enzyme activity of is assayed with cell lysates. Enzyme activity is measured indirectly by measuring the amount of glucose released from glycogen substrate using glucose reagent (Infinity™; Cat No. TR15421) from ThermoScientific (Fisher Diagnostics, Middletown, VA, USA).

Fifth, at about 2-3 months of age, the mice (wild-type and PHKG2^{-/-}) receive either PBS or AAV gene therapy (using AAV8, AAVhum.8, or AAV9) via intravenous tail vein injection. AAV gene therapy (directed at delivered PHKG2 transgene) is delivered first at a dose between 5×10¹² vg/kg-1×10¹³ vg/kg. Dosage is de-escalated or escalated based on the benefit assessed at this first dose. Mice are euthanized at either 1, 3, 6, 9, or 12 months of age.

Sixth, liver disease burden is quantified using the morphological measurements, blood and urine assays, tissue assays, and histological analysis described herein including at least the measurement of liver:body weight ratio (e.g., presence of hepatomegaly), western blot analysis, PhK enzymatic activity analysis, glycogen content analysis, histology (including assessment of fibrosis), and blood and urine analyses (e.g., detection of at least urine Hex4, liver enzymes like ALT and AST), and hypoglycemia). Liver weight is reported as a percent of body weight using the ratio of Liver weight/Body Weight (LW/BW)*100, a metric for hepatomegaly. AAV vector transduction efficiency is evaluated via biodistribution and transgene expression. Biodistribution in liver tissue lysate is quantified via qPCR with primers specific to the PHKG2 transgene and beta actin as a housekeeping gene. Transgene expression is evaluated at the RNA level via RT-qPCR using primers specific to the PHKG2 transgene and beta actin as a housekeeping gene as described above. Transgene protein levels are evaluated using immunofluorescence and immunohistochemistry using the anti-PHKG2 antibody (Proteintech, Rosemont, IL).

Statistical significance is determined by unpaired two-tailed Student's t-test using Prism software (GraphPad, La Jolla, CA). Data is presented as mean±standard deviation (SD). *p<0.05 is considered statistically significant. Calculations indicate that an estimate of at least 6 mice per group (divided between genders) is appropriate for molecular experiments.

### Example 15

### Generation and Characterization of PYGL Transgene Constructs

Building upon these data, several new constructs containing the original human PHKG2 gene as well as human codon optimized, CpG-depleted PYGL gene are generated using a pcDNA plasmid obtained from GenScript and various promoters. Table 8 shows the various combinations of constructs having the PYGL transgene used in these experiments.

TABLE 8
Combination of PYGL Transgene, Promoters, and Cells Lines

| Gene | Promoter | Cell Line |
|---|---|---|
| PYGL | Universal | HEK293 |
| PYGL | Liver Specific | Huh7 |
| PYGL | Endogenous | Huh7 |
| PYGL | Liver Specific | HepG2 |
| PYGL | Endogenous | HepG2 |
| PYGL^{CpG-free} | Universal | HEK293 |
| PYGL^{CpG-free} | Liver Specific | Huh7 |
| PYGL^{CpG-free} | Endogenous | Huh7 |
| PYGL^{CpG-free} | Liver Specific | HepG2 |
| PYGL^{CpG-free} | Endogenous | HepG2 |

First, the original human PYGL gene is placed under the control of at least three (3) different promoters—(i) a universal promoter, (ii) a liver-specific promoter (e.g., a thyroxin binding globulin (TBG) promoter flanked by AAV2 ITRs), and (iii) a endogenous promoter. The TBG promoter provides liver tissue specificity, evades a T and B cell immunological response to the transgene, and is well-established in clinical trials. (Sawamoto K, et al. (2020)).

Second, the human codon optimized, CpG-depleted PYGL gene is placed under the control of at least three (3) different promoters—(i) a universal promoter, (ii) a liver-specific promoter (e.g., a thyroxin binding globulin (TBG) promoter flanked by AAV2 ITRs), and (iii) a endogenous promoter. Deletion of CpG elements from the transgene cassette further helps evade an immunological response. (Faust S M, et al. (2013) J. Clin. Invest. 123:2994-3001; Xiang Z Q, et al. (2020) Mol. Ther. 28:771-783).

Third, using the triple plasmid transfection protocol previously described (Grieger J C et al. (2016); Havlik L P, et al. (2020)), AAV vectors are generated (including but not limited to AAV8 capsid, AAVhum.8 capsid, or AAV9).

Fourth, following transfection and harvest, virus preparations are purified via iodixonal gradient ultracentrifugation and endotoxin removal. Titers are determined via qPCR compared to a standard. Following transfection of each of these constructs, expression of the PYGL subunit is assessed. Western blot is performed while beta actin serves as the loading control. Similarly, enzyme activity of is assayed with cell lysates. Enzyme activity is measured indirectly by measuring the amount of glucose released from glycogen substrate using glucose reagent (Infinity™; Cat No. TR15421) from ThermoScientific (Fisher Diagnostics, Middletown, VA, USA).

Fifth, at about 2-3 months of age, the mice (wild-type and PYGL^{-/-}) receive either PBS or AAV gene therapy (using AAV8, AAVhum.8, or AAV9) via intravenous tail vein injection. AAV gene therapy (directed at delivered PYGL transgene) is delivered first at a dose between 5×10¹² vector genome (vg/kg) and 1×10¹³ vg/kg. Dosage is de-escalated or escalated based on the benefit assessed at this first dose. Mice are euthanized at either 1, 3, 6, 9, or 12 months of age.

Sixth, liver disease burden is quantified using the morphological measurements, blood and urine assays, tissue assays, and histological analysis described herein including at least the measurement of liver:body weight ratio (e.g., presence of hepatomegaly), western blot analysis, PhK enzymatic activity analysis, glycogen content analysis, histology (including assessment of fibrosis), and blood and urine analyses (e.g., detection of at least urine Hex4, liver enzymes like ALT and AST), and hypoglycemia). Liver weight is reported as a percent of body weight using the ratio of Liver weight/Body Weight (LW/BW)*100, a metric for hepatomegaly. AAV vector transduction efficiency is evaluated via biodistribution and transgene expression. Biodistribution in liver tissue lysate is quantified via qPCR with primers specific to the PYGL transgene and beta actin as a housekeeping gene. Transgene expression is evaluated at the RNA level via RT-qPCR using primers specific to the PHKG2 transgene and beta actin as a housekeeping gene as described herein. Transgene protein levels are evaluated using immunofluorescence and immunohistochemistry using the anti-PYGL antibody (Proteintech, Rosemont, IL).

Statistical significance is determined by unpaired two-tailed Student's t-test using Prism software (GraphPad, La Jolla, CA). Data is presented as mean±standard deviation (SD). *p<0.05 is considered statistically significant. Calculations indicate that an estimate of at least 6 mice per group (divided between genders) is appropriate for molecular experiments.

### Example 16

### Overexpression of Various PHKG2 Constructs in HEK293 Cells

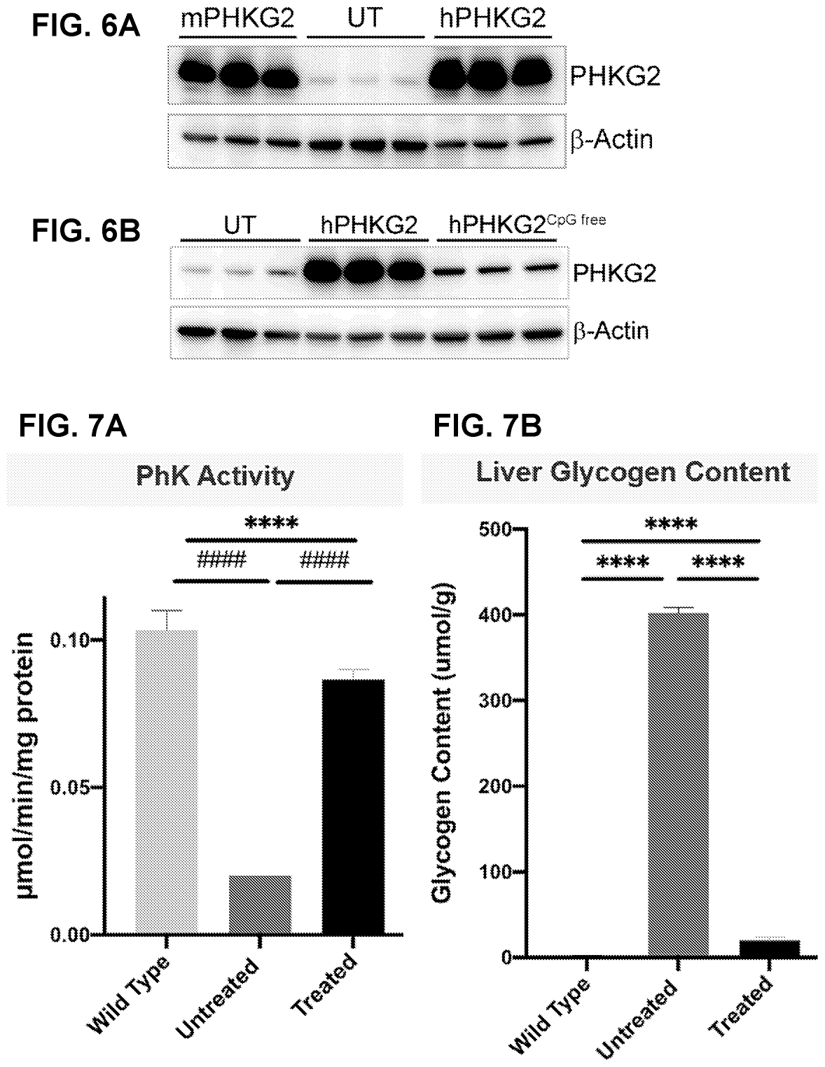

To examine the expression of hPHKG2 or mPhkg2 driven by a CB promoter, HEK293T cells were transfected with pAV-CB-hPHKG2 (FIG. 20B) or pAV-CB-mPhkg2 (FIG. 20A) for 48 hr. The Western blot shows that the untreated animals had no appreciable amount of PHKG2 whereas the expression of both mPhkg2 and hPHKG2 generated significant PHKG2 expression (FIG. 6A). Similarly, the overexpression of human PHKG2 (hPHKG2) or CpG optimized hPHKG2 (hPHKG2^{CpGfree}) in HEK293T cells was examined. Here, the HEK293 were transfected with pAV-CB-hPHKG2 (FIG. 20B) or pAV-CB-hPHKG2^{CpGfree} (FIG. 20C) for 48 hr. The expression of hPHKG2 was detected by anti-PHKG2 antibodies. R-Actin was used as a loading control. As shown in FIG. 6B, hPHKG2 generated a greater amount of PHKG2 than did hPHKG2^{CpGfree} (which was greater than that of the untreated mice).

### Example 17

### Avv9 Vector Comprising mPhkg2 Normalized PhK Enzyme Activity and Reduced Levels of Liver Glycogen and Percent Liver Weight

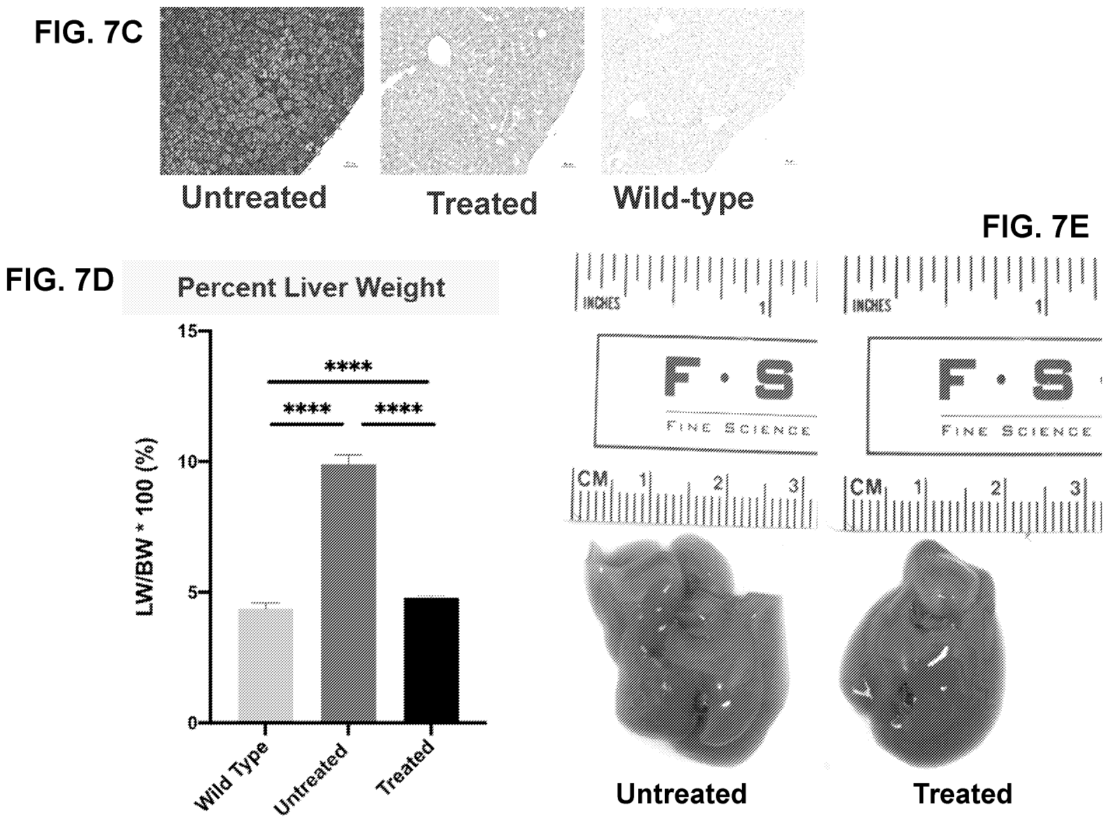

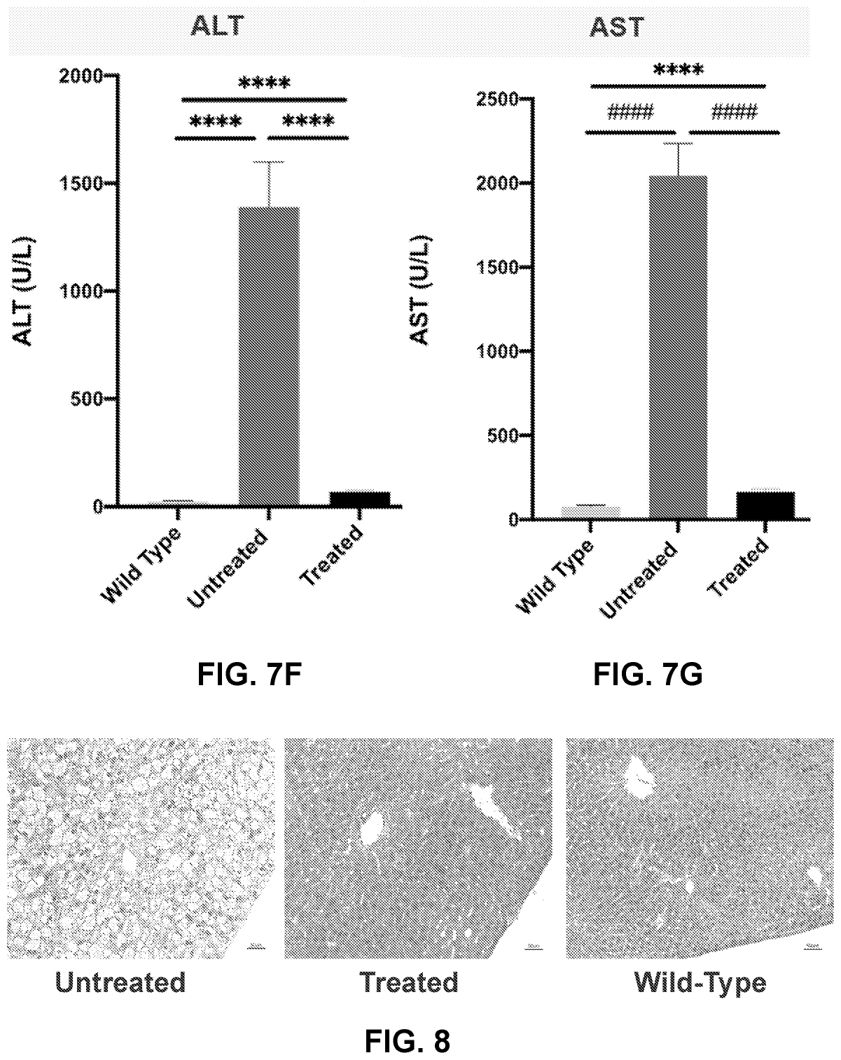

A short-term in vivo experiment using AAV9-LSP-mPhkg2 was performed. AAV9-LSP-mPhkg2 was administered (5×10¹² vg/kg) to 3-month old GSD IX γ2 mice (n=7) in a 2-week treatment protocol. Results were compared to that from untreated mice (n=4) and wild-type mice (n=3). The treatment normalized the level of PhK activity and the level of liver glycogen content. FIG. 7A shows that the treated GSD IX γ2 mice had a PhK activity level that was similar to that of the wild-type mice and significantly greater than untreated mice. FIG. 7B shows that the treated GSD IX γ2 mice had a level of liver glycogen content that was similar to that of the wild-type mice and significantly less than that of the untreated mice. The PAS staining of wild-type mice (left panel), treated GSD IX γ2 mice (middle panel), and untreated mice (right panel) in FIG. 7C supported the liver content analysis in FIG. 7B. FIG. 7D shows that the treated GSD IX γ2 mice (treated with AAV-LSP-mPhkg2) had significantly reduced percent liver weight (LW/BW×100) when compared to untreated mice. The percent liver weight of the treated GSD IX γ2 mice (mPhkg2) was the same or nearly the same as that of the wild-type mice. FIG. 7E show a representative liver from the untreated group (left) and a representative liver from the AAV9-LSP-mPhkg2 treated group (right). FIG. 7F shows that the treated GSD IX γ2 mice had serum ALT levels similar to that of wild-type mice, which significantly less than that of untreated mice. FIG. 7G show that GSD IX γ2 mice treated with AAV-LSP_mPhkg2 had reduced serum levels of AST. Here, treated GSD IX γ2 mice and to wild-type mice had similar AST levels, which were significantly decreased from that of the untreated mice.

FIG. 8 shows that GSD IX γ2 mice treated with AAV-LSP-mPhkg2 experienced a restoration of hepatocyte architecture.

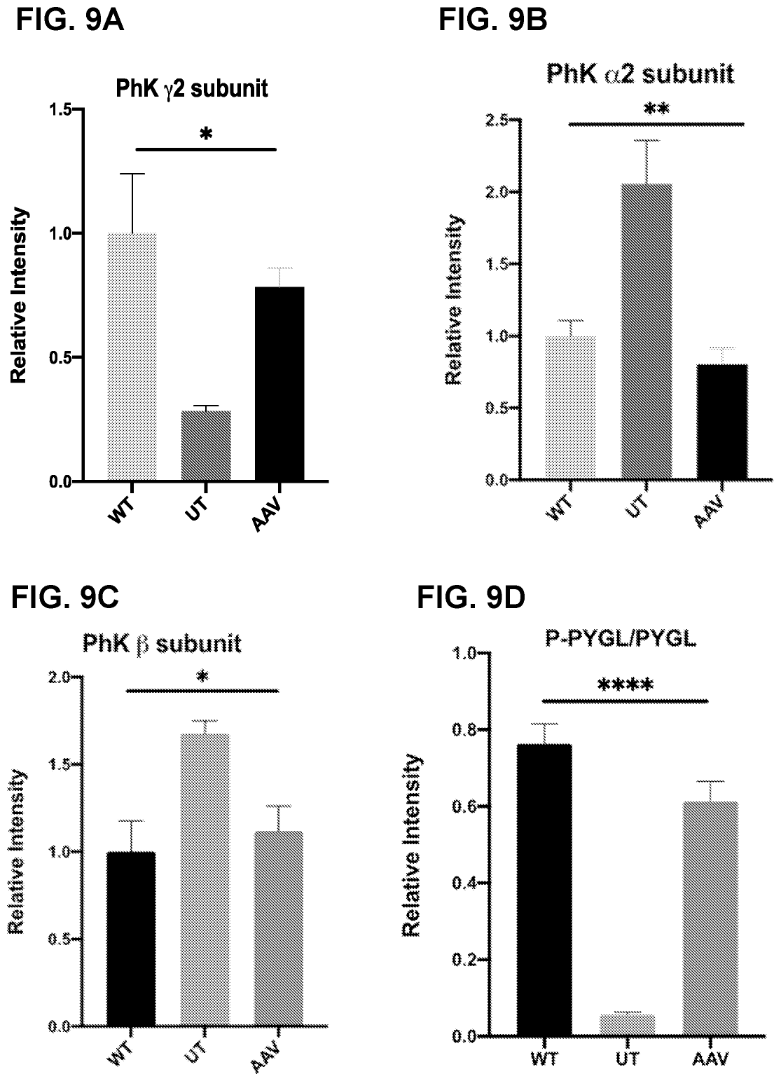

As shown in FIG. 9A-FIG. 9C, the 2-week treatment with AAV-LSP-mPhkg2 restored glycogenolysis metabolic pathway. The relative intensity of the PhK γ2 subunit, PhK α2 subunit, and PhK β subunit was examined in 2-week treated mice, untreated mice, and wild-type mice. FIG. 9A shows that the treated and wild-type mice had significantly greater relative intensity of the PhK γ2 subunit when compared to the untreated mice. FIG. 9B shows that the treated and wild-type mice had significantly less relative intensity of the PhK α2 subunit when compared to the untreated mice. FIG. 9C shows that the treated and wild-type mice had significantly less relative intensity of PhK β subunit when compared to the untreated group. FIG. 9D shows that the treated and wild-type mice had a relative intensity for P-PYGL/PYGL that was significantly greater than that of the untreated mice.

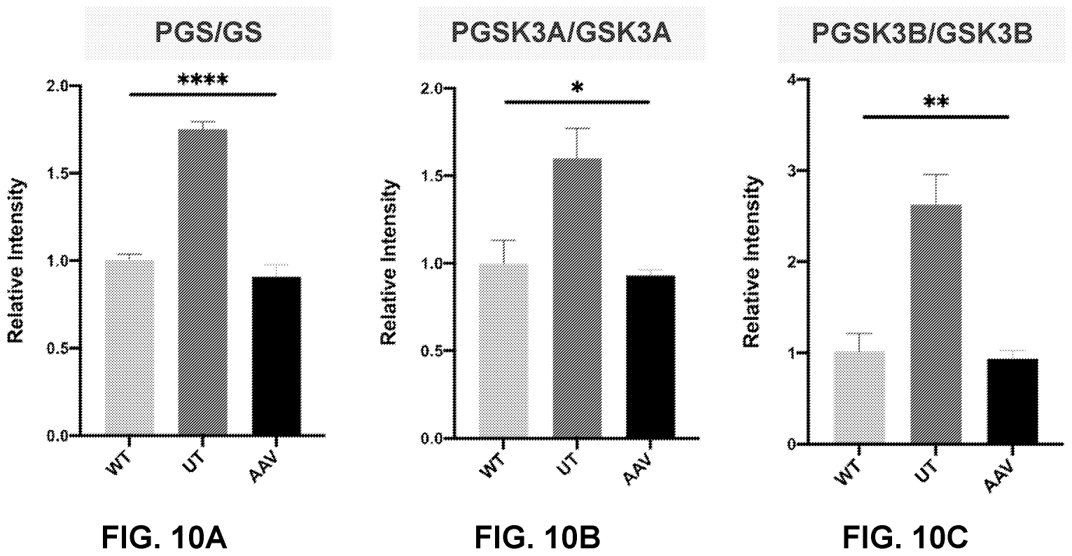

Moreover, FIG. 10A-FIG. 10C provides additional evidence of the restoration of the glycogenesis metabolic pathway. FIG. 10A shows that AAV9-LSP-mPHKG2 treated GSD IX γ2 mice had a PGS/GS level approaching that of wild-type and significantly less than the untreated group. FIG. 10B shows that the treated GSD IX γ2 mice had a PGSK3A/GSK3A level similar to the wild-type level and significantly less than the untreated group. Finally, FIG. 10C shows that the treated mice and the wild-type mice had a similar level of PGSK3B/GSK3B, which was significantly less than the untreated group.

### Example 18

### Avv9 Vector Comprising mPhkg2 Improved Multiple Outcome Measures in a Prolonged In Vivo Experiment

A long-term in vivo experiment using AAV9-LSP-mPhkg2 was performed. AAV9-LSP-mPhkg2 was administered (5×10¹² vg/kg) to 3-month old GSD IX γ2 mice (n=4) in a 3-month treatment protocol. Mice were euthanized at 6 months of age and results were compared to that of untreated (n=5) and wild-type (n=4) mice.

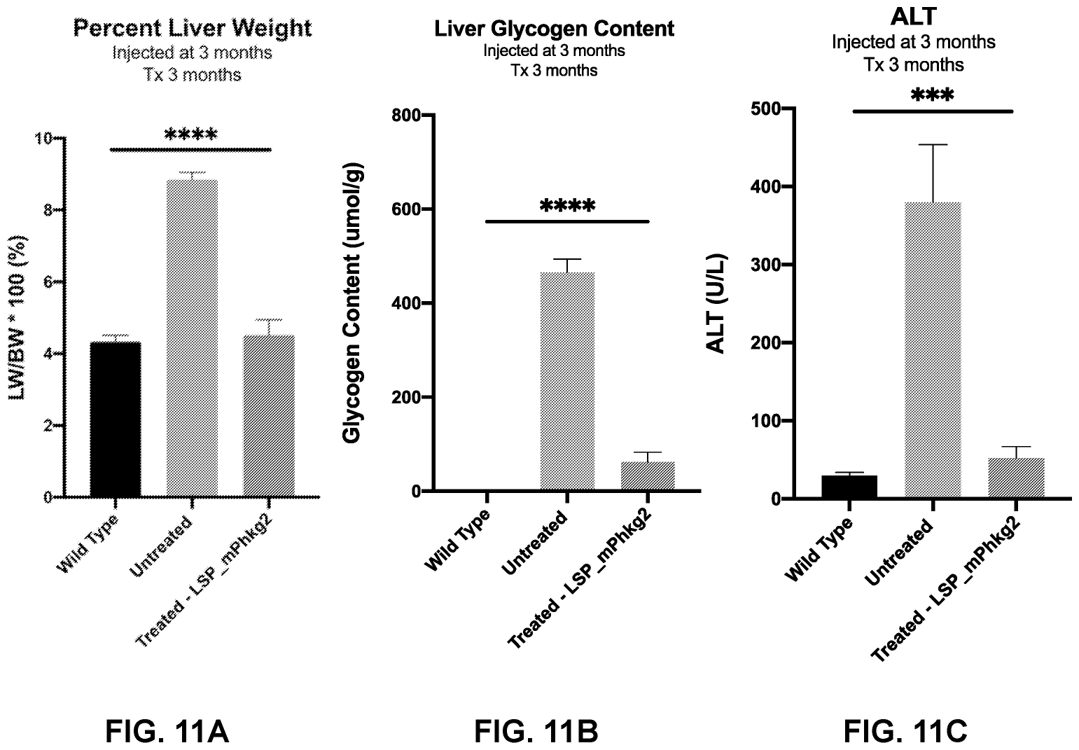

FIG. 11A shows that the GSD IX γ2 mice treated with AAV9-LSP-mPhkg2 had significantly reduced percent liver weight (LW/BW) when compared to untreated mice. The percent liver weight of the treated GSD IX γ2 mice was the same or nearly the same as that of the wild-type mice. FIG. 11B shows that the treated GSD IX γ2 mice had reduced liver glycogen content such that the level of glycogen content approached that of wild-type mice. The treated mice had a level of glycogen content that was significantly less than the untreated mice. FIG. 11C shows that the AAV9-LSP-mPhkg2 treatment protocol improved the level of serum ALT in GSD IX γ2 mice. The treated and wild-type groups had a similar level of serum ALT, which were significantly less than that of the untreated group. A long-term in vivo experiment using AAV9-LSP-mPhkg2 was performed. AAV9-LSP-mPhkg2 was administered (5×10¹² vg/kg) to 6-month old GSD IX γ2 mice (n=5) in a 3-month treatment protocol. Mice were euthanized at 9 months of age and results were compared to that of untreated (n=5) and wild-type (n=3) mice.

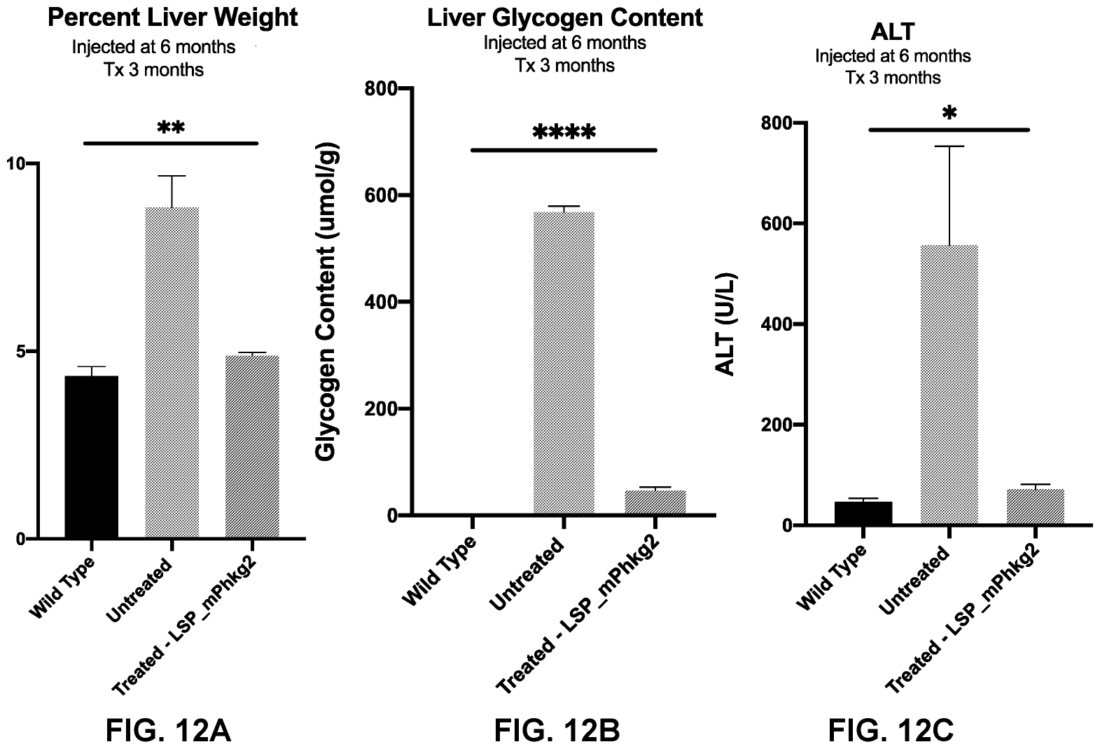

FIG. 12A shows that the GSD IX γ2 mice treated with AAV9-LSP-mPhkg2 had significantly reduced percent liver weight (LW/BW) when compared to that of untreated mice. The percent liver weight of the treated GSD IX γ2 mice was the same or nearly the same as that of the wild-type mice. FIG. 12B shows that the treated GSD IX γ2 mice had reduced liver glycogen content such that the glycogen content was similar to that of wild-type mice and was significantly less than the untreated mice. FIG. 12C shows that the treatment protocol improved the level of serum ALT. The treated and wild-type groups had a similar level of serum ALT, which was significantly less than the untreated group.

### Example 19

### Avv9 Vector Comprising hPHKG2 Normalized PhK Enzyme Activity and Reduced Levels of Liver Glycogen and Percent Liver Weight

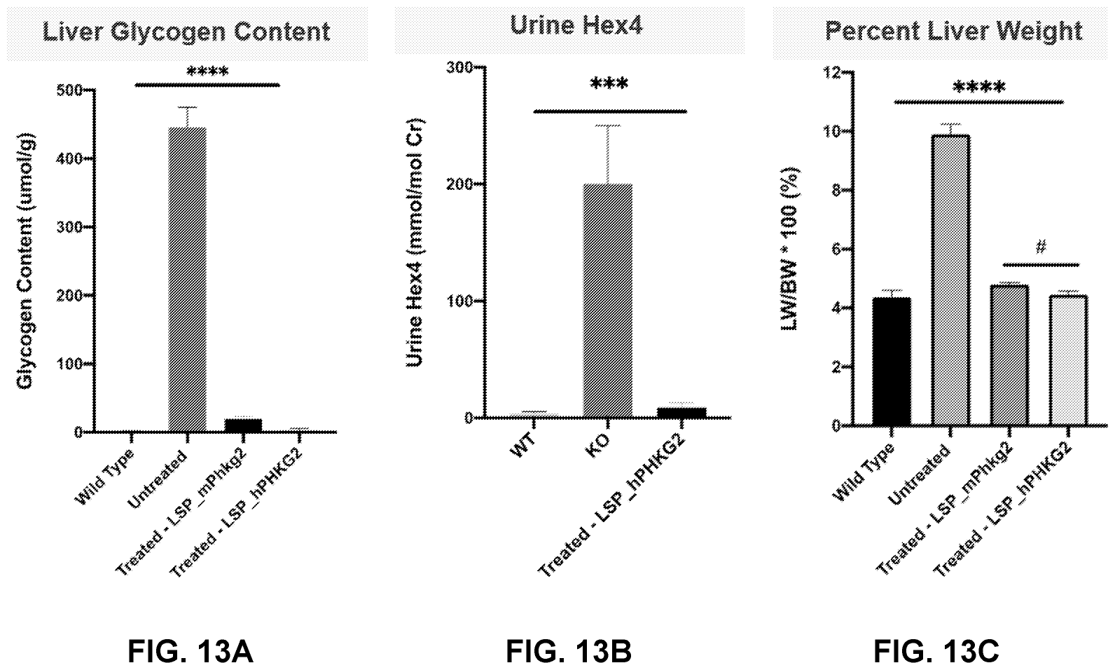

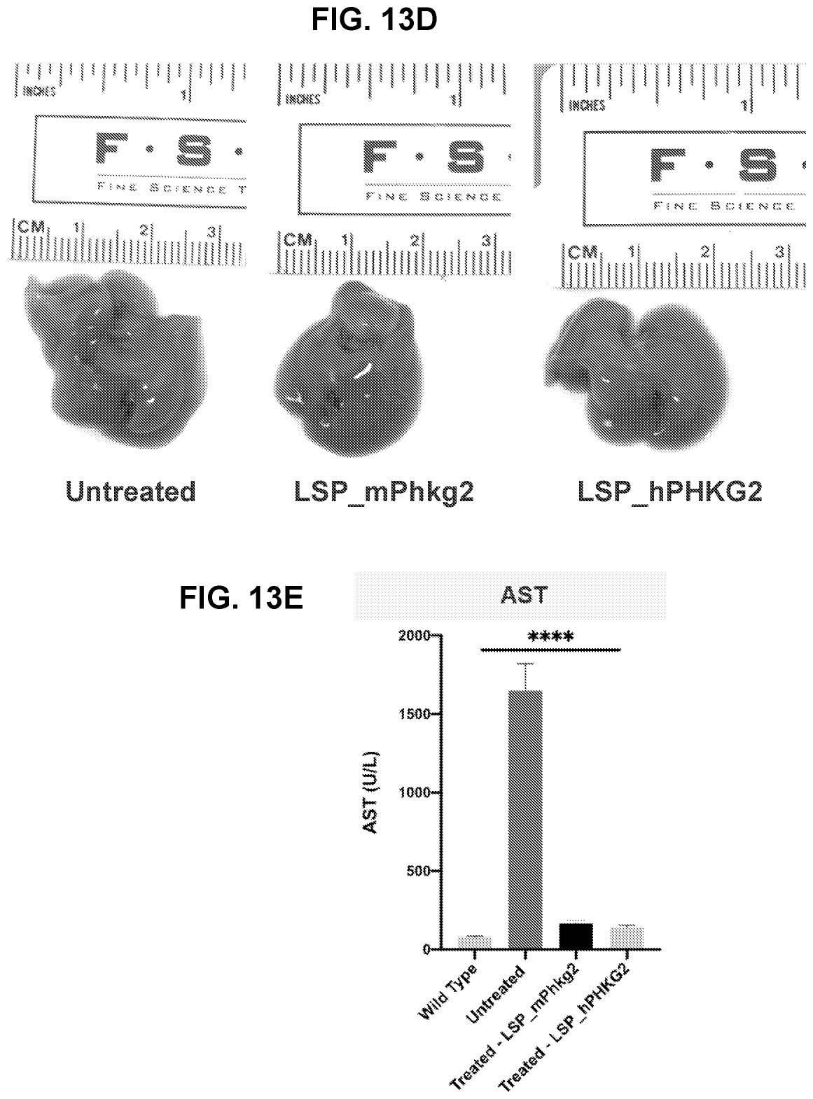

A short-term in vivo experiment using AAV9-LSP-hPHKG2 was performed. AAV9-LSP-hPHKG2 was administered (5×10¹² vg/kg) to 3-month old GSD IX γ2 mice (n=5) in a 2-week treatment protocol. Results were compared to that of untreated (n=3) and wild-type (n=3) mice. FIG. 13A shows that the treated GSD IX γ2 mice (treated with AAV-LSP-mPhkg2 or AAV-LSP-hPHKG2) had reduced liver glycogen content such that the level of glycogen content was similar to that of wild-type mice and significantly less than that of the untreated mice. FIG. 13B shows that treatment with AAV-LSP-hPHKG2 reduced urine Hex4 levels when compared to untreated mice (KO). FIG. 13C shows that the treated GSD IX γ2 mice (treated with AAV-LSP-mPhkg2 or AAV-LSP-hPHKG2) had significantly reduced liver weight (LW/BW) when compared to the untreated mice. The percent liver weight of GSD IX γ2 mice treated with either mPhkg2 or hPHKG2 was the same or nearly the same as that of the wild-type mice. FIG. 13D show a representative liver from the untreated group (left), a representative liver from the AAV9-LSP-mPhkg2 treated group (middle), and a representative liver from the AAV9-LSP-hPHKG2 treated group (right). FIG. 13E show that treated GSD IX γ2 mice treated with either AAV-LSP-mPhkg2 or AAV-LSP-hPHKG2 had a reduced level of serum AST. Here, the serum AST level of treated GSD IX γ2 mice was similar to the level of wild-type mice and significantly decreased from the untreated mice.

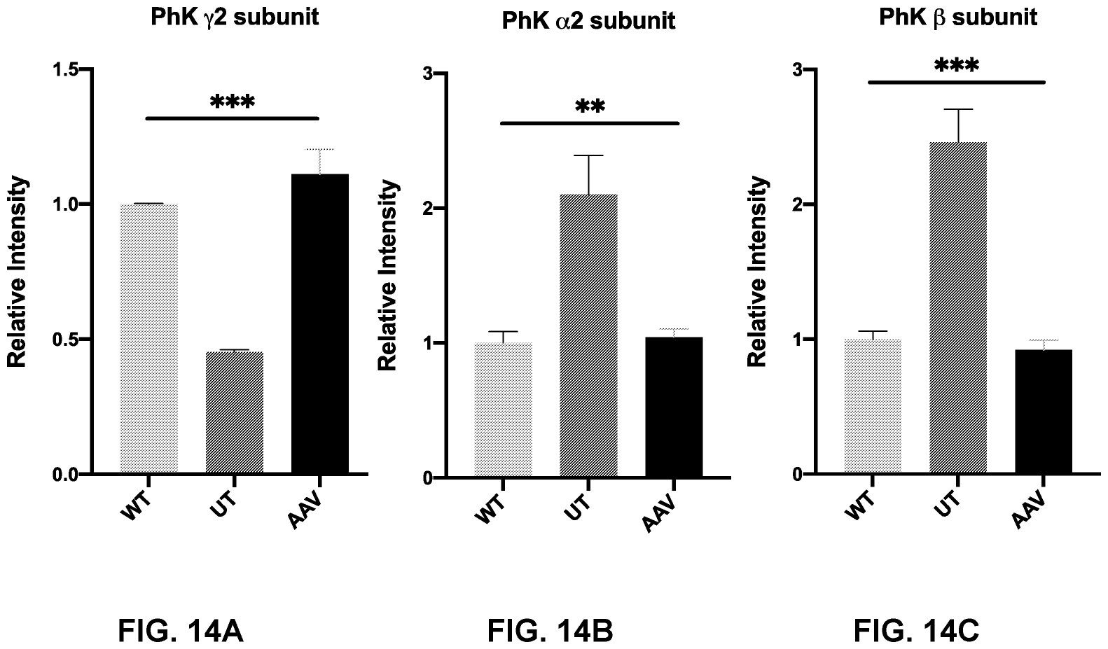

As shown in FIG. 14A-FIG. 14B, the 2-week treatment with AAV-LSP-hPHKG2 restored glycogenolysis signaling pathway. The relative intensity of the PhK γ2 subunit, the PhK α2 subunit, and the PhK 3 subunit was examined in treated mice, untreated mice, and wild-type mice. FIG. 14A shows that the treated mice and wild-type mice had significantly greater relative intensity of the PhK γ2 subunit when compared to untreated mice. FIG. 14B shows that the treated and wild-type mice had significantly less relative intensity of the PhK α2 subunit when compared to the untreated mice. Finally, FIG. 14C shows that the treated and wild-type mice had significantly less relative intensity of the PhK 3 subunit when compared to the untreated mice. The AAV9-LSP-hPHKG2 treatment restored PhK enzyme activity to wild-type levels and reduced serum ALT.

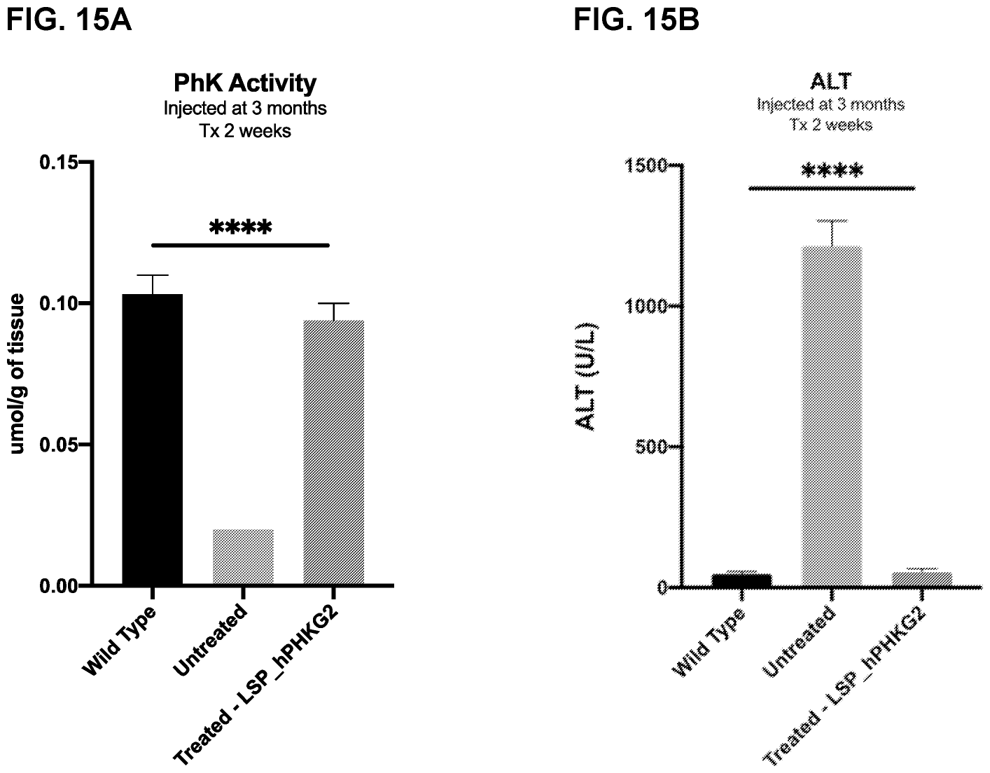

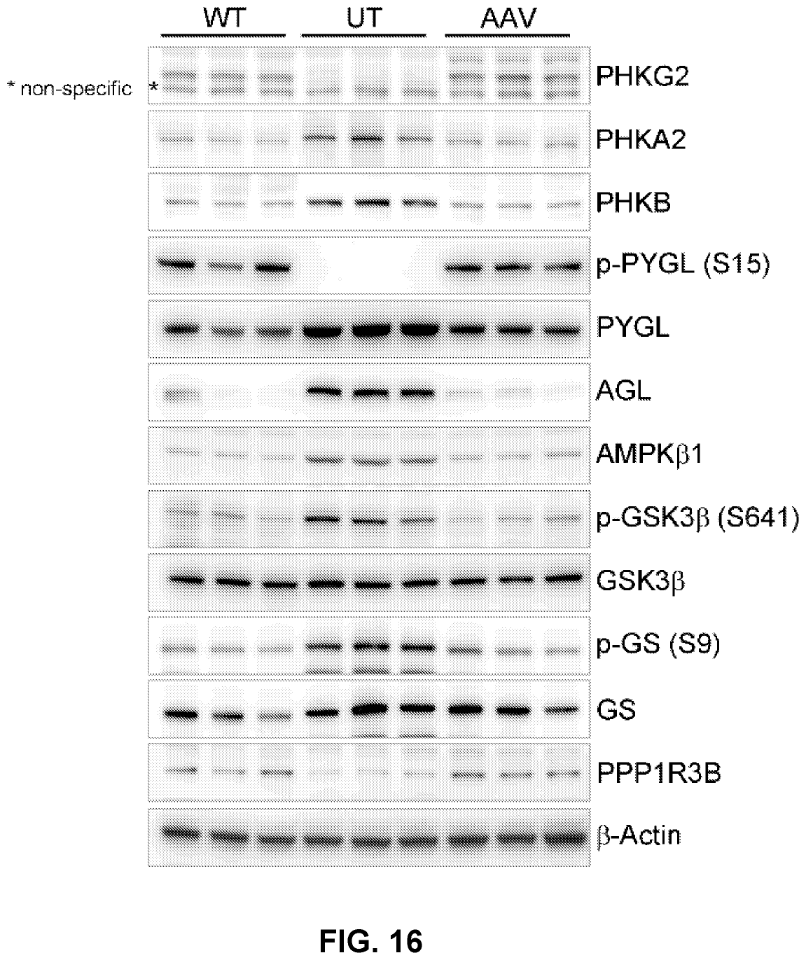

FIG. 15A shows that the treated GSD IX γ2 mice had PhK activity similar to that of wild-type mice. Treated and wild-type mice had a level of PhK activity that was significantly greater than that of the untreated mice. FIG. 15B shows that the AAV9-LSP-hPHKG2 treatment reduced the level of serum ALT. The treatment group and the wild-type group had nearly the same level of serum ALT, which was significantly reduced to that of the untreated group. FIG. 16 shows a series of Western blots comparing the hepatic protein levels for multiple members of the glycogen metabolic pathway, indicating that AAV9 treated GSD IX γ2 mice had levels similar to the levels wild-type mice. Each lane corresponds to one (1) mouse liver.

### Example 20

### AVV9 Vector Comprising hPHKG2^{CpG-Fr}ee Reduced Levels of Liver Glycogen Content and Percent Liver Weight

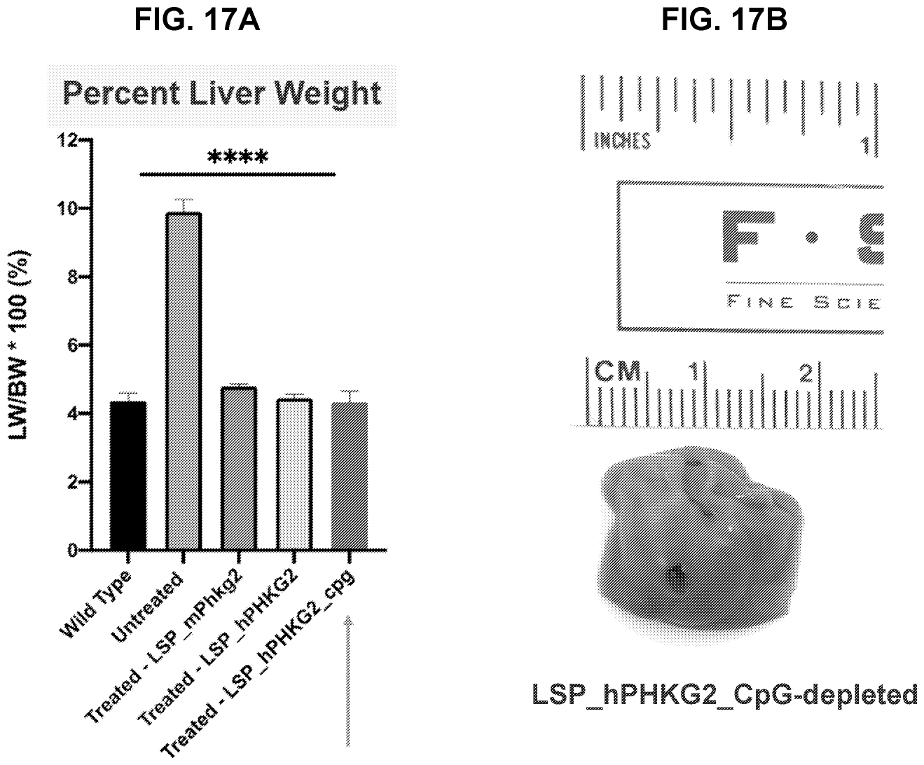

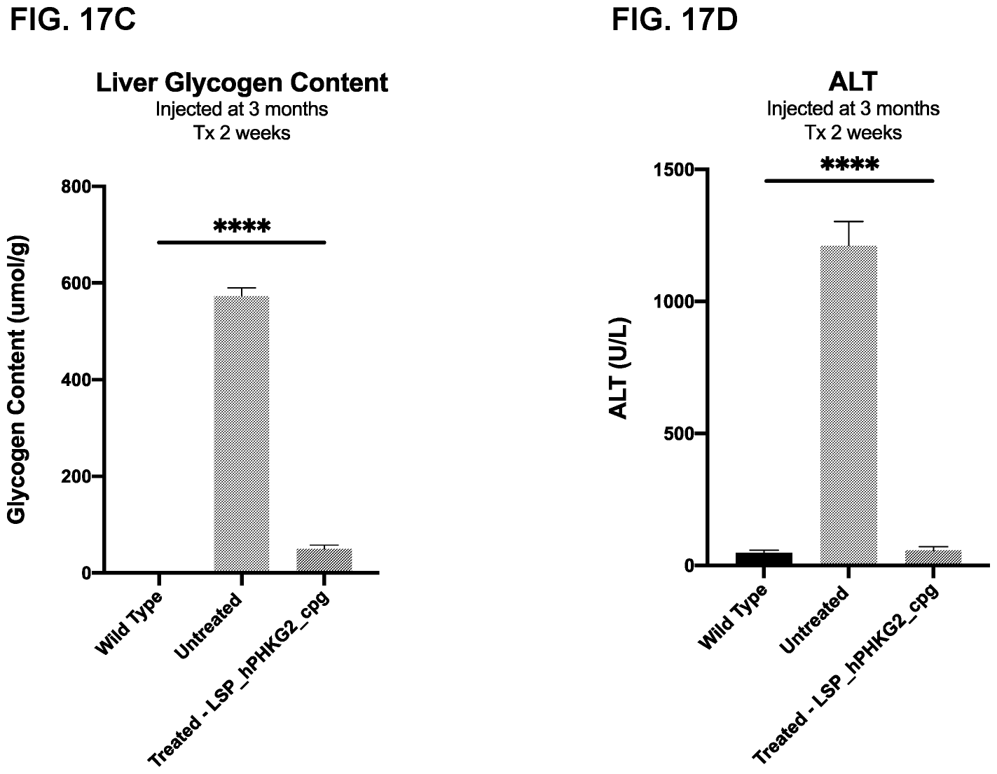

A short-term in vivo experiment using AAV9-LSP-hPHKG2^{CPGFree} was performed. AAV9-LSP-hPHKG2^{CpG-Free} was administered (5×10¹² vg/kg) to 3-month old GSD IX γ2 mice (n=5) in a 2-week treatment protocol. Results were compared to that of untreated (n=3) and wild-type (n=5) mice. FIG. 17A shows that the GSD IX γ2 mice treated with AAV9-LSP-hPHKG2^{CpG-Free} had significantly reduced percent liver weight (LW/BW) when compared to untreated mice. In fact, AAV9-LSP-hPHKG2^{CpG-Free} treated mice had a percent liver weight similar to that of GSD IX γ2 mice treated with either AAV9-LSP-hPHKG2 or AAV9-LSP-mPhkg. All three groups of treated mice had a percent liver weight the same or nearly the same as the wild-type group. FIG. 17B show a representative liver from the AAV9-LSP-hPHKG2^{CpG-Free} group. FIG. 17C shows that GSD IX γ2 mice treated with AAV-LSP-hPHKG2^{CpG-Free} had a level of liver glycogen content similar to that of wild-type mice. Both treated and wild-type mice had a liver glycogen content that was significantly less than the untreated group. Similarly, FIG. 17D shows that treated GSD IX γ2 mice had a serum ALT level similar to that of wild-type mice, both of which were significantly less than that of untreated mice.

### Example 21

### Overexpression of Various PHKG2 Constructs in HEK293 Cells

Western blot results show the overexpression of human glycogen phosphorylase L (hPYGL) and human phosphorylase kinase regulatory subunit alpha 2 (hPHKA2) in HEK293T cells. The cells were transfected with pAV-CB-hPYGL (FIG. 20E) or pAV-CB-hPHKA2 (FIG. 20F) for 48 hr. The expression of hPYGL or hPHKA2 was detected by anti-PYGL or anti-PHKA2 antibodies. β-Actin was used as a loading control.

The overexpression of human phosphorylase kinase regulatory subunit alpha (hPHKA2) or human glycogen phosphorylase L (hPYGL) in HEK293T cells was examined. Here, the HEK293 were transfected with pAV-CB-hPHKA2 or pAV-CB-hPYGL for 48 hr. The expression of hPHKA2 was detected by anti-PHKA2 antibodies while the expression of hPYGL was detected with anti-PYGL antibodies. R-Actin was used as a loading control. Western blot shows that the untreated animals had no appreciable amount of PHKA2 (FIG. 19A) or PYGL (FIG. 19B). Conversely, the treated HEK293 cells had demonstrable expression of PHKA2 (FIG. 19A) and PYGL (FIG. 19B).

### Example 22

### Generation of GSD VI Mice (Pgyl^{-/-})

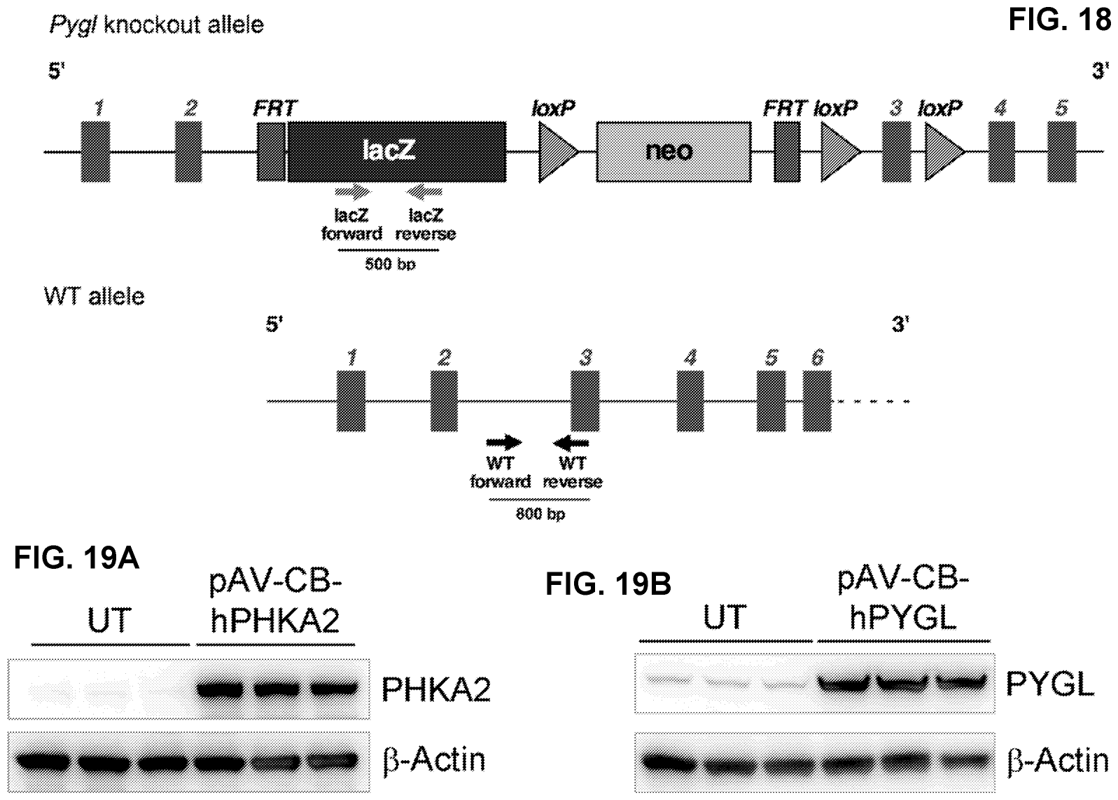

A Pygl knockout allele mouse (Pygl^{targeted mutation 1a [tm1a/+]} or Pygl^{+/-}-C57BL/6N background) line was created with the Knockout Mouse Project (KOMP) Repository (#CSD23397) at the University of California, Davis, using the “knockout first (promoter driven)” strategy. To generate the knockout allele, a cassette containing flippase recognition target (FRT), locus of X(cross)-over in P1 (loxP) sequences, engrailed 2 splice acceptor (En2 SA), beta-galactosidase (lacZ), neomycin, and FRT and loxP sites was inserted in the intron between exon 2 and exon 3 of the Pygl gene. The allele of Pygl^{tm1a} was initially in a nonexpressive form with conditional potential. Embryonic stem cells containing the targeted allele (JMN8.4 subline, C57BL/6N background, KOMP repository, Pygl H02) were introduced into donor blastocysts (BALB/c). Chimerism of 50% or greater was identified by coat color, and chimeric male mice were bred with C57BL/6N female mice. Desired heterozygous mice from the bred cross (Pygl^{tm1a} allele) were genotyped to confirm germline transmission. For all experiments, heterozygous mating (Pygl^{+/-}) pairs were used to generate Pygl-deficient mice (Pygl^{-/-}). Human and mouse PYGL proteins share 94% of sequence similarity (the Ensembl genome browser www.ensem bl.org). FIG. 18 shows the generation of Pygl-deficient mice via a schematic representation of the Pygl-knockout and WT alleles. Insertion of the FRT/loxP cassette in the intron between exon 2-3 disrupts Pygl mRNA expression. Arrows indicate WT or lacZ primers used for genotyping.

1. An isolated nucleic acid molecule, comprising: a nucleic acid sequence encoding a polypeptide capable of preventing glycogen accumulation and/or degrading accumulated glycogen, wherein the encoded polypeptide comprises phosphorylase kinase regulatory subunit alpha 2 (PhK α2) or phosphorylase kinase catalytic subunit gamma 2 (PhK γ2).

2. The isolated nucleic acid molecule of claim 1, wherein the nucleic acid sequence is CpG depleted and codon-optimized for expression in a human cell.

3. The isolated nucleic acid molecule of claim 1, wherein the encoded polypeptide comprises at least 80% identity to the sequence set forth in any one of SEQ ID NO:04, SEQ ID NO:08, or SEQ ID NO:09.

4. The isolated nucleic acid molecule of claim 1, wherein the nucleic acid sequence comprises at least 80% identity to the sequence set forth in any one of SEQ ID NO:15, SEQ ID NO:16, SEQ ID NO:18, SEQ ID NO:19, or SEQ ID NO:20.

5.-8. (canceled)

9. An AAV vector, comprising: the isolated nucleic acid molecule of claim 1.

10. The AAV vector of claim 9, wherein the AAV vector comprises AAV1, AAV2, AAV3 (including 3a and 3b), AAV4, AAV5, AAV6, AAV7, AAV8, AAV.hum8, AAVrh8, AAV9, AAV10, AAVrh10, AAV11, AAV12, AAV13, AAVrh39, AAVrh43, AAVcy.7. AAV-DJ, AAV-HAE1, AAV-HAE2, AAVM41, AAV-1829, AAV2 Y/F, AAV2 T/V, AAV2i8, AAV2.5, AAV9.45, AAV9.61, AAV-B1, AAV-AS, AAV9.45A-String (e.g., AAV9.45-AS), AAV9.45Angiopep, AAV9.47-Angiopep, AAV9.47-AS, AAV-PHP.B, AAV-PHP.eB, AAV-PHP.S, AAV-F, AAVcc.47, or AAVcc.81.

11.-12. (canceled)

13. The AAV vector of claim 9, wherein the AAV vector comprises a liver-specific promoter operably linked to the isolated nucleic acid molecule.

14. The AAV vector of claim 11, wherein the liver-specific promoter comprises the thyroxin binding globulin (TBG) promoter, the α1-microglobulin/bikunin enhancer/thyroid hormone-binding globulin promoter, the human albumin (hALB) promoter, the thyroid hormone-binding globulin promoter, the α-1-anti-trypsin promoter, the bovine albumin (bAlb) promoter, the murine albumin (mAlb) promoter, the human α1-antitrypsin (hAAT) promoter, the ApoEhAAT promoter comprising the ApoE enhancer and the hAAT promoter, the transthyretin (TTR) promoter, the liver fatty acid binding protein promoter, the hepatitis B virus (HBV) promoter, the DC172 promoter comprising the hAAT promoter and the α1-microglobulin enhancer, the DC190 promoter comprising the human albumin promoter and the prothrombin enhancer, or any other natural or synthetic liver-specific promoter.

15. A pharmaceutical formulation comprising a theoretically effective amount of the AAV vector of claim 9 in a pharmaceutically acceptable carrier.

16. (canceled)

17. A method of preventing glycogen accumulation and/or degrading accumulated glycogen, the method comprising: administering to a human subject having GSD IX the pharmaceutical formulation of claim 15.

18. The method of claim 17, wherein the pharmaceutical formulation is administered intravenously.

19. (canceled)

20. The method of claim 17, further comprising administering to the subject a therapeutically effective amount of an agent for reducing the expression level and/or activity level of glycogen synthase (GYS), wherein the agent is a gene therapy, RNAi, microRNA, ASO, gene editing, or a small molecule.

21. The method of claim 20, wherein the GYS comprises GYS2.

22. The method of claim 17, further comprising administering one or more immune modulators, wherein the one or more immune modulators comprise methotrexate, rituximab, intravenous gamma globulin, SVP-Rapamycin, Tacrolimus, bortezomib, or a combination thereof.

23.-24. (canceled)

25. The method of claim 17, wherein the therapeutically effective amount of the AAV vector comprises about 1×10¹⁰ vg/kg to about 2×10¹⁴ vg/kg.

26. The method of claim 17, further comprising monitoring the subject for adverse effects.

27. The method of claim 26, wherein the presence of adverse effects, the method further comprises modifying an aspect of the method.

28. The method of claim 17, wherein one or more aspects of cellular homeostasis and/or cellular functionality are restored.

29. The method of claim 28, wherein restoration of one or more aspects of cellular homeostasis and/or cellular functionality comprises
(i) correction of cell starvation in one or more cell types;
(ii) normalization of aspects of the autophagy pathway;
(iii) improvement and/or restoration of mitochondrial functionality and/or structural integrity;
(iv) improvement and/or restoration of organelle functionality and/or structural integrity;
(v) prevention and/or slowing of hypoglycemia, ketosis, and/or other liver abnormalities;
(vi) correction of liver enzyme dysregulation;
(vii) prevention and/or slowing of the rate of progression of the multi-systemic manifestations of GSD IX;
(viii) prevention and/or slowing of the rate of progression of liver disease including fibrosis, cirrhosis, hepatic adenomas, and/or liver hepatocellular carcinoma,
(ix) restoration of the balance of glycogen metabolism, including glycogen synthesis and breakdown,
(x) restoration of PhK functionality and/or structural integrity, or
(xi) any combination thereof.

30. The method of claim 20, wherein the small molecule is guaiacol.
